(19) United States
(12) Patent Application Publication
(10) Pub. No.: US 2022/0202811 A1
(43) Pub. Date: Jun. 30, 2022

(54) TREATMENT FOR RETINOIC ACID RECEPTOR-RELATED ORPHAN RECEPTOR &#404; (ROR&#404;)-DEPENDENT CANCERS

(71) Applicant: The Regents of the University of California, Oakland, CA (US)

(72) Inventors: Tannishtha Reya, San Diego, CA (US); Nikki Lytle, La Jolla, CA (US)

(21) Appl. No.: 17/432,485

(22) PCT Filed: Feb. 20, 2020
(86) PCT No.: PCT/US20/19118

§ 371 Date: Aug. 19, 2021

Related U.S. Application Data
(60) Provisional application No. 62/808,231, filed on Feb. 20, 2019.
(60) Provisional application No. 62/881,890, filed on Aug. 1, 2019.
(60) Provisional application No. 62/897,202, filed on Sep. 6, 2019.
(60) Provisional application No. 62/903,595, filed on Sep. 20, 2019.
(60) Provisional application No. 62/959,607, filed on Jan. 10, 2020.

(51) Int. Cl. A61K 31/496; A61K 45/06; A61K 31/4155; A61K 31/4035; A61P 35/00

(52) U.S. Cl. CPC A61K 31/496; A61K 45/06; A61P 35/00; A61K 31/4035; A61K 31/4155

(57) ABSTRACT

Described are compositions and methods for the treatment of an RORγ-dependent cancer, including pancreatic cancer, lung cancer, leukemia, etc. In some example implementations, pharmaceutical compositions for cancer treatment comprising RORγ inhibitors and optionally other therapeutic agents, as well as methods of treating cancer using the pharmaceutical compositions are disclosed.

### CROSS-REFERENCE TO RELATED APPLICATIONS

This application claims the benefit of U.S. Provisional Patent Application No. 62/808,231 filed on Feb. 20, 2019, 62/881,890 filed on Aug. 1, 2019, 62/897,202 filed on Sep. 6, 2019, 62/903,595 filed on Sep. 20, 2019, and 62/959,607 filed on Jan. 10, 2020. The contents of these provisional applications are incorporated by reference in their entirety.

### STATEMENT OF GOVERNMENT INTEREST

This invention was made with government support under Grant Numbers R01 CA186043 and R01 CA197699, awarded by the National Institutes of Health. The government has certain rights in the invention.

### SEQUENCE LISTING

This application contains a Sequence Listing, which was submitted in ASCII format via USPTO EFS-Web, and is hereby incorporated by reference in its entirety. The ASCII copy, created on Feb. 20, 2020, is named Sequence-Listing_009062-8398WO_ST25 and is 13 kilobytes in size.

### TECHNICAL FIELD

This application relates to the treatment of various types of retinoic acid receptor-related orphan receptor gamma (RORγ)-dependent cancer.

### BACKGROUND

Many types of cancer are highly resistant to current treatments and thus remain a lethal disease. Development of more effective therapeutic strategies is critically dependent on identification of factors that contribute to tumor growth and maintenance. Some types of cancer share molecular dependency on cancer stem cells and have similar molecular signaling pathways. Therefore, new and effective therapeutic approaches for targeting common molecular signaling pathways lead to additional cancer therapies.

### SUMMARY

In one aspect, provided herein is a method of treating an RORγ-dependent cancer. The method entails administrating to a subject in need a therapeutically effective amount of a composition comprising one or more RORγ inhibitors. In certain embodiments, the subject suffers from a RORγ-dependent cancer such as pancreatic cancer, leukemia, and lung cancer including small cell lung cancer (SCLC) and nonsmall cell lung cancer (NSCLC). In certain embodiments, the subject suffers from a metastatic cancer. In certain embodiments, the RORγ inhibitor includes SR2211, JTE-151, JTE-151A, and AZD-0284, or an analog or derivative thereof represented by any one of formulae I, II, III, IIIA, and IV. In certain embodiments, the method further entails administering to the subject one or more chemotherapeutic agents. The composition comprising one or more RORγ inhibitors may be administered before or after administration of the one or more chemotherapeutic agents. Alternatively, the composition comprising one or more RORγ inhibitors and the one or more chemotherapeutic agents may be administered simultaneously. In certain embodiments, the method further entails administering to the subject one or more radiotherapies before, after, or during administration of the composition comprising one or more RORγ inhibitors.

In another aspect, disclosed herein is a pharmaceutical composition for treating a RORγ-dependent cancer. The pharmaceutical composition comprises a therapeutically effective amount of one or more RORγ inhibitors. In certain embodiments, the RORγ-dependent cancer includes pancreatic cancer, leukemia, and lung cancer including small cell lung cancer (SCLC) and nonsmall cell lung cancer (NSCLC). In certain embodiments, the cancer is a metastatic cancer. In certain embodiments, the RORγ inhibitor includes SR2211, JTE-151, JTE-151A, and AZD-0284, or an analog or derivative thereof represented by any one of formulae I, II, III, IIIA, and IV. In certain embodiments, the pharmaceutical composition further comprises a therapeutically effective amount of one or more chemotherapeutic agents. In certain embodiments, the pharmaceutical composition further comprises one or more pharmaceutically acceptable carriers, excipients, preservatives, diluent, buffer, or a combination thereof.

In yet another aspect, provided herein is a combinational therapy for a RORγ-dependent cancer. The combinational therapy comprises performing surgery, administering one or more chemotherapeutic agents, administering one or more radiotherapies, and/or administering one or more of immunotherapies to a subject in need thereof before, during, or after administering a composition comprising one or more RORγ inhibitors. In certain embodiments, the RORγ-dependent cancer includes pancreatic cancer, leukemia, and lung cancer including small cell lung cancer (SCLC) and nonsmall cell lung cancer (NSCLC). In certain embodiments, the cancer is a metastatic cancer. In certain embodiments, the RORγ inhibitor includes SR2211, JTE-151, JTE-151A, and AZD-0284, or an analog or derivative thereof represented by any one of formulae I, II, III, IIIA, and IV. In certain embodiments, the surgery, chemotherapy, radiotherapy, and/or immunotherapy is performed or administered to the subject before, during, after administering the composition comprising one or more RORγ inhibitor.

In yet another aspect, disclosed herein is a method of inhibiting cancer cell growth comprising contacting one or more cancer cells with an effective amount of one or more RORγ inhibitors in vivo, in vitro, or ex vivo. In certain embodiments, the RORγ-dependent cancer cell includes cells of pancreatic cancer, leukemia, and lung cancer including small cell lung cancer (SCLC) and nonsmall cell lung cancer (NSCLC). In certain embodiments, the cancer cell is a metastatic cancer cell. In certain embodiments, the RORγ inhibitor includes SR2211, JTE-151, JTE-151A, and AZD-0284, or an analog or derivative thereof represented by any one of formulae I, II, III, IIIA, and IV.

In yet another aspect, disclosed herein is a method of detecting a cancer, progression of cancer, or cancer metastasis in a subject comprising comparing the level of RORγ in a biological sample such as blood circulating tumor cells, a biopsy sample, or urine from the subject with the average level of RORγ of a population of healthy subjects, wherein an elevated level of RORγ indicates that the subject suffers from the cancer or cancer metastasis.

In yet another aspect, disclosed herein is a method of determining the prognosis of a subject receiving a cancer treatment comprising comparing the level of RORγ in a biological sample such as blood circulating tumor cells, a biopsy sample, or urine from the subject before and after receiving the cancer treatment, wherein a reduced level of RORγ indicates that the cancer treatment is effective for the subject.

### BRIEF DESCRIPTION OF THE DRAWINGS

This application contains at least one drawing executed in color. Copies of this application with color drawing(s) will be provided by the Office upon request and payment of the necessary fees.

FIGS. 1A-1P show that transcriptomic and epigenetic map of pancreatic cancer cells reveals a unique stem cell state. FIG. 1A: Schematic of overall strategy for RNA-seq and ChIP-seq of EpCAM+GFP+ (stem) and EpCAM+GFP− (non-stem) tumor cells from REM2-KP^{f/f}C mice (n=3 for RNA-seq, n=1 for ChIP-seq). FIG. 1B: Principal components analysis of KP^{f/f}C stem (purple) and non-stem (gray) cells. The variance contributed by PC1 and PC2 is 72.1% and 11.1% respectively. FIG. 1C: Transcripts enriched in stem cells (red, pink) and non-stem cells (dark blue, light blue). Pink, light blue, lfdr<0.3; red, dark blue, lfdr<0.1. FIGS. 1D-1K: Gene set enrichment analysis (GSEA) of stem and non-stem gene signatures. Cell states, and corresponding heat-maps of selected genes, associated with development and stem cells (FIGS. 1D and 1E), cell cycle (FIGS. 1F and 1G), metabolism (FIGS. 1H and 1I), and cancer relapse (FIGS. 1J and 1K). FIGS. 1D, 1F, 1H, and 1J: Red denotes overlapping gene signatures; blue denotes non-overlapping gene signatures. FIGS. 1E, 1G, 1I, and 1K: Red, over-represented gene expression; blue, under-represented gene expression; shades denote fold change from median values. FIGS. 1L and 1M: Hockey stick plots of H3K27ac occupancy, ranked by signal density. Super-enhancers in stem cells (FIG. 1L) or shared in stem and non-stem cells (FIG. 1M) are demarcated by highest ranking and intensity signals, above and to the right of dotted gray lines. Names of selected genes linked to super-enhancers are annotated. FIGS. 1N-1P: H3K27ac ChIP-seq read counts across selected genes marked by super-enhancers unique to stem cells (FIG. 1N), shared in stem and non-stem cells (FIG. 1O), or unique to non-stem cells (FIG. 1P).

FIGS. 2A-2F show that genome-scale CRISPR screen identifies core stem cell programs in pancreatic cancer. FIG. 2A: Schematic of CRISPR screen. Three independent primary KP^{f/f}C lines were generated from end-stage REM2-KP^{f/f}C tumors and transduced with lentiviral GeCKO V2 library (MOI 0.3). Cells were plated in standard 2D conditions under puromycin selection, then in 3D stem cell conditions. FIG. 2B: Number of guides detected in each replicate following lentiviral infection (gray bars), after puromycin selection in 2D (red bars), and after 3D sphere formation (blue bars). FIGS. 2C and 2D: Volcano plots of guides depleted in 2D (FIG. 2C) and 3D (FIG. 2D). Genes indicated on plots, p<0.005. FIG. 2E: Network propagation analysis integrating transcriptomic, epigenetic and functional analysis of stem cells. Genes enriched in stem cells by RNA-seq (stem/non-stem log₂ fold-change>2) and depleted in 3D stem cell growth conditions (FDR<0.5) were used to seed the network (triangles), then analyzed for known and predicted protein-protein interactions. Each node represents a single gene; node color is mapped to the RNA-seq fold change; stem cell enriched genes, red; non-stem cell enriched genes, blue; genes not significantly differentially expressed, gray. Labels are shown for genes which are enriched in stem cells by RNA-seq and ChIP-seq (Up/Up) or enriched in non-stem cells by RNA-seq and ChIP-seq (Down/Down); RNA log₂ fold change absolute value greater than 2.0, ChIP-seq FDR<0.01. Seven core programs were defined by groups of genes with high interconnectivity; each core program is annotated by Gene Ontology analysis (FDR<0.05). Essential genes within the core programs are listed in Table 1. FIG. 2F: Network propagation analysis from FIG. 2E restricted to genes enriched in stem cells by RNA-seq (stem/non-stem log₂ fold-change>2).

FIGS. 3A-3W show identification of novel pathway dependencies of pancreatic cancer stem cells. FIGS. 3A-3D: Functional impact of selected network genes on KP^{f/f}C cell growth in vitro and in vivo. Genes from stem and developmental processes (FIG. 3A, Onecut3, Tdrd3, Dusp9), lipid metabolism (FIG. 3B, Lpin, Sptssb), and cell adhesion, motility, and matrix components (FIGS. 3C and 3D, Myo10, Sftpd, Lama5, Pkp1, Myo5b) were inhibited via shRNA in KP^{f/f}C cells, and impact on tumor propagation assessed by stem cell sphere assays in vitro or by tracking flank transplants in vivo. Sphere formation, n=3-6 per conditions; flank tumor transplant, n=4 per condition. FIGS. 3E-3I: Identification of preferential dependence on MEGF family of adhesion proteins. FIG. 3E: Heat map of relative RNA expression of MEGF family and related (*Celsr1) genes in KP^{f/f}C stem and non-stem cells. Red, over-represented; blue, under-represented; color denotes fold change from median values. Impact of inhibiting Celsr1, Celsr2, and Pear1 in KP^{f/f}C cells in sphere forming assays in vitro (FIG. 3F) and flank transplants in vivo (FIGS. 3G-3I). Sphere formation, n=3-6 per condition; flank tumor transplant, n=4 per condition. FIGS. 3J-3K: Pear1 was inhibited via shRNA in KP^{f/f}C cells and impact on stem content (J, p=0.0629) and apoptosis (FIG. 3K) in sphere culture as marked by frequency of Msi2-GFP (FIG. 3J) or Annexin-V (K)-expressing cells was assessed by FACS, n=3 per condition. FIG. 3L: Pear1 was inhibited via shRNA delivery in human pancreatic cancer cells (FG cell line), and impact on tumor propagation assessed by stem cell sphere assays in vitro or by tracking flank transplants in vivo. Sphere formation, n=3; flank tumor transplant, n=4 per condition. FIG. 3M: Table summarizing identification of key new dependencies of pancreatic cancer growth and propagation. Checkmark indicates significant impact in the indicated assays following shRNA inhibition. FIG. 3N: Heat map of relative RNA expression of cytokines and related receptors in KP^{f/f}C stem and non-stem cells. Red, over-represented; blue, under-represented; color denotes fold change from median values. FIG. 30: Cell types mapped from single-cell sequencing of KP^{R172H/+}C tumors (left) and KP^{R172H/+}C tumor cells expressing IL10Rβ, IL34, and Csf1R. CAF, cancer-associated fibroblasts (red); EMT, mesenchymal tumor cells (yellow/green); Endo, endothelial cells (green); ETC, epithelial tumor cells (blue); TAM, tumor-associated macrophages (magenta). FIGS. 3P-3Q: KP^{R172H/+}C tumor single-cell sequencing map of cells expressing Msi2 within the EpCAM+ tumor cell fraction (FIG. 3P). KP^{R172H/+}C tumor single-cell sequencing map of cells expressing IL10Rβ (left), IL34 (middle), and Csf1R (right) within the EpCAM+Msi2+ stem cell fraction (FIG. 3Q). FIGS. 3R-3T: IL-10rβ and Csf1R were inhibited via shRNA delivery in KP^{f/f}C cells, and impact on tumor propagation assessed by stem cell sphere assays in vitro (FIG. 3R) or by tracking flank transplants in vivo (FIGS. 3S, 3T). Sphere formation, n=3-6 per condition; flank tumor transplant, n=4 per condition. FIG. 3U: IL-10 and IL-34 were inhibited via shRNA delivery in KP^{f/f}C cells, and impact on tumor propagation assessed by stem cell sphere assays in vitro, n=3 per shRNA. FIG. 3V: IL-10rβ and Csf1R were inhibited via shRNA delivery in KP^{f/f}C cells, and impact on stem content (Msi2-GFP+ cells) in sphere culture assessed by FACS, n=3 per condition. FIG. 3W: IL10Rβ was inhibited via shRNA delivery in human pancreatic cancer cells (FG cells), and impact on tumor propagation assessed by stem cell sphere assays in vitro or by tracking flank transplants in vivo. Sphere formation, n=3; flank tumor transplant, n=4 per condition. Data represented as mean+/−S.E.M. *p<0.05, **p<0.01, ***p<0.001 by Student's t-test or One-way ANOVA.

FIGS. 4A-4R show that the immuno-regulatory gene RORγ is a critical dependency of pancreatic cancer propagation. FIG. 4A: qPCR analysis of RORγ expression in stem and non-stem tumor cells isolated from primary KP^{f/f}C tumors. Tumors 1, 2, and 3 represent biological replicates from REM2-KP^{f/f}C mice. FIG. 4B: KP^{f/f}C tumor single-cell sequencing map of cells expressing RORγ within the EpCAM+Msi2+ cell fraction (n=3 mice represented). FIG. 4C: Representative image of RORγ expression in KP^{R172H/+}C tumor sections. RORγ (green), Keratin (red). FIG. 4D: Representative images of RORγ expression in normal adjacent human pancreas (left), PanINs (middle), and PDAC (right). RORγ (green), E-Cadherin (red), Dapi (blue). FIGS. 4E and 4F: Quantification of RORγ expression in patient samples by immunofluorescence analysis. Primary patient tumors were stained for RORγ and E-cadherin and frequency of RORγ+ cells within the tumor (FIG. 4E) and the E-Cadherin+ epithelial cell fraction (FIG. 4F) were determined. Normal adjacent, n=3; pancreatitis, n=8; PanIN 1, n=10; PanIN 2, n=6; PDAC, n=8. FIGS. 4G-4H: RORγ was inhibited via shRNA delivery in KP^{R172H/+}C (FIG. 4G) and KP^{f/f}C (FIG. 4H) cells, and impact on colony or sphere forming capacity was assessed, n=3 per shRNA. FIGS. 4I-4K: RORγ was inhibited via shRNA delivery in KP^{f/f}C cells and impact on Msi2-GFP stem content (FIG. 4I), BrdU (FIG. 4J), and Annexin-V (FIG. 4K) in sphere culture assessed by FACS n=3 per condition. FIG. 4L: RORγ was inhibited via shRNA delivery in KP^{f/f}C cells, and impact on tumor propagation assessed by tracking flank transplants in vivo, n=4 per condition. FIGS. 4M and 4N: Heat maps of relative RNA expression of stem cell programs (FIG. 4M) and pro-tumor factors (FIG. 4N) in KP^{f/f}C cells transduced with shCtrl or shRorc. Red, over-represented; blue, under-represented; color denotes fold change from median values. FIG. 4O: Venn diagram of genes downregulated with loss of RORγ (q-value<0.05, purple), super-enhancer-associated genes specific to stem cells (green), and genes associated with open chromatin regions containing RORγ consensus binding sites (orange). FIG. 4P: Distribution of RORγ consensus binding sites across the genome. Left, percent of genome associated with super-enhancers specific to stem cells; right, frequency of RORγ consensus binding sites in stem cell-associated super-enhancers. FIG. 4Q: Heat map of relative RNA expression of super-enhancer-associated oncogenes in KP^{f/f}C cells transduced with shCtrl or shRorc. Red, over-represented; blue, under-represented; color denotes fold change from median values. FIG. 4R: H3K27ac ChIP-seq read counts for genes marked by super-enhancers in stem cells that are downregulated in RORγ-depleted KP^{f/f}C cells. Data represented as mean+/−S.E.M. *p<0.05, **p<0.01, ***p<0.001 by Student's t-test or One-way ANOVA.

FIGS. 5A-5X show that pharmacologic targeting of RORγ impairs progression and improves survival in mouse models of pancreatic cancer. FIGS. 5A and 5B: Sphere forming capacity of KP^{f/f}C cells (FIG. 5A) and colony forming assay of KP^{R172H/+}C cells (FIG. 5B) in the presence of the RORγ inverse agonist SR2211 or vehicle (n=3 per condition). FIGS. 5C and 5D: Organoid forming capacity of low-passage KP^{f/f}C tumor cells grown in the presence of SR2211 or vehicle. Representative organoid images (FIG. 5C) and quantification of organoid formation (FIG. 5D). FIGS. 5E-5I: Analysis of flank KP^{f/f}C tumor-bearing mice treated with SR2211 or vehicle for 3 weeks. (FIG. 5E) Schematic of tumor establishment and therapeutic approach. Total live cells (FIG. 5F), total EpCAM+ tumor epithelial cells (FIG. 5G), total EpCAM+/CD133+ stem cells (FIG. 5H), and total EpCAM+/Msi2+ stem cells (FIG. 5I) (n=4 for vehicle, n=2 for vehicle+gemcitabine, n=4 for SR2211, n=3 for SR2211+gemcitabine). FIG. 5J: Survival of KP^{f/f}C mice treated daily with vehicle (gray) or SR2211 (black). Tumor-bearing mice were enrolled into treatment at 8 weeks of age and continuously treated until moribund (p=0.051, Hazard ratio=0.16, Median survival: vehicle=18 days, SR2211=38.5 days). FIG. 5K: Live imaging of REM2-KP^{f/f}C mice with established tumors treated with vehicle or SR2211 for 8 days (n=2 per condition). Msi2-reporter (green), VE-Cadherin (magenta), Hoecsht (blue); Msi2-reporter+ stem cells, gray box. FIG. 5L: Quantification of stem cell clusters from REM2-KP^{f/f}C live imaging (n=2 per condition; 6-10 frames analyzed per mouse). FIG. 5M-5N: Analysis of flank KP^{f/f}C tumor-bearing NSG mice treated with SR2211 or vehicle for 2 weeks. Schematic of tumor establishment and therapeutic approach: KP^{f/f}C tumor cells were transplanted into flanks of NSG mice (which lack Th17 cells) prior to treatment (FIG. 5M). Tumor growth rate of flank tumors following treatment with either vehicle or SR2211 for 2 weeks (FIG. 5N). Fold change of tumor volume is relative to volume at the start of treatment. (n=4-6 per treatment group). FIGS. 5O-5P: Analysis of KP^{f/f}C flank tumor growth in WT or RORγ-knockout recipient mice; RORγ-knockout recipients are depleted for T cell populations in the microenvironment. Schematic of tumor establishment (FIG. 5O). Tumor growth rate of flank tumors in WT or RORγ knockout recipient mice (FIG. 5P) (n=3-4 per condition). FIGS. 5Q-5X: Analysis of WT or RORγ-knockout recipient mice bearing transplanted KP^{f/f}C tumors and treated with SR2211 or vehicle for 2 weeks. Schematic of tumor establishment and experimental strategy (FIG. 5Q). Tumor growth rate of flank tumors in WT recipient mice treated with either vehicle or SR2211 for 2 weeks (FIG. 5R). Tumor growth rate of flank tumors in RORγ-knockout recipient mice treated with either vehicle or SR2211 for 2 weeks (FIG. 5S). Final tumor mass (FIG. 5T), total live cells (FIG. 5U), total EpCAM+ tumor epithelial cells (FIG. 5V), total EpCAM+/CD133+ stem cells (FIG. 5W), and total Th17 cells (FIG. 5X) in WT and RORγ-knockout recipient mice (n=5-7 per condition). Data represented as mean+/−S.E.M. *p<0.05, **p<0.01, ***p<0.001 by Student's t-test or One-way ANOVA.

FIGS. 6A-6K show function of RORγ in human pancreatic cancer. FIG. 6A: Colony forming capacity of human pancreatic cancer cell line following knockdown of RORC using 5 independent CRISPR guides. FIG. 6B: Representative images of human pancreatic cancer line flank tumors RORγ (green), E-Cadherin (red), Dapi (blue). FIG. 6C: Growth rate of tumors derived from human pancreatic cancer lines in mice treated with gemcitabine and either vehicle or SR2211 for 2.5 weeks. Fold change of tumor volume is relative to volume at the start of treatment. FIGS. 6D and 6E: Primary patient organoid growth in the presence of vehicle or SR2211. Representative images of organoids following recovery from Matrigel (FIG. 6D) and quantification of organoid circumference (FIG. 6E, left) or organoid volume (FIG. 6E, right). FIG. 6F: Growth rate of primary patient-derived tumors in xenografts treated with vehicle or SR2211 for 1.5 weeks (n=4). FIG. 6G: RORC amplification in tumors of patients diagnosed with various malignancies. FIGS. 6H-6K: Analysis of RORγ staining in patient tissue microarrays. IHC staining of RORγ in patient tissue microarrays of PDAC and matched PanINs illustrating TMA scoring for negative, cytoplasmic, and cytoplasmic+nuclear RORγ staining (FIG. 6H). Correlation between RORγ staining and tumor stage (FIG. 6I), lymphatic invasion (FIG. 6J), and lymph node status (FIG. 6K). Data represented as mean+/−S.E.M. *p<0.05, **p<0.01, ***p<0.001 by Student's t-test or One-way ANOVA.

FIGS. 7A-7C show that Musashi2+ tumor cells are enriched for organoid-forming capacity, related to FIG. 1. FIG. 7A: Tumor organoid formation from primary isolated Musashi2+ (REM2+) and Musashi2− (REM2−) KP^{f/f}C tumor cells. Number of cells plated is indicated above representative images. FIG. 7B: Limiting dilution frequency (left) calculated for REM2+ (black) an REM2− (red) organoid formation. Table (right) indicates cell doses tested in biological replicates. FIG. 7C: Frequency of proliferating (Ki67+) REM2+ (left) and REM2− (right) tumor cells in untreated 10-12 week old REM2-KP^{f/f}C mice (n=3), or treated with gemcitabine for 72 hours (n=1) or 6 days (n=1) prior to analysis; 200 mg/kg gemcitabine i.p. was delivered every 72 hours.

FIGS. 8A-8E show that H3K27ac-marked regions are congruent with RNA expression in primary stem and non-stem KP^{f/f}C cells, related to FIGS. 1A-1P. FIG. 8A: Overlap of H3K27ac peaks and genomic features. For each genomic feature, frequency of H3K27ac peaks in stem cells (blue) and non-stem cells (gray) are represented as ratio of observed peak distribution/expected random genomic distribution. FIGS. 8B and 8C: Concordance of H3K27ac peaks with RNA expression in stem cells (FIG. 8B; p=7.1×10−14) and non-stem cells (FIG. 8C; p<22×10−16). FIGS. 8D and 8E: Ratio of observed/expected overlap in gene expression and H3K27ac enrichment comparing stem and non-stem cells. Down/Up, gene expression enriched in non-stem/H3K27ac enriched in stem; Up/Down, gene expression enriched in stem/H3K27ac enriched in non-stem; Down/Down, both gene expression and H3K27ac enriched in non-stem; Up/Up, both gene expression and H3K27ac enriched in stem.

FIGS. 9A-9C show enriched sgRNA in standard and stem cell growth conditions, related to FIGS. 2A-2F. FIG. 9A: Establishment of three independent REM2-KP^{f/f}C cell lines from end-stage REM2-KP^{f/f}C mice for genome-wide CRISPR-screen analysis. Stem cell content of freshly-dissociated REM2-KP^{f/f}C tumors (FIG. 9A, left), and after puromycin selection in standard growth conditions (FIG. 9A, right). FIGS. 9B and 9C: Volcano plots of guides enriched in 2D (FIG. 9B, tumor suppressors) and 3D (FIG. 9C, negative regulators of stem cells). Genes indicated on plots, p<0.005.

FIGS. 10A-10C show identification of novel regulators of pancreatic cancer stem cells, related to FIGS. 3A-3W. FIGS. 10A and 10B: Sphere forming capacity of KP^{f/f}C cells following shRNA knockdown. Selected genes involved in stem and developmental processes (FIG. 10A) or cell adhesion, cell motility, and matrix components (FIG. 10B). Data represented as mean+/−S.E.M. *p<0.05, **p<0.01, by Student's t-test or One-way ANOVA. FIG. 10C: Single cell RNA expression maps from KP^{R172H/+}C tumors. Tumor cells defined by expression of EpCAM (far left), Krt19 (left center), Cdh1 (right center), and Cdh2 (far right).

FIGS. 11A-11C show protein validation of stem cell enriched genes identified by RNA Seq, related to FIGS. 3A-3W and 4A-4R. Immunofluorescence analysis of Celsr1 (FIG. 11A), Celsr2 (FIG. 11B), and RORγ (FIG. 11C) in EpCAM+ stem (CD133+) and non-stem (CD133−) primary tumor cells isolated from KP^{f/f}C mice. Three frames were analyzed per slide, and the frequency of Celsr1-high, Celsr2-high, or RORγ-high cells determined. Data represented as mean+/−S.E.M. *p<0.05, **p<0.01 by Student's t-test or One-way ANOVA.

FIGS. 12A and 12B show Westerns confirming protein knockdown of target genes, related to FIGS. 3A-3W and 4A-4R. KP^{f/f}C cells were infected with shRNA against Pear1 (FIG. 12A) or RORγ (FIG. 12B) and protein knockdown efficiency was determined five days post-transduction by western blot. Relative expression is quantified relative to tubulin loading control.

FIGS. 13A-13F show independent replicates of in vivo experiments validating dropouts identified in genome wide CRISPR Screen, related to FIGS. 3A-3W and 4A-4R. Celsr1 (FIG. 13A), Celsr2 (FIG. 13B), Pear1 (FIG. 13C), IL10Rb (FIG. 13D), CSF1R (FIG. 13E), and RORγ (FIG. 13F) were inhibited via shRNA delivery in KP^{f/f}C cells, and impact on tumor propagation was assessed by tracking flank transplants in vivo, n=4 per condition. Data represented as mean+/−S.E.M. *p<0.05, **p<0.01, ***p<0.001 by Student's t-test or One-way ANOVA.

FIG. 14 shows the impact of cytokine receptor inhibition on apoptosis in KP^{f/f}C cells, related to FIGS. 3A-3W. Cytokine receptors IL10Rb and CSF1R were inhibited by shRNA delivery in KP^{f/f}C cells and plated in sphere culture for one week. Increased apoptosis of KP^{f/f}C cells was seen with shIL10Rb (p<0.05) and shCSF1R (trend). Frequency of apoptotic cells determined by Annexin-V staining and FACS analysis, n=3 per condition. Data represented as mean+/−S.E.M. *p<0.05, **p<0.01, ***p<0.001 by Student's t-test and One-way ANOVA.

FIGS. 15A-15C show cytokine expression in KP^{f/f}C cells and media in vitro, related to FIGS. 3A-3W. Concentration of cytokines IL-10, IL-34, and CSF-1 in media and KP^{f/f}C cells were quantified by ELISA (Quantikine, R&D Systems), Standard curves used for quantitation (FIG. 15A). Cytokines were quantified in fresh sphere culture media, KP^{f/f}C stem and non-stem cell conditioned media (FIG. 15B), and KP^{f/f}C epithelial cell lysate (FIG. 15C). Conditioned media was generated by culturing sorted CD133− or CD133+ KP^{f/f}C cells in sphere media for 48 hours; media was filtered and assayed immediately. Cell lysate was collected in RIPA buffer and assayed at 2 mg/mL for ELISA. n=3 per condition.

FIGS. 16A-16C show epithelial-specific programs downstream of RORγ related to FIGS. 4A-4R. FIG. 16A: Heat map of relative RNA expression in KP^{f/f}C stem and non-stem cells of transcription factors identified as possible pancreatic cancer stem cell dependencies within the network map (see FIG. 2E). Red, over-represented; blue, under-represented; color denotes fold change from median values. FIG. 16B: Analysis of RORγ consensus binding site distribution in genomic regions associated with H3K27ac. Down/Down, both gene expression and H3K27ac enriched in non-stem cells; Up/Up, both gene expression and H3K27ac enriched in stem cells. FIG. 16C: Quantification of RORγ expression within E-Cadherin− stromal cells of patient samples. Data represented as mean+/−S.E.M. *p<0.05, **p<0.01, ***p<0.001 by Student's t-test or One-way ANOVA.

FIG. 17 shows regulation of RORγ expression by IL-1R1, related to FIGS. 4A-4R. IL1 R1 was inhibited by CRISPR-mediated deletion in KP^{f/f}C cells, and impact on RORγ expression assessed by qPCR. Two distinct guide RNAs (sgIL1r1-1 and sgIL1r1-2) were used to knockout IL1 R1; expression was quantified by qPCR and is shown relative to control (non-targeting guide RNA), n=3 per condition. Data represented as mean+/−S.E.M. *p<0.05, **p<0.01, ***p<0.001 by Student's t-test or One-way ANOVA.

FIGS. 18A-18C show the impact of RORγ knockdown on stem cell super-enhancer landscape, related to FIGS. 4A-4R. KP^{f/f}C cell lines were infected with shRorc and used for H3K27ac ChIP-seq and super-enhancer analysis, schematic (FIG. 18A). H3K27ac peaks were analyzed to assess SE overlap in shCtrl and shRorc samples (FIG. 18B). Super-enhancers lost in shRorc samples were crossed to stem-enriched and stem-unique super-enhancers identified in primary Msi2-GFP+ KP^{f/f}C tumors cells, and further restricted to SEs containing RORγ binding motifs (FIG. 18C). Majority of super-enhancer landscape remained unchanged with RORγ loss, and landscape changes that did occur were not enriched in SEs with RORγ binding sites. ChIP-seq analysis was conducted in two independent KP^{f/f}C cell lines.

FIGS. 19A-19C show pharmacologic targeting of RORγ related to FIGS. 5A-5X and 6A-6K. FIG. 19A: Size of flank KP^{f/f}C tumors in immunocompetent mice prior to enrollment into RORγ targeted therapy. Group 1, vehicle; group 2, SR2211; group 3, vehicle+gemcitabine; group 4, SR2211+gemcitabine. FIG. 19B: Representative images of primary patient organoids grown in the presence of vehicle (left) or SR2211 (right). FIG. 19C: Analysis of CRISPR guide depletion in stem cell conditions for super-enhancer-associated genes expressed in stem or non-stem cells. Data represented as mean+/−S.E.M. *p<0.05, **p<0.01, ***p<0.001 by Student's t-test or One-way ANOVA.

FIGS. 20A-20D show target engagement following RORγ inhibition in vivo, related to FIGS. 5A-5X. FIGS. 20A and 20B: Tumor-bearing KP^{f/f}C mice 9.5 weeks of age were treated with vehicle or SR2211 for two weeks (midpoint), after which tumors were isolated, fixed, and analyzed for target engagement of Hmga2 in epithelial cells by immunofluorescence. Quantification of Hmga2-positive epithelial cells in vehicle or SR2211 treated tumors (FIG. 20A) representative images (FIG. 20B). FIGS. 20C and 20D: Tumor-bearing KP^{f/f}C mice were treated from 8 weeks of age to endpoint with either vehicle or SR2211. Quantification of Hmga2-positive epithelial cells in vehicle or SR2211 treated tumors (FIG. 20C), representative images (FIG. 20D). Four frames were analyzed per mouse, n=2-4 mice per condition, Hmga2 (red), Keratin (green). Data represented as mean+/−S.E.M. *p<0.05, **p<0.01, ***p<0.001 by Student's t-test or One-way ANOVA. Grubb's test (p=0.1), was used to remove an outlier from the midpoint SR2211 treated group.

FIGS. 21A-21D show that T cell subsets are depleted in KP^{f/f}C tumors transplanted into RORγ-knockout recipient mice, related to FIGS. 5A-5X. Analysis of T cell subsets in KP^{f/f}C tumors transplanted into wild-type or RORγ-knockout recipient mice (control treated groups shown). Frequencies and absolute cell numbers of the following populations were evaluated: CD45+ cells (FIG. 21A), CD45+/CD3+ T cells (FIG. 21B), CD45+/CD3+/CD8+ or CD4+ T cells (FIG. 21C), CD45+/CD3+/CD4+/IL-17+Th17 cells (FIG. 21D); frequencies are calculated as total frequency in the tumor (n=5-7 per condition). Data represented as mean+/−S.E.M. *p<0.05, **p<0.01, ***p<0.001 by Student's t-test or One-way ANOVA.

FIGS. 22A-22J show the impact of SR2211 on vasculature and non-neoplastic cells in KP^{f/f}C mice related to FIGS. 5A-5X. FIGS. 22A-22I: FACS analysis of non-neoplastic cell populations in autochthonous tumors from KP^{f/f}C mice treated with vehicle or SR2211 for 1 week. Frequencies and absolute cell numbers of the following populations were evaluated: CD45+ cells (FIG. 22A), CD31+ cells (endothelial) (FIG. 22B), CD11b/F480+ cells (macrophage) (FIG. 22C), CD11b/Gr-1+ cells (MDSC) (FIG. 22D), CD11c+ cells (dendritic) (FIG. 22E), CD45+/CD3+ T cells (FIG. 22F), CD3+/CD8+ T cells (FIG. 22G), CD3+/CD4+ T cells (FIG. 22H), CD4+/IL-17+Th17 cells (FIG. 22I). (n=3 per condition). FIG. 22J: In vivo imaging of the vasculature of KP^{f/f}C mice treated with vehicle or SR2211, vasculature is marked by in vivo delivery of anti-VE-Cadherin. Data represented as mean+/−S.E.M. *p<0.05 by Student's t-test or One-way ANOVA.

FIGS. 23A-23D show the analysis of downstream targets of RORγ in murine and human pancreatic cancer cells identifies shared pro-tumorigenic cytokine pathways related to FIGS. 4A-4R and 6A-6K. Gene ontology and gene set enrichment analysis of RNA-seq in human and mouse pancreatic cancer cells to identify common genes and pathways regulated by RORγ. Gene ontology analysis of KP^{f/f}C RNA-seq showing genes downregulated with shRorc were enriched for cytokine-mediated signaling pathway GO term (FIG. 23A). Specific differentially expressed genes in KP^{f/f}C within cytokine-mediated signaling pathway (FIG. 23B) were crossed with differentially expressed genes identified by RNA-seq analysis of human pancreatic cancer cells (FG) where RORγ was knocked out using CRISPR. Gene set enrichment analysis of mouse and human RNA-seq shows common cytokine gene sets regulated by RORγ across species (FIG. 23D).

FIGS. 24A-24G show the efficiency of RNA knockdown for all functionally tested genes, related to FIGS. 3A-3W and 4A-4R. FIGS. 24A-24F: KP^{f/f}C cells were infected with shRNA against the indicated genes and knockdown efficiency was determined. Developmental processes (Onecut3, Tdrd3, Dusp9, En1, Car2, Ano1) (FIG. 24A), metabolism (Sptssb, Lpin2) (FIG. 24B), cell adhesion, cell motility, matrix components (Myo10, Sftpd, Pkp1, Lama5, Myo5b, Muc4, Elmo3, Tff1, Muc1, Ctgf) (FIG. 24C), MEGF family (Megf10, Celsr1, Celsr2, Pear1) (FIG. 24D), cytokine receptors, immune signals (Csf1R, IL10Rb, IL10, IL34) (FIG. 24E), RORγ (FIG. 24F). n=3 per condition. FIG. 24G: Human FG cells were infected with shRNA against IL10Rb or Pearl, and knockdown efficiency was determined. n=3 per condition. Data represented as mean+/−S.E.M. *p<0.05, **p<0.01, ***p<0.001, ****p<0.0001 by Student's t-test or One-way ANOVA.

FIGS. 25A and 25B show that overexpression of Msi2 partially rescues sphere-formation of shRorc KP^{f/f}C tumor cells. FIG. 25A: KP^{f/f}C cell lines were transduced with lentiviral shRorc or shCtrl and either control over-expression or Msi2 over-expression vector. Double-infected cells were sorted (on green and red) and plated in sphere culture for one week. FIG. 25B: qPCR analysis showing Msi2 overexpression in shRorc and shCtrl infected cells and knockdown of Msi2 in shRorc control cells.

FIGS. 26A and 26B show no difference in phagocytosis of SR2211 treated KP^{f/f}C cells. KP^{f/f}C cell lines were transduced with lentiviral GFP over-expression vector and transplanted into the flank of immunocompetent littermates. After establishment, tumors were treated with SR2211 or vehicle; tumors were then analyzed by FACS for GFP-expressing macrophages as a measure of phagocytosis (n=2-4 per condition).

FIG. 27 shows TPM values for cytokine receptors and signals, related to FIGS. 3A-3W. Average RNA-Seq TPM values are shown for cytokine and immune signals in Msi2− and Msi2+ cells.

FIG. 28 shows the analysis of RORc-null KP^{f/f}C mouse. Tumor mass and cell count for wild type, RORC^{+/-} and RORC^{-/-} KP^{f/f}C mice, n=1 per condition.

FIG. 29 shows that RORc deletion impairs bcCML growth.

FIG. 30 shows that AZD-0284 treatment in combination with gemcitabine inhibited KP^{f/f}C organoid growth.

FIG. 31 shows that AZD-0284 treatment at higher dose, either alone or in combination with gemcitabine, inhibited KP^{f/f}C organoid growth.

FIG. 32 shows dose-dependent effects of AZD-0284, either alone or in combination with gemcitabine, at inhibiting KP^{f/f}C organoid growth.

FIG. 33 shows results of experiments testing the impact of AZD-0284 in vivo on tumor-bearing KP^{f/f}C mice using different tumor parameters.

FIG. 34 shows results of experiments testing the impact of AZD-0284 in vivo on tumor-bearing KP^{f/f}C mice using different tumor parameters.

FIG. 35 shows significant inhibition of primary patient-derived PDX1535 organoid growth by a combination of AZD-0284 and gemcitabine.

FIG. 36 shows that AZD-0284 treatment at higher dose, either alone or in combination with gemcitabine, inhibited primary patient-derived PDX1535 organoid growth.

FIG. 37 shows dose-dependent effects of AZD-0284, either alone or in combination with gemcitabine, at inhibiting primary patient-derived PDX1535 organoid growth.

FIG. 38 shows that AZD-0284 at lower dose, either alone or in combination with gemcitabine, effectively inhibited primary patient-derived PDX1356 organoid growth.

FIG. 39 shows that AZD-0284 at higher dose, either alone or in combination with gemcitabine, effectively inhibited primary patient-derived PDX1356 organoid growth.

FIG. 40 is a compilation of data showing the inhibitory effect of AZD-0284 at different dosage on primary patient-derived organoid growth.

FIG. 41 shows results of experiments testing the impact of AZD-0284 in vivo on primary patient-derived xenografts using different tumor parameters.

FIG. 42 shows results of experiments testing the impact of AZD-0284 in vivo on primary patient-derived xenografts using different tumor parameters.

FIG. 43 shows results of experiments testing the impact of AZD-0284 in vivo on primary patient-derived xenografts using different tumor parameters.

FIG. 44 shows compilations of data showing the anti-cancer effect of AZD-0284 in vivo on primary patient-derived xenografts.

FIG. 45 shows compilations of data showing the anti-cancer effect of AZD-0284 in vivo on primary patient-derived xenografts.

FIG. 46 shows effects of different doses of AZD-0284 at inhibiting colony formation of human leukemia k562 cells.

FIG. 47 is a schematic of organoid studies using pancreatic cancer cells derived from a non-germline genetically engineered mouse model (GEMM).

FIG. 48 is a schematic of organoid studies using pancreatic cancer cells derived from a germ line genetically engineered mouse model (GEMM).

FIG. 49 shows that JTE-151 treatment inhibited non-germline KRAS/p53 organoid growth.

FIG. 50 shows that JTE-151 treatment inhibited germline KP^{f/f}C organoid growth.

FIG. 51 is a schematic of in vivo studies of JTE-151 treatment of tumors using tumor-bearing KP^{f/f}C mice or primary pancreatic cancer patient-derived xenografts.

FIG. 52 is a compilation of data from tumor-bearing KP^{f/f}C mice treated with 30 mg/kg JTE-151.

FIG. 53 shows results of individual experiments where tumor-bearing KP^{f/f}C mice were treated with 90 mg/kg JTE-151.

FIG. 54 shows results of individual experiments where tumor-bearing KP^{f/f}C mice were treated with 90 mg/kg JTE-151.

FIG. 55 shows results of individual experiments where tumor-bearing KP^{f/f}C mice were treated with 90 mg/kg JTE-151.

FIG. 56 shows results of individual experiments where tumor-bearing KP^{f/f}C mice were treated with 90 mg/kg JTE-151.

FIG. 57 is a compilation of data from tumor-bearing KP^{f/f}C mice treated with 90 mg/kg JTE-151.

FIG. 58 is a compilation of data from tumor-bearing KP^{f/f}C mice treated with 30 mg/kg or 90 mg/kg JTE-151.

FIG. 59 shows results of individual experiments where tumor-bearing KP^{f/f}C mice were treated with 120 mg/kg JTE-151.

FIG. 60 shows results of individual experiments where tumor-bearing KP^{f/f}C mice were treated with 120 mg/kg JTE-151.

FIG. 61 shows results of individual experiments where tumor-bearing KP^{f/f}C mice were treated with 120 mg/kg JTE-151.

FIG. 62 is a schematic of organoid studies using pancreatic cancer cells derived from a mouse model bearing patient-derived xenograft tumor.

FIG. 63 shows that JTE-151 treatment, either alone or in combination with gemcitabine, inhibited primary patient-derived PDX1535 organoid growth.

FIG. 64 shows dose-dependent effects of JTE-151, either alone or in combination with gemcitabine, at inhibiting primary patient-derived PDX1535 organoid growth.

FIG. 65 shows that JTE-151 treatment, either alone or in combination with gemcitabine, inhibited primary patient-derived PDX1356 organoid growth.

FIG. 66 shows that JTE-151 treatment at a higher dose, either alone or in combination with gemcitabine, inhibited primary patient-derived PDX1356 organoid growth.

FIG. 67 shows that JTE-151 treatment alone or in combination with gemcitabine inhibited primary patient-derived PDX202 and PDX204 organoid growth.

FIG. 68 is a compilation of data from primary patient-derived organoids treated with JTE-151 at different doses.

FIG. 69 is a compilation of data from human Fasting Growing (FG) organoids treated with JTE-151 at different doses, either alone or in combination with gemcitabine.

FIG. 70 shows the anti-cancer effect of JTE-151 in vivo on primary patient-derived PDX1356 xenografts.

FIG. 71 shows the anti-cancer effect of JTE-151 in vivo on primary patient-derived PDX1356 xenografts.

FIG. 72 shows the anti-cancer effect of JTE-151 in vivo on primary patient-derived PDX1356 xenografts.

FIG. 73 shows the anti-cancer effect of JTE-151 in vivo on primary patient-derived PDX1356 xenografts.

FIG. 74 shows the anti-cancer effect of JTE-151 in vivo on primary patient-derived PDX1535 xenografts.

FIG. 75 shows the anti-cancer effect of JTE-151 in vivo on primary patient-derived PDX1535 xenografts.

FIG. 76 shows the anti-cancer effect of JTE-151 in vivo on primary patient-derived PDX1424 xenografts.

FIG. 77 shows the anti-cancer effect of JTE-151 in vivo on primary patient-derived PDX1424 xenografts.

FIG. 78 is a compilation of data from mice bearing primary patient-derived xenografts treated with JTE-151.

FIG. 79 shows that Msi2-Cre^{ER}/LSL-Myc mice develop different types of pancreatic cancer following induction of Myc.

FIG. 80 shows that RORγ is expressed in adenosquamous and acinar carcinoma. RORγ: red; keratin: green; DAPI: blue.

FIG. 81 shows that pancreatic adenosquamous carcinoma is sensitive to SR2211.

FIGS. 82A-82B show that acinar tumor-derived organoids are sensitive to RORγ inhibitors.

FIG. 83 shows dosage-dependent effects of SR2211 at inhibiting LcCA KP lung cancer cell growth.

### DETAILED DESCRIPTION

Disclosed herein in various embodiments are techniques of identifying a cancer target common for several types of cancer, such as RORγ, therapeutic uses, diagnostic uses, and prognostic uses of the small molecule compounds inhibiting the cancer target, combinational therapy using the RORγ inhibitors in combination with one or more other cancer therapies, as well as pharmaceutical compositions comprising the RORγ inhibitors.

### Identification of Cancer Target

Drug resistance and resultant relapse remain key challenges in pancreatic cancer and are in part driven by the inherent heterogeneity of the tumor that prevents effective targeting of all malignant cells. To better understand the pathways that confer an aggressive phenotype and drug resistance, a combination of RNA-seq, ChIP-seq and genome-wide CRISPR screening was utilized to systematically map molecular dependencies of pancreatic cancer stem cells, which are highly drug resistant cells that are also enriched in the capacity to drive tumor progression. Integration of these data revealed an unexpected role for immuno-regulatory pathways in stem cell self-renewal and maintenance in autochthonous tumors. In particular, RORγ, a nuclear hormone receptor known for its role in inflammatory cytokine responses and T cell differentiation, emerged as a key regulator of stem cells. RORγ transcriptional levels increased during pancreatic cancer progression, and the locus was amplified in a subset of pancreatic cancer patients. Functionally RORγ inhibition, whether achieved via genetic or pharmacologic approaches, led to a striking defect in pancreatic cancer growth in vitro and in vivo, and improved survival in genetically engineered models. Finally, a large-scale retrospective analysis of patient samples revealed that RORγ expression in PanIn lesions was positively correlated with advanced disease, lymphatic vessel invasion and lymph node metastasis, suggesting that RORγ expression could be a useful marker to predict pancreatic cancer aggressiveness. Collectively, these data reveal an unexpected co-option of immuno-regulatory signals by pancreatic cancer stem cells and suggest that therapeutics currently being used for autoimmune indications should be evaluated as a novel treatment strategy for pancreatic cancer patients.

While cytotoxic agents remain the standard of care for most cancers, their use is often associated with initial efficacy, followed by disease progression. This is particularly true for pancreatic cancer, a highly aggressive disease, where current multidrug chemotherapy regimens result in tumor regression in 30% of patients, quickly followed by disease progression in the vast majority of cases. This progression is largely due to the inability of chemotherapy to successfully eradicate all tumor cells, leaving behind subpopulations that can trigger tumor re-growth. Thus, identifying the cells that are preferentially drug resistant, and understanding their vulnerabilities, is critical to improving patient outcome and response to current therapies.

Previous work has focused on identifying the most tumorigenic populations within pancreatic cancer. Through this, subpopulations of cells marked by expression of CD24+/CD44+/ESA+, cMet, CD133, Nestin, ALDH, and more recently DCLK1 and Musashi, have been shown to harbor “stem cell” characteristics, in being enriched for the capacity to drive tumorigenesis and recreate the heterogeneity of the original tumor. Importantly, these tumor propagating cells or “cancer stem cells” have been shown to be highly resistant to cytotoxic therapies, such as gemcitabine, consistent with the finding that cancer patients with a high cancer stem cell signature have poorer prognosis relative to those with a low stem cell signature. Although pancreatic cancer stem cells are epithelial in origin, these cells frequently express EMT-associated programs, which may in part explain their over-representation in circulation and propensity to seed metastatic sites. Because these studies define stem cells as a population that present a particularly high risk for disease progression, defining the molecular signals that sustain them remains an essential goal for achieving complete and durable responses.

A combination of RNA-seq, ChIP-seq and genome-wide CRISPR screening was used to define the molecular framework that sustains the aggressive nature of pancreatic cancer stem cells. These data identified a network of key nodes regulating pancreatic cancer stem cells, and revealed an unanticipated role for immuno-regulatory genes in pancreatic cancer stem cell self-renewal and maintenance. Among these, RORγ, a nuclear hormone receptor known for its role in Th17 cell specification and regulation of inflammatory cytokine production, emerged as a key regulator of stem cells. RORγ expression increased with progression and blockade of RORγ signaling via genetic or pharmacological approaches depleted the cancer stem cell pool and profoundly inhibited human and mouse tumor propagation, in part by triggering the collapse of a super-enhancer-associated oncogenic network. Finally, sustained treatment with RORγ inhibitor led to a significant improvement in autochthonous models of pancreatic cancer. Together, these data offered a unique comprehensive map of pancreatic cancer stem cells and identified critical vulnerabilities that may be exploited to improve therapeutic targeting of aggressive, drug resistant pancreatic cells.

As disclosed herein, the molecular dependencies of pancreatic cancer stem cells have been systematically mapped out, including highly drug resistant cells that are also enriched in the capacity to drive progression. A sub-population of cells within pancreatic cancer that harbor stem cell characteristics and display preferential capacity to drive lethality and therapy resistance was identified. Because this work showed that these cancer stem cells were preferentially drug resistant and drove lethality, networks and cellular programs critical for the maintenance and function of these aggressive pancreatic cancer cells were identified. A combination of RNA-Seq, ChIP Seq and genome-wide CRISPR screening was used to develop a network map of core programs regulating pancreatic cancer and a unique multiscale map of programs that represent the core dependencies of pancreatic cancer stem cells. This analysis revealed an unexpected role for immunoregulatory genes in stem cell function and pancreatic cancer growth. In particular, retinoic acid receptor-related orphan receptor gamma (RORγ) emerged as a key regulator of pancreatic cancer stem cells.

As demonstrated in the working examples, RORγ expression was shown to be low in normal pancreatic cells but significantly increased in epithelial tumor cells with disease progression. ShRNA-mediated knockdown confirmed the role of RORγ identified by the genetic CRISPR-based screen as it led to a decrease in sphere formation of pancreatic cancer cells in vitro, and dramatically suppressed tumor initiation and propagation in vivo. Consistent with this, inhibition of RORγ resulted in a dose-dependent reduction in the number of pancreatic cancer spheroids in vitro, and combined delivery of RORγ inhibitor and gemcitabine in KPC mice with advanced pancreatic cancer led to depletion of the stem cell pool and lowered the tumor burden by half. Further, RORγ expression was low in normal human pancreas and in pancreatitis and rose with human pancreatic cancer progression. Blocking RORγ in human pancreatic cancer reduced growth in vitro and in vivo, suggesting that it plays an important role in human disease as well.

Leukemia and pancreatic cancer stem cells have some common features and shared molecular dependencies. As demonstrated in the working examples, KLS cells were isolated from WT and RORγ knockout (RORc^{-/-}) mice, retrovirally transduced with BCR-ABL and Nup98-HOXA9, and cultured in primary and secondary colony assays in vitro. A significant decrease in both colony number and overall colony area in primary and secondary colony assays was observed, indicating that growth and propagation of blast crisis CML is critically dependent on RORγ. In addition, an impact on acute myelogenous leukemia (AML) growth as well as RORγ expression in lymphoid tumors was observed, suggesting a role for RORγ signaling in these cancers as well.

The RORγ pathway also emerged as a key regulator of stem cells, as its expression was low in non-stem cells both at the RNA and protein levels but enriched in stem cell populations. RORγ was found to regulate potent oncogenes marked by super enhancers in stem cells and was shown to correlate to the aggressive nature of pancreatic cancer stem cells. Blockade of RORγ signaling via genetic or pharmacological approaches depleted the cancer stem cell pool and profoundly inhibited pancreatic tumor progression. Therapeutic, genetic, or CRISPR-based inhibition of RORγ has also proven to be effective in reducing cancer cell growth in leukemia and lung cancer. Moreover, given that the above identified roles of RORγ in cancer stem cell functions may not be particularly limited to one type of cancer, there is reason to believe that the RORγ pathway can be broadly utilized to epithelial and other types of cancers that share similar molecular dependencies of cancer stem cells. Taken together, it suggests that RORγ signaling play an important in cancer stem cells, and that targeting the RORγ pathway would be effective at inhibiting stem cell-driven cancers where RORγ expression level is high.

### RORγ Inhibitors, Analogs and Derivatives Thereof

Various RORγ inhibitors, as well as their analogs and derivatives, may be used in treating an RORγ-dependent cancer. For example, SR2211 is a selective synthetic RORγ modulator and an inverse agonist, represented by the following chemical structure:

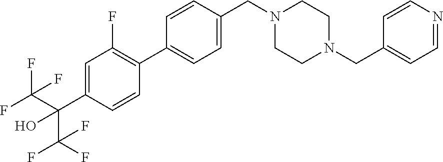 [SMILES: OC(c1ccc(-c2ccc(CN3CCN(Cc4ccncc4)CC3)cc2)c(F)c1)(C(F)(F)F)C(F)(F)F]

In certain embodiments, the RORγ inhibitor is an analog and/or derivative of SR2211. For example, the RORγ inhibitor may have a structure of Formula I:

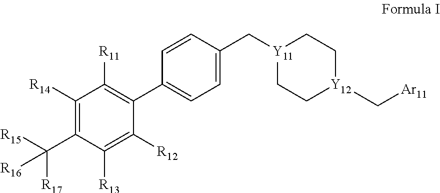

including pharmaceutically acceptable salts thereof, pharmaceutically acceptable isomers thereof, and pharmaceutically acceptable derivatives thereof, wherein:
R11, R12, R13, and R14 are independently selected from the group consisting of H, F, Cl, Br, and I, and can be the same or different, with the proviso that at least one of R11, R12, R13, and R14 is not H;
R15 and R17 are independently selected from the group consisting of H, alkyl, haloalkyl and alkoxy and can be the same or different;
R16 is selected from the group consisting of H, F, Cl, Br, I, hydroxyl, hydroxyalkyl, thiol, thiolalkyl, amino, and aminoalkyl;
Y11 and Y12 are independently selected from the group consisting of N, O, and S and can be the same or different; and
Ar11 is aryl or heteroaryl.

In certain embodiments, the RORγ inhibitor has a structure of Formula I, including pharmaceutically acceptable salts thereof, pharmaceutically acceptable isomers thereof, and pharmaceutically acceptable derivatives thereof, wherein:
R11, R12, R13, and R14 are independently selected from the group consisting of H, F, Cl, Br, and I, and can be the same or different, with the proviso that at least one of R11, R12, R13, and R14 is not H;
R15 and R17 are independently selected from the group consisting of H, —CH3, —CH2CH3, —CF3, and —OCH3, and can be the same or different;
R16 is selected from the group consisting of H, OH, SH, F, Cl, Br, and I;
Y11 and Y12 are N; and
Ar11 is selected from the group consisting of phenyl, 4-pyridinyl, 3-pyridinyl, 2-pyridinyl, and 4-amino-phenyl.

Another example of an RORγ inhibitor is AZD-0284, another inverse agonist, represented by the following chemical structure:

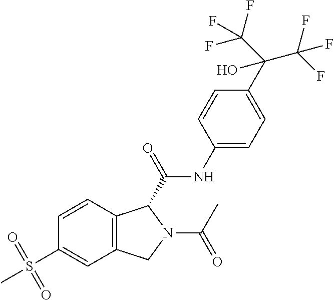 [SMILES: CC(=O)N1Cc2cc(S(C)(=O)=O)ccc2[C@@H]1C(=O)Nc1ccc(C(O)(C(F)(F)F)C(F)(F)F)cc1]

In certain embodiments, the RORγ inhibitor is an analog and/or derivative of AZD-0284. For example, the RORγ inhibitor may have a structure of Formula II:

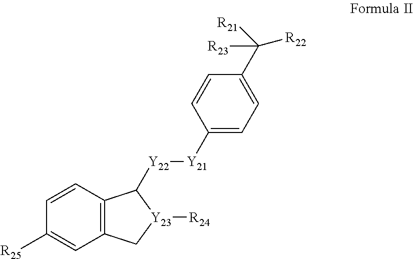

including pharmaceutically acceptable salts thereof, pharmaceutically acceptable isomers thereof, and pharmaceutically acceptable derivatives thereof, wherein:
R21 and R22 are selected from the group consisting of H, alkyl, haloalkyl, and alkoxy, and can be the same or different;
R23 is selected from the group consisting of H, F, Cl, Br, hydroxyl, hydroxyalkyl, thiol, thiolalkyl, amino, and aminoalkyl;
R24 is selected from the group consisting of H, alkyl, alkylcarbonyl, hydroxyalkyl, and alkylimino;
R25 is selected from the group consisting of H, alkylsulfonyl, and haloalkylsulfonyl; and
Y21 and Y22 are independently selected from the group consisting of —NH—, S, O, and C═O, with the proviso that at least one of Y21 and Y22 is C═O.

In certain embodiments, the RORγ inhibitor has a structure of Formula II, including pharmaceutically acceptable salts thereof, pharmaceutically acceptable isomers thereof, and pharmaceutically acceptable derivatives thereof, wherein:
R21 and R22 are selected from the group consisting of H, —CH3, —CH2CH3, —CF3, and —OCH3, and can be the same or different;
R23 is selected from the group consisting of H, OH, SH, F, Cl, Br, and I;
R24 is selected from the group consisting of H, CH3, acetyl, propionyl, —CH2-CH2-OH, C(═NH)—CH3, and C(═N—OH)—CH3;
R25 is selected from the group consisting of H, methylsulfonyl, trifluoromethylsulfonyl, and ethylsulfonyl; and
Y21 and Y22 are different and are independently selected from the group consisting of —NH— and C═O.

In certain embodiments, the RORγ inhibitor is a racemic mixture of AZD-0284 (rac-AZD-0284) represented by the following chemical structure:

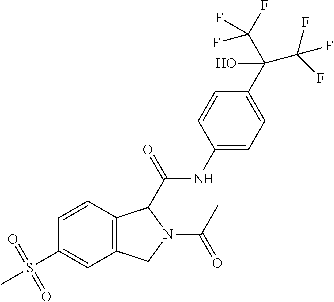 [SMILES: CC(=O)N1Cc2cc(S(C)(=O)=O)ccc2C1C(=O)Nc1ccc(C(O)(C(F)(F)F)C(F)(F)F)cc1]

In certain embodiments, the RORγ inhibitor is a racemic mixture of an inverse amide derivative of AZD-0284 represented by the following chemical structure:

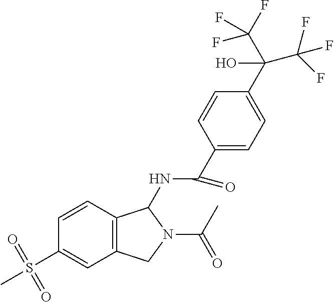 [SMILES: CC(=O)N1Cc2cc(S(C)(=O)=O)ccc2C1NC(=O)c1ccc(C(O)(C(F)(F)F)C(F)(F)F)cc1]

Yet another example of an RORγ inhibitor is JTE-151, disclosed as Compound A-58 in U.S. Pat. No. 8,604,069, and its chemical name is (4S)-6-[(2-chloro-4-methylphenyl)amino]-4-{4-cyclopropyl-5-[cis-3-(2,2-dimethylpropyl)cyclobutyl]isoxazol-3-yl}-6-oxohexanoic acid, represented by the following chemical structure:

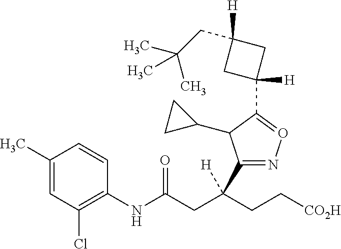

Another example of an RORγ inhibitor is JTE-151A, represented by the following chemical structure:

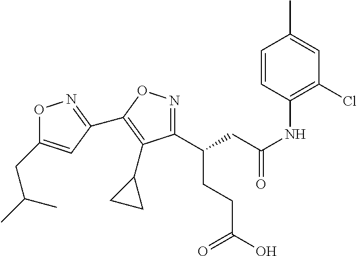 [SMILES: Cc1ccc(NC(=O)C[C@H](CCC(=O)O)c2noc(-c3cc(CC(C)C)on3)c2C2CC2)c(Cl)c1]

In certain embodiments, the RORγ inhibitor is an analog and/or derivative of JTE-151 or JTE-151A. For example, the RORγ inhibitor may have a structure of Formula III:

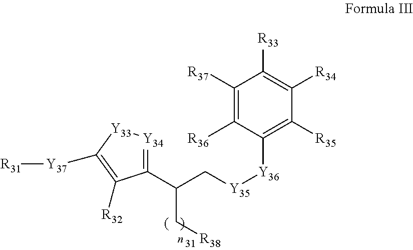

including pharmaceutically acceptable salts thereof, pharmaceutically acceptable isomers thereof, and pharmaceutically acceptable derivatives thereof, wherein:
R31, R32, and R33 are independently selected from the group consisting of H, alkyl, haloalkyl, alkoxy, and aryl;
R34, R35, R36, and R37 are independently selected from the group consisting of H, F, Cl, Br, and I, and can be the same or different, with the proviso that at least one of R34, R35, R36, and R37 is not H;
R38 is selected from the group consisting of —C(═O)—OR, C(═O)NR(R′), —C(═S)—OR, and —C(═O)—SR;
Y37 is

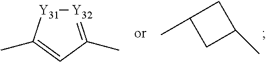

Y31, Y32, Y33 and Y34 are independently selected from the group consisting of O, N, and S, and can be the same or different;
Y35 and Y36 are independently selected from the group consisting of —NH—, S, O, and C═O, with the proviso that at least one of Y35 and Y36 is C═O;
n31 is 0, 1, 2, 3, 4, 5, or 6; and
R and R′ are independently selected from the group consisting of H and alkyl.

In certain embodiments, the RORγ inhibitor has a structure of Formula III, including pharmaceutically acceptable salts thereof, pharmaceutically acceptable isomers thereof, and pharmaceutically acceptable derivatives thereof, wherein:
Y37 is

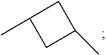

R31, R32, and R33 are independently selected from the group consisting of H, alkyl, haloalkyl, alkoxy, and aryl;
R34, R35, R36, and R37 are independently selected from the group consisting of H, F, Cl, Br, and I, and can be the same or different, with the proviso that at least one of R34, R35, R36, and R37 is not H;
R38 is selected from the group consisting of —C(═O)—OR, C(═O)NR(R′), —C(═S)—OR, and —C(═O)—SR;
Y33 and Y34 are independently selected from the group consisting of O, N, and S, and can be the same or different;
Y35 and Y36 are independently selected from the group consisting of —NH—, S, O, and C═O, with the proviso that at least one of Y35 and Y36 is C═O;
n31 iso, 1, 2, 3, 4, 5, or 6; and
R and R′ are independently selected from the group consisting of H and alkyl.

In certain embodiments, the RORγ inhibitor has a structure of Formula III, including pharmaceutically acceptable salts thereof, pharmaceutically acceptable isomers thereof, and pharmaceutically acceptable derivatives thereof, wherein:
Y37 is

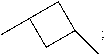

R31 is selected from the group consisting of H, CH3, CF3, ethyl, propyl, isopropyl, cyclopropyl, butyl, isobutyl, cyclobutyl, cyclopentyl, tert-butyl, neopentyl, cyclohexyl, and phenyl;
R32 is selected from the group consisting of H, CH3, CF3, ethyl, propyl, isopropyl, cyclopropyl, isobutyl, cyclobutyl, and cyclopentyl;
R33 is selected from the group consisting of H, CH3, CH2CH3, CF3, and OCH3;
R34, R35, R36, and R37 are independently selected from the group consisting of H, F, Cl, Br, and I, and can be the same or different, with the proviso that at least one of R34, R35, R36, and R37 is not H;
R38 is —C(═O)—OH;
Y31 and Y33 are O;
Y32 and Y34 are N;
Y35 and Y36 are different and are independently selected from the group consisting of —NH— and C═O; and
n31 is 1, 2, or 3.

In certain embodiments, the RORγ inhibitor is a racemic mixture of JTE-151 (rac-JTE-151) represented by the following chemical structure:

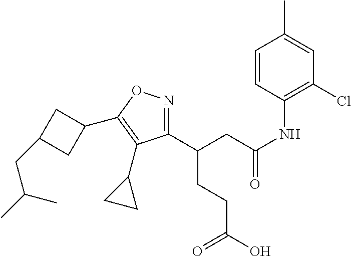 [SMILES: Cc1ccc(NC(=O)CC(CCC(=O)O)c2noc(C3CC(CC(C)C)C3)c2C2CC2)c(Cl)c1]

In certain embodiments, the RORγ inhibitor is a racemic mixture of an inverse amide derivative of JTE-151 represented by the following chemical structure:

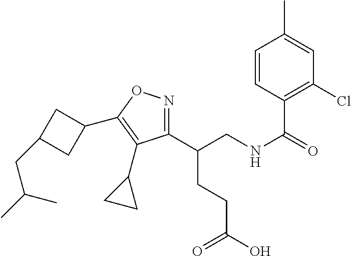 [SMILES: Cc1ccc(C(=O)NCC(CCC(=O)O)c2noc(C3CC(CC(C)C)C3)c2C2CC2)c(Cl)c1]

In certain embodiments, the RORγ inhibitor is an analog and/or derivative of JTE-151 having a structure of Formula IV:

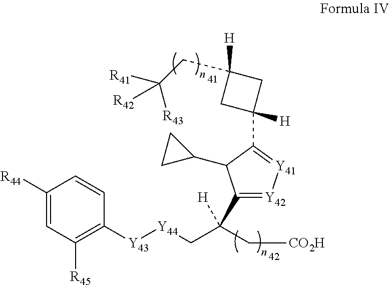

including pharmaceutically acceptable salts thereof, pharmaceutically acceptable isomers thereof, and pharmaceutically acceptable derivatives thereof, wherein:
R41, R42, R43, and R44 are alkyl and can be the same or different;
R45 is halogen, preferably selected from the group consisting of F, Cl, Br, and I;
Y41 and Y42 are independently selected from the group consisting of N, O, and S and can be the same or different;
Y43 and Y44 are independently selected from the group consisting of —NH—, S, O, and carbonyl, with the proviso that at least one of Y43 and Y44 is carbonyl;
n41 is 0, 1, 2, 3, 4, 5, or 6; and
n42 is 0, 1, 2, 3, 4, 5, or 6.

In certain embodiments, the RORγ inhibitor is an analog and/or derivative of JTE-151A. For example, the RORγ inhibitor may have a structure of Formula IIIA:

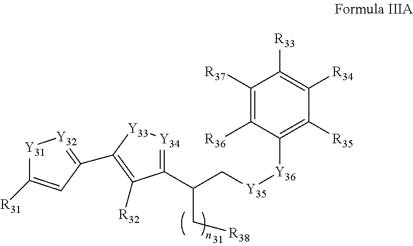

including pharmaceutically acceptable salts thereof, pharmaceutically acceptable isomers thereof, and pharmaceutically acceptable derivatives thereof, wherein:
R31, R32, and R33 are independently selected from the group consisting of H, alkyl, haloalkyl, alkoxy, and aryl;
R34, R35, R36, and R37 are independently selected from the group consisting of H, F, Cl, Br, and I, and can be the same or different, with the proviso that at least one of R34, R35, R36, and R37 is not H;
R38 is selected from the group consisting of —C(═O)—OR, C(═O)NR(R′), —C(═S)—OR, and —C(═O)—SR;
Y31, Y32, Y33 and Y34 are independently selected from the group consisting of O, N, and S, and can be the same or different;
Y35 and Y36 are independently selected from the group consisting of —NH—, S, O, and C═O, with the proviso that at least one of Y35 and Y36 is C═O;
n31 is 0, 1, 2, 3, 4, 5, or 6; and
R and R′ are independently selected from the group consisting of H and alkyl.

In certain embodiments, the RORγ inhibitor has a structure of Formula IIIA, including pharmaceutically acceptable salts thereof, pharmaceutically acceptable isomers thereof, and pharmaceutically acceptable derivatives thereof, wherein:
R31 is selected from the group consisting of H, CH3, CF3, ethyl, propyl, isopropyl, cyclopropyl, butyl, isobutyl, cyclobutyl, cyclopentyl, tert-butyl, neopentyl, cyclohexyl, and phenyl;
R32 is selected from the group consisting of H, CH3, CF3, ethyl, propyl, isopropyl, cyclopropyl, isobutyl, cyclobutyl, and cyclopentyl;
R33 is selected from the group consisting of H, CH3, CH2CH3, CF3, and OCH3;
R34, R35, R36, and R37 are independently selected from the group consisting of H, F, Cl, Br, and I, and can be the same or different, with the proviso that at least one of R34, R35, R36, and R37 is not H;
R38 is —C(═O)—OH;
Y31 and Y33 are O;
Y32 and Y34 are N;
Y35 and Y36 are different and are independently selected from the group consisting of —NH— and C═O; and
n31 is 1, 2, or 3.

In certain embodiments, the RORγ inhibitor is a racemic mixture of JTE-151A (rac-JTE-151A) represented by the following chemical structure:

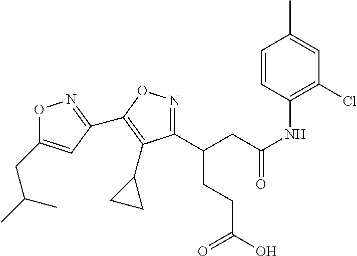 [SMILES: Cc1ccc(NC(=O)CC(CCC(=O)O)c2noc(-c3cc(CC(C)C)on3)c2C2CC2)c(Cl)c1]

In certain embodiments, the RORγ inhibitor is a racemic mixture of an inverse amide derivative of JTE-151A represented by the following chemical structure:

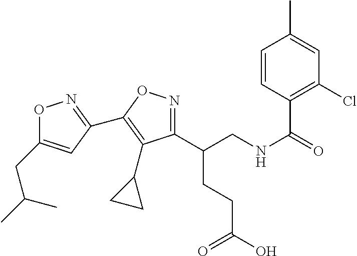 [SMILES: Cc1ccc(C(=O)NCC(CCC(=O)O)c2noc(-c3cc(CC(C)C)on3)c2C2CC2)c(Cl)c1]

The term “alkyl” refers to a straight or branched or cyclic chain hydrocarbon radical or combinations thereof, which can be completely saturated, mono- or polyunsaturated and can include di- and multivalent radicals. Examples of hydrocarbon radicals include, but are not limited to, groups such as methyl, ethyl, n-propyl, isopropyl, n-butyl, t-butyl, isobutyl, sec-butyl, n-pentyl, neopentyl, n-hexyl, n-heptyl, n-octyl, cyclopropyl, cyclobutyl, cyclopentyl, cyclohexyl, (cyclohexyl) methyl, cyclopropylmethyl, and the like.

The term “haloalkyl” refers to an alkyl group with 1, 2, 3, 4, 5, or 6 hydrogens substituted with the same or different halogen, preferably a halogen selected from the group consisting of F, Cl, Br, and I. Examples of haloalkyl groups include, without limitation, halomethyl (e.g., CF3), haloethyl, halopropyl, halobutyl, halopentyl, and halohexyl. Examples of halomethyl groups may have a structure of —C(X2)(X3)-X1 wherein X1 is selected from the group consisting of F, Cl, Br, and I; and X2 and X3 can be the same or different and are independently selected from the group consisting of H, F, Cl, Br, and I.

The term “hydroxyalkyl” refers to an alkyl group with 1, 2, 3, 4, 5, or 6 hydrogens substituted with hydroxyl groups. Examples of hydroxyalkyl groups include, without limitation, hydroxymethyl, hydroxyethyl, hydroxypropyl, hydroxybutyl, hydroxypentyl, and hydroxyhexyl. Examples of hydroxymethyl groups may have a structure of —C(X12)(X13)-X11 wherein X11 is OH; and X12 and X13 can be the same or different and are independently selected from the group consisting of H and OH.

The term “aminoalkyl” refers to an alkyl group with 1, 2, 3, 4, 5, or 6 hydrogens substituted with amino groups. Examples of aminoalkyl groups include, without limitation, aminomethyl, aminoethyl, aminopropyl, aminobutyl, aminopentyl, and aminohexyl. Examples of aminomethyl groups may have a structure of —C(X22)(X23)-X21 wherein X21 is amino; and X22 and X23 can be the same or different and are independently selected from the group consisting of H and amino.

The term “thiolalkyl” refers to an alkyl group with 1, 2, 3, 4, 5, or 6 hydrogens substituted with thiol groups. Examples of thiolalkyl groups include, without limitation, thiolmethyl, thiolethyl, thiolpropyl, thiolbutyl, thiolpentyl, and thiolhexyl. Examples of thiolmethyl groups may have a structure of —C(X32)(X33)-X31 wherein X31 is thio; and X32, and X33 can be the same or different and are independently selected from the group consisting of H and thiol.

The term “alkylcarbonyl” refers to —C(═O)—X41 wherein X41 is an alkyl group as defined herein. Examples of alkylcarbonyl groups include, without limitation, acetyl, propionyl, butyrionyl, pentanonyl, and hexanonyl.

The term “alkylimino” refers to —C(═N—X51)-X52 wherein X51 is H or OH; and X52 is an alkyl group as defined herein. Examples of alkylimino groups include, without limitation, —C(═NH)CH3, and —C(═N—OH)CH3.

The term “aryl” refers to aromatic groups that have only carbon ring atoms, optionally substituted with one or more substitution groups selected from the group consisting of halo, alkyl, amino, and hydroxyl. Examples of aryl groups include, without limitation, phenyl and naphthyl.

The term “heteroaryl” refers to aromatic groups having 1, 2, 3, or 4 heteroatoms as ring atoms, optionally substituted with one or more substitution groups selected from the group consisting of halo, alkyl, amino, and hydroxyl. Suitable heteroatoms include, without limitation, O, S, and N. Examples of heteroaryl groups include, without limitation, pyridyl, pyridazyl, pyrimidyl, pyrazinyl, thienyl, pyrrolyl, and imidazolyl.

The analogs and derivatives of the small molecule compounds disclosed herein have improved activities or retain at least partial activities in inhibiting RORγ and have other improved properties such as less toxicity for a subject receiving the compounds, analogs and derivatives thereof.

Examples of pharmaceutically acceptable salts include, without limitation, non-toxic inorganic and organic acid addition salts such as hydrochloride derived from hydrochloric acid, hydrobromide derived from hydrobromic acid, nitrate derived from nitric acid, perchlorate derived from perchloric acid, phosphate derived from phosphoric acid, sulphate derived from sulphuric acid, formate derived from formic acid, acetate derived from acetic acid, aconate derived from aconitic acid, ascorbate derived from ascorbic acid, benzenesulphonate derived from benzensulphonic acid, benzoate derived from benzoic acid, cinnamate derived from cinnamic acid, citrate derived from citric acid, embonate derived from embonic acid, enantate derived from enanthic acid, fumarate derived from fumaric acid, glutamate derived from glutamic acid, glycolate derived from glycolic acid, lactate derived from lactic acid, maleate derived from maleic acid, malonate derived from malonic acid, mandelate derived from mandelic acid, methanesulphonate derived from methane sulphonic acid, naphthalene-2-sulphonate derived from naphtalene-2-sulphonic acid, phthalate derived from phthalic acid, salicylate derived from salicylic acid, sorbate derived from sorbic acid, stearate derived from stearic acid, succinate derived from succinic acid, tartrate derived from tartaric acid, toluene-p-sulphonate derived from p-toluene sulphonic acid, and the like. Such salts may be formed by procedures well known and described in the art. Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable salts also include, without limitation, non-toxic inorganic and organic cationic salts such as the sodium salts, potassium salts, calcium salts, magnesium salts, zinc salts, aluminium salts, lithium salts, choline salts, lysine salts, and ammonium salts, and the like, of a chemical compound disclosed herein containing an anionic group. Such cationic salts may be formed by suitable procedures in the art.

Examples of pharmaceutically acceptable derivatives include, without limitation, ester derivatives, amide derivatives, ether derivatives, thioether derivatives, carbonate derivatives, carbamate derivatives, phosphate derivatives, etc.

### Combinational Therapy

Also disclosed herein are methods of treating cancer using one or more RORγ inhibitors or a composition comprising one or more RORγ inhibitors disclosed herein in combination with one or more other cancer therapies targeting a specific type of the cancer. The RORγ inhibitors or a composition comprising one or more RORγ inhibitors can be administered sequentially or simultaneously with one or more other cancer therapies over an extended period of time. Such methods may be used to treat any RORγ-dependent cancer or tumor cell type, including but not limited to primary, recurrent, and metastatic pancreatic cancer, lung cancer, and leukemia.

The RORγ inhibitors and compositions comprising the RORγ inhibitors disclosed herein can be used in combination with other conventional cancer therapies such as surgery, immunotherapy, radiotherapy, and/or chemotherapy to obtain improved or synergistic therapeutic effects. For example, surgery, chemotherapy, radiotherapy, and/or immunotherapy can be performed or administered before, during, or after the administration of the RORγ inhibitors or compositions comprising the RORγ inhibitors. As one of ordinary skill in the art would understand, the chemotherapy, immunotherapy, radiotherapy, and/or the RORγ inhibitors or compositions comprising the RORγ inhibitors can be administered to a subject in need thereof one or more times at the same or different doses, depending on the diagnosis and prognosis of the cancer. One skilled in the art would be able to combine one or more of these therapies in different orders to achieve the desired therapeutic results. In certain embodiments, the combinational therapy achieves synergist effects in comparison to any of the treatments administered alone.

Depending on the cancer type, various chemotherapeutic agents can be selected for use in combination with one or more RORγ inhibitors or a composition comprising one or more RORγ inhibitors disclosed herein. In certain embodiments, the chemotherapeutic agents for pancreatic cancer include but are not limited to gemcitabine (Gemzar), 5-fluorouracil (5-FU), irinotecan (Camptosar), oxaliplatin (Eloxatin), albumin-bound paclitaxel (Abraxane), capecitabine (Xeloda), cisplatin, paclitaxel (Taxol), docetaxel (Taxotere), and irinotecan liposome (Onivyde). In certain embodiments, the chemotherapeutic agents for leukemia include but are not limited to vincristine or liposomal vincristine (Marc daunorubicin or daunomycin (Cerubidine), doxorubicin (Adriamycin), cytarabine or cytosine arabinoside (ara-C) (Cytosar-U), L-asparaginase or PEG-L-asparaginase or pegaspargase (Oncaspar), 6-mercaptopurine (6-MP) (Purinethol), methotrexate (Xatmep, Trexall, Otrexup, Rasuvo), cyclophosphamide (Cytoxan, Neosar), prednisone (Deltasone, Prednisone Intensol, Rayos), imatinib mesylate (Gleevec), and nelarabine (Arranon). In certain embodiments, the chemotherapeutic agents for lung cancer include but are not limited to cisplatin (Platinol), carboplatin (Paraplatin), docetaxel (Taxotere), gemcitabine (Gemzar), paclitaxel (Taxol), vinorelbine (Navelbine), pemetrexed (Alimta), albumin-bound paclitaxel (Abraxane), etoposide (VePesid or Etopophos), doxorubicin (Adriamycin), ifosfamide (Ifex), irinotecan (Camptosar), paclitaxel (Taxol), topotecan (Hycamtin), vinblastine (Oncovir), and vincristine (Oncovin).

In certain embodiments, the combinational therapy leads to improved clinical outcome and/or higher survival rate for cancer patients, especially for metastatic cancer patients. In certain embodiments, the combinational therapy achieves the same therapeutic effect, a better therapeutic effect, or even a synergistic effect when administered at a lower dose and/or for a short period of time than any of the treatments administered alone. For example, when an RORγ inhibitor and a chemotherapeutic agent are used in a combinational therapeutic, either or both may be administered at a lower dose than the RORγ inhibitor or the chemotherapeutic agent administered alone. In another example, when an RORγ inhibitor and a radiotherapy are used in a combinational therapeutic, either or both may be administered at a lower dose or the radiotherapy may be administered for a shorter period than the RORγ inhibitor or the chemotherapeutic agent administered alone. This advantage of the combinational therapy has a significant impact on the clinical outcome because the toxicity, drug resistance, and/or other undesirable side effects caused by the treatment are reduced due to the reduced dose and/or reduced treatment period. One hurdle of cancer therapy is that many cancer patients have to discontinue the treatment due to the severity of the side effects, which sometimes even cause complications.

In certain embodiments, multiple doses of one or more RORγ inhibitors or compositions comprising one or more RORγ inhibitors are administered in combination with multiple doses or multiple cycles of other cancer therapies. In these embodiments, the RORγ inhibitors and other cancer therapies can be administered simultaneously or sequentially at any desirable intervals. In certain embodiments, the RORγ inhibitors and other cancer therapies can be administered in alternate cycles, e.g., administration of one or more doses of the RORγ inhibitor disclosed herein followed by administration of one or more doses of a chemotherapeutic agent.

### Method of Prevention/Treatment Using the RORγ Inhibitors

Provided herein is a method of treating and/or preventing a RORγ-dependent cancer in a subject. The method entails administering a therapeutically effective amount of one or more RORγ inhibitors or a composition comprising one or more RORγ inhibitors provided herein to the subject. In certain embodiments, the method further entails administering one or more other cancer therapies such as surgery, immunotherapy, radiotherapy, and/or chemotherapy to the subject sequentially or simultaneously.

Also provided herein is a method of preventing or delaying progression of an RORγ-dependent benign tumor to a malignant tumor in a subject. The method entails administering an effective amount of one or more RORγ inhibitors or a composition comprising one or more RORγ inhibitors provided herein to the subject. In certain embodiments, the method further entails administering one or more other therapies such as such as surgery, immunotherapy, radiotherapy, and/or chemotherapy to the subject sequentially or simultaneously.

As used herein, the term “subject” refers to a mammalian subject, preferably a human. A “subject in need thereof” refers to a subject who has been diagnosed with cancer, or is at an elevated risk of developing cancer. The phrases “subject” and “patient” are used interchangeably herein.

The terms “treat,” “treating,” and “treatment” as used herein with regard to cancer refers to alleviating the cancer partially or entirely, preventing the cancer, decreasing the likelihood of occurrence or recurrence of the cancer, slowing the progression or development of the cancer, or eliminating, reducing, or slowing the development of one or more symptoms associated with the cancer. For example, “treating” may refer to preventing or slowing the existing tumor from growing larger, preventing or slowing the formation or metastasis of cancer, and/or slowing the development of certain symptoms of the cancer. In some embodiments, the term “treat,” “treating,” or “treatment” means that the subject has a reduced number or size of tumor comparing to a subject without being administered with the treatment. In some embodiments, the term “treat,” “treating,” or “treatment” means that one or more symptoms of the cancer are alleviated in a subject receiving the RORγ inhibitors or pharmaceutical compositions comprising the RORγ inhibitors as disclosed herein and/or other cancer therapies comparing to a subject who does not receive such treatment.

A “therapeutically effective amount” of one or more RORγ inhibitors or the pharmaceutical composition comprising one or more RORγ inhibitors as used herein is an amount of the RORγ inhibitor or pharmaceutical composition that produces a desired effect in a subject for treating and/or preventing cancer. In certain embodiments, the therapeutically effective amount is an amount of the RORγ inhibitor or pharmaceutical composition that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the RORγ inhibitor or the pharmaceutical composition and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, N.Y., 2011, the entire disclosures of which are incorporated by reference herein.

In some embodiments, a therapeutically effective amount of an RORγ inhibitor disclosed herein is in the range from about 10 mg/kg to about 150 mg/kg, from 30 mg/kg to about 120 mg/kg, from 60 mg/kg to about 90 mg/kg. In some embodiments, a therapeutically effective amount of an RORγ inhibitor disclosed herein is about 15 mg/kg, about 30 mg/kg, about 45 mg/kg, about 60 mg/kg, about 75 mg/kg, about 90 mg/kg, about 105 mg/kg, about 120 mg/kg, about 135 mg/kg, or about 150 mg/kg. A single dose or multiple doses of an RORγ inhibitor may be administered to a subject. In some embodiments, the RORγ inhibitor is administered twice a day.

It is within the purview of one of ordinary skill in the art to select a suitable administration route, such as oral administration, subcutaneous administration, intravenous administration, intramuscular administration, intradermal administration, intrathecal administration, or intraperitoneal administration. For treating a subject in need thereof, the RORγ inhibitor or pharmaceutical composition can be administered continuously or intermittently, for an immediate release, controlled release or sustained release. Additionally, the RORγ inhibitor or pharmaceutical composition can be administered three times a day, twice a day, or once a day for a period of 3 days, 5 days, 7 days, 10 days, 2 weeks, 3 weeks, or 4 weeks. In certain embodiments, the RORγ inhibitor or pharmaceutical composition can be administered every day, every other day, or every three days. The RORγ inhibitor or pharmaceutical composition may be administered over a pre-determined time period. Alternatively, the RORγ inhibitor or pharmaceutical composition may be administered until a particular therapeutic benchmark is reached. In certain embodiments, the methods provided herein include a step of evaluating one or more therapeutic benchmarks such as the level of RORγ in a biological sample such as blood circulating tumor cells, a biopsy sample, or urine to determine whether to continue administration of the RORγ inhibitor or pharmaceutical composition.

### Pharmaceutical Compositions

One or more RORγ inhibitors disclosed herein can be formulated into pharmaceutical compositions. In some embodiments, the pharmaceutical composition comprises only one RORγ inhibitor. In some embodiments, the pharmaceutical composition comprises two or more RORγ inhibitors. The pharmaceutical compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

The pharmaceutical compositions can have various formulations, e.g., injectable formulations, lyophilized formulations, liquid formulations, oral formulations, etc. depending on the administration routes disclosed in the foregoing paragraphs.

In certain embodiments, the pharmaceutical composition may further comprise one or more additional therapeutic agents such as one or more chemotherapeutic agents or one or more radiation therapeutic agents. The one or more additional therapeutic agents may be formulated into the same pharmaceutical composition comprising the RORγ inhibitor disclosed herein or into separate pharmaceutical compositions for combinational therapy. Depending on the cancer type, various chemotherapeutic agents can be selected for use in combination with one or more RORγ inhibitors or a composition comprising one or more RORγ inhibitors disclosed herein. In certain embodiments, the chemotherapeutic agents for pancreatic cancer include but are not limited to gemcitabine (Gemzar), 5-fluorouracil (5-FU), irinotecan (Camptosar), oxaliplatin (Eloxatin), albumin-bound paclitaxel (Abraxane), capecitabine (Xeloda), cisplatin, paclitaxel (Taxol), docetaxel (Taxotere), and irinotecan liposome (Onivyde). In certain embodiments, the chemotherapeutic agents for leukemia include but are not limited to vincristine or liposomal vincristine (Marqibo), daunorubicin or daunomycin (Cerubidine), doxorubicin (Adriamycin), cytarabine or cytosine arabinoside (ara-C) (Cytosar-U), L-asparaginase or PEG-L-asparaginase or pegaspargase (Oncaspar), 6-mercaptopurine (6-MP) (Purinethol), methotrexate (Xatmep, Trexall, Otrexup, Rasuvo), cyclophosphamide (Cytoxan, Neosar), prednisone (Deltasone, Prednisone Intensol, Rayos), imatinib mesylate (Gleevec), and nelarabine (Arranon). In certain embodiments, the chemotherapeutic agents for lung cancer include but are not limited to cisplatin (Platinol), carboplatin (Paraplatin), docetaxel (Taxotere), gemcitabine (Gemzar), paclitaxel (Taxol), vinorelbine (Navelbine), pemetrexed (Alimta), albumin-bound paclitaxel (Abraxane), etoposide (VePesid or Etopophos), doxorubicin (Adriamycin), ifosfamide (Ifex), irinotecan (Camptosar), paclitaxel (Taxol), topotecan (Hycamtin), vinblastine (Oncovir), and vincristine (Oncovin).

The following examples are intended to illustrate various embodiments of the invention. As such, the specific embodiments discussed or any specific materials and methods disclosed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

This working example demonstrates the novel identification and characterization of pathways involving RORγ in pancreatic cancer. This working example further demonstrates that pharmacologic blockade of RORγ using SR2211, an inhibitor of RORγ, can effectively inhibit pancreatic cancer growth both in vitro and in vivo. Collectively, the data demonstrate that the RORγ pathway presents novel molecular targets for the treatment of cancer and may lead to the development of new classes of therapeutics that can be used in cancer treatment.

A. Transcriptomic and Epigenetic Map of Pancreatic Cancer Cells Reveals a Unique Stem Cell State

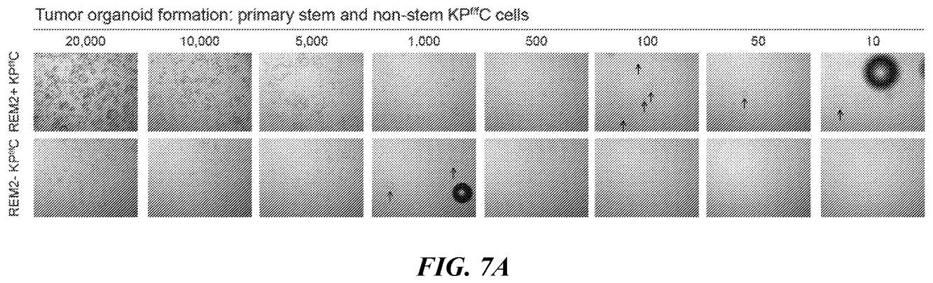

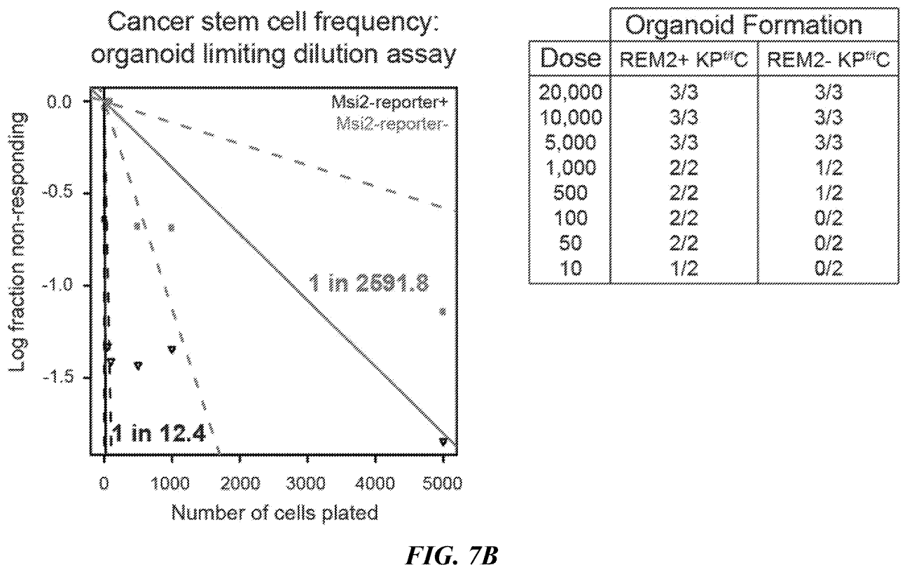

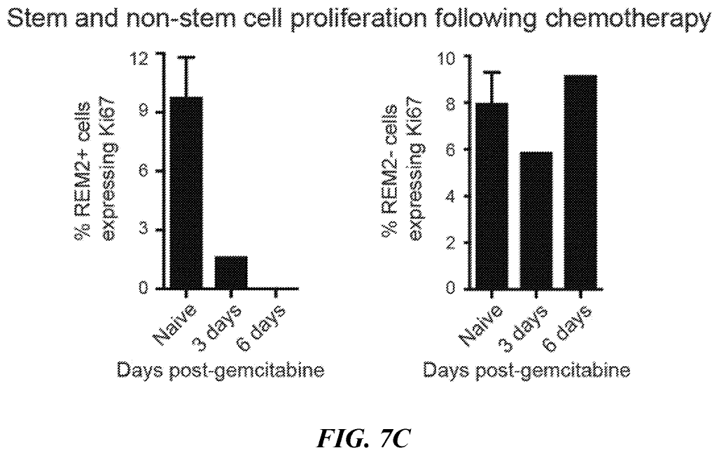

The KP^{f/f}C mouse model of pancreatic ductal adenocarcinoma (PDAC) was used to show that a reporter mouse designed to mirror expression of the stem cell signal Musashi (Msi) could effectively identify tumor cells that preferentially harbor capacity for drug resistance and tumor re-growth. Further, Msi2+ tumor cells were 209-fold enriched in the ability to give rise to organoids in limiting dilution assays (FIGS. 7A-7B). Because Msi+ cells were preferentially enriched for tumor propagation and drug resistance—classically defined properties of cancer stem cells—it was postulated that Msi reporters could be used as a tool to understand the molecular underpinnings of this aggressive subpopulation within pancreatic cancer.

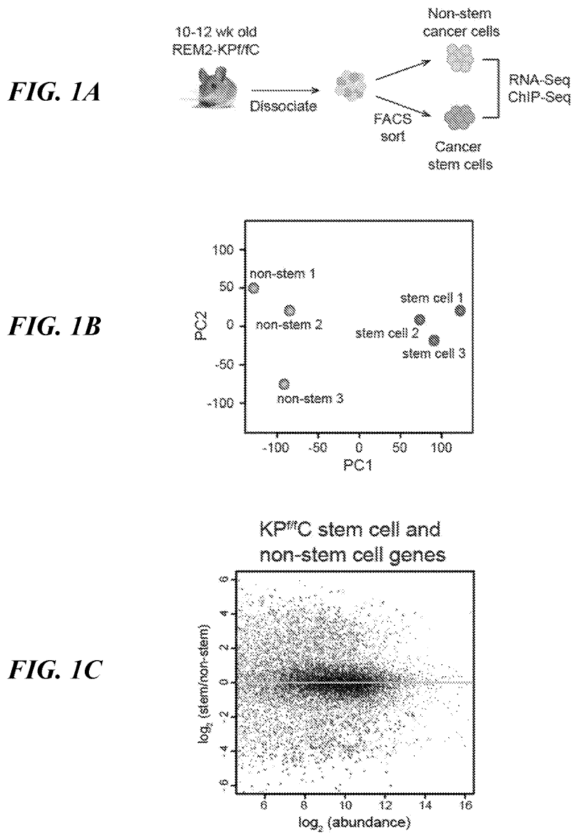

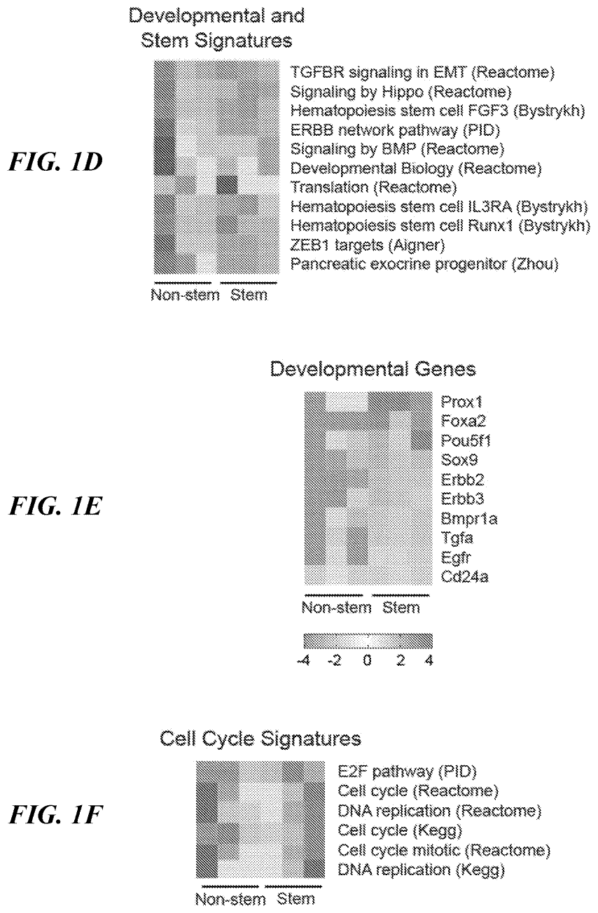

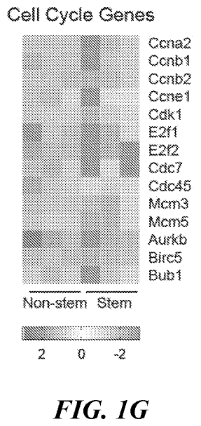

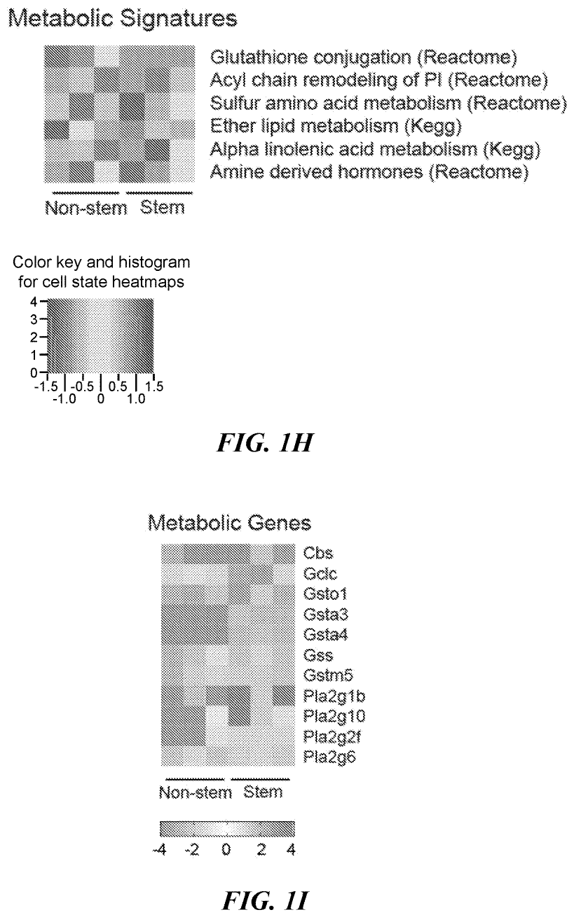

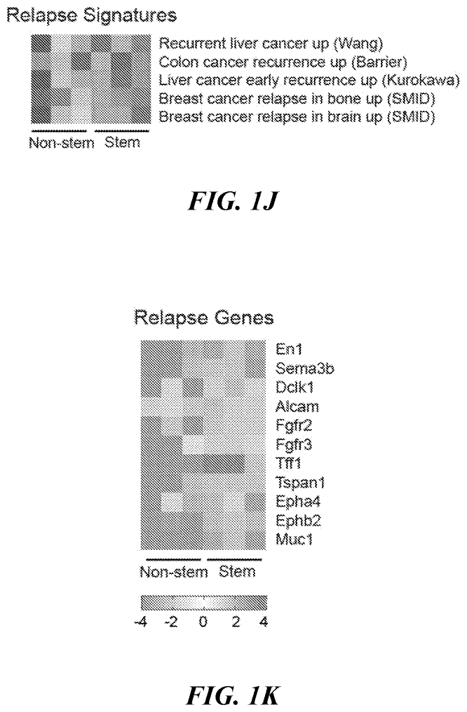

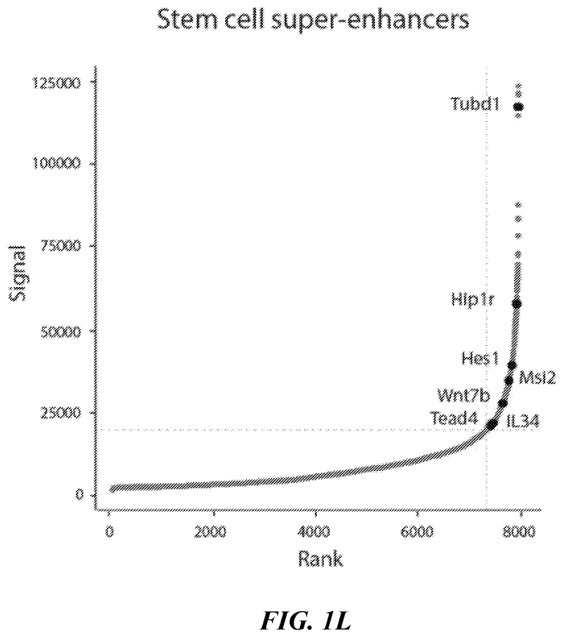

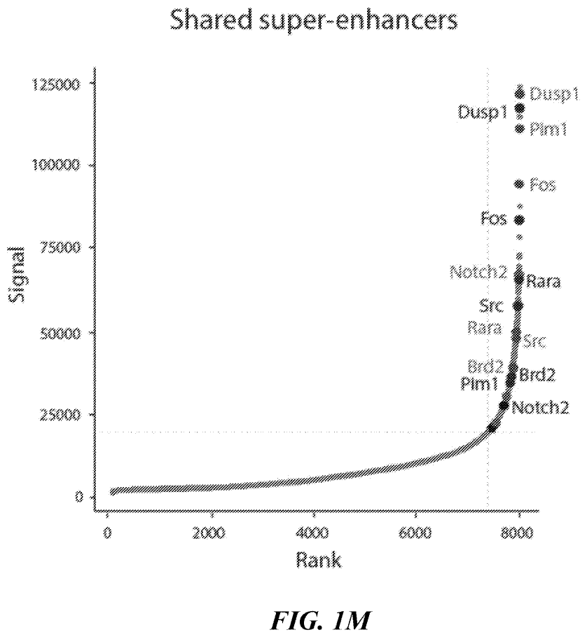

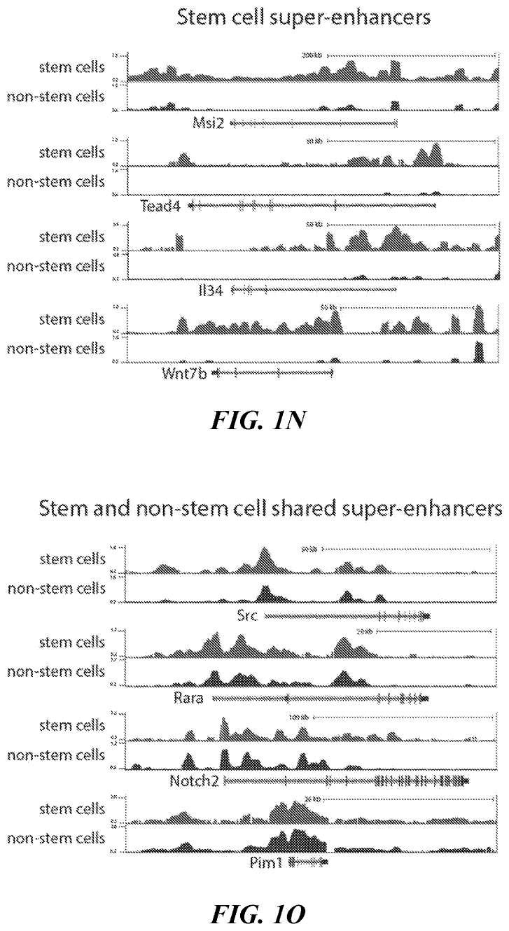

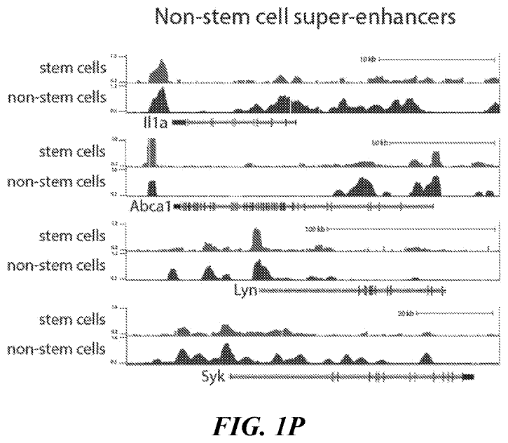

To map the functional landscape of the stem cell state, a combination of RNA-seq, ChIP-seq and genome-wide CRISPR screening was utilized. Pancreatic cancer cells were isolated from Msi2-reporter (REM2) KP^{f/f}C mice based on GFP and EpCAM expression and analyzed by RNA-seq (FIG. 1A). Principal component analysis showed that KP^{f/f}C reporter+ tumor cells were strikingly distinct from reporter− tumor cells at a global transcriptional level, indicating that they were functionally driven by a unique set of programs defined by differential expression of over a thousand genes (FIGS. 1B-1C). The genes enriched in stem cells (lfdr<0.2) were focused upon in order to understand the transcriptional programs that may functionally maintain the stem cell phenotype. Gene Set Enrichment Analysis (GSEA) was used to compare this PDAC stem cell transcriptome signature with other cell signatures. This revealed that the transcriptional state of PDAC stem cells mapped closely with other developmental and stem cell states, indicating molecular features aligned with their observed functional traits (FIGS. 1D-1E). Additionally, the transcriptional signature of PDAC stem cells was inversely correlated with cell proliferation signatures (FIGS. 1F-1G), consistent with the finding that stem cells are largely quiescent following chemotherapy while non-stem cells continue to cycle (FIG. 7C). Moreover, stem cells were characterized by metabolic signatures associated with tumor aggressiveness including increased sulfur amino acid metabolism, and enhanced glutathione synthesis, which can enable survival following radiation and chemotherapy (FIGS. 1H-1I). Finally, the PDAC stem cell transcriptome bore striking similarities to signatures from relapsed cancers of the breast, liver, and colon, programs that may underlie the ability of these cells to survive chemotherapy and drive tumor re-growth (FIGS. 1J-1K).

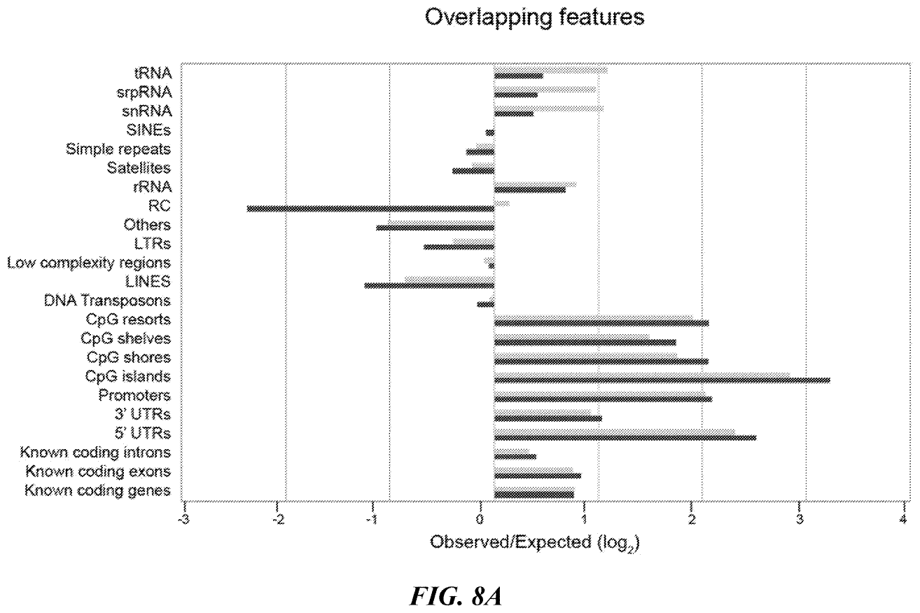

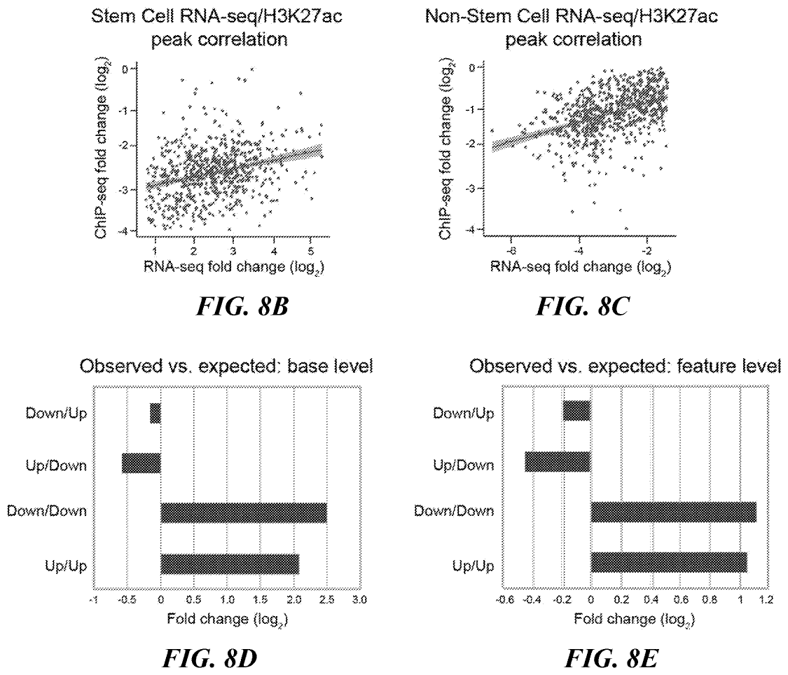

Consistent with the significant molecular differences found in stem cells by transcriptomic analysis, the distribution of H3 lysine-27 acetylation (H3K27ac, FIGS. 1A, 8A), a histone mark associated with active enhancers, revealed that the differential gene expression programs were driven by changes at the chromatin level. Thus, genomic regions enriched for H3K27ac specifically in either stem cells or non-stem cells coincided with regions where gene expression was increased in each cell type (FIGS. 8B-8E; correlation for stem cells: R²=0.28, p=7.1×10⁻¹⁴, non-stem cells R²=0.46, p=22×10⁻¹⁶). Because super-enhancers have been proposed to be key drivers of cell identity, shared and unique super-enhancers were mapped in stem and non-stem cells (FIGS. 1L-1P). This revealed that not all epigenetic changes were equivalently different between the two populations: while most promoter and enhancer-associated H3K27ac marks were shared in both stem and non-stem tumor cells, with less than 5% being unique, super-enhancer associated H3K27ac marks were much more frequently restricted, with 65% of all super-enhancers being unique to each population, with 364 super-enhancers being unique to stem cells and 388 being unique to non-stem cells. Further, super-enhancers in the stem cell population were clearly demarcated by peaks with substantially greater peak intensity and strength (FIG. 1N) while those in non-stem cells were either shared with stem cells or only marginally more enriched in H3K27Ac than those in stem cells (FIG. 1P). These data suggest that stem cells in pancreatic cancer have a more defined super-enhancer landscape than non-stem cells and raise the possibility that super-enhancers and their upstream transcriptional regulators may be preferential effectors of stem cell identity in pancreatic cancer. In support of this, key transcription factors and programs that underlie developmental and stem cell states, such as Klf7, Foxp1, Hmga1, Meis2, Tead4, Wnt7b and Msi2, were associated with super-enhancers in KP^{f/f}C stem cells (FIGS. 1L, 1N).

B. Genome-Scale CRISPR Screen Identifies Core Functional Programs in Pancreatic Cancer

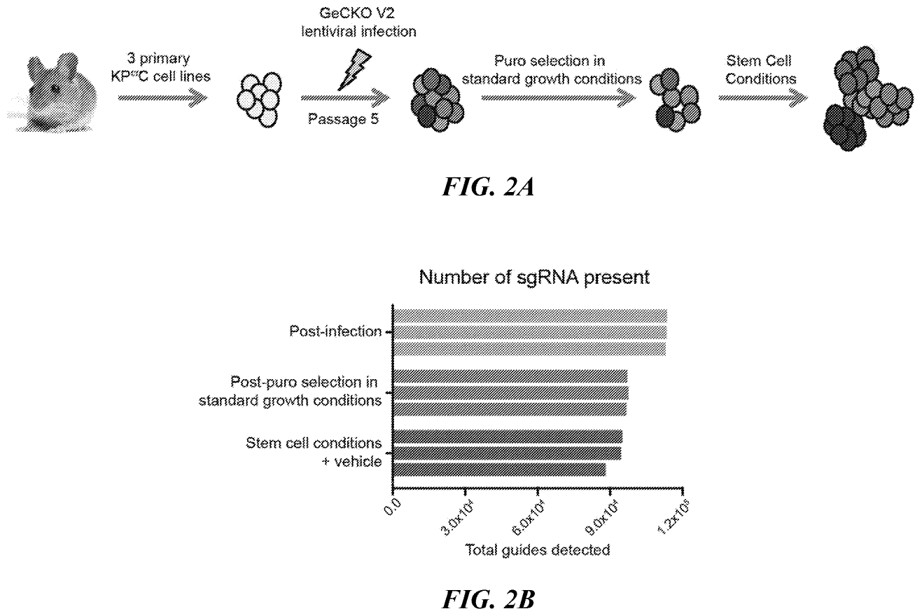

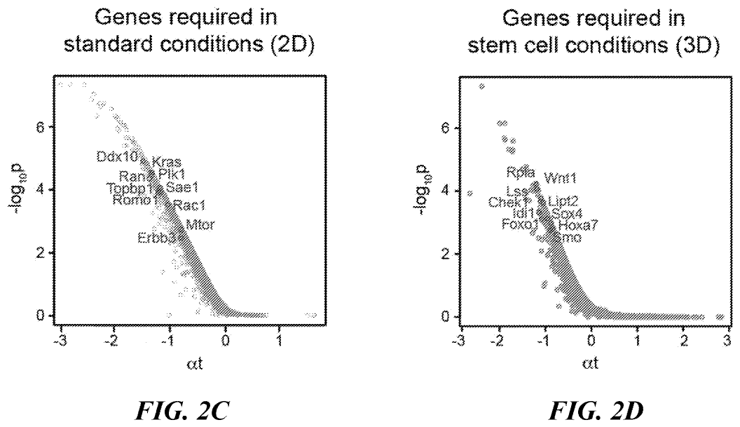

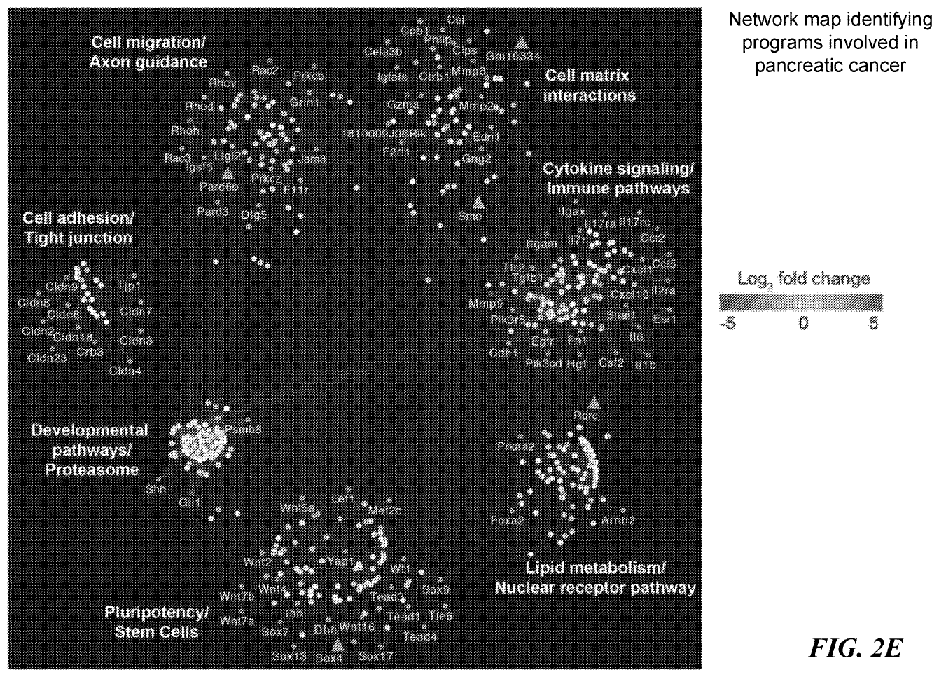

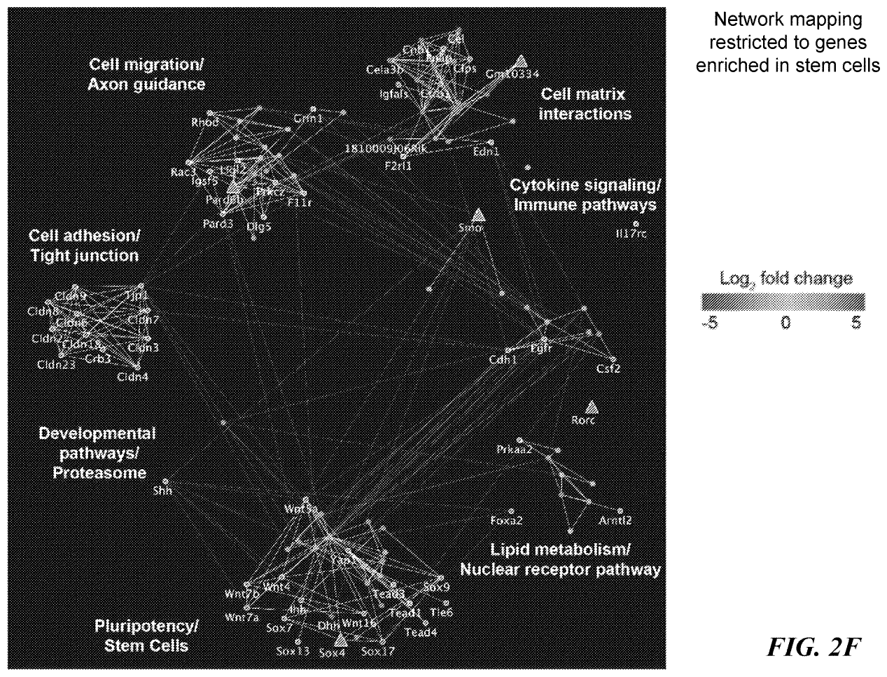

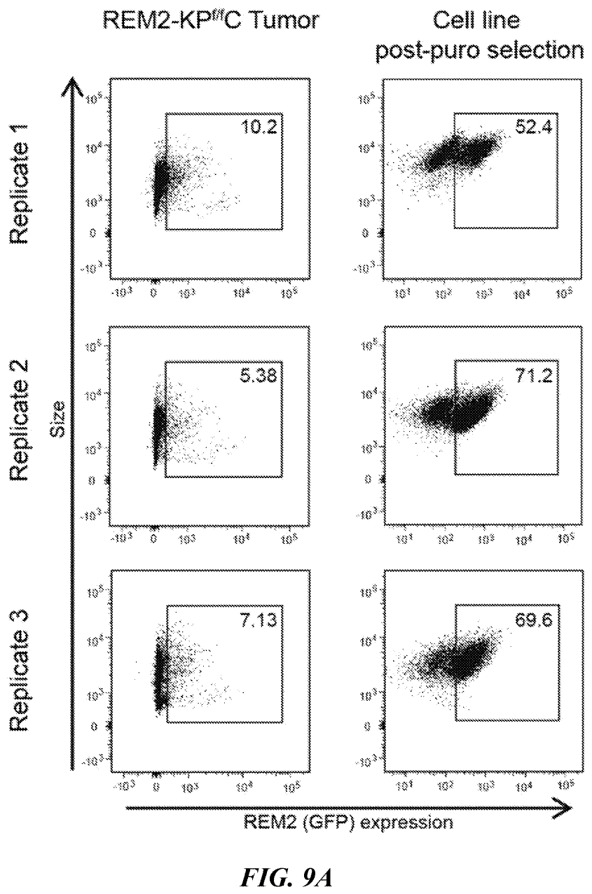

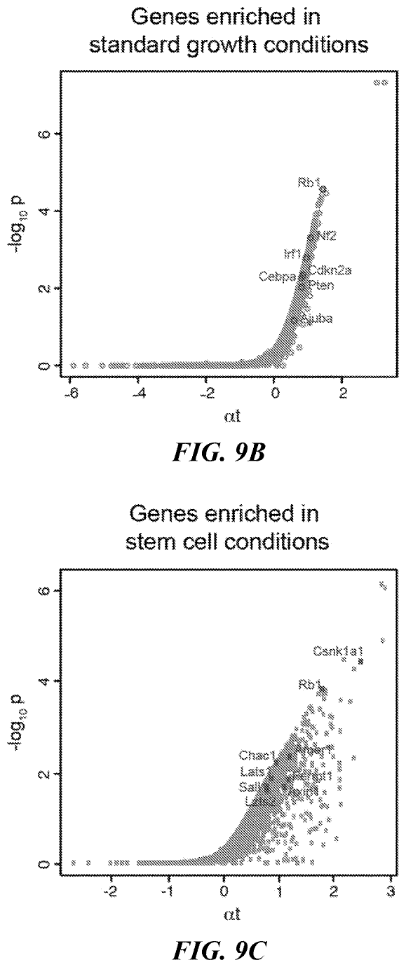

In some embodiments, a genome-wide CRISPR screen was carried out to define which of the programs uncovered by the transcriptional and epigenetic analyses represented true functional dependencies of stem cells. Primary cell cultures highly enriched for stem cells (FIG. 9A) from Msi reporter-KP^{f/f}C mice and transduced them with the mouse GeCKO CRISPRv2 sgRNA library (FIG. 2A). The screen was designed to be multiplexed in order to identify genes required in conventional 2-dimensional cultures, as well as in 3-dimensional sphere cultures that selectively allow stem cell growth (FIG. 2A). The screens showed clear evidence of selection, with 807 genes depleted (and thus essential) in conventional cultures (FIGS. 2B-2C, p<0.005) and an additional 178 in stem cell conditions (FIGS. 2B, 2D, p<0.005). Importantly, the screens showed a loss of oncogenes and an enrichment of tumor suppressors in conventional cultures (FIGS. 2C, 9B), and a loss of stem cell signals and gain of negative regulators of stem signals in stem cell conditions (FIGS. 2D, 9C).

Computational integration of the transcriptomic and CRISPR-based functional genomic data was carried out using a network propagation method similar to one developed previously. First, the network was seeded with genes that were preferentially enriched in stem cells RNAseq log FC>2 and also identified as essential for stem cell growth FDR<0.5 in 3-dimensional sphere cultures in the CRISPR assay (FIG. 2E). The genes most proximal to the seeds were then determined using the mouse STRING interactome based on known and predicted protein-protein interactions using network propagation. Fold-change in RNA expression from the RNAseq data was overlaid onto the resulting subnetwork. The network was subsequently clustered into functional communities based on high interconnectivity between genes, and gene set over-representation analysis was performed on each community; this analysis identified seven subnetworks built around distinct biological pathways, thus providing a higher order view of ‘core programs’ that may be involved in driving pancreatic cancer growth. These core programs identified stem and pluripotency pathways, developmental and proteasome signals, lipid metabolism/nuclear receptors, cell adhesion/cell-matrix/cell migration, and immuno-regulatory signaling as pathways integral to the stem cell state (FIGS. 2E, 2F).

C. Hijacked Immunorequlatory Programs as Direct Regulators of Pancreatic Cancer Cells

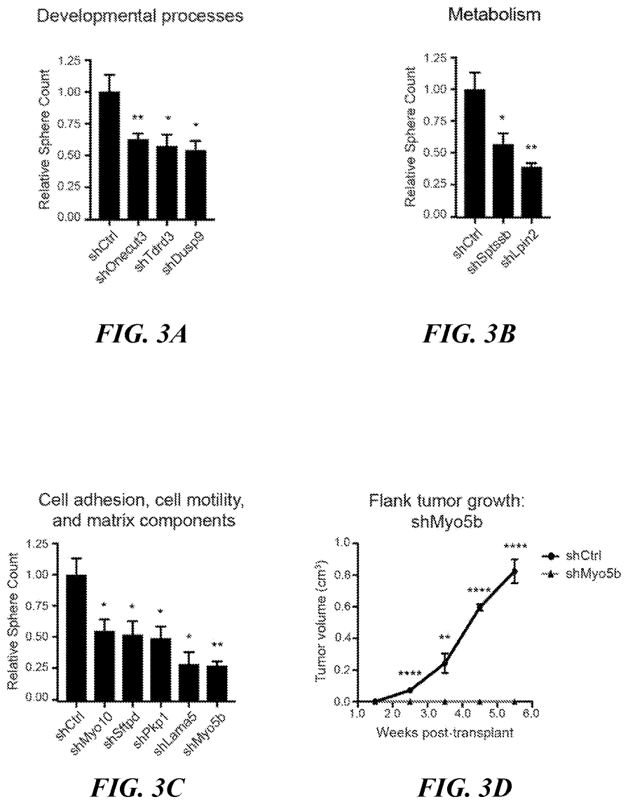

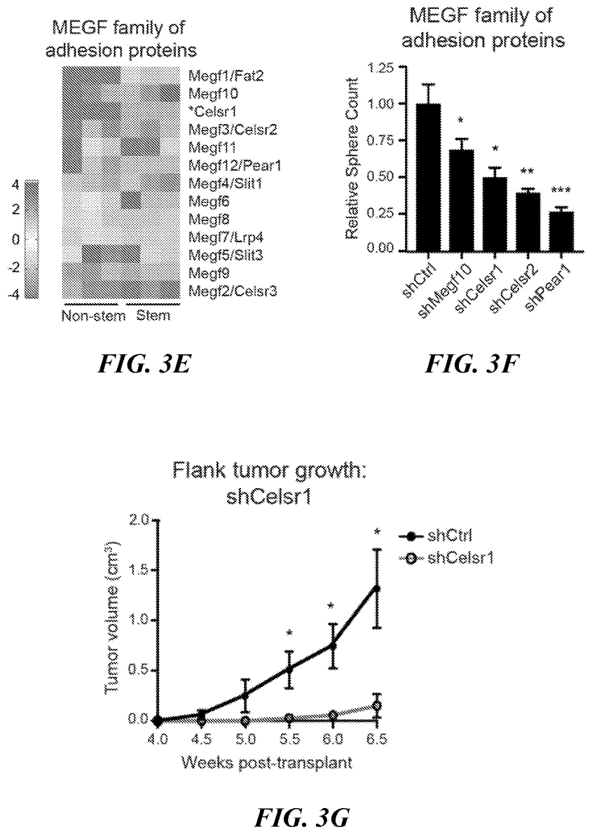

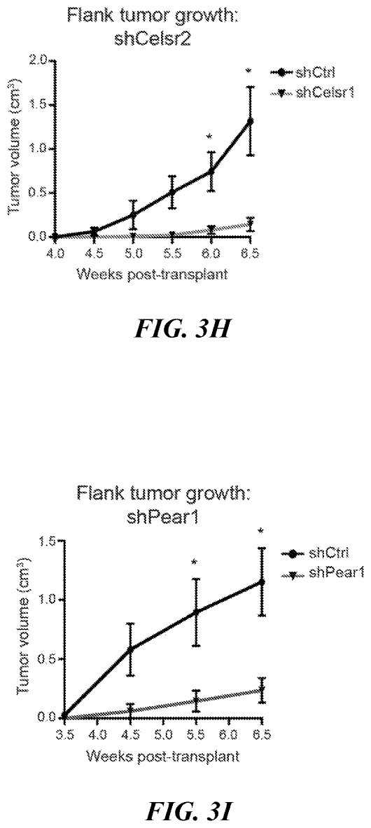

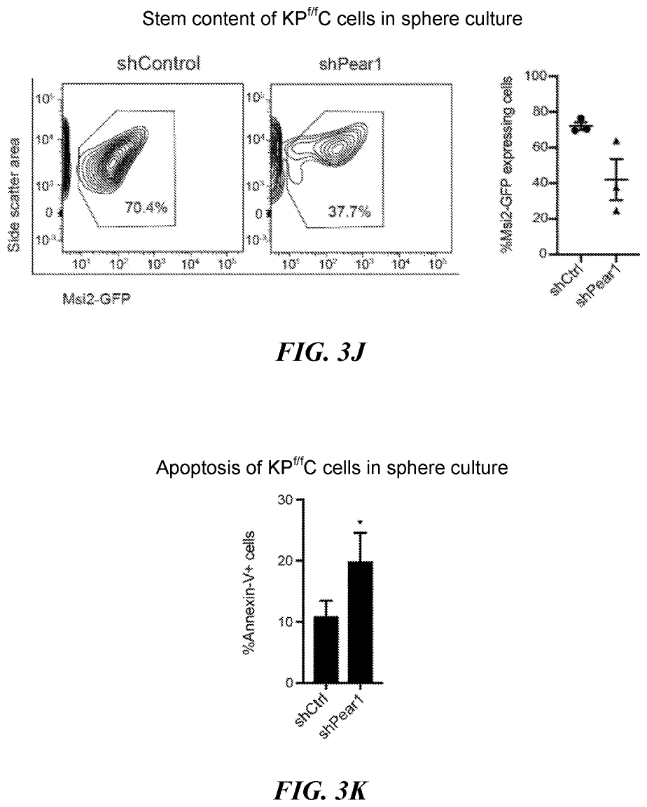

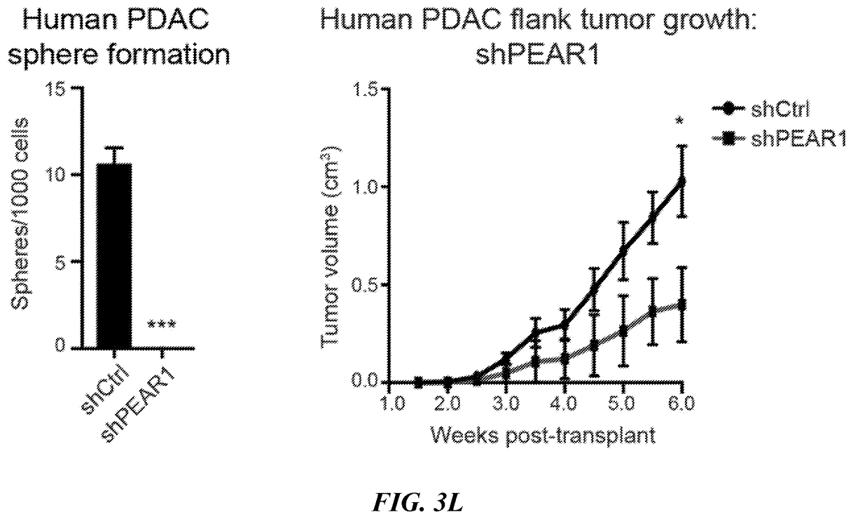

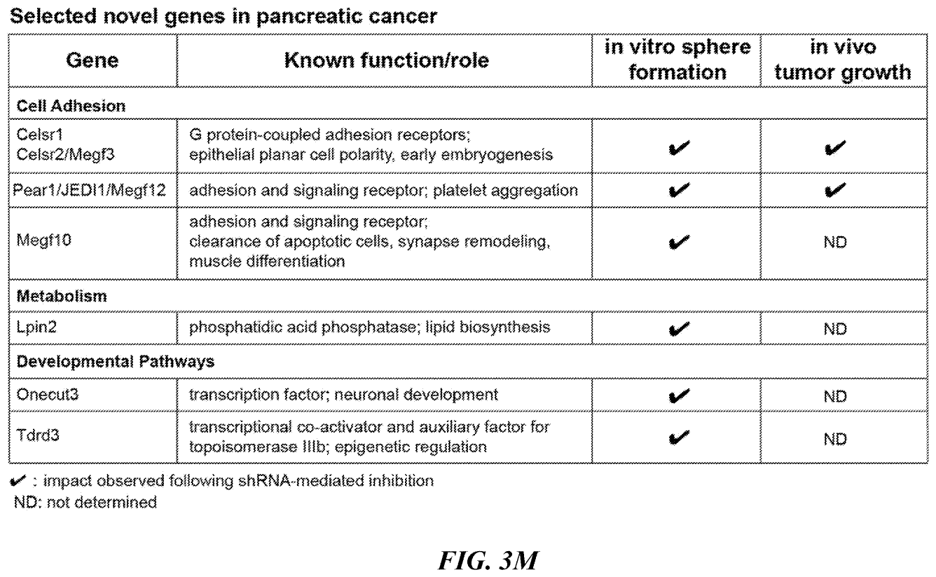

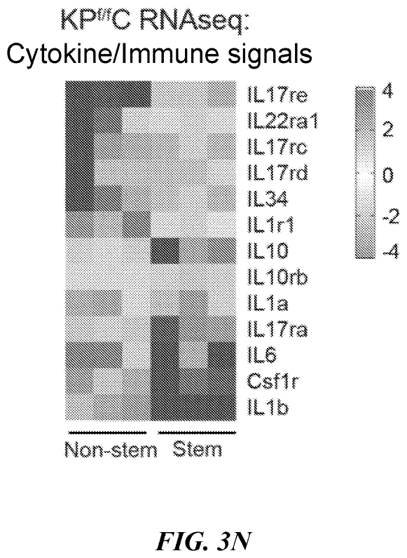

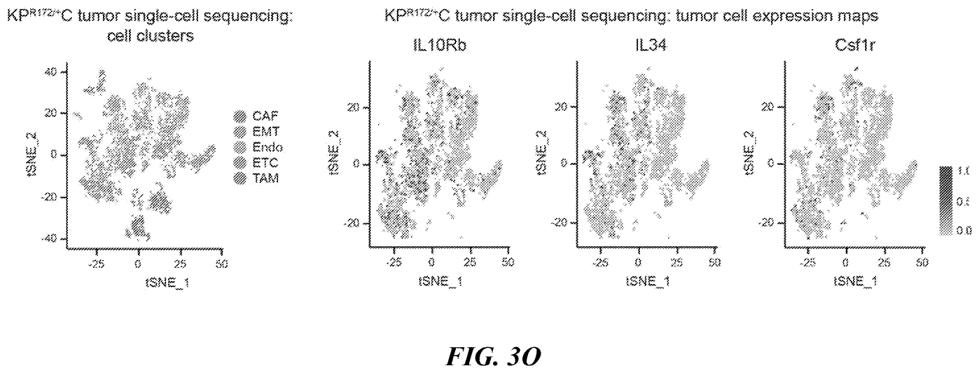

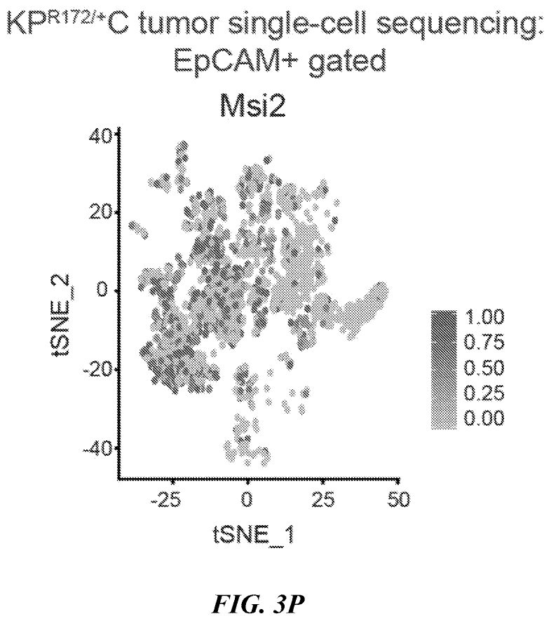

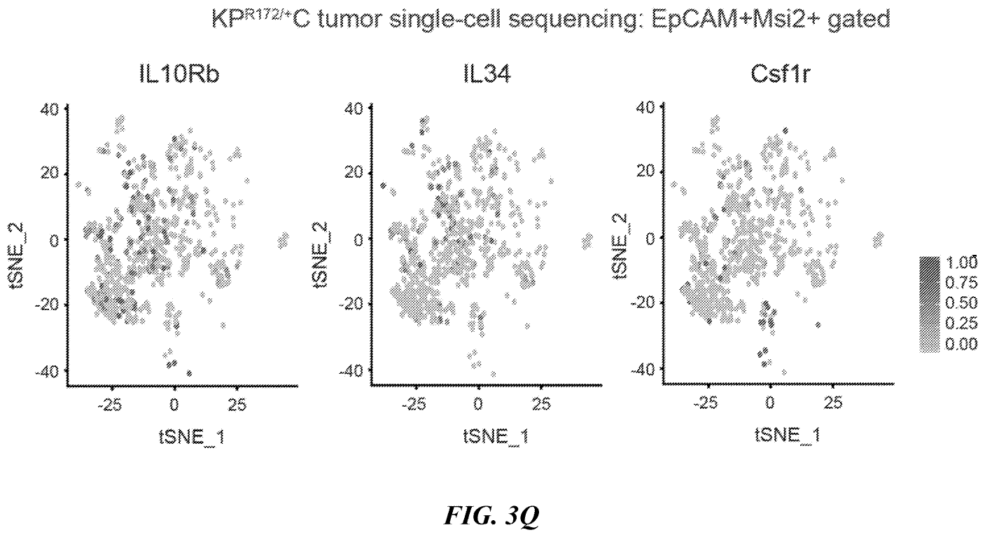

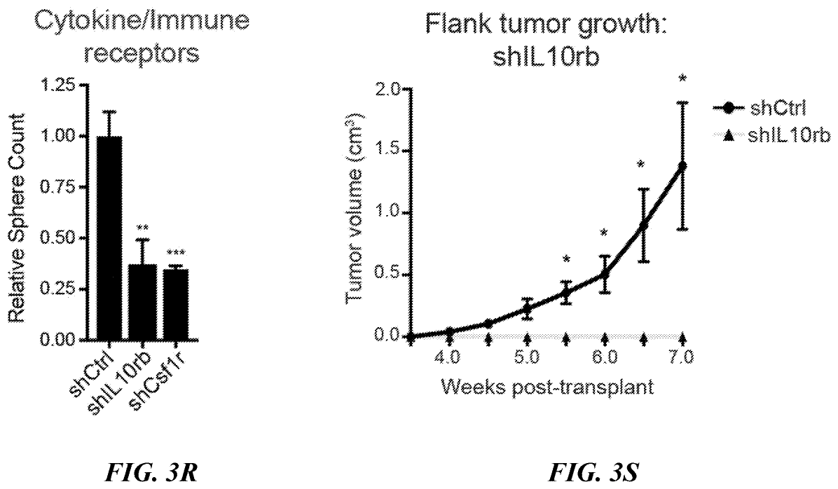

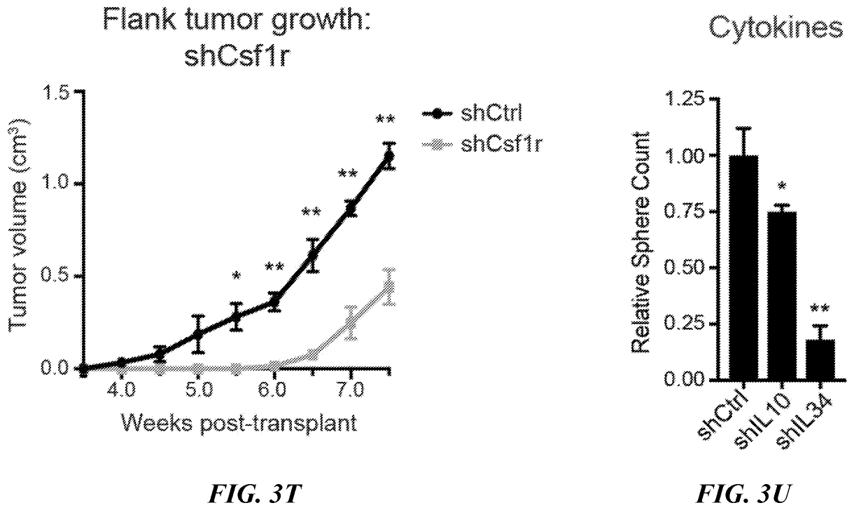

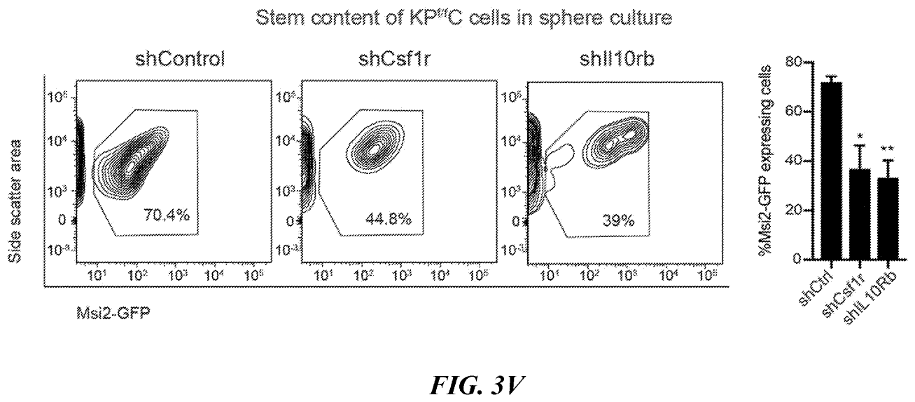

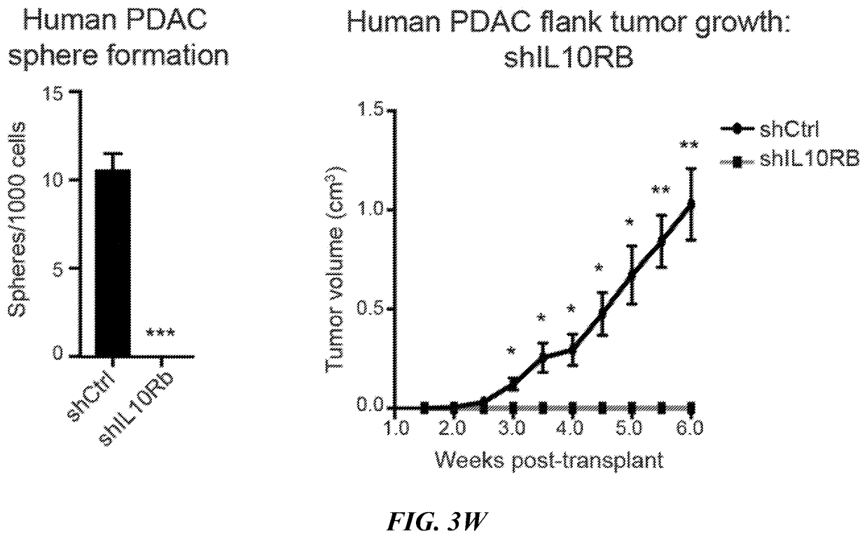

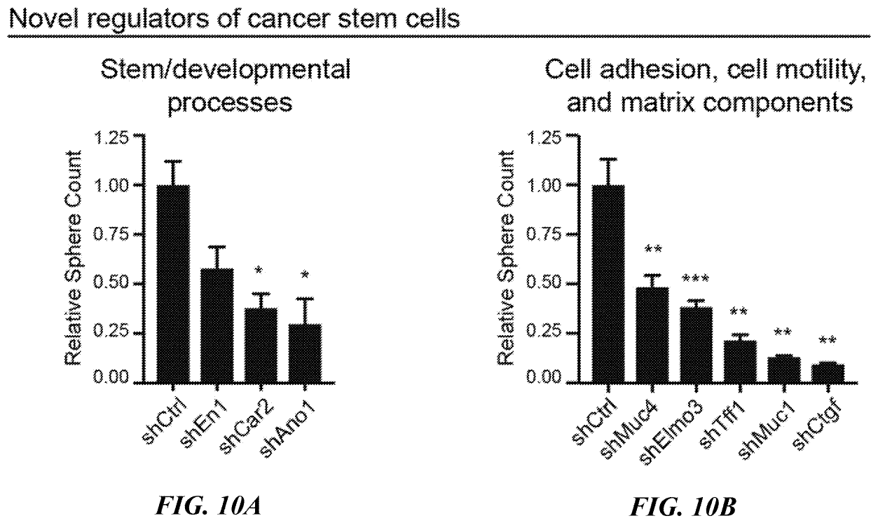

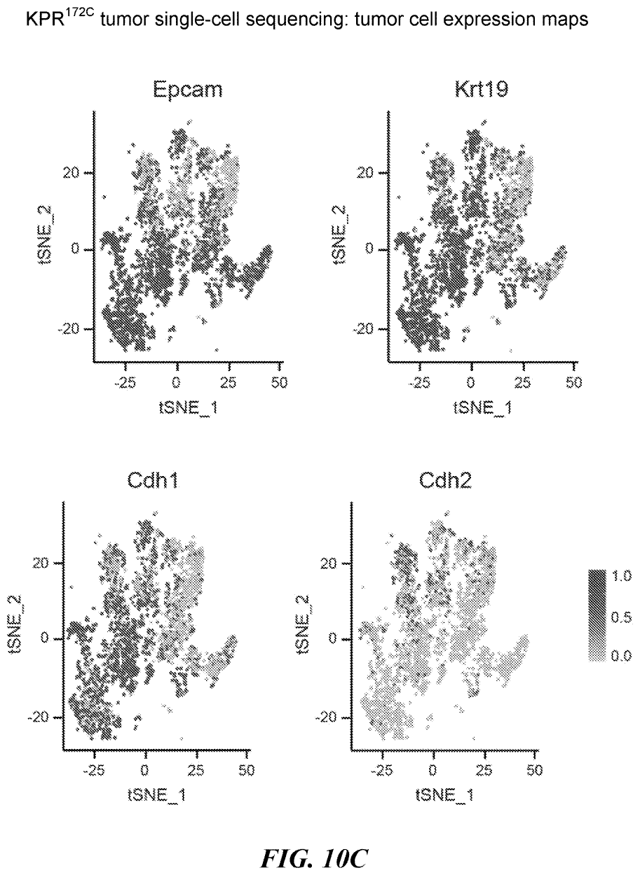

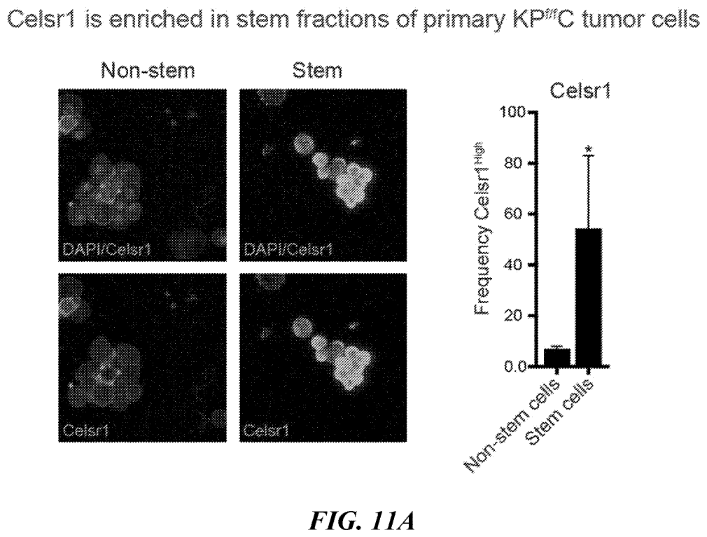

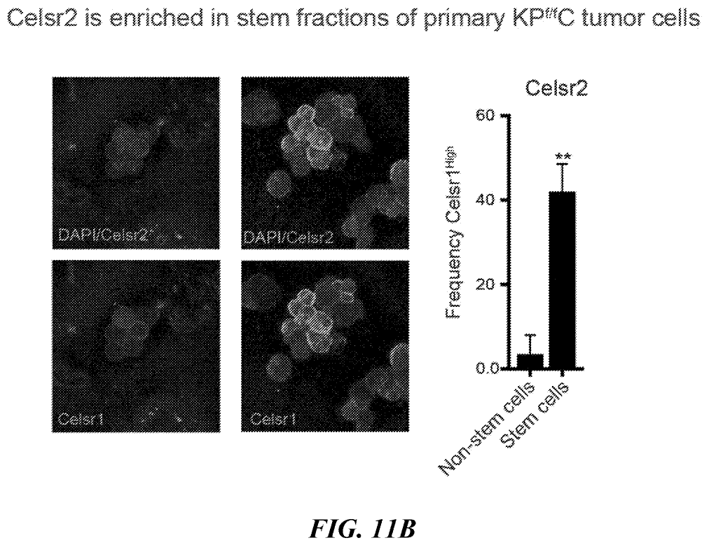

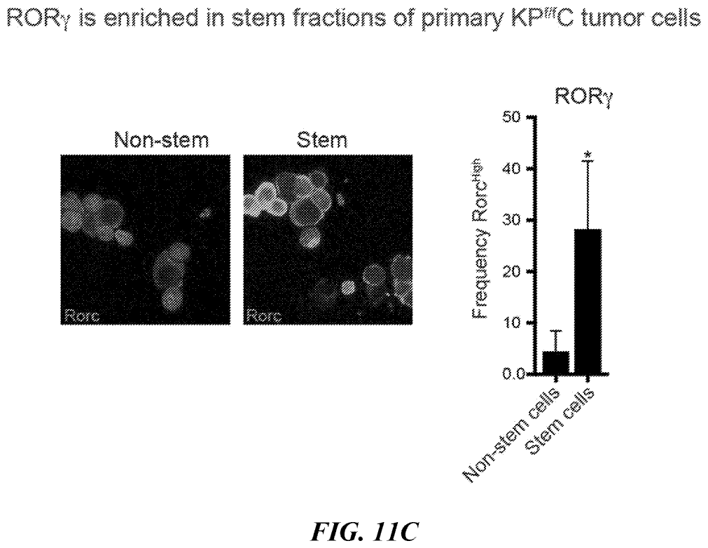

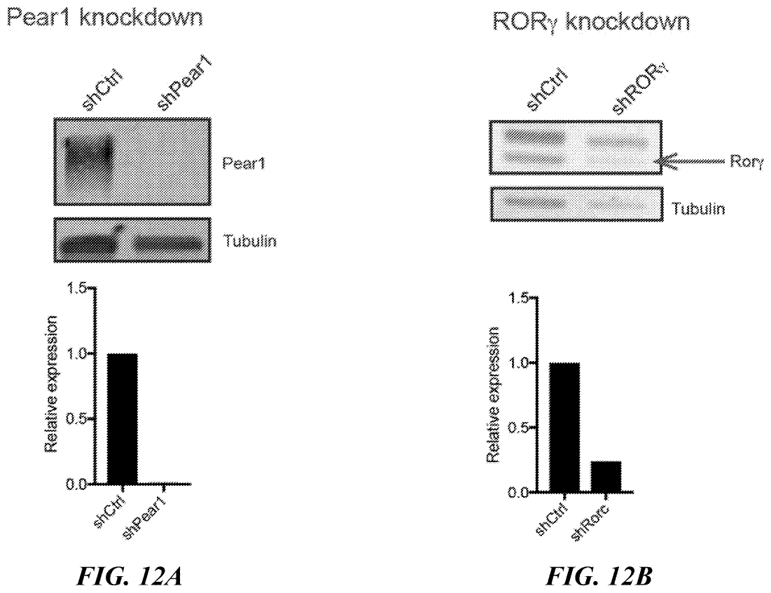

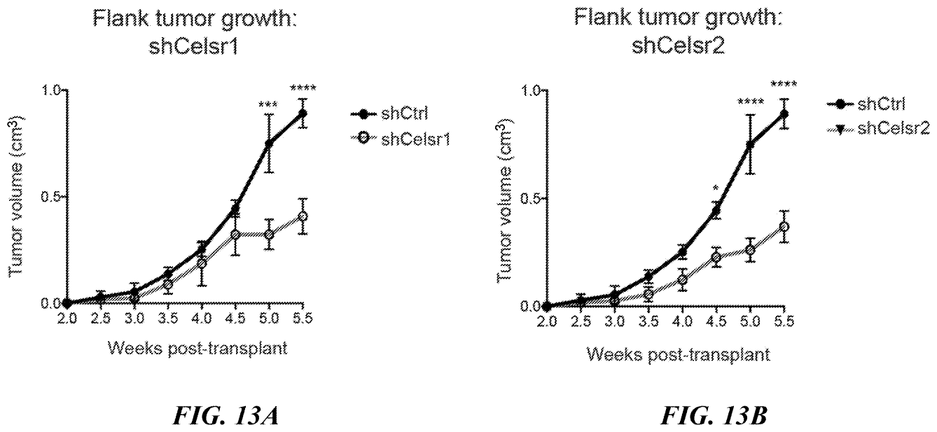

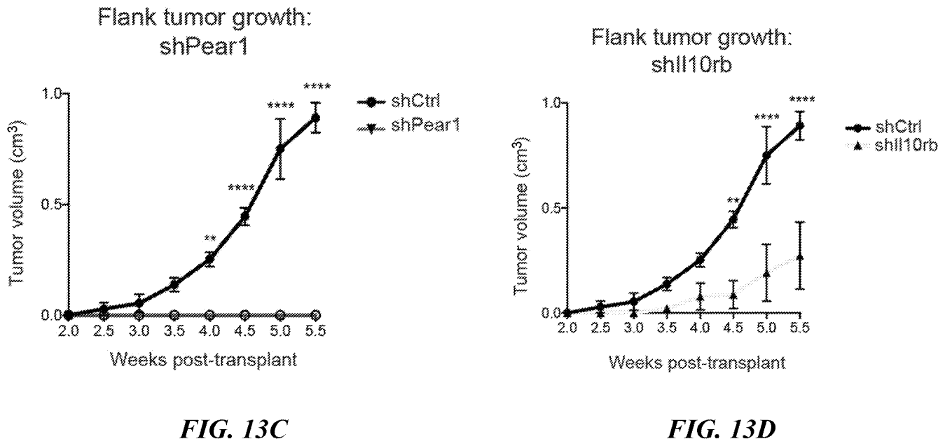

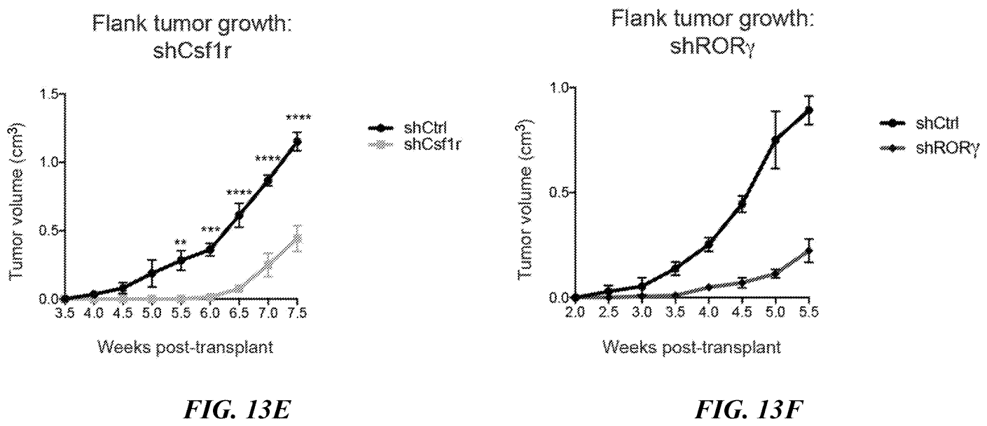

Ultimately the power of such a map is the ability to provide a systems level view of new dependencies. Thus, in some embodiments, the network map was used as a framework to select an integrated gene set based on the transcriptomic, epigenomic and the CRISPR functional genomic analysis (Table 1). Selected genes were subsequently inhibited via viral shRNA delivery into KP^{f/f}C cells, and the impact on pancreatic cancer propagation assessed by stem cell sphere assays in vitro or by tracking tumor growth in vivo. For example, while many genes within the pluripotency and developmental core program were known to be important in pancreatic cancer (e.g., elements of the Wnt, Hedgehog and Hippo pathways), others had not yet been explored, and presented new opportunities for discovery (FIGS. 3A, 3M, 10A) and investigation as novel targets (Table 2). In addition, novel metabolic factors such as Sptssb, a key contributor to sphingolipid metabolism, and Lpin2, an enzyme involved in generation of pro-inflammatory very-low density lipoproteins, were found to be critical new stem cell dependencies, implicating lipid metabolism as a key point of control (FIGS. 3B, 3M). The integrated analysis also identified new gene families as having broad regulatory patterns in pancreatic cancer: thus within the adhesion/cell-matrix core program (FIGS. 3C-3M, 10B), several members of the multiple EGF repeat (MEGF) subfamily of orphan adhesion G protein coupled receptors (8 of 12 preferentially expressed in stem cells, FIG. 3E) such as Celsr1, Celsr2 (FIG. 11A, 11B), and Pear1/Jedi emerged as new regulators of pancreatic cancer propagation as their inhibition (FIG. 12A) potently blocked cancer propagation in vitro and in vivo (FIGS. 3F-3M, independent replicates shown in FIGS. 13A-13C), driven by an increase in cell death and decrease in Msi+ stem cell content (FIGS. 3J, 3K).

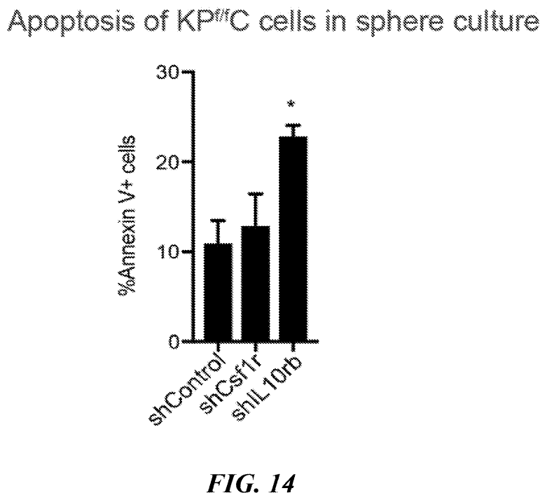

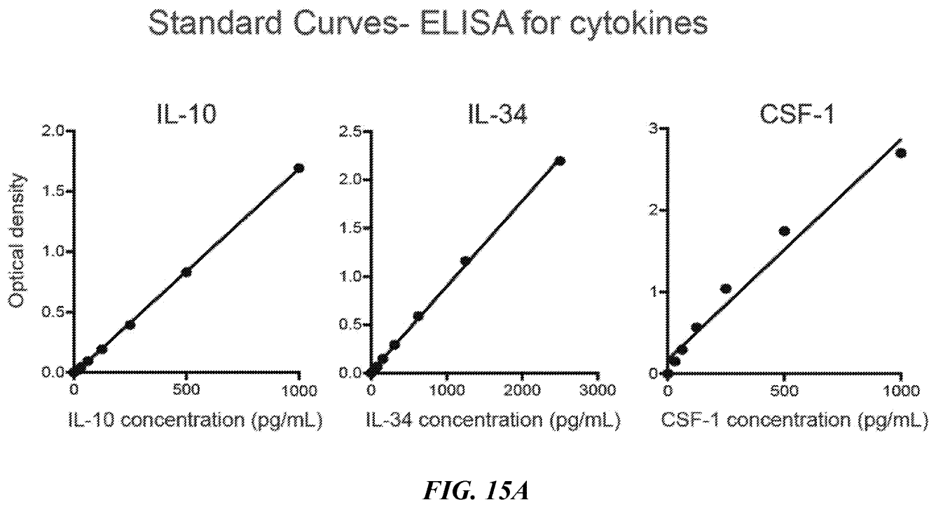

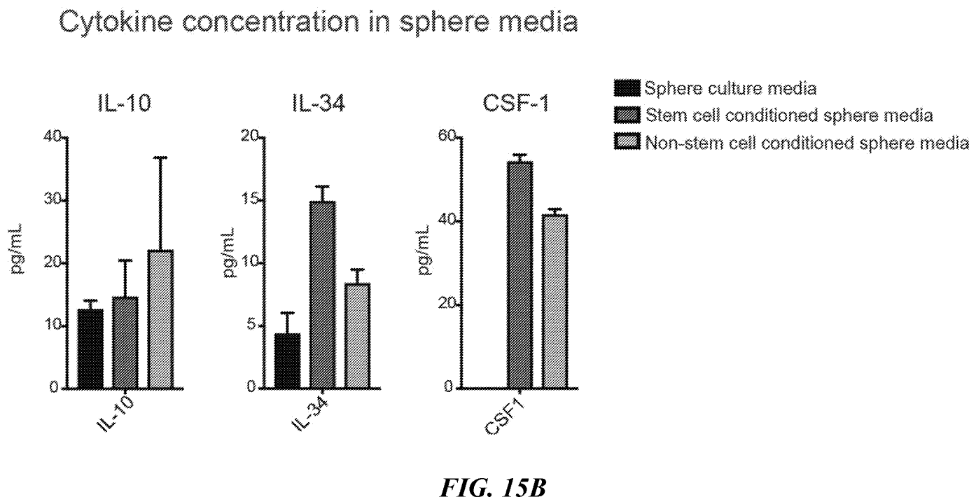

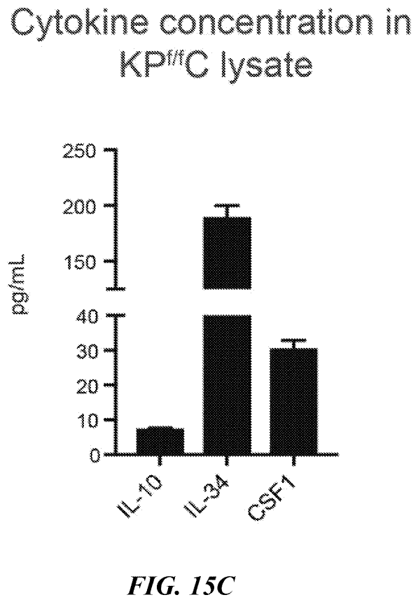

An unexpected discovery from this map was the identification of immune pathways/cytokine signaling as a core program. In line with this, retrospective analysis of the RNA-seq and ChIP-seq analysis revealed that multiple immuno-regulatory cytokine receptors and their associated ligands were expressed in tumor epithelial cells, both in stem and non-stem cells (FIG. 3N). This was of particular interest because many genes associated with this program, such as interleukin-10 (IL-10), interleukin-34 (IL-34) and colony stimulating factor 1 receptor (CSF1R), have been studied primarily in context of the tumor microenvironment, but have not been reported to be produced by, or to functionally impact, pancreatic epithelial cells directly. To more definitively identify whether these cytokines and cytokine receptors were expressed in epithelial cells, single-cell RNA-seq was carried out from KP^{R172H/+}C tumor cells, an independent model of pancreatic cancer. This confirmed the presence of IL10Rβ, IL34 and Csf1R in epithelial tumor cells (FIGS. 3O, 10C). Additionally, co-expression analysis revealed that IL10Rβ, IL34 and Csf1R were expressed in KP^{R172H/+}C stem cells marked by Msi2 expression (FIGS. 3P, 3Q). ShRNA-mediated inhibition of IL10Rβ and CSF1R led to a striking loss of sphere forming capacity (FIG. 3R), and impaired tumor growth and propagation in vivo (FIGS. 3S, 3T, 3W, independent replicates shown in FIGS. 13D, 13E). Inhibition of IL10Rβ and CSF1R may impact tumor growth and propagation by triggering cell death (FIG. 14) and reducing Msi+ stem cell (FIG. 3V). The fact that shRNA mediated inhibition of the ligands, IL10 and IL34, had a similar impact suggested ligand dependent activity (FIG. 3U). Consistent with this, IL-10, CSF and IL-34 were expressed by epithelial cells (FIG. 15) though other sources of these ligands are likely to be present in vivo. Collectively, these findings demonstrate an intriguing orthogonal co-option of inflammatory mediators by pancreatic cancer stem cells and suggest that agents that modulate cytokine networks may directly impact their function in pancreatic cancer propagation.

D. RORγ, a Mediator of T Cell Fate, is a Critical Dependency in Pancreatic Cancer

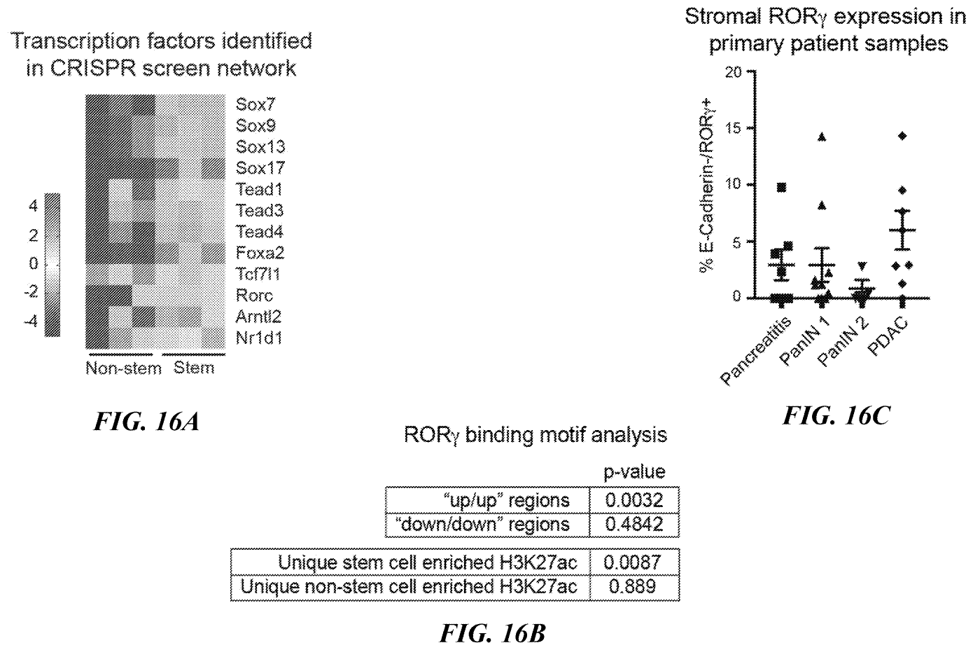

In some embodiments, to understand how the gene networks defined above are controlled, transcription factors were focused on because of their powerful role in regulating broad hierarchical programs key to cell fate and identity. Of the 53 transcription factors identified within the map, 12 were found to be enriched in stem cells by transcriptomic and epigenetic parameters (FIG. 16A), and included several pioneer factors known to promote tumorigenesis, such as Sox9 and Foxa2. Among transcription factors with no known role in pancreatic cancer (Arntl2, Nr1d1, and RORγ), only RORγ was potentially actionable with clinical-grade antagonists available. Importantly, at the molecular level, motif enrichment analysis revealed that RORγ sites were preferentially enriched in chromatin regions uniquely open in stem cells relative to non-stem cells (p=0.0087, FIG. 16B) and in open chromatin regions that corresponded with high gene expression in stem cells (p=0.0032, FIG. 16B). These findings are consistent with the possibility that RORγ may be important in controlling gene expression programs that are important for defining a stem cell state in pancreatic cancer.

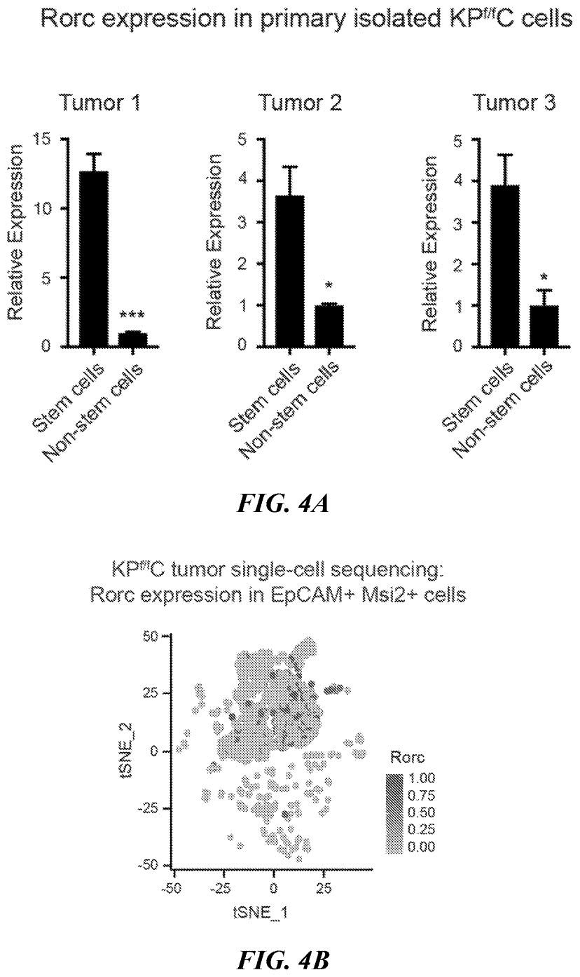

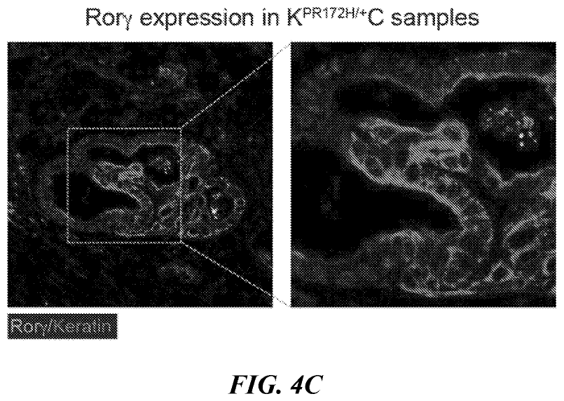

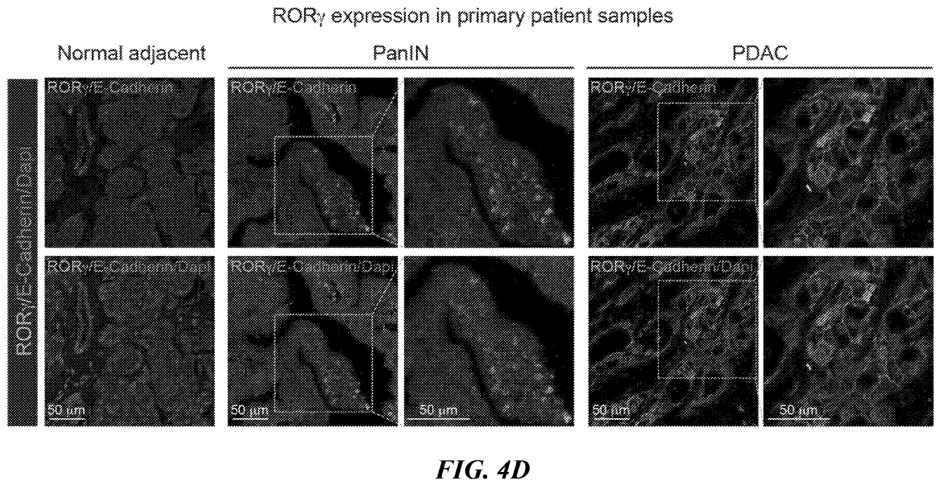

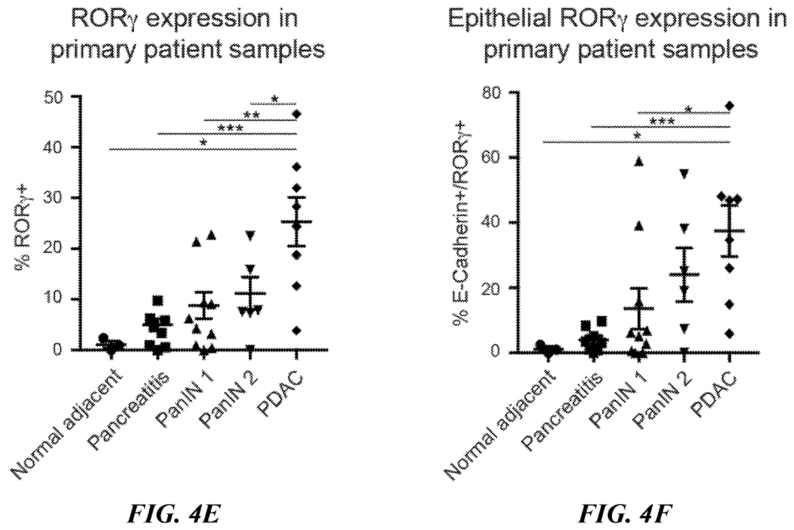

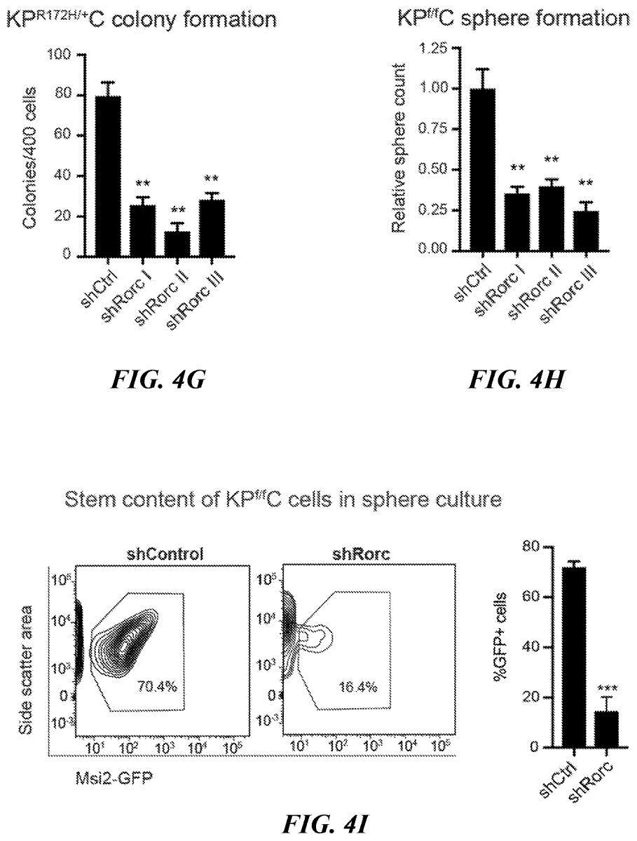

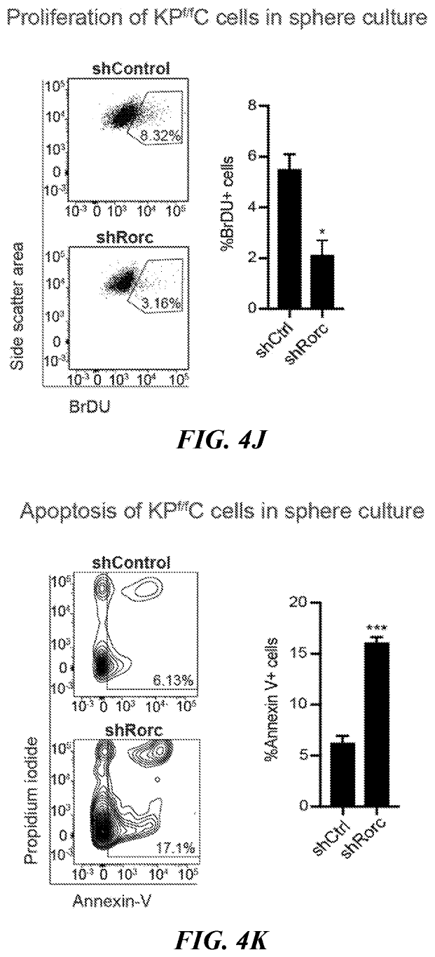

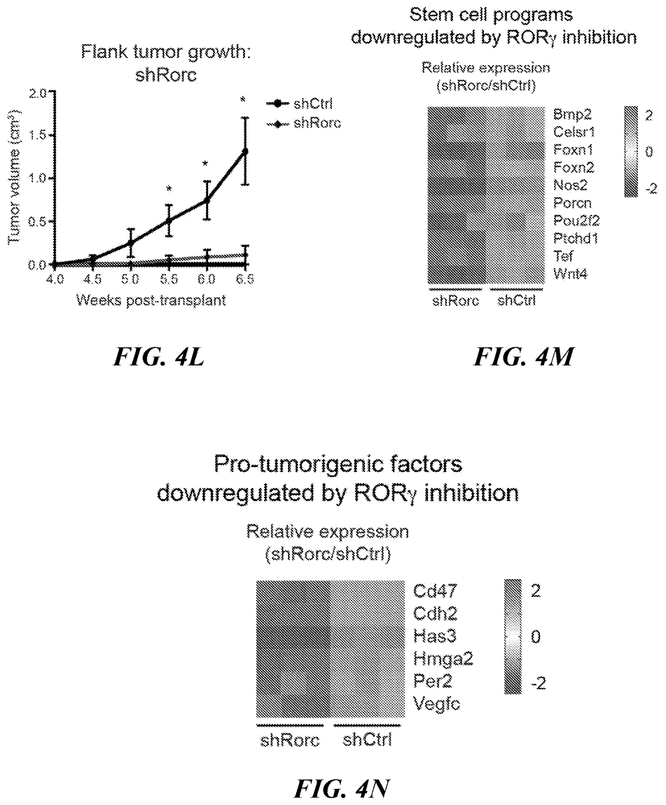

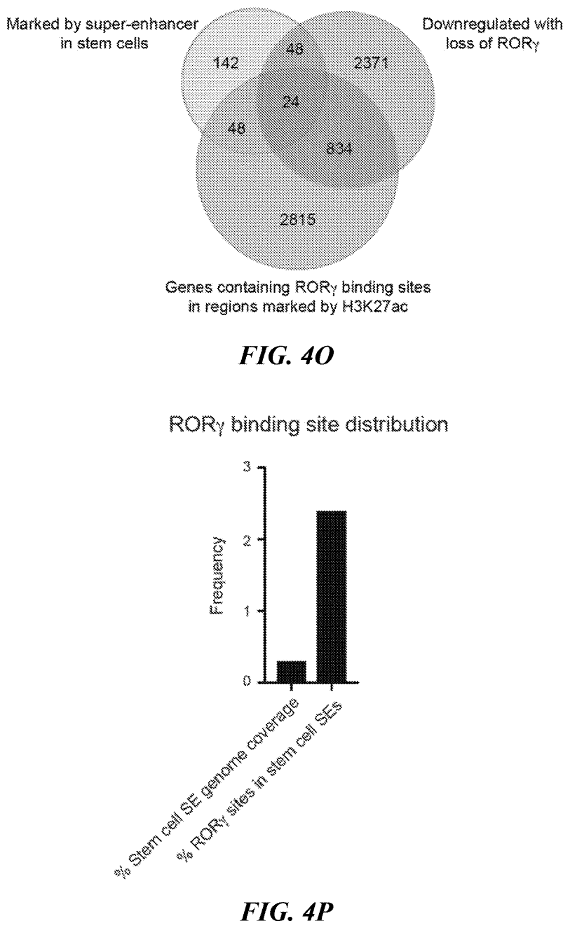

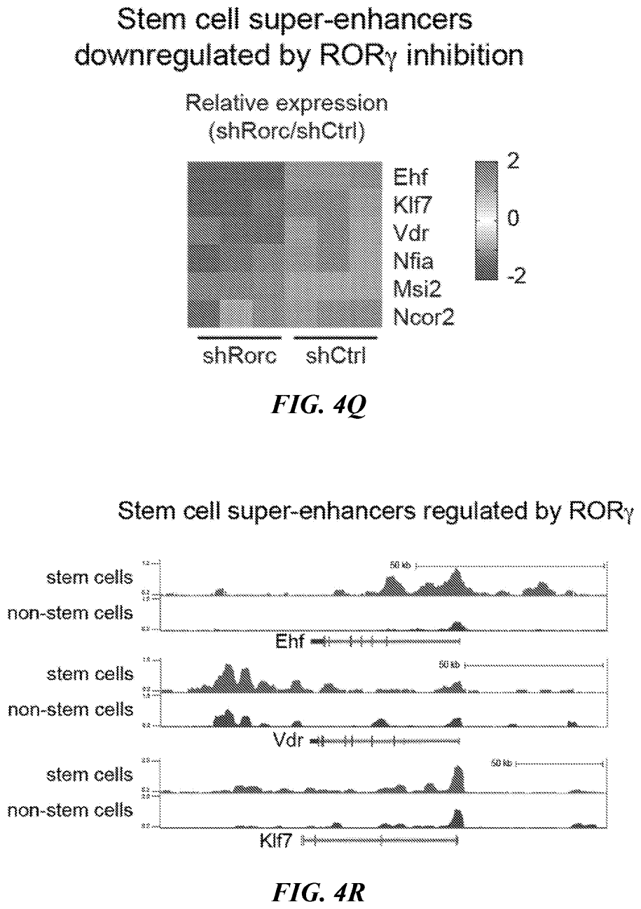

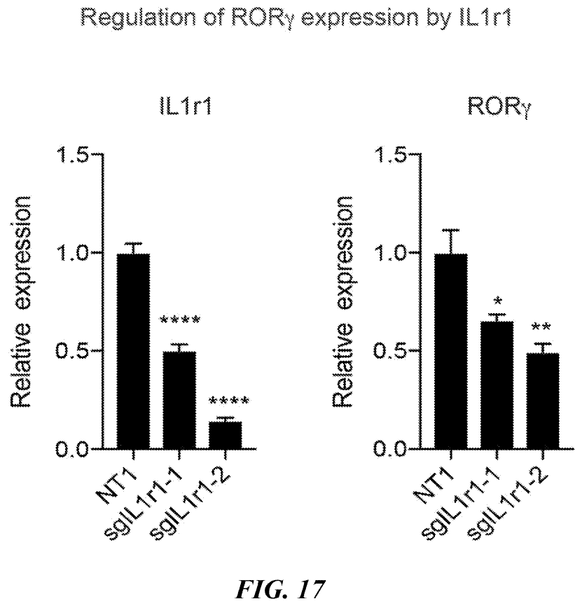

RORγ was an unanticipated dependency as it is a nuclear hormone receptor that has been predominantly studied in the context of Th17 cell differentiation as well as lipid and glucose metabolism in the context of circadian rhythm. Consistent with this, it mapped to both the hijacked cytokine signaling/immune subnetwork and the nuclear receptor/metabolism subnetwork (FIGS. 2E, 2cF). RORγ expression was low in normal murine pancreas but increased in KP^{f/f}C tumors; within primary epithelial cells, RORγ was enriched in stem cell populations, and expressed at low levels in non-stem cells both at the RNA and protein levels (FIGS. 4A, 11C), and expressed in EpCAM+Msi+ cells by single cell RNA Seq analysis (FIG. 4B). RORγ was also expressed in KP^{R172H/+}C tumor cells by immunohistochemistry (FIG. 4C) suggesting that it was not limited to one particular model of pancreatic cancer. Importantly, RORγ expression in mouse models was predictive of expression in human pancreatic cancer. Thus, while RORγ expression was low in normal human pancreas and in pancreatitis, its expression increased significantly in epithelial tumor cells with disease progression (FIGS. 4D-4F, 16C). Functionally shRNA-mediated knockdown (FIG. 12B) confirmed the role of RORγ identified by the genetic CRISPR-based screen as it leads to a decrease in stem cell sphere formation in both KP^{R172H/+}C and KP^{f/f}C cells (FIGS. 4G-4H). RORγ knockdown led to a 3-fold increase in cell death (Annexin) and proliferation (BrDU) and a consequent 5-fold decrease in Msi+ stem cells in Msi reporter KP^{f/f}C spheres (FIGS. 4I-4K). Importantly, KP^{f/f}C tumor cells lacking RORγ showed a striking defect in tumor initiation and propagation in vivo, with a 11-fold reduction in final tumor volume (FIG. 4L, Independent replicates shown in FIG. 13F). To test if pathways regulating RORγ are important in pancreatic cancer, URI was deleted in KP^{f/f}C cells, which resulted in a 50% reduction in RORγ expression (FIG. 17). This suggested that the mechanism by which RORγ is regulated in pancreatic cancer cells may be shared, at least in part, with the mechanism by which RORγ is regulated in Th17 cells.

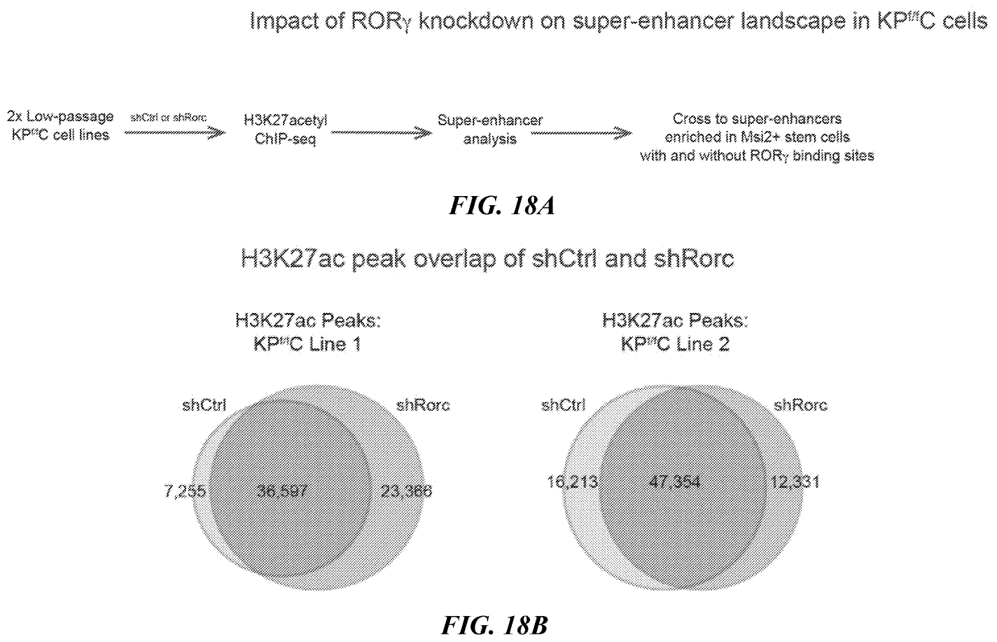

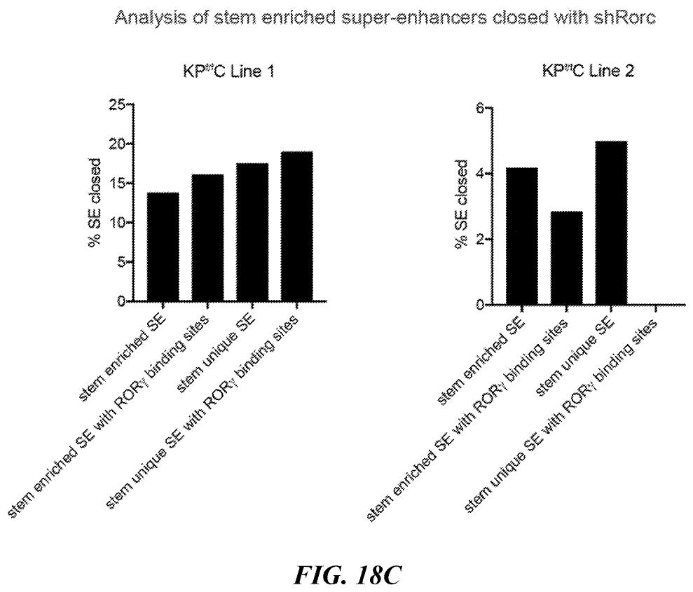

To define the transcriptional programs RORγ controls in pancreatic cancer cells, a combination of ChIP-seq and RNA-seq was used to map the molecular changes triggered by RORγ loss. Loss of RORγ led to extensive modifications in transcriptional programs key to driving cancer growth, including stem cell signals such as Wnt, BMP, and Fox (FIG. 4M), and signals implicated in tumorigenesis such as Hmga2 (FIG. 4N). Interestingly, this transcriptional analysis showed that 28% of stem cell super-enhancer linked genes were downregulated in cells lacking RORγ (FIG. 4O). Consistent with this, ChIP-seq analysis of active chromatin regions identified RORγ binding sites as disproportionately present in stem cell super-enhancers (FIG. 4P). Additional super-enhancer-associated stem cell genes regulated by RORγ included Msi2, Klf7 and Ehf (FIGS. 4Q-4R), potent oncogenic signals that can control cell fate. Mechanistically, loss of RORγ did not markedly impact the stem cell super-enhancer landscape in two independent KP^{f/f}C derived lines (FIG. 18), suggesting that it may instead bind a preexisting landscape to preferentially impact transcriptional changes. These data collectively suggest that RORγ is an upstream regulator of a powerful oncogenic effector network controlled by super-enhancers in pancreatic cancer stem cells.

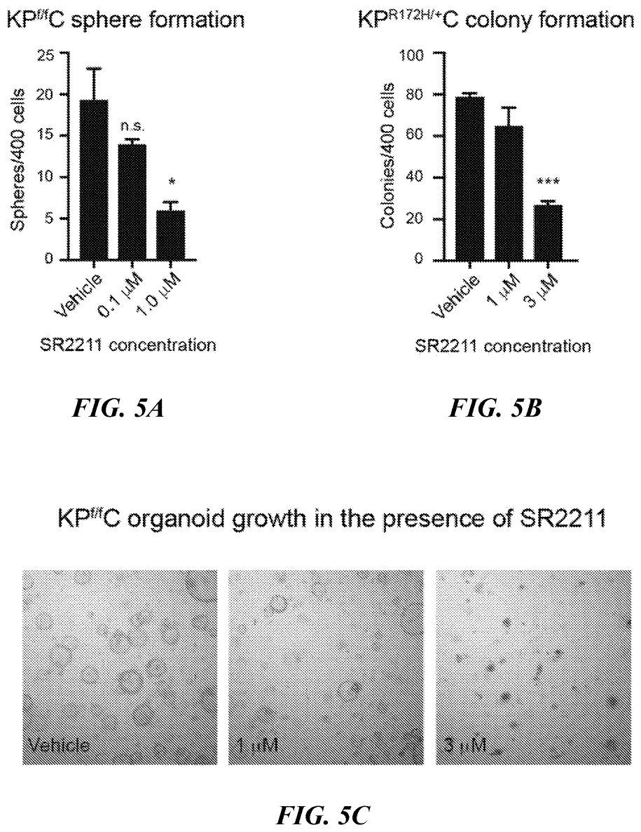

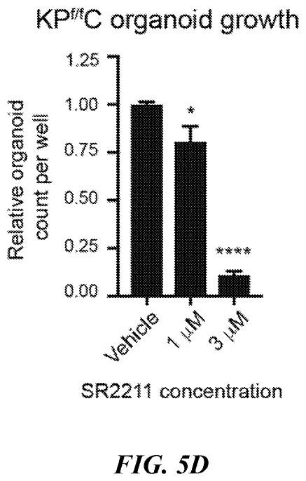

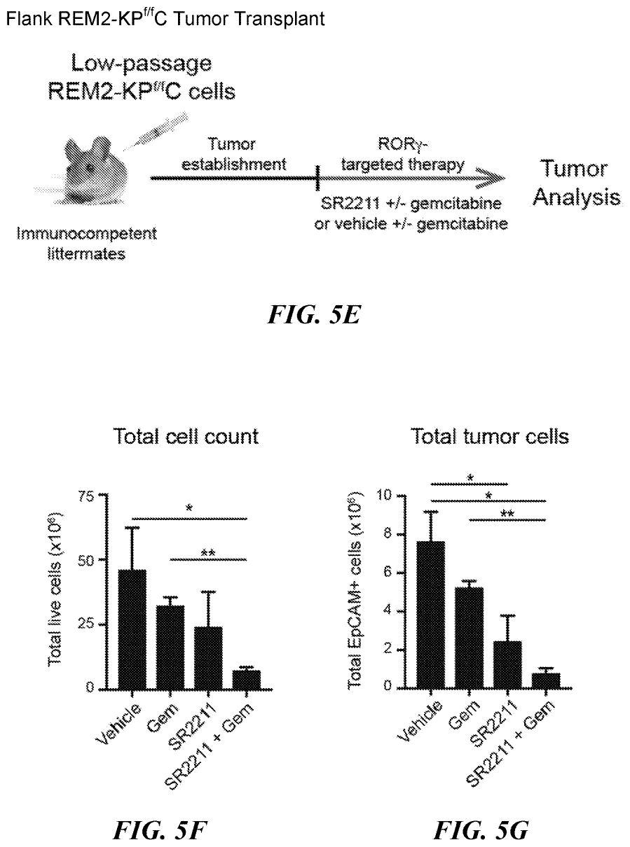

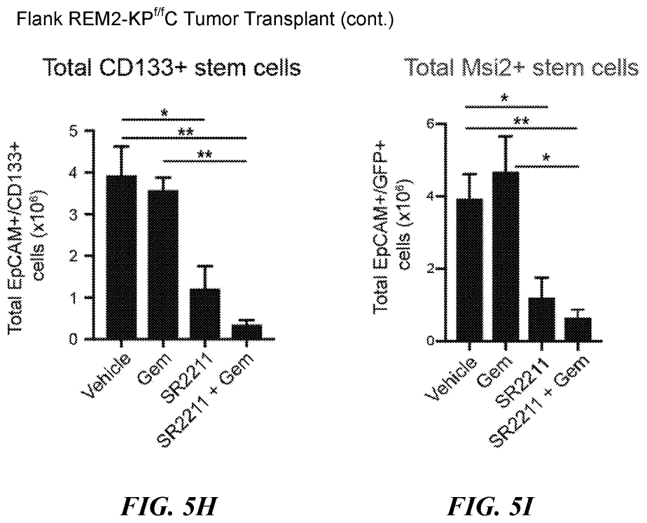

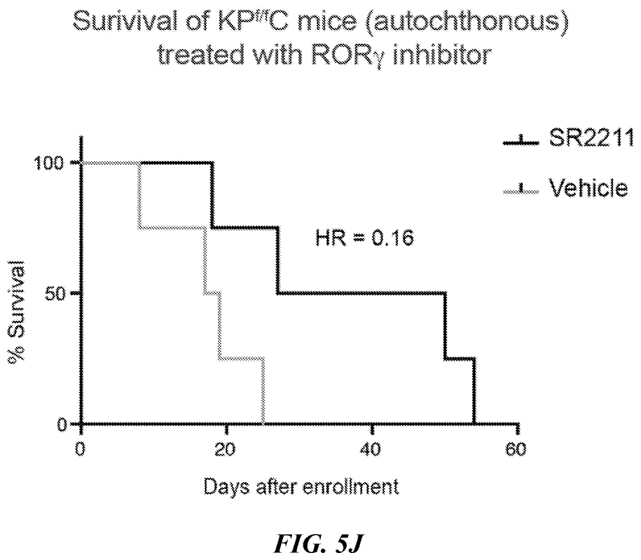

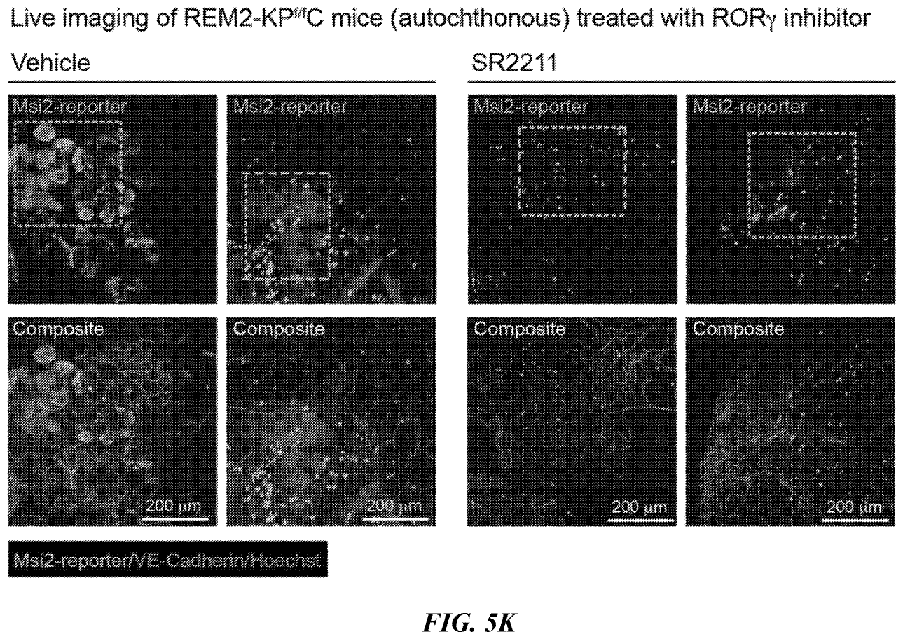

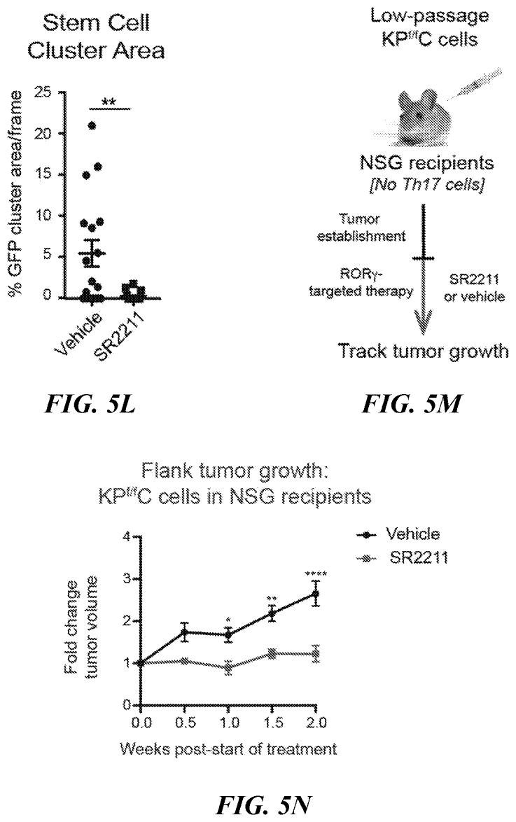

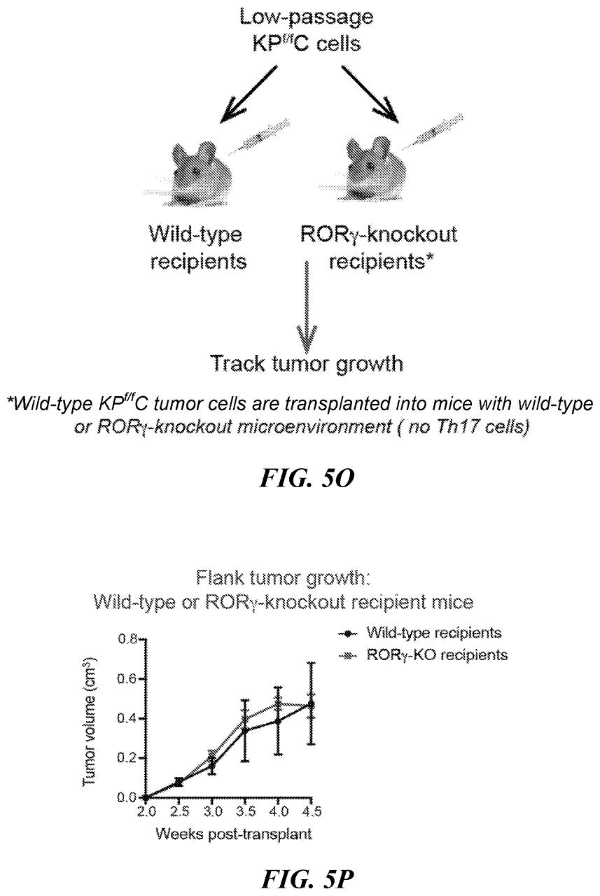

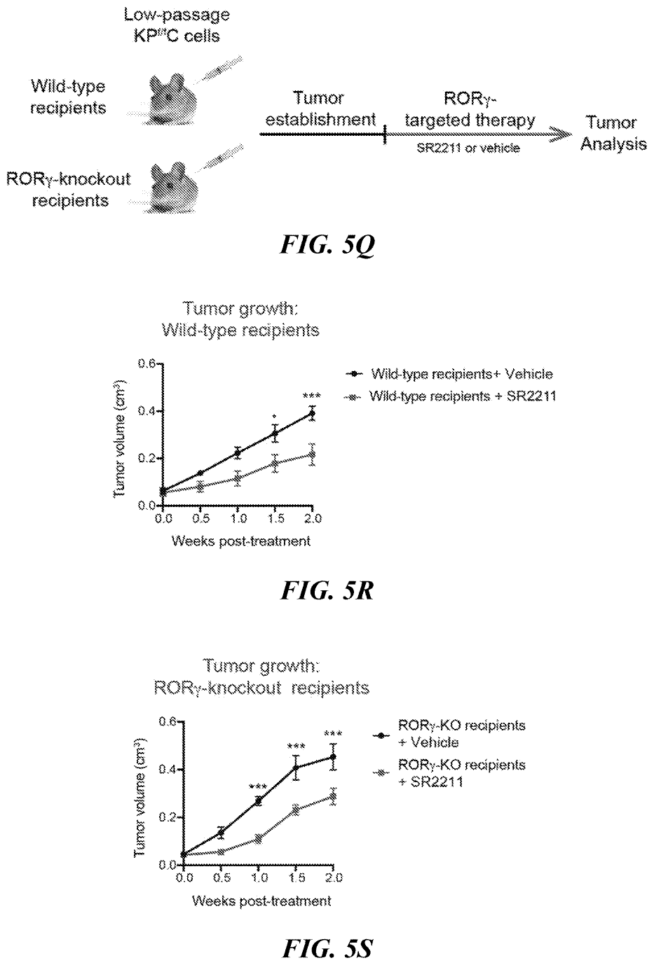

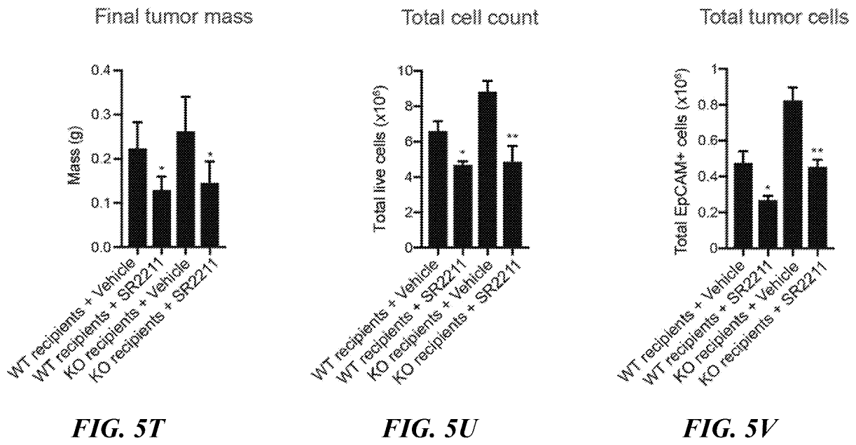

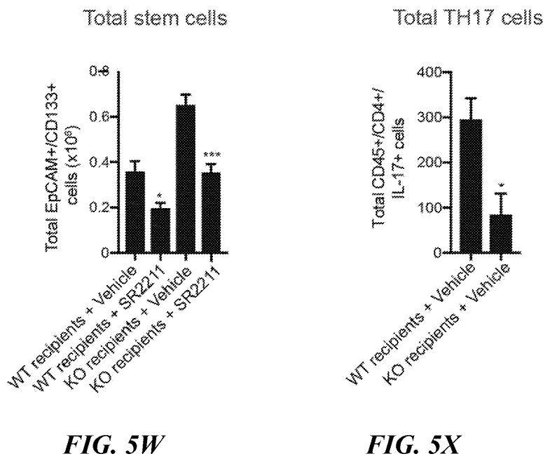

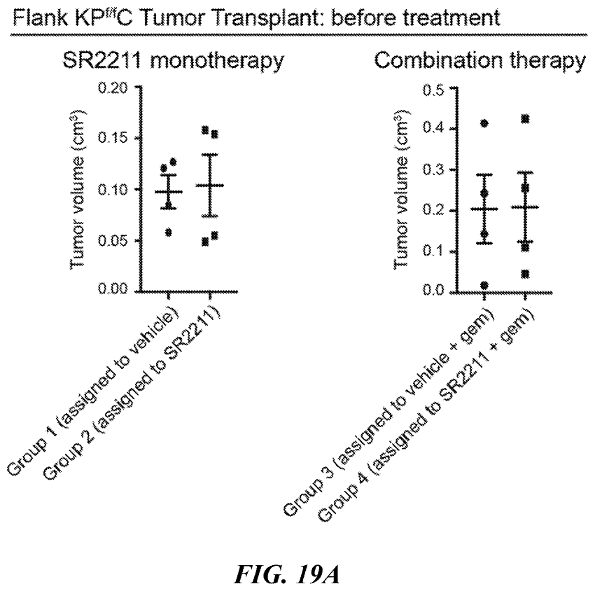

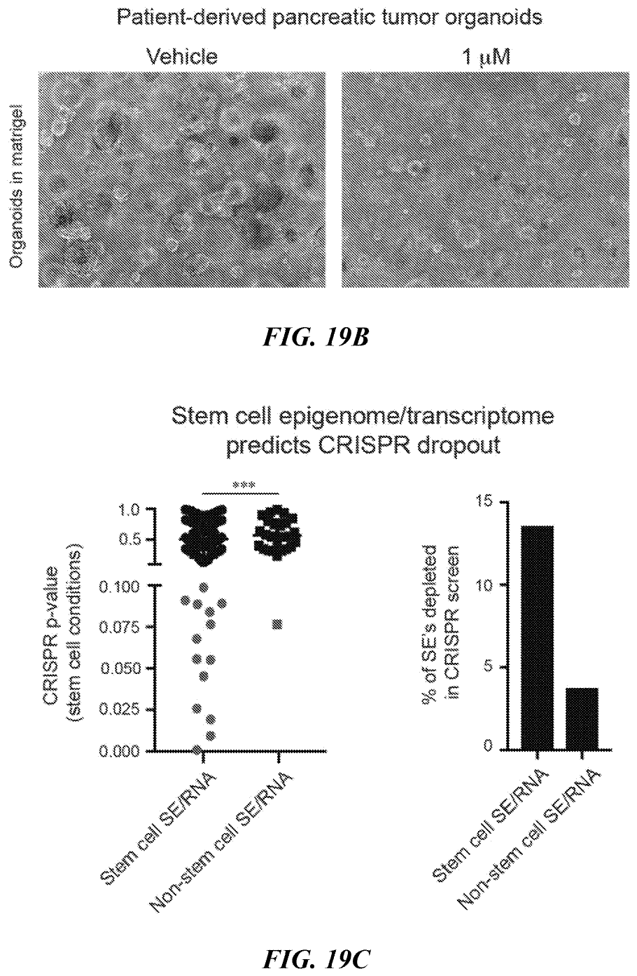

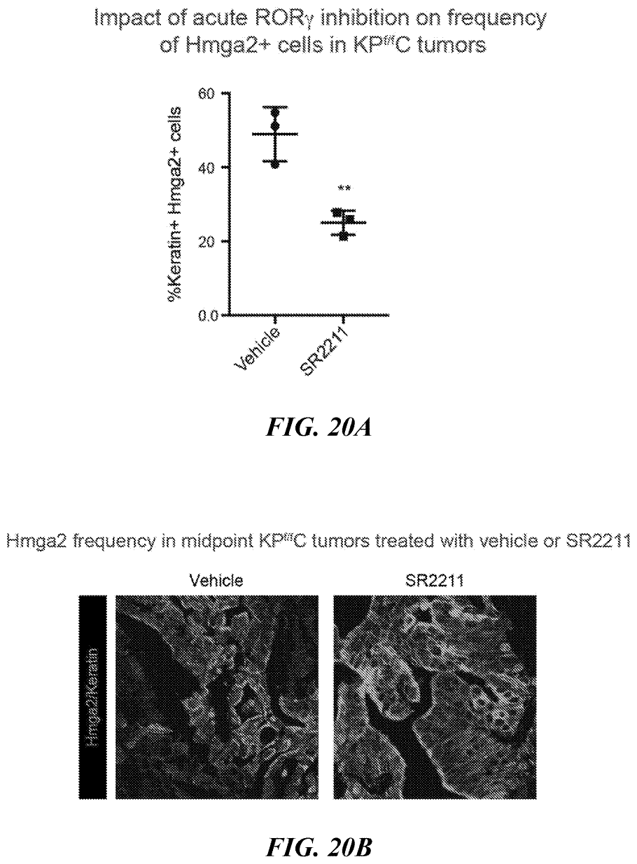

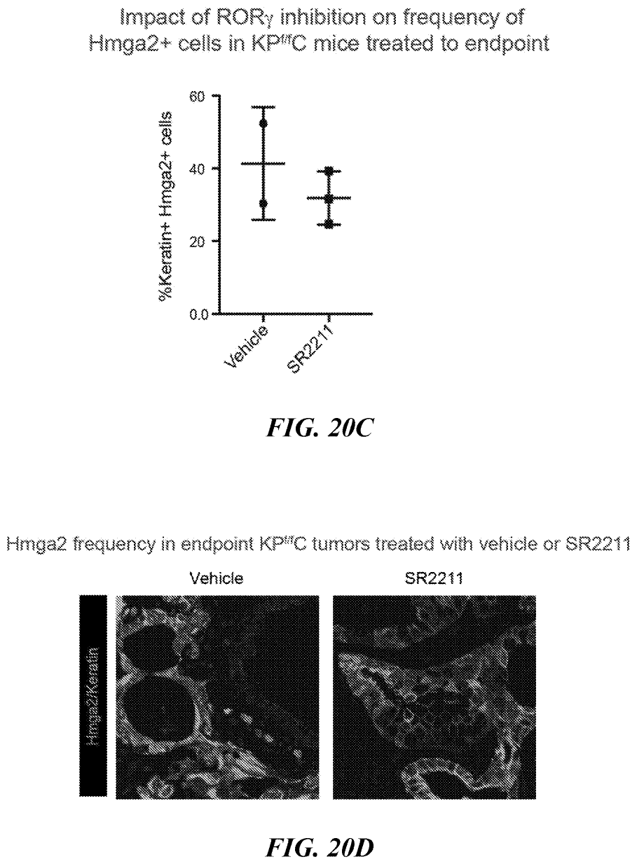

The finding that RORγ is a key dependency in pancreatic cancer was important, as multiple inhibitors have been developed to target this pathway in autoimmune disease. Pharmacologic blockade of RORγ using the inverse agonist SR2211 decreased sphere and organoid formation in both KP^{f/f}C and KP^{R172H/+}C cells (FIGS. 5A-5D). To assess the impact of the inhibitor in vivo, SR2211 alone or in combination with gemcitabine was delivered to immunocompetent mice bearing established flank tumors derived from KP^{f/f}C cells (FIGS. 5E, 19A). SR2211 significantly reduced the growth of KP^{f/f}C derived flank tumors as a single agent (FIGS. 5F-5G). Importantly, while gemcitabine alone had no impact on cancer stem cell burden, SR2211 alone triggered a 3-fold depletion in CD133+ and Msi+ cells, and in combination with gemcitabine led to an 11-fold depletion of CD133+ and 6-fold depletion of Msi2+ cells (FIGS. 5H, 5I). This suggests the possibility that SR2211 can eradicate chemotherapy resistant cells (FIGS. 5H, 5I). Finally, to assess any impact on survival, the RORγ inhibitor was delivered in autochthonous, tumor-bearing KP^{f/f}C mice; while none of the vehicle-treated mice were alive 25 days after the initiation of treatment, 75% of mice that received SR2211 were still alive at this point and 50% were alive even at 45 days after treatment initiation. Further, the median survival was 18 days for vehicle-treated mice and 38.5 days for SR2211-treated mice; SR2211 also led to a 6-fold decreased risk of death (FIG. 5J, Hazard Ratio=0.16). Hmga2, identified originally from the RNA-Seq as a downstream target, was downregulated in pancreatic epithelial cells following SR2211 delivery in vivo, suggesting effective target engagement at least at mid-point during the treatment regimen; however in tumors from end stage mice Hmga2 expression was similar to that in control tumors, indicating a potential loss of target engagement, or activation of compensatory pathways (FIG. 20). Collectively, these data show that pancreatic cancer stem cells are profoundly dependent on RORγ expression and suggest that its inhibition may lead to a significant improvement in disease control. Further, the fact that its impact on tumor burden was amplified several fold when combined with gemcitabine suggests that it may synergize with chemotherapy to more effectively control tumors that are normally refractory to therapy.

To visualize whether RORγ blockade impacts tumor progression by targeting stem cells, SR2211 was delivered in REM2-KP^{f/f}C mice with late-stage autochthonous tumors and responses were subsequently tracked via live imaging. In vehicle-treated mice, large stem cell clusters could be readily identified throughout the tumor based on GFP expression driven by the Msi reporter (FIGS. 5K-5L). SR2211 led to a striking depletion of the majority of large stem cell clusters within 1 week of treatment (FIGS. 5K-5L), with no increased necrosis observed in surrounding tissues. This provided a unique spatiotemporal view of the impact of RORγ signal inhibition in vivo and suggested that stem cell depletion is an early consequence of RORγ blockade.

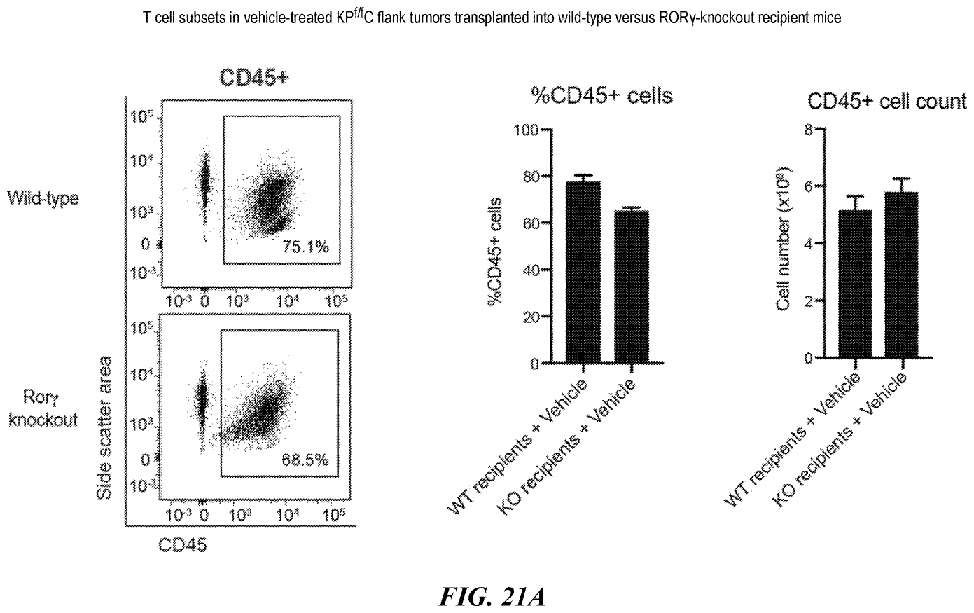

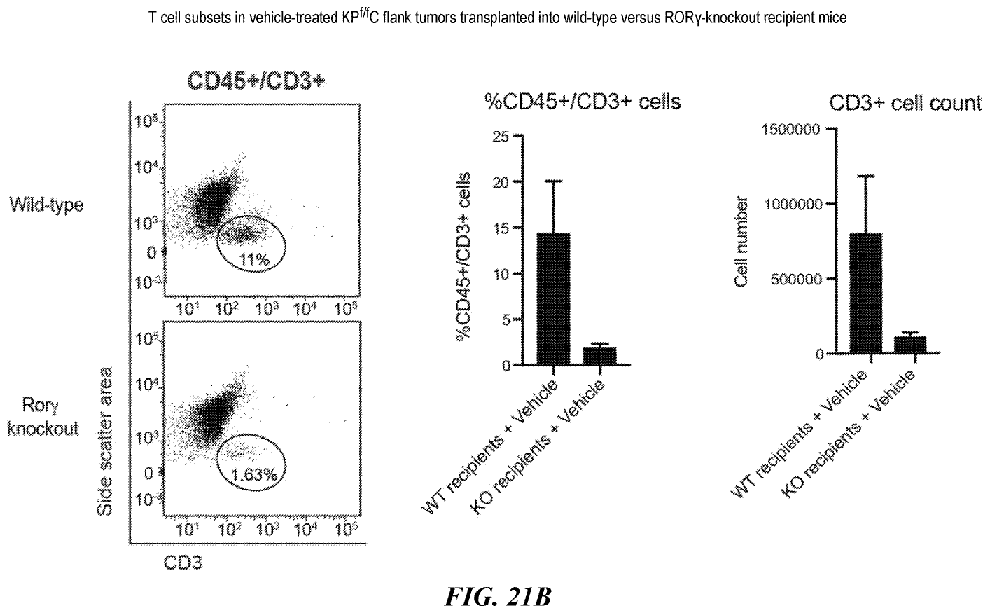

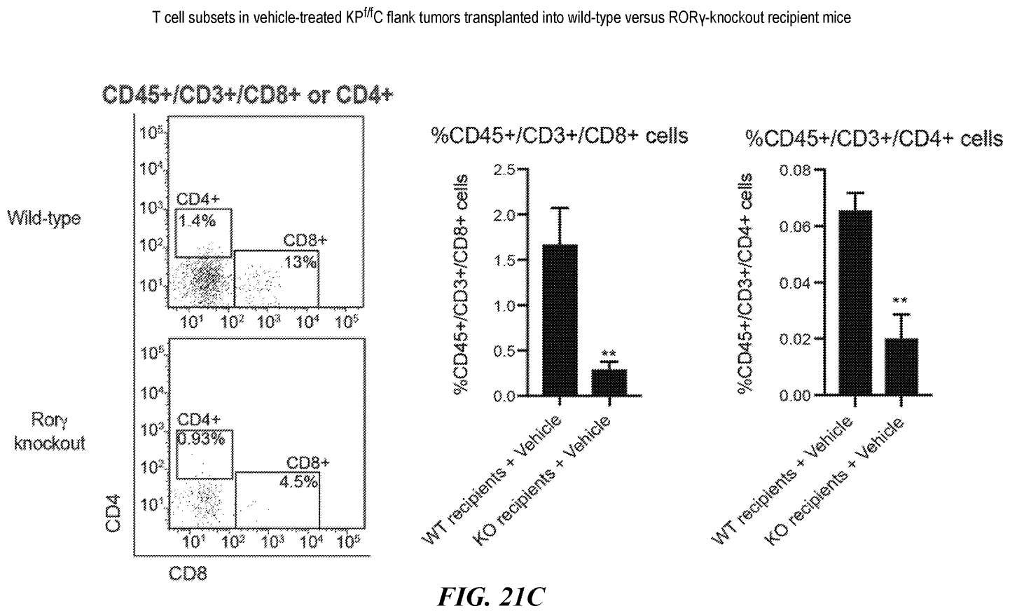

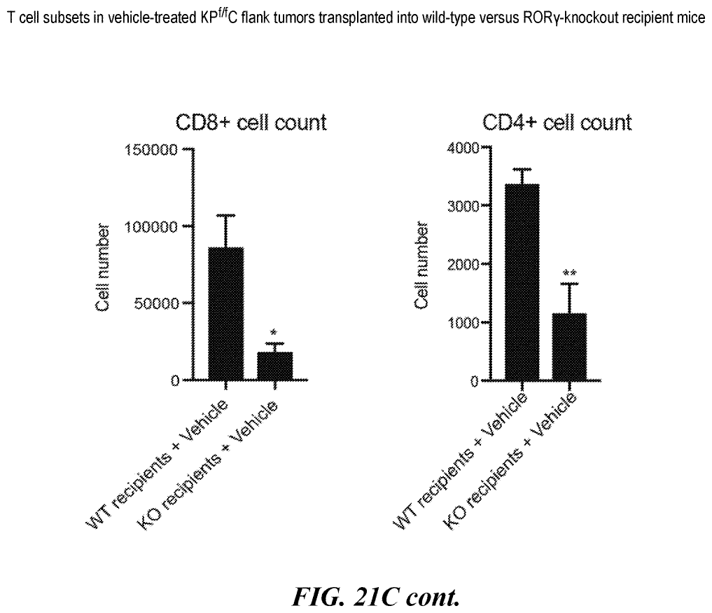

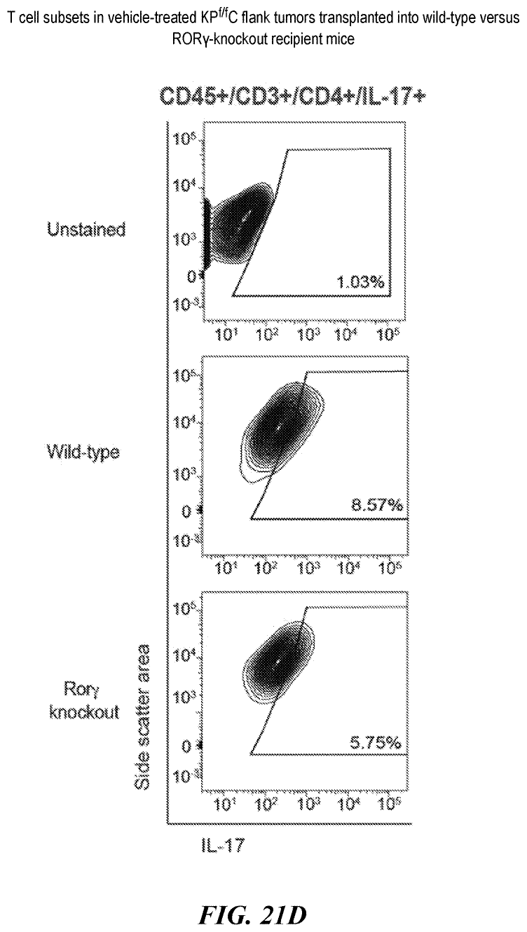

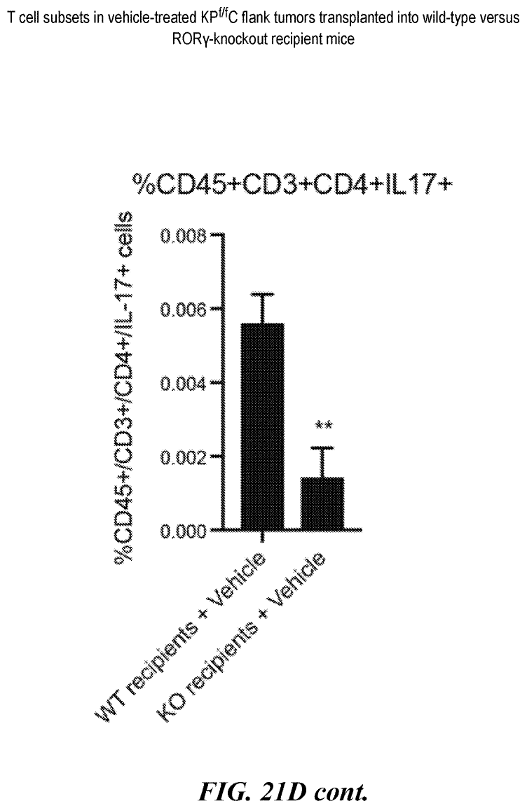

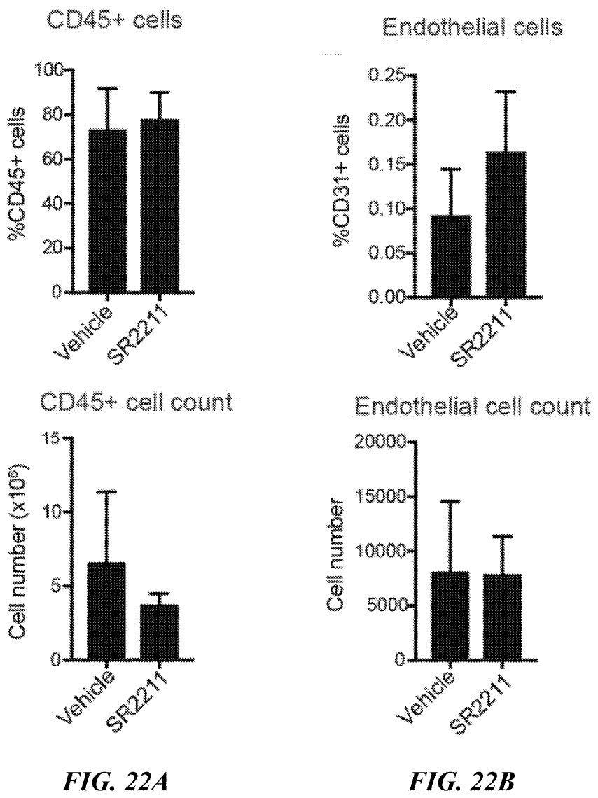

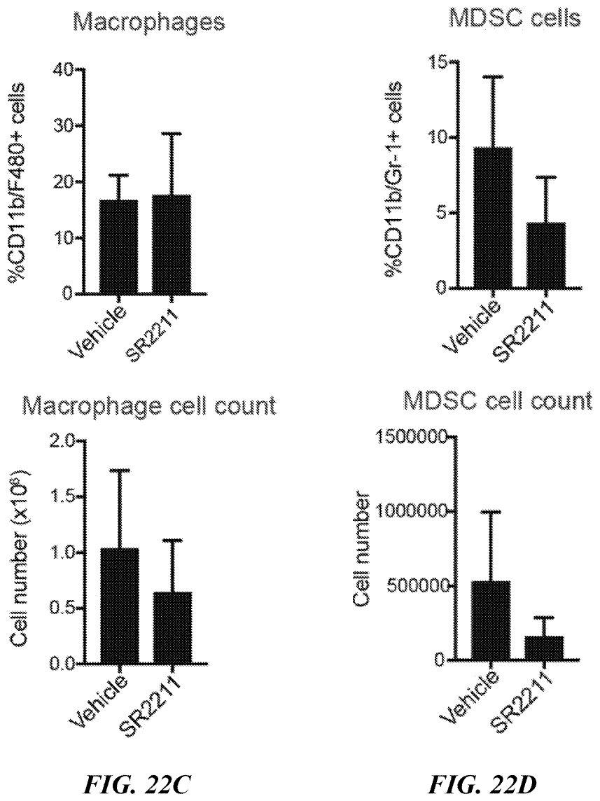

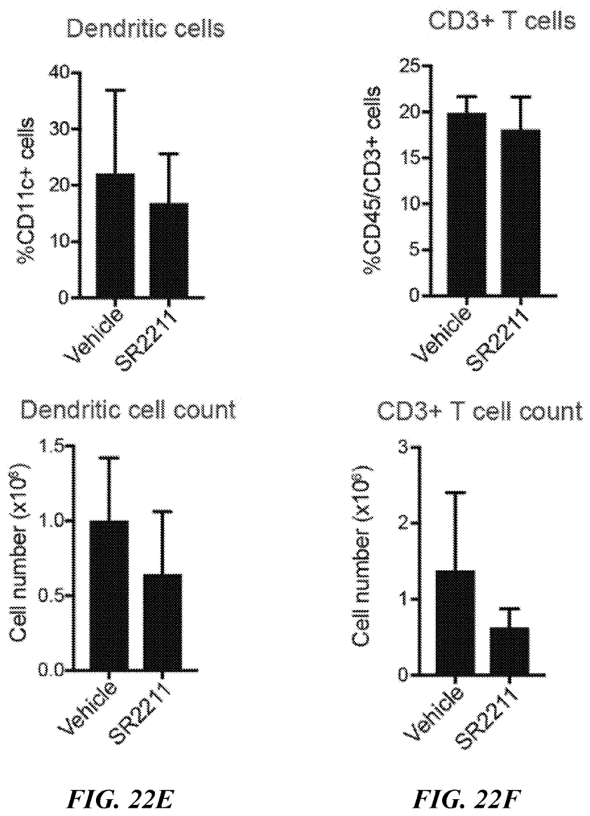

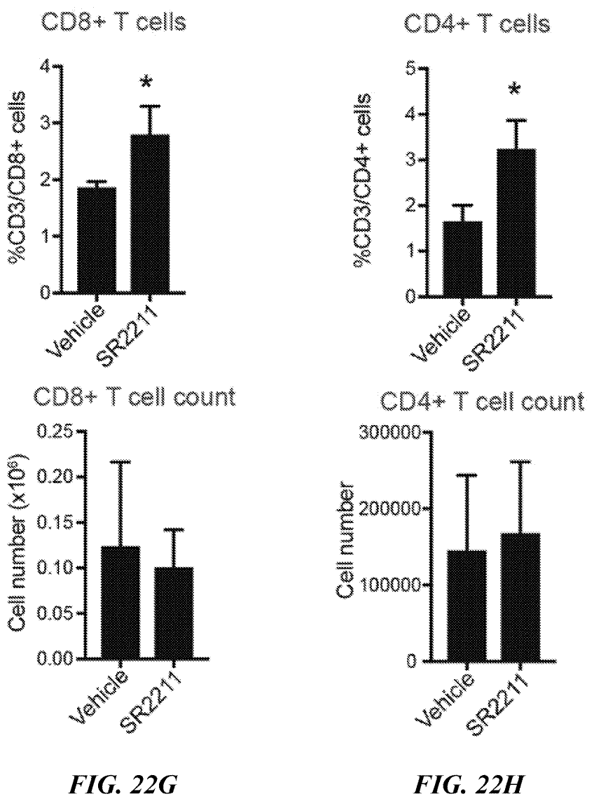

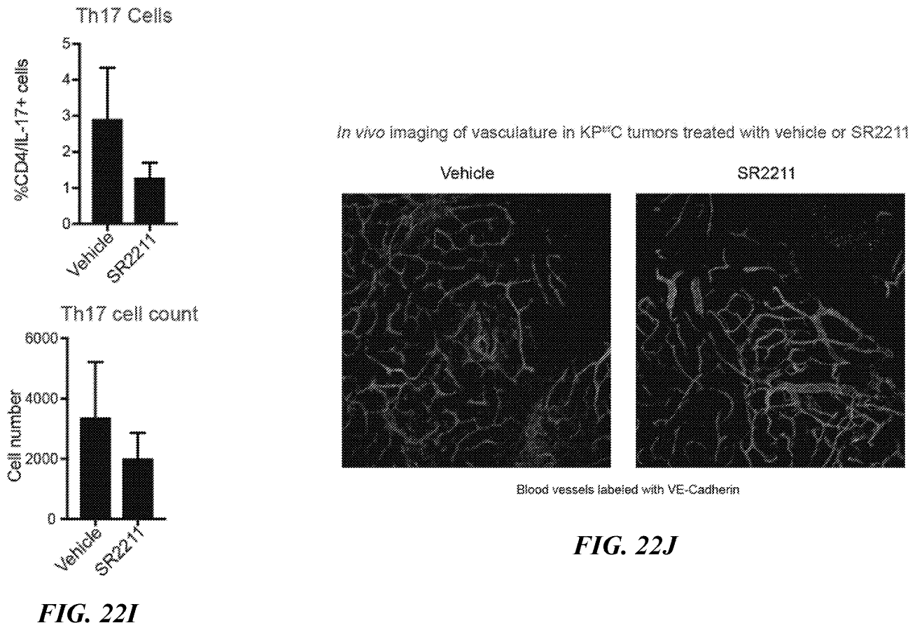

Since treatment with the inhibitor in immunocompetent mice or in patients in vivo could have an impact on both cancer cells and immune cells, such as Th17 cells, the effect of SR2211 was tested in immunocompromised mice. As shown in FIGS. 5M-5N, SR2211 significantly impacted growth of KP^{f/f}C tumors in an immunodeficient background, suggesting that inflammatory T cells were not necessary for its effect. To test whether RORγ inhibition in an immunocompetent setting could slow tumor growth by influencing Th17 cells, chimeric mice were generated. Wild type tumors transplanted into wild type or RORγ null recipients grew equivalently (FIGS. 5O-5P), suggesting that loss of RORγ in only the immune cells and micro-environment (as in the knockout recipients) had no detectable impact on tumor growth. Finally, SR2211 was delivered into these chimeric mice to test if RORγ antagonists influence tumor growth via Th17 cells, and the impact of SR2211 on tumor growth, cellularity, and stem cell content was equivalent in chimeric wild type and RORγ recipient mice. These data collectively suggest that most of the observed effect of RORγ inhibition is tumor cell specific and not via an environmental/Th17 dependence on RORγ (FIGS. 5Q-5W); as a control it was found that RORγ deletion did lead to reduced CD8, CD4 and Th17 cells as predicted (FIGS. 5X, 21). Significant impact of SR2211 was not detected on cellularity of non-neoplastic cells such as CD45+, T cell, CD31+, MDSCs, macrophages, and dendritic within the tumors including at 7 days (FIG. 22).

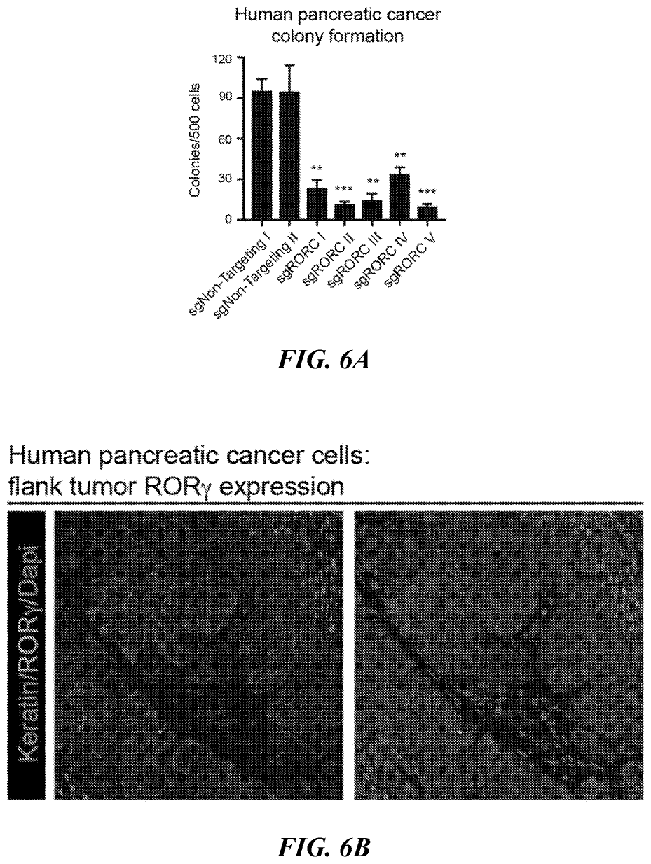

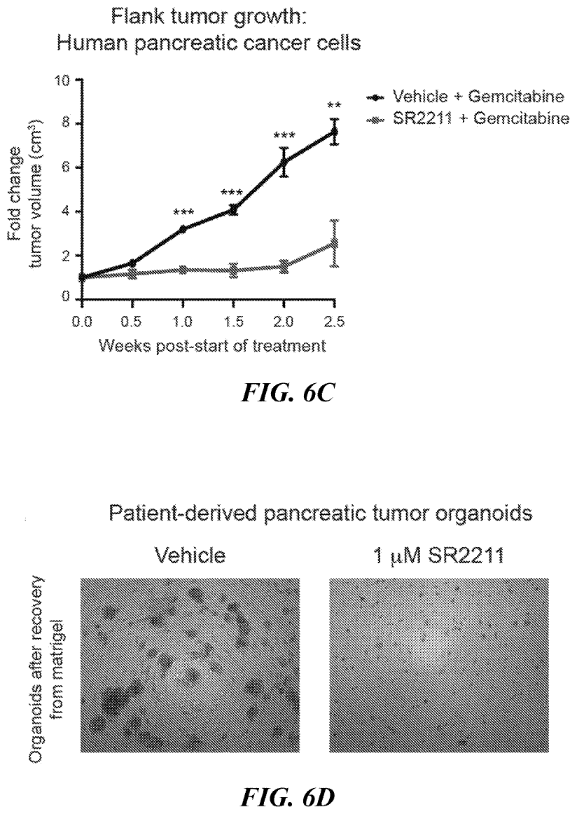

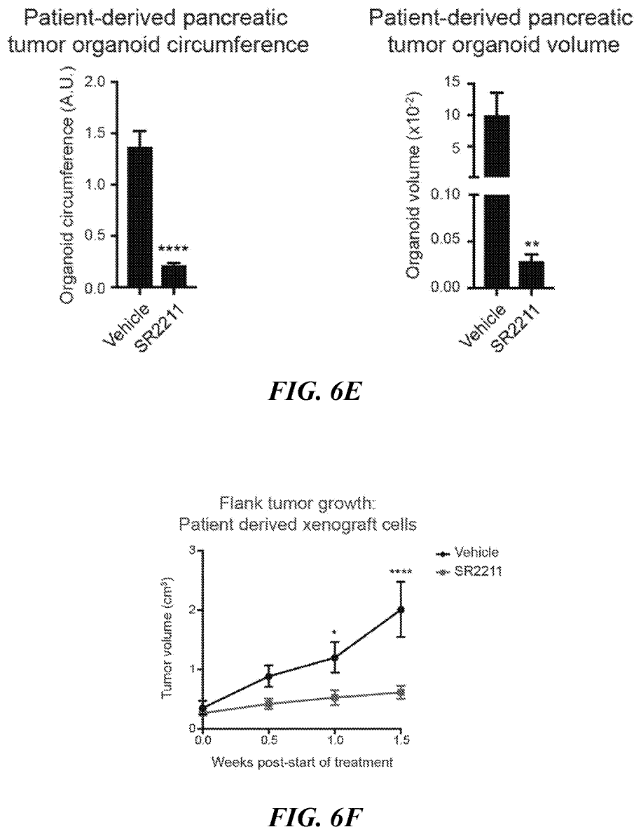

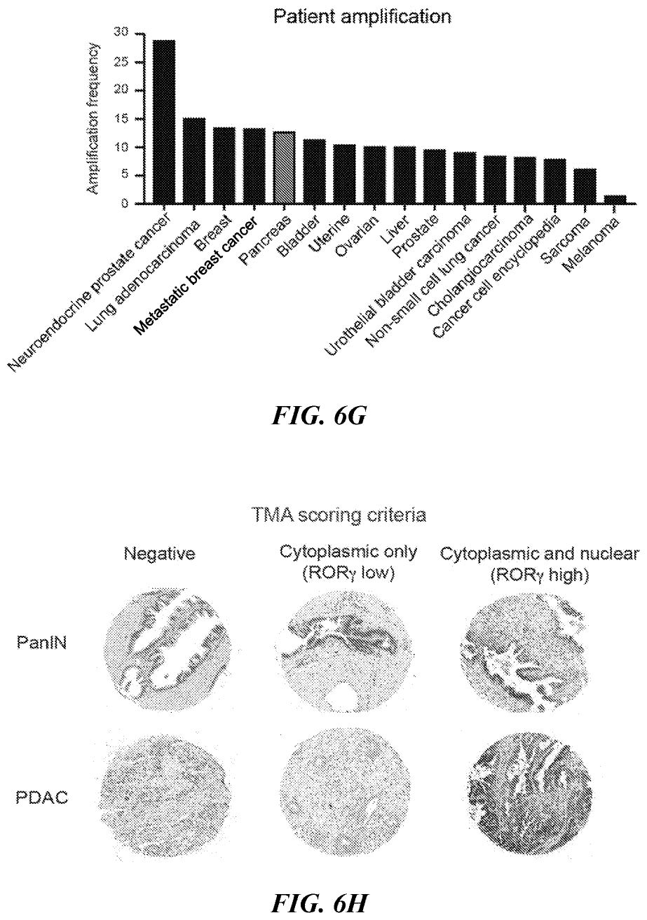

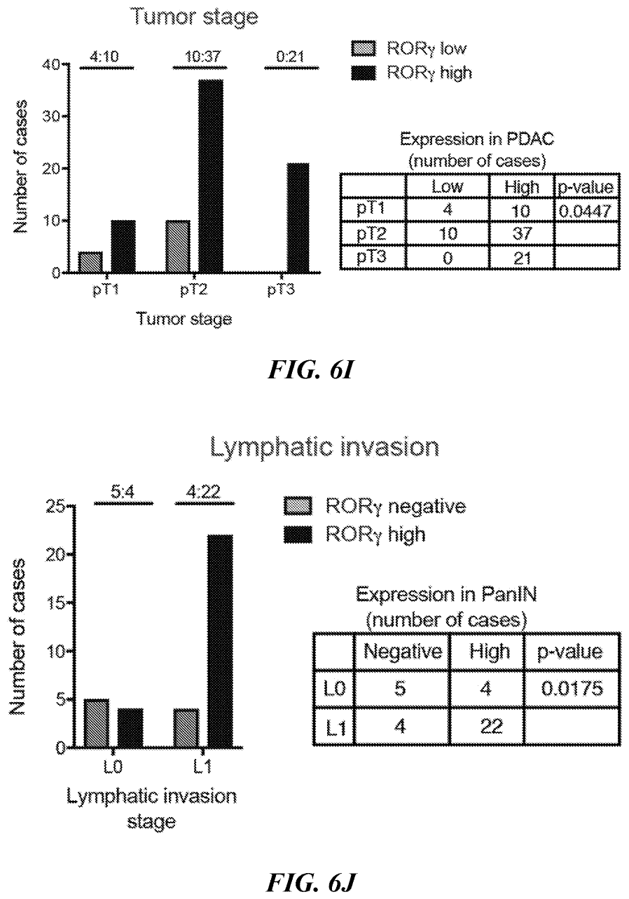

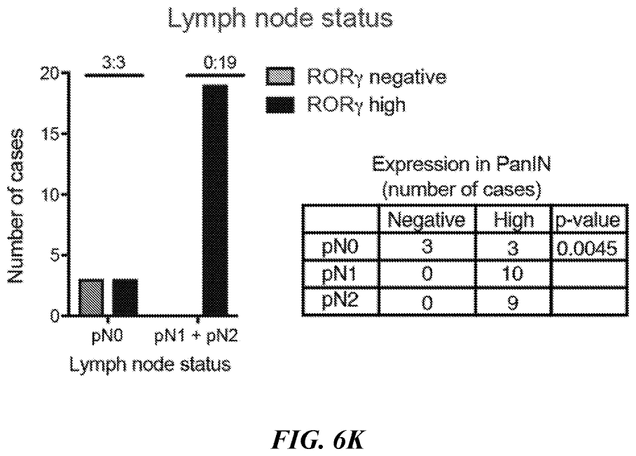

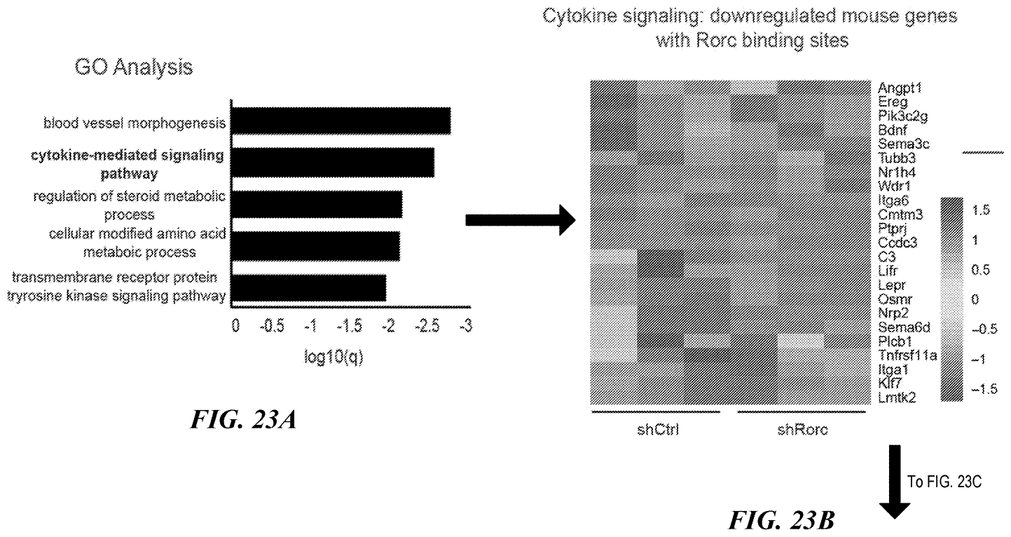

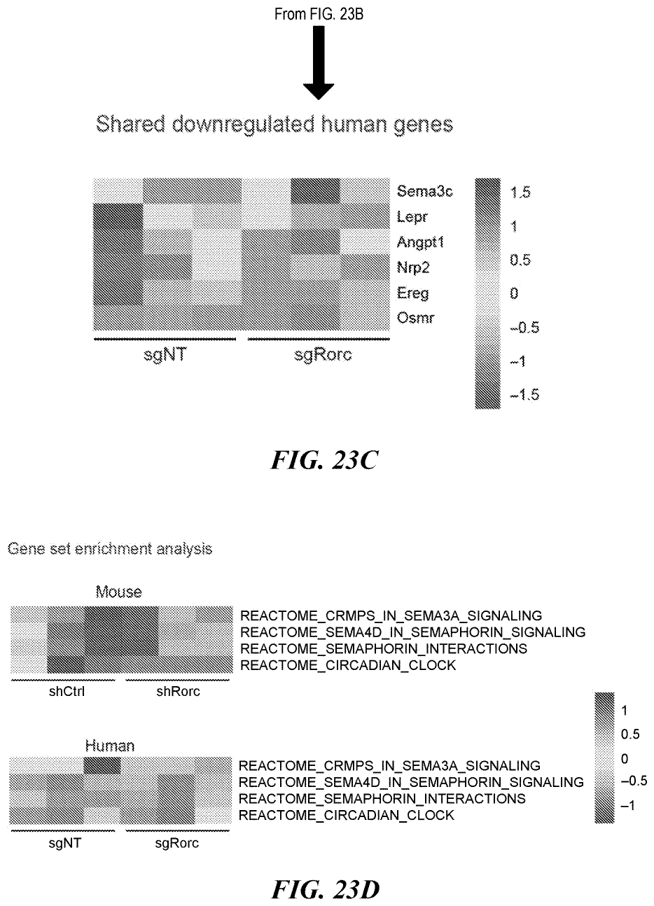

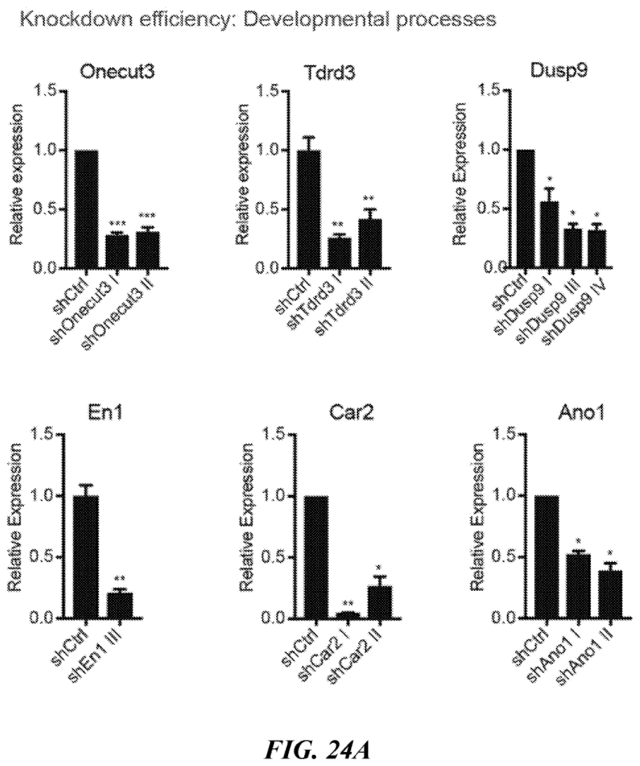

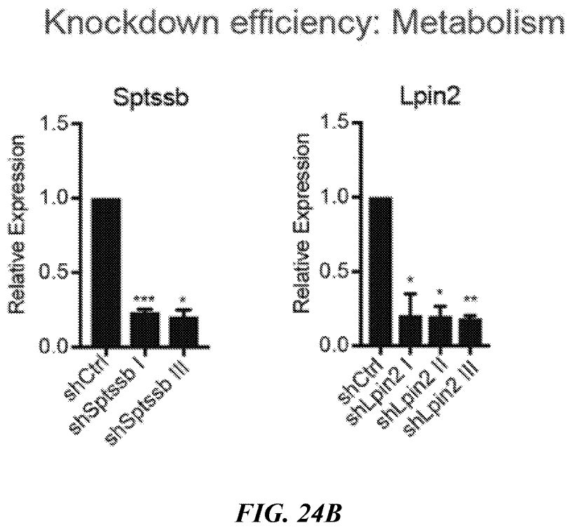

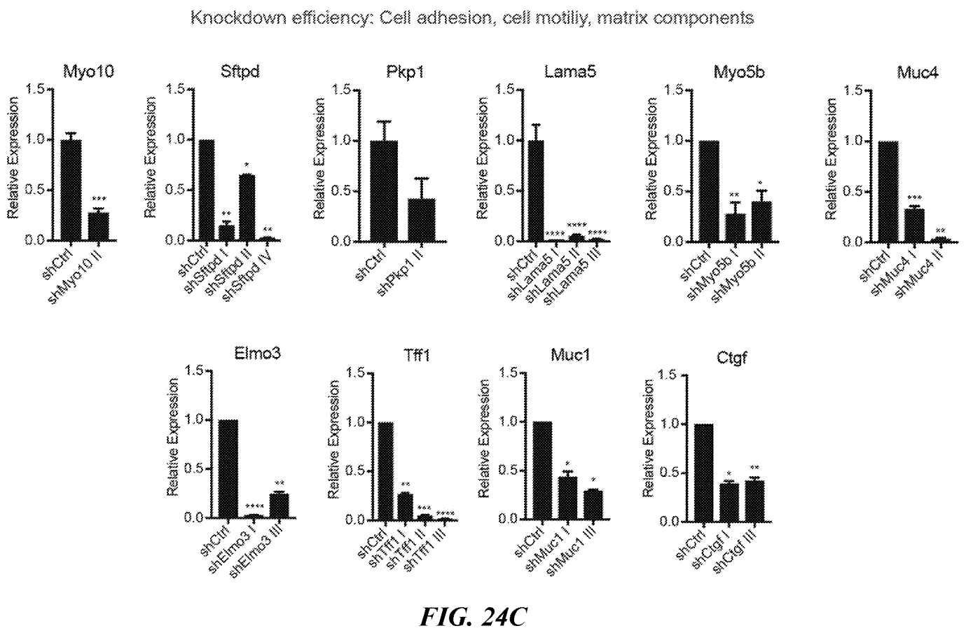

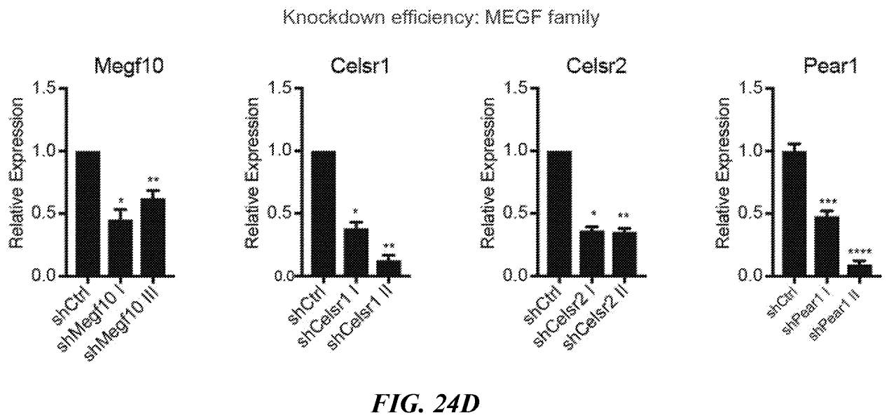

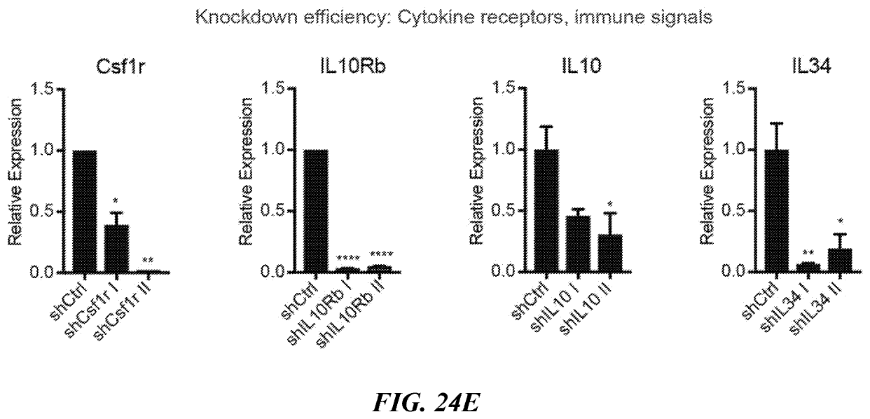

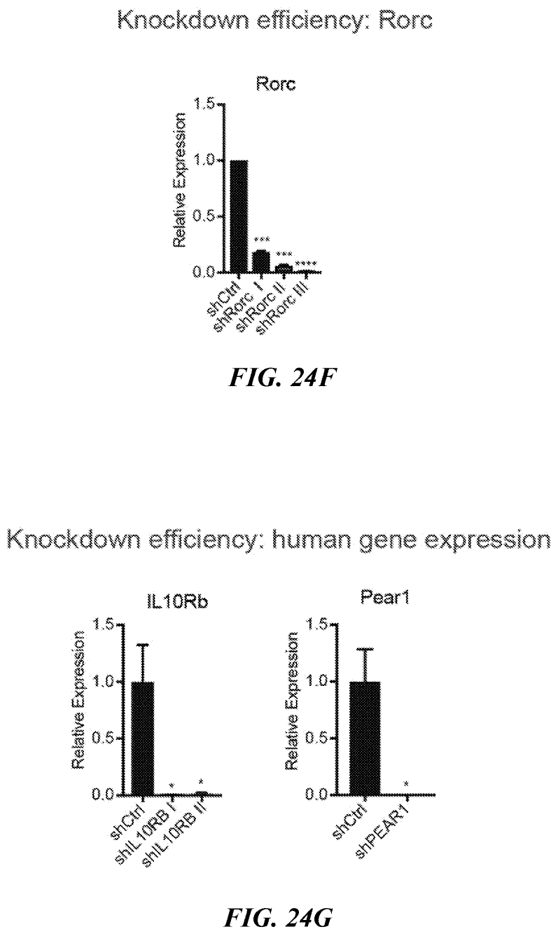

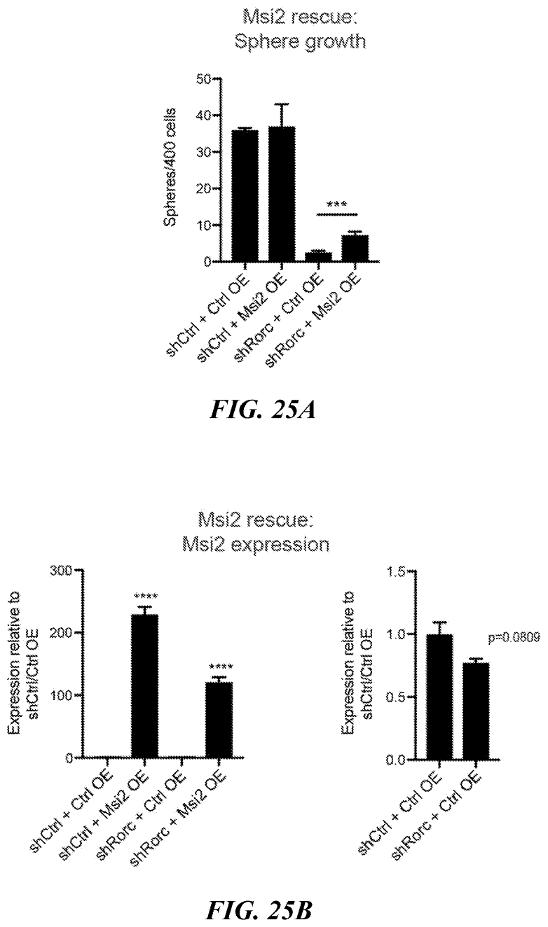

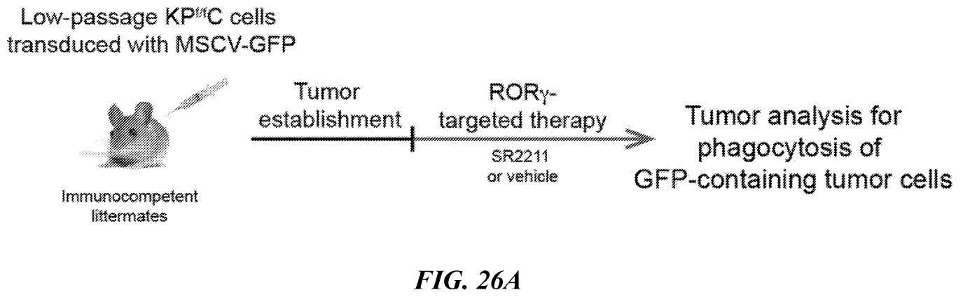

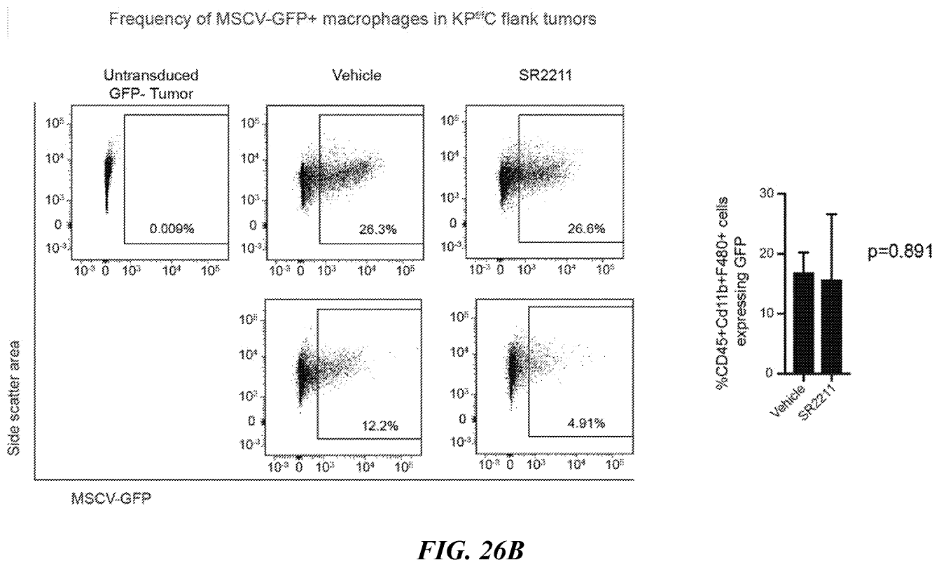

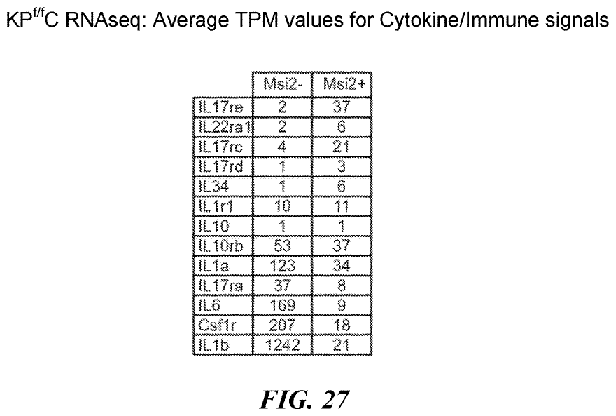

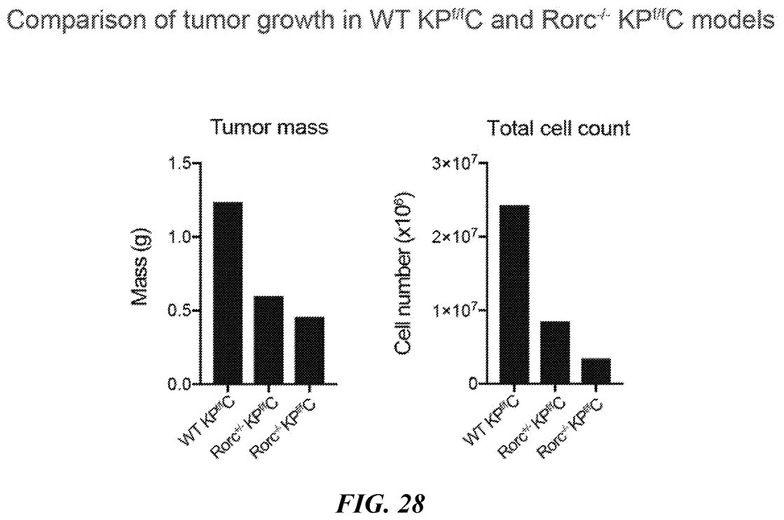

To further explore the functional relevance of RORγ to human pancreatic cancer, RORγ was inhibited both genetically and through pharmacologic inhibitors in human PDAC cells. CRISPR based disruption of RORγ using 5 independent guides led to a ˜3 to 9-fold loss of colony formation (FIG. 6A). To test if RORγ inhibition could block human tumor growth in vivo, human PDAC cells were transplanted into the flank region of immunocompromised mice, and tumors were allowed to become palpable before treatment began (FIG. 6B). Compared to vehicle-treatment, SR2211 delivery was highly effective and tumor growth was essentially extinguished with a nearly 6-fold reduction in growth in mice receiving SR2211 (FIG. 6C). Primary patient-derived organoids were also strikingly sensitive to RORγ blockade, with a ˜300-fold reduction in total organoid volume following SR2211 treatment (FIGS. 6D-6E, photo in methylcellulose shown in FIG. 19B). Importantly, delivery of SR2211 in primary patient derived xenografts led to a marked reduction of tumor growth in vivo (FIG. 6F). Interestingly, RNA-seq and Gene Ontology analysis of human FG and KPC cells identified a set of cytokines/growth factors as key common RORγ driven programs; e.g. Semaphorin 3c, its receptor Neuropilin2, Oncostatin M, and Angiopoietin, all highly pro-tumorigenic factors bearing RORγ binding motifs were identified as shared targets of RORγ in both mouse and human pancreatic cancer cells (FIG. 23). These data are particularly exciting in light of the fact that analysis of pancreatic cancer patients revealed genomic amplification of RORC in ˜12% of pancreatic cancer patients (FIG. 6G), raising the intriguing possibility that RORC amplification could serve as a biomarker for patients who may be particularly responsive to RORC inhibition.

Finally, to determine whether expression of RORγ could serve as a prognostic for specific clinicopathologic features, RORγ immunohistochemistry was performed on tissue microarrays from a clinically annotated retrospective cohort of 116 PDAC patients (Table 3). For 69 patients, matched pancreatic intraepithelial neoplasia (PanIN) lesions were available. RORγ protein was detectable (cytoplasmic expression only/low or cytoplasmic and nuclear expression/high, FIG. 6H) in 113 PDAC cases and 55 PanIN cases, respectively, and absent in 3 PDAC cases and 14 PanIN cases, respectively. Compared to cytoplasmic expression only, nuclear RORγ expression in PDAC cases was significantly correlated with higher pathological tumor (pT) stages at diagnosis (FIG. 6I). In addition, RORγ expression in PanIN lesions was positively correlated with lymphatic vessel invasion (L1, FIG. 6J) and lymph node metastasis (pN1, pN2, FIG. 6K) by the invasive carcinoma. However, no significant correlation of RORγ expression with overall or disease-free survival was observed, although potential treatment disparities may confound analysis of such patterns. These results indicate that RORγ expression in PanIN lesions and nuclear RORγ localization in invasive carcinoma could be useful markers to predict PDAC aggressiveness.

The most common outcome for pancreatic cancer patients following a response to cytotoxic therapy is not cure, but eventual disease progression and death driven by drug resistant stem cell-enriched populations. The presently disclosed technology has allowed one to develop a comprehensive molecular map of the core dependencies of pancreatic cancer stem cells by integrating their epigenetic, transcriptomic and functional genomic landscape. The data thus provide a novel resource for understanding therapeutic resistance and relapse, and for discovering new vulnerabilities in pancreatic cancer. As an example, the MEGF family of orphan receptors represent a potentially actionable family of adhesion GPCRs, as this class of signaling receptors have been considered druggable in cancer and other diseases. Importantly, the presently disclosed epigenetic analyses revealed a significant relationship between super-enhancer-associated genes and functional dependencies in stem cell conditions; stem cell-unique super-enhancer associated genes were more likely to drop out in the CRISPR screen in stem cell conditions compared to super-enhancer associated genes in non-stem cells (FIG. 19C). This provides additional evidence for the epigenetic and transcriptomic link to functional dependencies in cancer stem cells, and further supports previous findings that super-enhancer linked genes may be more important for maintaining the cell state and more sensitive to perturbation.

The presently disclosed screens identified an unexpected dependence of KP^{f/f}C stem cells on inflammatory and immune mediators, such as the CSF1R/IL-34 axis and IL-10R signaling. While these have been previously thought to act primarily on immune cells in the microenvironment, the data presented here suggest that stem cells may have evolved to co-opt this cytokine-rich milieu, allowing them to resist effective immune-based elimination. These findings also suggest that agents targeting CSF1R, which are under investigation for pancreatic cancer, may act not only on the tumor microenvironment but also directly on pancreatic epithelial cells themselves. These data also raise the possibility that therapies designed to activate the immune system to attack tumors may have effects on tumor cells directly: just as chemotherapy can kill tumor cells but may also impair the immune system, therapies designed to activate the immune system such as IL-10 may also promote the growth of tumor cells. This dichotomy of action will need to be considered in order to better optimize immunomodulatory treatment strategies.

A major new discovery driven by the network map was the identification of RORγ as a key immuno-regulatory pathway hijacked in pancreatic cancer. This together with the implication of RORγ in prostate cancer models suggests that this pathway may not be restricted to pancreatic cancer but may be more broadly utilized in other epithelial cancers. Interestingly, while cytokines such as IL17, IL21, IL22, and CSF2 are known targets of RORγ in Th17 cells, none of these were downregulated in RORc-deficient pancreatic tumor cells. The fact that RORγ regulated potent oncogenes marked by super-enhancers in stem cells, suggest it may be critical for defining the stem cell state in pancreatic cancer. In addition, the network of genes impacted by RORγ inhibition included other immune-modulators such as CD47, raising the possibility that it may also mediate interaction with the surrounding niche and immune system cells. Finally, one particularly exciting aspect of this work is the possibility that RORγ represents a potential therapeutic target for pancreatic cancer. Given that inhibitors of RORγ are currently in Phase II trials for autoimmune diseases, repositioning these agents as pancreatic cancer therapies warrants further investigation.

E. Experimental Model, Subject, and Method Details

Mice

REM2 (Msi2^{eGFP/+}) reporter mice were generated as previously described (Fox et al., 2016); all of the reporter mice used in experiments were heterozygous for the Msi2 allele. The LSL-KrasG12D mouse, B6.129S4-Kras^{tm4Tyj/J} (Stock No: 008179), the p53flox/flox mouse, B6.129P2-Trp53^{tm1Brn/J} (Stock No: 008462), and the RORγ-knockout mouse (Stock No: 007571), were purchased from The Jackson Laboratory. Dr. Chris Wright provided Ptf1a-Cre mice as previously described (Kawaguchi et al., 2002). LSL-R172H mutant p53, Trp53^{R172H} mice were provided by Dr. Tyler Jacks as previously described (Olive et al., 2004) (JAX Stock No: 008183). The mice listed above are immunocompetent, with the exception of RORγ-knockout mice which are known to lack TH17 T-cells as described previously (Ivanov et al., 2006); these mice were maintained on antibiotic water (sulfamethoxazole and trimethoprim) when enrolled in flank transplantation and drug studies as outlined below. Immune compromised NOD/SCID (NOD.CB17-Prkdc^{scid}/J, Stock No: 001303) and NSG (NOD.Cg-Prkdc^{scid}IL2rg^{tm1Wji}/SzJ, Stock No: 005557) mice purchased from The Jackson Laboratory. All mice were specific-pathogen free and bred and maintained in the animal care facilities at the University of California San Diego. Animals had access to food and water ad libitum and were housed in ventilated cages under controlled temperature and humidity with a 12-hour light-dark cycle. All animal experiments were performed according to protocols approved by the University of California San Diego Institutional Animal Care and Use Committee. No sexual dimorphism was noted in all mouse models. Therefore, males and females of each strain were equally used for experimental purposes and both sexes are represented in all data sets. All mice enrolled in experimental studies were treatment-naïve and not previously enrolled in any other experimental study.

Both REM2-KP^{f/f}C and WT-KP^{f/f}C mice (REM2; LSL-KraG^{G12D/+}; Trp53^{f/f}; Ptf1a-Cre and LSL-Kras^{G12D/+}; Trp53^{f/f}; Ptf1a-Cre respectively) were used for isolation of tumor cells, establishment of primary mouse tumor cell and organoid lines, and autochthonous drug studies as described below. REM2-KP^{f/f}C and KP^{f/f}C mice were enrolled in drug studies between 8 to 11 weeks of age and were used for tumor cell sorting and establishment of cell lines when they reached end-stage disease between 10 and 12 weeks of age. REM2-KP^{f/f}C mice were used for in vivo imaging studies between 9.5-10.5 weeks of age. KP^{R172H}C (LSL-Kras^{G12D/+}; Trp53^{R172h/+}; Ptf1a-Cre) mice were used for cell sorting and establishment of tumor cell lines when they reached end-stage disease between 16-20 weeks of age. In some studies, KP^{f/f}C-derived tumor cells were transplanted into the flanks of immunocompetent littermates between 5-8 weeks of age. Littermate recipients (WT or REM2-LSL-Kras^{G12D/+}; Trp53^{f/f} or Trp53^{f/f} mice) do not develop disease or express Cre. NOD/SCID and NSG mice were enrolled in flank transplantation studies between 5 to 8 weeks of age; KP^{f/f}C derived cell lines and human FG cells were transplanted subcutaneously for tumor propagation studies in NOD/SCID recipients and patient-derived xenografts and KP^{f/f}C derived cell lines were transplanted subcutaneously in NSG recipients as described in detail below.

Human and Mouse Pancreatic Cancer Cell Lines

Mouse primary pancreatic cancer cell lines and organoids were established from end-stage, treatment-naïve KP^{R172H}C and WT- and REM2-KP^{f/f}C mice as follows: tumors from endpoint mice (10-12 weeks of age for KP^{f/f}C or 16-20 weeks of age for KP^{R172H}C mice) were isolated and dissociated into single cell suspension as described below. Cells were then either plated in 3D sphere or organoid culture conditions detailed below or plated in 2D in 1× DMEM containing 10% FBS, 1× pen/strep, and 1× non-essential amino acids. At the first passage in 2D, cells were collected and resuspended in HBSS (Gibco, Life Technologies) containing 2.5% FBS and 2 mM EDTA, then stained with FC block followed by 0.2 μg/10⁶ cells anti-EpCAM APC (eBioscience). EpCAM+ tumor cells were sorted then re-plated for at least one additional passage. To evaluate any cellular contamination and validate the epithelial nature of these lines, cells were analyzed by flow cytometry again at the second passage for markers of blood cells (CD45-PeCy7, eBioscience), endothelial cells (CD31-PE, eBioscience), and fibroblasts (PDGFR-PacBlue, Biolegend). Cell lines were derived from both female and male KP^{R172H}C and WT- and REM2-KP^{f/f}C mice equivalently; both sexes are equally represented in the cell-based studies outlined below. Functional studies were performed using cell lines between passage 2 and passage 6. Human FG cells were originally derived from a PDAC metastasis and have been previously validated and described (Morgan et al., 1980). Patient-derived xenograft cells and organoids were derived from originally-consented (now deceased) PDAC patients and use was approved by UCSD's IRB; cells were de-identified and therefore no further information on patient status, treatment or otherwise, is available. FG cell lines were cultured in 2D conditions in lx DMEM (Gibco, Life Technologies) containing 10% FBS, 1× pen/strep (Gibco, Life Technologies), and 1× non-essential amino acids (Gibco, Life Technologies). 3D in vitro culture conditions for all cells and organoids are detailed below.

Patient Cohort for PDAC Tissue Microarray

The PDAC patient cohort and corresponding TMAs used for RORγ immunohistochemical staining and analysis have been reported previously (Wartenberg et al., 2018). Patient characteristics are detailed in Table 3. Briefly, a total of 4 TMAs with 0.6 mm core size was constructed: three TMAs for PDACs, with samples from the tumor center and invasive front (mean number of spots per patient: 10.5, range: 2-27) and one TMA for matching PanINs (mean number of spots per patient: 3.7, range: 1-6). Tumor samples from 116 patients (53 females and 63 males; mean age: 64.1 years, range: 34-84 years) with a diagnosis of PDAC were included. Matched PanIN samples were available for 69 patients. 99 of these patients received some form of chemotherapy; 14 received radiotherapy. No sexual dimorphism was observed in any of the parameters assessed, including overall survival (p=0.227), disease-free interval (p=0.3489) or RORγ expression in PDAC (p=0.9284) or PanINs (p=0.3579). The creation and use of the TMAs were reviewed and approved by the Ethics Committee at the University of Athens, Greece, and the University of Bern, Switzerland, and included written informed consent from the patients or their living relatives.

Tissue Dissociation, Cell Isolation, and FACS Analysis

Mouse pancreatic tumors were washed in MEM (Gibco, Life Technologies) and cut into 1-2 mm pieces immediately following resection. Tumor pieces were collected into a 50 ml Falcon tube containing 10 ml Gey's balanced salt solution (Sigma), 5 mg Collagenase P (Roche), 2 mg Pronase (Roche), and 0.2 μg DNAse I (Roche). Samples were incubated for 20 minutes at 37° C., then pipetted up and down 10 times and returned to 37° C. After 15 more minutes, samples were pipetted up and down 5 times, then passaged through a 100 μm nylon mesh (Corning). Red blood cells were lysed using RBC Lysis Buffer (eBioscience) and the remaining tumor cells were washed, then resuspended in HBSS (Gibco, Life Technologies) containing 2.5% FBS and 2 mM EDTA for staining, FACS analysis, and cell sorting. Analysis and cell sorting were carried out on a FACSAria III machine (Becton Dickinson), and data were analyzed with FlowJo software (Tree Star). For analysis of cell surface markers by flow cytometry, 5×10⁵ cells were resuspended in HBSS containing 2.5% FBS and 2 mM EDTA, then stained with FC block followed by 0.5 μl of each antibody. For intracellular staining, cells were fixed and permeabilized using the BrdU flow cytometry kit (BD Biosciences); Annexin V apoptosis kit was used for analysis of apoptotic cells (eBioscience). The following rat antibodies were used: anti-mouse EpCAM-APC (eBioscience), anti-mouse CD133-PE (eBioscience), anti-mouse CD45-PE and PE/Cy7 (eBioscience), anti-mouse CD31-PE (BD Bioscience), anti-mouse Gr-1-FITC (eBioscience), anti-mouse F4/80-PE (Invitrogen), anti-mouse CD11b-APC (Affymetrix), anti-mouse CD11c-BV421 (Biolegend), anti-mouse CD4-FITC (eBioscience) and CD4-Pacific blue (Bioglegend), anti-mouse CD8-PE (eBioscience), anti-mouse IL-17-APC (Biolegend), anti-mouse BrdU-APC (BD Biosciences), and anti-mouse Annexin-V-APC (eBioscience). Propidium-iodide (Life Technologies) was used to stain for dead cells.

In Vitro Growth Assays

Described below are the distinct growth assays used for pancreatic cancer cells. Colony formation is an assay in Matrigel (thus adherent/semi-adherent conditions), while tumorsphere formation is an assay in non-adherent conditions. Cell types from different sources grow better in different conditions. For example, the murine KP^{R172H/+}C and the human FG cell lines grow much better in Matrigel, while KP^{f/f}C cell lines often grow well in non-adherent, sphere conditions (though they can also grow in Matrigel).

Pancreatic Tumorsphere Formation Assay

Pancreatic tumorsphere formation assays were performed and modified from (Rovira et al., 2010). Briefly, low-passage (<6 passages) WT or REM2-KP^{f/f}C cell lines were infected with lentiviral particles containing shRNAs; positively infected (red) cells were sorted 72 hours after transduction. 100-300 infected cells were suspended in tumorsphere media: 100 μl DMEM F-12 (Gibco, Life Technologies) containing 1× B-27 supplement (Gibco, Life Technologies), 3% FBS, 100 μM B-mercaptoethanol (Gibco, Life Technologies), 1× non-essential amino acids (Gibco, Life Technologies), 1× N2 supplement (Gibco, Life Technologies), 20 ng/ml EGF (Gibco, Life Technologies), 20 ng/ml bFGF2 (Gibco, Life Technologies), and 10 ng/ml ESGRO mLIF (Thermo Fisher). Cells in media were plated in 96-well ultra-low adhesion culture plates (Costar) and incubated at 37° C. for 7 days. KP^{f/f}C in vitro tumorsphere formation studies were conducted at a minimum of n=3 independent wells per cell line across two independent shRNA of n=3 wells; however, the majority of these experiments were additionally completed in >1 independently-derived cell lines n=3, at n=3 wells per shRNA.

Matrigel Colony Assay

For FG and KP^{R172H/+}C cells, 300-500 cells were resuspended in 50 μl tumorsphere media as described below, then mixed with Matrigel (BD Biosciences, 354230) at a 1:1 ratio and plated in 96-well ultra-low adhesion culture plates (Costar). After incubation at 37° C. for 5 min, 50 μl tumorsphere media was placed over the Matrigel layer. Colonies were counted 7 days later. For RORγ inhibitor studies, SR2211 or vehicle was added to cells in tumorsphere media, then mixed 1:1 with Matrigel and plated. SR2211 or vehicle was also added to the media that was placed over the solidified Matrigel layer. For FG colony formation, n=5 independent wells across 5 independent CRISPR sgRNA and two independent non-targeting gRNA. KP^{R172H/+}C cells were plated at n=3 wells per shRNA from one cell line.

Organoid Culture Assays

Tumors from 10-12 week old end stage REM2-KP^{f/f}C mice were harvested and dissociated into a single cell suspension as described above. Tumor cells were stained with FC block then 0.2 μg/10⁶ cells anti-EpCAM APC (eBioscience). Msi2+/EpCAM+ (stem) and Msi2−/EpCAM+ (non-stem) cells were sorted, resuspended in 20 μl Matrigel (BD Biosciences, 354230). For limiting dilution assay, single cells were resuspended in matrigel at the indicated numbers from 20,000 to 10 cells/20 μL and were plated as a dome in a pre-warmed 48 well plate. After incubation at 37° C. for 5 min, domes were covered with 300 μl PancreaCult Organoid Growth Media (Stemcell Technologies). Organoids were imaged and quantified 6 days later. Limiting dilution analysis for stemness assessment was performed using web based-extreme limiting dilution analysis (ELDA) software (Hu and Smyth, 2009). Msi2+/EpCAM+ (stem) and Msi2−/EpCAM+ (non-stem) organoids were derived from n=3 independent mice and plated at the indicated cell numbers.

Organoids from REM2-KP^{f/f}C were passaged at ˜1:2 as previously described (Boj et al., 2015). Briefly, organoids were isolated using Cell Recovery Solution (Corning 354253), then dissociated using Accumax Cell Dissociation Solution (Innovative Cell Technologies AM105), and plated in 20 μl matrigel (BD Biosciences, 354230) domes on a pre-warmed 48-well plate. After incubation at 37° C. for 5 min, domes were covered with 300 μl PancreaCult Organoid Growth Media (Stemcell Technologies). SR2211 (Cayman Chemicals 11972) was resuspended in DMSO at 20 mg/ml, diluted 1:10 in 0.9% NaCl containing 0.2% acetic acid, and further diluted in PancreaCult Organoid Media (Stemcell Technologies) to the indicated dilutions. Organoids were grown in the presence of vehicle or SR2211 for 4 days, then imaged and quantified, n=3 independent wells plated per dose per treatment group.

Primary patient organoids were established and provided by Dr. Andrew Lowy. Briefly, patient-derived xenografts were digested for 1 hour at 37° C. in RPMI containing 2.5% FBS, 5 mg/ml Collagenase II, and 1.25 mg/ml Dispase II, then passaged through a 70 μM mesh filter. Cells were plated at a density of 1.5×10⁵ cells per 50 μl Matrigel. After domes were solidified, growth medium was added as follows: RPMI containing 50% Wnt3a conditioned media, 10% R-Spondinl-conditioned media, 2.5% FBS, 50 ng/ml EGF, 5 mg/ml Insulin, 12.5 ng/ml hydrocortisone, and 14 μM Rho Kinase Inhibitor. After establishment, organoids were passaged and maintained as previously described (Boj et al., 2015). Briefly, organoids were isolated using Cell Recovery Solution (Corning 354253), then dissociated into single cell suspensions with TrypLE Express (ThermoFisher 12604) supplemented with 25 μg/ml DNase I (Roche) and 14 μM Rho Kinase Inhibitor (Y-27632, Sigma). Cells were split 1:2 into 20 μl domes plated on pre-warmed 48 well plates. Domes were incubated at 37° C. for 5 min, then covered with human complete organoid feeding media (Boj et al., 2015) without Wnt3a-conditioned media. SR2211 was prepared as described above, added at the indicated doses, and refreshed every 3 days. Organoids were grown in the presence of vehicle or SR2211 for 7 days, then imaged and quantified, n=3 independent wells plated per dose per treatment group. All images were acquired on a Zeiss Axiovert 40 CFL. Organoids were counted and measured using ImageJ 1.51s software.

Flank Tumor Transplantation Studies

For the flank transplantation studies outlined below, investigators blinded themselves when possible to the assigned treatment group of each tumor for analysis; mice were de-identified after completion of flow cytometry analysis. The number of tumors transplanted for each study is based on past experience with studies of this nature, where a group size of 10 is sufficient to determine if pancreatic cancer growth is significantly affected when a regulatory signal is perturbed (see Fox et al., 2016).

For shRNA-infected pancreatic tumor cell propagation in vivo, cells were infected with lentiviral particles containing shRNAs and positively infected (red) cells were sorted 72 hours after transduction. 1000 low passage, shRNA-infected KP^{f/f}C, or 2×10⁵ shRNA-infected FG cells were resuspended in 50 μl culture media, then mixed 1:1 with matrigel (BD Biosciences). Cells were injected subcutaneously into the left or right flank of 5-8 week-old NOD/SCID recipient mice. Subcutaneous tumor dimensions were measured with calipers 1-2× weekly for 6-8 weeks, and two independent transplant experiments were conducted for each shRNA at n=4 independent tumors per group.

For drug-treated KP^{f/f}C flank tumors, 2×10⁴ low passage REM2-KP^{f/f}C tumor cells were resuspended in 50 μl culture media, then mixed 1:1 with matrigel (BD Biosciences). Cells were injected subcutaneously into the left or right flank of 5-8 week-old non-tumor bearing, immunocompetent littermates or NSG mice. Tumor growth was monitored twice weekly; when tumors reached 0.1-0.3 cm³, mice were randomly enrolled in treatment groups and were treated for 3 weeks as described below. After 3 weeks of therapy, tumors were removed, weighed, dissociated, and analyzed by flow cytometry. Tumor volume was calculated using the standard modified ellipsoid formula ½ (Length×Width²); n=2-4 tumors per treatment group in immunocompetent littermate recipients and n=4-6 tumors per treatment group in NSG recipients.

For chimeric transplantation studies, 2×10⁴ low passage REM2-KP^{f/f}C tumor cells were resuspended in 50 μl culture media, then mixed 1:1 with matrigel (BD Biosciences). Cells were injected subcutaneously into the left or right flank of 5-8 week-old RORγ-knockout or wild-type recipients; recipient mice were maintained on antibiotic water (sulfamethoxazole and trimethoprim). Tumor growth was monitored twice weekly; when tumors reached 0.1-0.3 cm³, mice were randomly enrolled in treatment groups and were treated for 3 weeks as described below. After 3 weeks of therapy, tumors were removed, weighed, dissociated, and analyzed by flow cytometry. Tumor volume was calculated using the standard modified ellipsoid formula ½ (Length×Width²); n=5-7 tumors per treatment group.

For drug-treated human pancreatic tumors 2×10⁴ human pancreatic FG cancer cells or 2×10⁶ patient-derived xenograft cells were resuspended in 50 μl culture media, then mixed 1:1 with matrigel (BD Biosciences). Cells were injected subcutaneously into the left or right flank of 5-8 week-old NSG recipient mice. Mice were randomly enrolled in treatment groups and were treated for 3 weeks as described below. After 3 weeks of therapy, tumors were removed, weighed, and dissociated. Subcutaneous tumor dimensions were measured with calipers 1-2× weekly. Tumor volume was calculated using the standard modified ellipsoid formula ½ (Length×Width²); at minimum n=4 tumors per treatment group.

In Vivo and In Vitro Drug Therapy

The RORγ inverse agonists SR2211 (Cayman Chemicals, 11972, or Tocris, 4869) was resuspended in DMSO at 20 mg/ml or 50 mg/ml, respectively, then mixed 1:20 in 8% Tween80-PBS prior to use. Gemcitabine (Sigma, G6423) was resuspended in H₂O at 20 mg/ml. For in vitro drug studies, low passage (<6 passage) WT- or REM2-KP^{f/f}C cells, (<10 passage) KP^{R172H/+}C cells, or FG cells were plated in non-adherent tumorsphere conditions or Matrigel colony conditions for 1 week in the presence of SR2211 or vehicle. For KP^{f/f}C littermate, NSG mice, and RORγ-knockout mice bearing KP^{f/f}C-derived flank tumors and for NSG mice bearing flank patient-derived xenograft tumors, mice were treated with either vehicle (PBS) or gemcitabine (25 mg/kg i.p., 1× weekly) alone or in combination with vehicle (5% DMSO, 8% Tween80-PBS) or SR2211 (10 mg/kg i.p., daily) for 3 weeks. RORγ-knockout mice and paired wild-type littermates were maintained on antibiotic water (sulfamethoxazole and trimethoprim). For NOD/SCID mice bearing flank FG tumors, mice were treated with either vehicle (5% DMSO in corn oil) or SR2211 (10 mg/kg i.p., daily) for 2.5 weeks. All flank tumors were measured 2× weekly and mice were sacrificed if tumors were >2 cm³, in accordance with IACUC protocol. For KP^{f/f}C autochthonous survival studies, 8 week old tumor-bearing KP^{f/f}C mice were enrolled in either vehicle (10% DMSO, 0.9% NaCl with 0.2% acetic acid) or SR2211 (20 mg/kg i.p., daily) treatment groups, and treated until moribund, where n=4 separate mice per treatment group. For all drug studies, tumor-bearing mice were randomly assigned into drug treatment groups; treatment group size was determined based on previous studies (Fox et al., 2016).

Immunofluorescence Staining

Pancreatic cancer tissue from KP^{f/f}C mice was fixed in Z-fix (Anatech Ltd, Fisher Scientific) and paraffin embedded at the UCSD Histology and Immunohistochemistry Core at The Sanford Consortium for Regenerative Medicine according to standard protocols. 5 μm sections were obtained and deparaffinized in xylene. The human pancreas paraffin embedded tissue array was acquired from US Biomax, Inc (BIC14011a). For paraffin embedded mouse and human pancreas tissues, antigen retrieval was performed for 40 minutes in 95-100° C. 1× Citrate Buffer, pH 6.0 (eBioscience). Sections were blocked in PBS containing 0.1% Triton X100 (Sigma-Aldrich), 10% Goat Serum (Fisher Scientific), and 5% bovine serum albumin (Invitrogen).

KP^{f/f}C cells and human pancreatic cancer cell lines were suspended in DMEM (Gibco, Life Technologies) supplemented with 50% FBS and adhered to slides by centrifugation at 500 rpm. 24 hours later, cells were fixed with Z-fix (Anatech Ltd, Fisher Scientific), washed in PBS, and blocked with PBS containing 0.1% Triton X-100 (Sigma-Aldrich), 10% Goat serum (Fisher Scientific), and 5% bovine serum albumin (Invitrogen). All incubations with primary antibodies were carried out overnight at 4° C. Incubation with Alexafluor-conjugated secondary antibodies (Molecular Probes) was performed for 1 hour at room temperature. DAPI (Molecular Probes) was used to detect DNA and images were obtained with a Confocal Leica TCS SP5 II (Leica Microsystems). The following primary antibodies were used: chicken anti-GFP (Abcam, ab13970) 1:500, rabbit anti-RORγ (Thermo Fisher, PA5-23148) 1:500, mouse anti-E-Cadherin (BD Biosciences, 610181) 1:500, anti-Keratin (Abcam, ab8068) 1:15, anti-Hmga2 (Abcam. Ab52039) 1:100, anti-Celsr1 (EMD Millipore abt119) 1:1000, anti-Celsr2 (BosterBio A06880) 1:250.

Tumor Imaging

9.5-10.5 week old REM2-KP^{f/f}C mice were treated either vehicle or SR2211 (10 mg/kg i.p., daily) for 8 days. For imaging, mice were anesthetized by intraperitoneal injection of ketamine and xylazine (100/20 mg/kg). In order to visualize blood vessels and nuclei, mice were injected retro-orbitally with AlexaFluor 647 anti-mouse CD144 (VE-cadherin) antibody and Hoechst 33342 immediately following anesthesia induction. After 25 minutes, pancreatic tumors were removed and placed in HBSS containing 5% FBS and 2 mM EDTA. 80-150 μm images in 1024×1024 format were acquired with an HCX APO L20× objective on an upright Leica SP5 confocal system using Leica LAS AF 1.8.2 software. GFP cluster sizes were measure using ImageJ 1.51s software. 2 mice per treatment group were analyzed in this study; 6-10 frames were analyzed per mouse.

Analysis of Tissue Microarrays, Immunohistochemistry (IHC) and Staining Analysis

TMAs were sectioned to 2.5 μm thickness. IHC staining was performed on a Leica BOND RX automated immunostainer using BOND primary antibody diluent and BOND Polymer Refine DAB Detection kit according to the manufacturer's instructions (Leica Biosystems). Pre-treatment was performed using citrate buffer at 100° C. for 30 min, and tissue was stained using rabbit anti-human RORγ(t) (polyclonal, #PA5-23148, Thermo Fisher Scientific) at a dilution of 1:4000. Stained slides were scanned using a Pannoramic P250 digital slide scanner (3DHistech). RORγ(t) staining of individual TMA spots was analyzed in an independent and randomized manner by two board-certified surgical pathologists (C.M.S and M.W.) using Scorenado, a custom-made online digital TMA analysis tool. Interpretation of staining results was in accordance with the “reporting recommendations for tumor marker prognostic studies” (REMARK) guidelines. Equivocal and discordant cases were re-analyzed jointly to reach a consensus. RORγ(t) staining in tumor cells was classified microscopically as 0 (absence of any cytoplasmic or nuclear staining), 1+ (cytoplasmic staining only), and 2+ (cytoplasmic and nuclear staining). For patients in whom multiple different scores were reported, only the highest score was used for further analysis. Spots/patients with no interpretable tissue (less than 10 intact, unequivocally identifiable tumor cells) or other artifacts were excluded.

Statistical Analysis of TMA Data

Descriptive statistics were performed for patients' characteristics. Frequencies, means, and range values are given. Association of RORγ(t) expression with categorical variables was performed using the Chi-square or Fisher's Exact test, where appropriate, while correlation with continuous values was tested using the non-parametric Kruskal-Wallis or Wilcoxon test. Univariate survival time differences were analyzed using the Kaplan-Meier method and log-rank test. All p-values were two-sided and considered significant if <0.05.

shRNA Lentiviral Constructs and Production

Short hairpin RNA (shRNA) constructs were designed and cloned into pLV-hU6-mPGK-red vector by Biosettia. Virus was produced in 293T cells transfected with 4 μg shRNA constructs along with 2 μg pRSV/REV, 2 μg pMDLg/pRRE, and 2 μg pHCMVG constructs (Dull et al., 1998; Sena-Esteves et al., 2004). Viral supernatants were collected for two days then concentrated by ultracentrifugation at 20,000 rpm for 2 hours at 4° C. Knockdown efficiency for the shRNA constructs used in this study varied from 45-95%.

RT-qPCR Analysis

RNA was isolated using RNeasy Micro and Mini kits (Qiagen) and converted to cDNA using Superscript III (Invitrogen). Quantitative real-time PCR was performed using an iCycler (BioRad) by mixing cDNAs, iQ SYBR Green Supermix (BioRad) and gene specific primers. Primer sequences are available in Table 4. All real time data was normalized to B2M or Gapdh.

Genome-Wide Profiling and Bioinformatic Analysis, Primary Msi2+ and Msi2− KP^{f/f}C RNA-seq, Data Analysis, and Visualization, Stem and Non-Stem Tumor Cell Isolation Followed by RNA-Sequencing

Tumors from three independent 10-12 week old REM2-KP^{f/f}C mice were harvested and dissociated into a single cell suspension as described above. Tumor cells were stained with FC block then 0.2 μg/10⁶ cells anti-EpCAM APC (eBioscience). 70,00-100,00 Msi2+/EpCAM+ (stem) and Msi2−/EpCAM+ (non-stem) cells were sorted and total RNA was isolated using RNeasy Micro kit (Qiagen). Total RNA was assessed for quality using an Agilent Tapestation, and all samples had RIN≥7.9. RNA libraries were generated from 65 ng of RNA using Illumina's TruSeq Stranded mRNA Sample Prep Kit following manufacturer's instructions, modifying the shear time to 5 minutes. RNA libraries were multiplexed and sequenced with 50 basepair (bp) single end reads (SR50) to a depth of approximately 30 million reads per sample on an Illumina HiSeq2500 using V4 sequencing chemistry.

RNA-seq Analysis

RNA-seq fastq files were processed into transcript-level summaries using kallisto (Bray et al., 2016), an ultrafast pseudo-alignment algorithm with expectation maximization. Transcript-level summaries were processed into gene-level summaries by adding all transcript counts from the same gene. Gene counts were normalized across samples using DESeq normalization (Anders and Huber 2010) and the gene list was filtered based on mean abundance, which left 13,787 genes for further analysis. Differential expression was assessed with an R package limma (Ritchie et al., 2015) applied to log₂-transformed counts. Statistical significance of each test was expressed in terms of local false discovery rate lfdr (Efron and Tibshirani, 2002) using the limma function eBayes (Lönnstedt, I., and Speed, T. 2002). lfdr, also called posterior error probability, is the probability that a particular gene is not differentially expressed, given the data.

Cell State Analysis

For cell state analysis, Gene Set Enrichment Analysis (GSEA) (Subramanian et al., 2005) was performed with the Bioconductor GSVA (Hänzelmann et al., 2013) and the Bioconductor GSVAdata c2BroadSets gene set collection, which is the C2 collection of canonical gene sets from MsigDB3.0 (Subramanian et al., 2005). Briefly, GSEA evaluates a ranked gene expression data-set against previously defined gene sets. GSEA was performed with the following parameters: mx.diff=TRUE, verbose=TRUE, parallel.sz=1, min.sz=5, max.sz=500, rnaseq=F.

Primary Msi2+ and Msi2− KP^{f/f}C ChIP-seq for Histone H3K27ac, Stem and Non-Stem Tumor Cell Isolation Followed by H3K27ac ChIP-Sequencing

70,000 Msi2+/EpCAM+ (stem) and Msi2−/EpCAM+ (non-stem) cells were freshly isolated from a single mouse as described above. ChIP was performed as described previously (Deshpande et al., 2014); cells were pelleted by centrifugation and crosslinked with 1% formalin in culture medium using the protocol described previously (Deshpande et al., 2014). Fixed cells were then lysed in SDS buffer and sonicated on a Covaris S2 ultrasonicator. The following settings were used: Duty factor: 20%, Intensity: 4 and 200 Cycles/burst, Duration: 60 seconds for a total of 10 cycles to shear chromatin with an average fragment size of 200-400 bp. ChIP for H3K27Acetyl was performed using the antibody ab4729 (Abcam, Cambridge, UK) specific to the H3K27Ac modification. Library preparation of eluted chromatin immunoprecipitated DNA fragments was performed using the NEBNext Ultra II DNA library prep kit (E7645S and E7600S-NEB) for Illumina as per the manufacturer's protocol. Library prepped DNA was then subjected to single-end, 75-nucleotide reads sequencing on the Illumina NexSeq500 sequencer at a sequencing depth of 20 million reads per sample.

H3K27ac Signal Quantification from ChIP-seq Data

Pre-processed H3K27ac ChIP sequencing data was aligned to the UCSC mm10 mouse genome using the Bowtie2 aligner (version 2.1.0 (Langmead and Salzberg, 2012), removing reads with quality scores of <15. Non-unique and duplicate reads were removed using samtools (version 0.1.16, Li et al., 2009) and Picard tools (version 1.98), respectively. Replicates were then combined using BEDTools (version 2.17.0). Absolute H3K27ac occupancy in stem cells and non-stem cells was determined using the SICER-df algorithm without an input control (version 1.1; (Zang et al., 2009), using a redundancy threshold of 1, a window size of 200 bp, a fragment size of 150, an effective genome fraction of 0.75, a gap size of 200 bp and an E-value of 1000. Relative H3K27ac occupancy in stem cells vs non-stem cells was determined as above, with the exception that the SICER-df-rb algorithm was used.

Determining the Overlap Between Peaks and Genomic Features

Genomic coordinates for features such as coding genes in the mouse mm10 build were obtained from the Ensembl 84 build (Ensembl BioMart). The observed vs expected number of overlapping features and bases between the experimental peaks and these genomic features (datasets A and B) was then determined computationally using a custom python script, as described in (Cole et al., 2017). Briefly, the number of base pairs within each region of A that overlapped with each region of B was computed. An expected background level of expected overlap was determined using permutation tests to randomly generate >1000 sets of regions with equivalent lengths and chromosomal distributions to dataset B, ensuring that only sequenced genomic regions were considered. The overlaps between the random datasets and experimental datasets were then determined, and p values and fold changes were estimated by comparing the overlap occurring by chance (expected) with that observed empirically (observed). This same process was used to determine the observed vs expected overlap of different experimental datasets.

RNA-Seq/ChIP-Seq Correlation, Overlap Between Gene Expression and H3K27ac Modification

Genes that were up- or down-regulated in stem cells were determined using the Cuffdiff algorithm, and H3K27ac peaks that were enriched or disfavoured in stem cells were determined using the SICER-df-rb algorithm. The H3K27ac peaks were then annotated at the gene level using the ‘ChippeakAnno’ (Zhu et al., 2010) and ‘org.Mm.eg.db’ packages in R, and genes with peaks that were either exclusively up-regulated or exclusively down-regulated (termed ‘unique up’ or ‘unique down’) were isolated. The correlation between up-regulated gene expression and up-regulated H3K27ac occupancy, or down-regulated gene expression and down-regulated H3K27ac occupancy, was then determined using the Spearman method in R.

Creation of Composite Plots

Composite plots showing RNA expression and H3K27ac signal across the length of the gene were created. Up- and down-regulated RNA peaks were determined using the FPKM output values from Tophat2 (Kim et al., 2013), and up- and down-regulated H3K27ac peaks were determined using the SICER algorithm. Peaks were annotated with nearest gene information, and their location relative to the TSS was calculated. Data were then pooled into bins covering gene length intervals of 5%. Overlapping up/up and down/down sets, containing either up- or down-regulated RNA and H3K27ac, respectively, were created, and the stem and non-stem peaks within these sets were plotted in Excel.

Super-Enhancer Identification

Enhancers in stem and non-stem cells were defined as regions with H3K27ac occupancy, as described in Hnisz et al. 2013. Peaks were obtained using the SICER-df algorithm before being indexed and converted to .gff format. H3K27ac Bowtie2 alignments for stem and non-stem cells were used to rank enhancers by signal density. Super-enhancers were then defined using the ROSE algorithm, with a stitching distance of 12.5 kb and a TSS exclusion zone of 2.5 kb. The resulting super-enhancers for stem or non-stem cells were then annotated at the gene level using the R packages ‘ChippeakAnno’ (Zhu et al., 2010) and ‘org.Mm.eg.db’, and overlapping peaks between the two sets were determined using ‘ChippeakAnno’. Super-enhancers that are unique to stem or non-stem cells were annotated to known biological pathways using the Gene Ontology (GO) over-representation analysis functionality of the tool WebGestalt (Wang et al., 2017).

Genome-Wide CRISPR Screen, CRISPR Library Amplification and Viral Preparation

The mouse GeCKO CRISPRv2 knockout pooled library (Sanjana et al., 2014) was acquired from Addgene (catalog #1000000052) as two half-libraries (A and B). Each library was amplified according to the Zhang lab library amplification protocol (Sanjana et al., 2014) and plasmid DNA was purified using NucleoBond Xtra Maxi DNA purification kit (Macherey-Nagel). For lentiviral production, 24×T225 flasks were plated with 21×10⁶ 293T each in 1× DMEM containing 10% FBS. 24 hours later, cells were transfected with pooled GeCKOv2 library and viral constructs. Briefly, media was removed and replaced with 12.5 ml warm OptiMEM (Gibco). Per plate, 200 μl PLUS reagent (Life Technologies), 10 μg library A, and 10 μg library B was mixed in 4 ml OptiMEM along with 10 μg pRSV/REV (Addgene), 10 μg pMDLg/pRRE (Addgene), and 10 μg pHCMVG (Addgene) constructs. Separately, 200 μl Lipofectamine (Life Technologies) was mixed with 4 ml OptiMEM. After 5 minutes, the plasmid mix was combined with Lipofectamine and left to incubate at room temperature for 20 minutes, then added dropwise to each flask. Transfection media was removed 22 hours later and replaced with DMEM containing 10% FBS, 5 mM MgCl₂, 1 U/ml DNase (Thermo Scientific), and 20 mM HEPES pH 7.4. Viral supernatants were collected at 24 and 48 hours, passaged through 0.45 μm filter (corning), and concentrated by ultracentrifugation at 20,000 rpm for 2 hours at 4° C. Viral particles were resuspended in DMEM containing 10% FBS, 5 mM MgCl₂, and 20 mM HEPES pH 7.4, and stored at −80° C.

CRISPR Screen in Primary KP^{f/f}C Cells

3 independent primary REM2-KP^{f/f}C cell lines were established as described above and maintained in DMEM containing 10% FBS, 1× non-essential amino acids, and 1× pen/strep. At passage 3, each cell line was tested for puromycin sensitivity and GeCKOv2 lentiviral titer was determined. At passage 5, 1.6×10⁸ cells from each cell line were transduced with GeCKOv2 lentivirus at an MOI of 0.3. 48 hours after transduction, 1×10⁸ cells were harvested for sequencing (“T0”) and 1.6×10⁸ were re-plated in the presence of puromycin according to previously tested puromycin sensitivity. Cells were passaged every 3-4 days for 3 weeks; at every passage, 5×10⁷ cells were re-plated to maintain library coverage. At 2 weeks post-transduction, cell lines were tested for sphere forming capacity. At 3 weeks, 3×10⁷ cells were harvested for sequencing (“2D; cell essential genes”), and 2.6×10⁷ cells were plated in sphere conditions as described above (“3D; stem cell essential genes”). After 1 week in sphere conditions, tumorspheres were harvested for sequencing.

Analysis of the 2D data sets revealed that while some genes were required for growth in 2D, other genes that were not (detectably) required for growth in 2D were still required for growth in 3D (for example, Rorc Sox4, Foxo1, Wnt1 and ROBO3). These findings suggested that growth in 3D is dependent on a distinct or additional set of pathways. Since only stem cells give rise to 3D spheres, targets within the 3D datasets were prioritized for subsequent analyses. Of the genes that significantly dropped out in 3D, some also dropped out in 2D either significantly or as a trend.

DNA Isolation, Library Preparation, and Sequencing

Cells pellets were stored at −20° C. until DNA isolation using Qiagen Blood and Cell Culture DNA Midi Kit (13343). Briefly, per 1.5×10⁷ cells, cell pellets were resuspended in 2 ml cold PBS, then mixed with 2 ml cold buffer C1 and 6 ml cold H₂O, and incubated on ice for 10 minutes. Samples were pelleted 1300×g for 15 minutes at 4° C., then resuspended in 1 ml cold buffer C1 with 3 ml cold H₂O, and centrifuged again. Pellets were then resuspended in 5 ml buffer G2 and treated with 100 μl RNAse A (Qiagen 1007885) for 2 minutes at room temperature followed by 95 μl Proteinase K for 1 hour at 50° C. DNA was extracted using Genomic-tip 100/G columns, eluted in 50° C. buffer QF, and spooled into 300 μl TE buffer pH 8.0. Genomic DNA was stored at 4° C. For sequencing, gRNAs were first amplified from total genomic DNA isolated from each replicate at T0, 2D, and 3D (PCR1). Per 50 μl reaction, 4 μg gDNA was mixed with 25 μl KAPA HiFi HotStart ReadyMIX (KAPA Biosystems), 1 μM reverse primer1, and 1 μM forward primer1 mix (including staggers). Primer sequences are available upon request. After amplification (98° C. 20 seconds, 66° C. 20 seconds, 72° C. 30 seconds, ×22 cycles), 50 μl of PCR1 products were cleaned up using QIAquick PCR Purification Kit (Qiagen). The resulting ˜200 bp products were then barcoded with IIlumina Adaptors by PCR2. 5 μl of each cleaned PCR1 product was mixed with 25 μl KAPA HiFi HotStart ReadyMIX (KAPA Biostystems), 10 μl H₂O, 1 μM reverse primer2, and 1 μM forward primer2. After amplification (98° C. 20 seconds, 72° C. 45 seconds, ×8 cycles), PCR2 products were gel purified, and eluted in 30 μl buffer EB. Final concentrations of the desired products were determined and equimolar amounts from each sample was pooled for Next Generation Sequencing.

Processing of the CRISPR Screen Data

Sequence read quality was assessed using fastqc (www.bioinformatics.babraham.ac.uk/proiects/fastqc/). Prior to alignment, 5′ and 3′ adapters flanking the sgRNA sequences were trimmed off using cutadapt v1.11 (Martin, 2011) with the 5′-adapter TCTTGTGGAAAGGACGAAACACCG (SEQ ID NO: 1) and the 3′ adapter GTTTTAGAGCTAGAAATAGCAAGTT (SEQ ID NO: 2), which came from the cloning protocols of the respective libraries deposited on Addgene (www.addgene.org/pooled-library/). Error tolerance for adapter identification was set to 0.25, and minimal required read length after trimming was set to 10 bp. Trimmed reads were aligned to the GeCKO mouse library using Bowtie2 in the—local mode with a seed length of 11, an allowed seed mismatch of 1 and the interval function set to ‘S,1,0.75’. After completion, alignments are classified as either unique, failed, tolerated or ambiguous based on the primary (‘AS’) and secondary (‘XS’) alignment scores reported by Bowtie2. Reads with the primary alignment score not exceeding the secondary score by at least 5 points were discarded as ambiguous matches. Read counts were normalized by using the “size-factor” method. All of this was done using implementations in the PinAPL-Py webtool, with detailed code available at github.com/LewisLabUCSD/PinAPL-Py.

gRNA Growth and Decay Analysis

A parametric method is used in which the cell population with damaged gene i grows as Nᵢ(t)=Nᵢ(0)e^{(α}^{₀}^{+δ}^{ₜ}^{)t}, where α₀ is the growth rate of unmodified cells and δᵢ is the change of the growth rate due to the gene deletion. Since the aliquot extracted at each time point is roughly the same and represents only a fraction of the entire population, the observed sgRNA counts nᵢ do not correspond to Nᵢ directly. The correspondence is only relative: if we define cᵢ≡nᵢ/Σₖnₖ as the compositional fraction of sgRNA species i, the correspondence is cᵢ=NᵢΣₖNₖ. As a result, the exponential can only be determined up to a multiplicative constant, e^{-δ}^{ᵢ}^{t}=A·cᵢ(0)/cᵢ(t). The constant is determined from the assumption that a gene deletion typically does not affect the growth rate. Mathematically, 1=A med[cᵢ(0)/cᵢ(t)]. The statistic that measures the effect of gene deletion is defined as xᵢ≡e^{-δ}^{ᵢ}^{t} and calculated for every gene i from

$${x}_{i} = A \frac{{c}_{i} ( 0 )}{{c}_{i} ( t )} . \qquad$$

Since we are interested in genes essential for growth, we performed a single-tailed test for xᵢ. We collect the three values of xᵢ, one from each biological replicate, into a vector xᵢ. A statistically significant effect will have all three values large (>1) and consistent. If xᵢ were to denote position of a point in a three-dimensional space, we would be interested in points that lie close to the body diagonal and far away from the origin. A suitable statistic is s=(x·n)²−[x−(x·n)n]², where n=(1,1,1)/\sqrt{3} is the unit vector in the direction of the body diagonal and · denotes scalar product. A q-value (false discovery rate) for each gene is estimated as the number of s-statistics not smaller than sᵢ expected in the null model divided by the observed number of S-statistics not smaller than sᵢ in the data. The null model is simulated numerically by permuting gene labels in xᵢ for every experimental replicate, independently of each other, repeated 10³ times.

STRING Interactome Network Analysis

The results from the CRISPR 3DV experiment were integrated with the RNA-seq results using a network approach. Likely CRISPR-essential genes were identified by filtering to include genes which had a false-discovery rate corrected p-value of less than 0.5, resulting in 94 genes. A relaxed filter was chosen here because the following filtering steps will help eliminate false positives, and the network analysis method helps to amplify weak signals. These genes were further filtered in two ways: first, we included only genes which were expressed in the RNA-seq data (this resulted in 57 genes), and second, we further restricted by genes which had enriched expression in stem cells by >2 log fold change in the RNA-seq (this resulted in 10 genes). These results are used to seed the network neighborhood exploration. We used the STRING mouse interactome as our background network, including only high confidence interactions (edge weight>700). The STRING interactome contains known and predicted functional protein-protein interactions. The interactions are assembled from a variety of sources, including genomic context predictions, high throughput lab experiments, and co-expression databases. Interaction confidence is a weighted combination of all lines of evidence, with higher quality experiments contributing more. The high confidence STRING interactome contains 13,863 genes, and 411,296 edges. Because not all genes are found in the interactome, our seed gene sets are further filtered when integrated with the network. This results in 39 CRISPR-essential, RNA-expressed seed genes, and 5 CRISPR-essential, RNA differentially-expressed seed genes. After integrating the seed genes with the background interactome, we employed a network propagation algorithm to explore the network neighborhood around these seed genes. Network propagation is a powerful method for amplifying weak signals by taking advantage of the fact that genes related to the same phenotype tend to interact. We implement the network propagation method that simulates how heat would diffuse, with loss, through the network by traversing the edges, starting from an initially hot set of ‘seed’ nodes. At each step, one unit of heat is added to the seed nodes, and is then spread to the neighbor nodes. A constant fraction of heat is then removed from each node, so that heat is conserved in the system. After a number of iterations, the heat on the nodes converges to a stable value. This final heat vector is a proxy for how close each node is to the seed set. For example, if a node was between two initially hot nodes, it would have an extremely high final heat value, and if a node was quite far from the initially hot seed nodes, it would have a very low final heat value. This process is described by the following as in (Vanunu et al., 2010):

F^{t}=W′F^{t-1}+(1−α)Y

Where F^{t} is the heat vector at time t, Y is the initial value of the heat vector, W′ is the normalized adjacency matrix, and α ∈ (0,1) represents the fraction of total heat which is dissipated at every timestep. We examine the results of the subnetwork composed of the 500 genes nearest to the seed genes after network propagation. This will be referred to as the ‘hot subnetwork’. In order to identify pathways and biological mechanisms related to the seed genes, we apply a clustering algorithm to the hot subnetwork, which partitions the network into groups of genes which are highly interconnected within the group, and sparsely connected to genes in other groups. We use a modularity maximization algorithm for clustering, which has proven effective in detecting modules, or clusters, in protein-protein interaction networks. These clusters are annotated to known biological pathways using the over-representation analysis functionality of the tool WebGestalt. We use the 500 genes in the hot subnetwork as the background reference gene set. To display the networks, we use a spring-embedded layout, which is modified by cluster membership (along with some manual adjustment to ensure non-overlapping labels). Genes belonging to each cluster are laid out radially along a circle, to emphasize the within cluster and between cluster connections. VisJS2jupyter was used for network propagation and visualization. Node color is mapped to the RNAseq log fold change, with down-regulated genes displayed in blue, upregulated genes displayed in red, and genes with small fold changes displayed in gray. Labels are shown for genes which have a log fold change with absolute value greater than 3.0. Seed genes are shown as triangles with white outlines, while all other genes in the hot subnetwork are circles. The clusters have been annotated by selecting representative pathways from the enrichment analysis.

KP^{R172H}C Single Cell Analysis

Freshly harvested tumors from two independent KP^{R172h}C mice were subjected to mechanical and enzymatic dissociation using a Miltenyi gentleMACS Tissue Dissociator to obtain single cells. The 10× Genomics Chromium Single Cell Solution was employed for capture, amplification and labeling of mRNA from single cells and for scRNA-Seq library preparation. Sequencing of libraries was performed on a Illumina HiSeq 2500 system. Sequencing data was input into the Cell Ranger analysis pipeline to align reads and generate gene-cell expression matrices. Finally, Custom R packages were used to perform gene-expression analyses and cell clustering projected using the t-SNE (t-Distributed Stochastic Neighbor Embedding) clustering algorithm. scRNA-seq datasets from the two independent KP^{R127h}C tumor tissues generated on 10× Genomics platform were merged and utilized to explore and validate the molecular signatures of the tumor cells under dynamic development. The tumor cells that were used to illustrate the signal of Il10rb, Il34 and Csf1r etc. were characterized from the heterogeneous cellular constituents using SuperCT method developed by Dr. Wei Lin and confirmed by the Seurat FindClusters with the enriched signal of Epcam, Krt19 and Prom1 etc. (Xie et al., 2018). The tSNE layout of the tumor cells was calculated by Seurat pipeline using the single-cell digital expression profiles.

KP^{f/f}C Single Cell Analysis

Three age-matched KP^{f/f}C pancreatic tumors were collected and freshly dissociated, as described above. Tumor cells were stained with rat anti-mouse CD45-PE/Cy7 (eBioscience), rat anti-mouse CD31-PE (eBioscience), and rat anti-mouse PDGFRα-PacBlue (eBioscience) and tumor cells negative for these three markers were sorted for analysis. Individual cells were isolated, barcoded, and libraries were constructed using the 10× genomics platform using the Chromium Single Cell 3′ GEM library and gel bead kit v2 per manufacturer's protocol. Libraries were sequenced on an Illumina HiSeq4000. The Cell Ranger software was used for alignment, filtering and barcode and UMI counting. The Seurat R package was used for further secondary analysis using default settings for unsupervised clustering and cell type discovery.

shRorc vs. shCtrl KP^{f/f}C RNA-seq

Primary WT-KP^{f/f}C cell lines were established as described above. WT-KP^{f/f}C cells derived from an individual low passage cell line (<6 passage) were plated and transduced in triplicate with lentiviral particles containing shCtrl or shRorc. Positively infected (red) cells were sorted 5 days after transduction. Total RNA was isolated using the RNeasy Micro Plus kit (Qiagen). RNA libraries were generated from 200 ng of RNA using Illumina's TruSeq Stranded mRNA Sample Prep Kit (Illumina) following manufacturer's instructions. Libraries were pooled and single end sequenced (1×75) on the Illumina NextSeq 500 using the High output V2 kit (Illumina Inc., San Diego Calif.).

Read data was processed in BaseSpace (basespace.illumina.com). Reads were aligned to Mus musculus genome (mm10) using STAR aligner (code.google.com/p/rna-star/) with default settings. Differential transcript expression was determined using the Cufflinks Cuffdiff package (Trapnell et al., 2012) (github.com/cole-trapnell-lab/cufflinks). Differential expression data was then filtered to represent only significantly differentially expressed genes (q value<0.05). This list was used for pathway analysis and heatmaps of specific significantly differentially regulated pathways.

shRorc vs. shCtrl KP^{f/f}C ChIP-seq for Histone H3K27ac

Primary WT-KP^{f/f}C cell lines were established as described above. Low passage (<6 passages) WT-KP^{f/f}C cells from two independent cell lines were plated and transduced in triplicate with lentiviral particles containing shCtrl or shRorc. Positively infected (red) cells were sorted 5 days after transduction. ChIP-seq for histone H3K27-ac, signal quantification, and determination of the overlap between peaks and genomic features was conducted as described above.

Super-enhancers in control and shRorc-treated KP^{f/f}C cell lines as well as Musashi stem cells were determined from H3K27ac ChlPseq data using the ROSE algorithm (younglab.wi.mit.edu/super enhancer code.html). The Musashi stem cell super-enhancer peaks were then further refined to include only those unique to the stem cell state (defined as present in stem cells but not non-stem cells) and/or those with RORγ binding sites within the peaks. Peak sequences were extracted using the ‘getSeq’ function from the ‘BSGenome.MMusculus.UCSC.mm10’ R package. RORγ binding sites were then mapped using the matrix RORG_MOUSE.H10MO.C.pcm (HOCOMOCO database) as a reference, along with the ‘matchPWM’ function in R at 90% stringency. Baseline peaks were then defined for each KP^{f/f}C cell line as those overlapping each of the four Musashi stem cell peaklists with each KPC control SE list, giving eight in total. The R packages ‘GenomicRanges’ and ‘ChIPpeakAnno’ were used to assess peak overlap with a minimum overlap of 1 bp used. To estimate the proportion of super-enhancers that are closed on RORC knockdown, divergence between each baseline condition and the corresponding KP^{f/f}C shRorc super-enhancer list was assessed by quantifying the peak overlap and then expressing this as a proportion of the baseline list (‘shared %’). The proportion of unique peaks in each condition was then calculated as 100%-shared % and plotted.

sgRORC vs sgNT Human RNA-seq

Human FG cells were plated and transduced in triplicate with lentiviral particles containing Cas9 and non-targeting guide RNA or guide RNA against Rorc. Positively infected (green) cells were sorted 5 days after transduction. Total RNA was isolated using the RNeasy Micro Plus kit (Qiagen). RNA libraries were generated from 200 ng of RNA using Illumina's TruSeq Stranded mRNA Sample Prep Kit (Illumina) following manufacturer's instructions. Libraries were pooled and single end sequenced (1×75) on the Illumina NextSeq 500 using the High output V2 kit (Illumina Inc., San Diego Calif.).

Comparative RNA-seq and Cell State Analysis

RORC knockdown and control RNA-seq fastq files in mouse KP^{f/f}C and human FG cells were processed into transcript-level summaries using kallisto (Bray et al., 2016). Transcript-level summaries were processed into gene-level summaries and differential gene expression was performed using sleuth with the Wald test (Pimentel et al., 2017). GSEA was performed as detailed above (Subramanian et al., 2005). Gene ontology analysis was performed using Metascape using a custom analysis with GO biological processes and default settings with genes with a FDR<5% and a beta value>0.5.

cBioportal

RORC genomic amplification data from cancer patients was collected from the Memorial Sloan Kettering Cancer Center cBioPortal for Cancer Genomics (www.cbioportal.org).

Quantification and Statistical Analysis

Statistical analyses were carried out using GraphPad Prism software version 7.0d (GraphPad Software Inc.). Sample sizes for in vivo drug studies were determined based on the variability of pancreatic tumor models used. For flank transplant and autochthonous drug studies, tumor bearing animals within each group were randomly assigned to treatment groups. Treatment sizes were determined based on previous studies (Fox et al., 2016). Data are shown as the mean±SEM. Two-tailed unpaired Student's t-tests with Welch's correction or One-way analysis of variance (ANOVA) for multiple comparisons when appropriate were used to determine statistical significance (*P<0.05, **P<0.01, ***P<0.001, ****P<0.0001).

The level of replication for each in vitro and in vivo study is noted in the figure legends for each figure and described in detail in the Method Details section above. However to summarize briefly, in vitro tumorsphere or colony formation studies were conducted with n=3 independent wells per cell line across two independent shRNA of n=3 wells; however, the majority of these experiments were additionally completed in >1 independently derived cell line, n=3 wells per shRNA. For limiting dilution assays, organoids were derived from 3 independent mice; drug-treated mouse and human organoids were plated at n=3 wells per dose per treatment condition. Flank shRNA studies were conducted twice independently, with n=4 tumors per group in each experiment. Flank drug studies were conducted at n=2-7 tumors per treatment group; autochthonous KP^{f/f}C survival studies were conducted with a minimum of 4 mice enrolled in each treatment group. Live imaging studies were carried out with two mice per treatment group.

Statistical considerations and bioinformatic analysis of large data-sets generated are explained in great detail above. In brief, primary KP^{f/f}C RNA-seq was performed using Msi2+ and Msi2− cells sorted independently from three different end-stage KP^{f/f}C mice. Primary KP^{f/f}C ChIP-seq was performed using Msi2+ and Msi2− cells sorted from an individual end-stage KP^{f/f}C mouse. The genome-wide CRISPR screen was conducted using three biologically independent cell lines (derived from three different KP^{f/f}C tumors). Single-cell analysis of tumors represents merged data from ˜10,000 cells across two KP^{R172H}C and three KP^{f/f}C mice. RNA-seq for shRorc and shCtrl KP^{f/f}C cells was conducted in triplicate, while ChIP-seq was conducted in single replicates from two biologically independent KP^{f/f}C cell lines.

### Example 2

This working example demonstrates that the RORγ pathway plays important roles in more aggressive subtypes of pancreatic cancer and can prevent cancer progression from benign to malignant state.

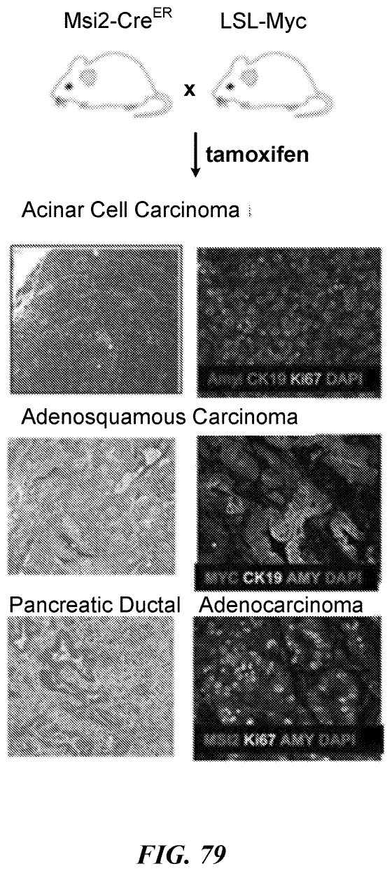

RORγ inhibition has been demonstrated to block growth of adenosquamous carcinoma of the pancreas (ASCP), the most aggressive subtype of pancreatic cancer. A new Msi2-Cre^{ER} mouse model of aggressive pancreatic cancer was created, in which Cre is driven off of the Msi2 promoter and can be conditionally triggered by tamoxifen delivery. This Msi2-Cre^{ER} driver can be crossed into mice bearing distinct mutations such as Ras (leading to myeloproliferative neoplasia), p53, or Myc. When the Msi2-Cre^{ER} driver was crossed into an LSL-MyC^{T58A} model developed by Dr. Robert Wechsler-Reya at SBP/Rady, La Jolla, Calif. (Mollaoglu et al., 2017) (FIG. 79), it produced multiple cancer types including small cell lung cancer, choroid plexus tumors, and early stage kidney tumors. In the pancreas, it resulted in adenosquamous carcinoma, an aggressive sub-type of pancreatic cancer with the worst clinical prognosis among all pancreatic cancers, as well as acinar cell carcinoma (ACC), a subtype enriched in pediatric patients and marked by frequent relapses.

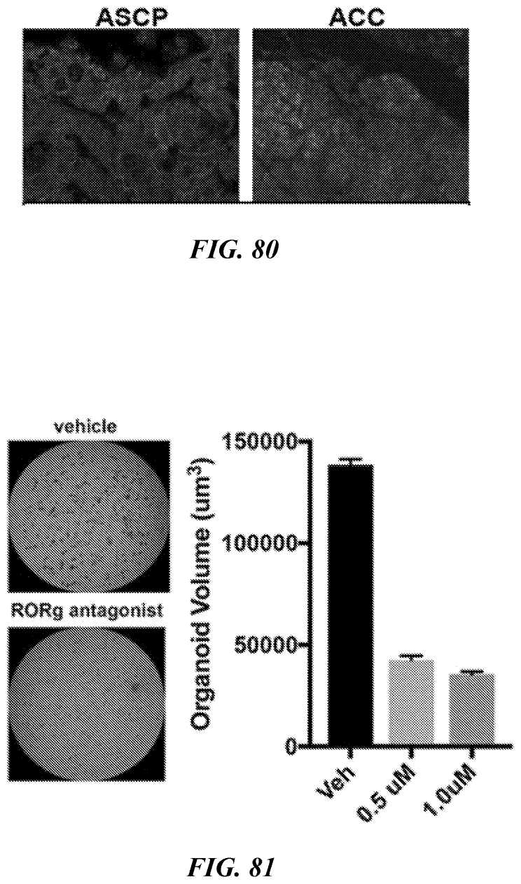

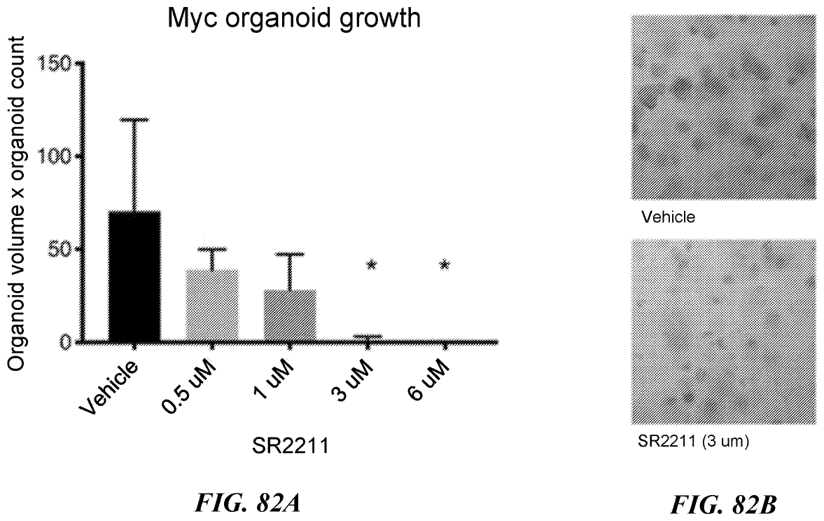

Using this model, high expression of RORγ was observed in ASCP and ACC tumors (FIG. 80), suggesting a role for RORγ in regulating tumor growth. Importantly, this data is supported by functional studies which showed that organoids derived from both adenosquamous tumors and acinar tumors are sensitive to SR2211, an inhibitor of RORγ (FIGS. 81, 82A, and 82B). FIG. 82A shows organoid growth in the presence of vehicle or increasing doses of SR2211, including 0.5 μM, 1 μM, 3 μM, and 6 μM. FIG. 82B shows representative images of organoids in the presence of vehicle or 3 μM SR2211. 3 μM or 6 μM SR2211 significantly reduced organoid growth. Collectively, these models and data suggest that RORγ is required more broadly for distinct pancreatic tumor sub-types, which may in turn expand the pool of patients who could benefit from a novel therapeutic approach targeting RORγ.

Moreover, RORγ inhibitor SR2211 can block the growth of benign pancreatic intraepithelial neoplasia (PanIN) lesions. The effect of SR2211 was tested on dissociated primary murine PanIN derived organoids. SR2211 reduced both organoid number and organoid volume, suggesting that RORγ inhibition may prevent cancer progression from benign to malignant state.

### Example 3

This working example demonstrates that RORγ also plays an important role in leukemia and presents a promising target in the treatment of leukemia potentially due to the similarities between leukemia and pancreatic cancer stem cells. The data suggests that inhibition of RORγ is effective at reducing leukemia cell growth and projects RORγ inhibitors as promising therapeutic agents for treating leukemia.

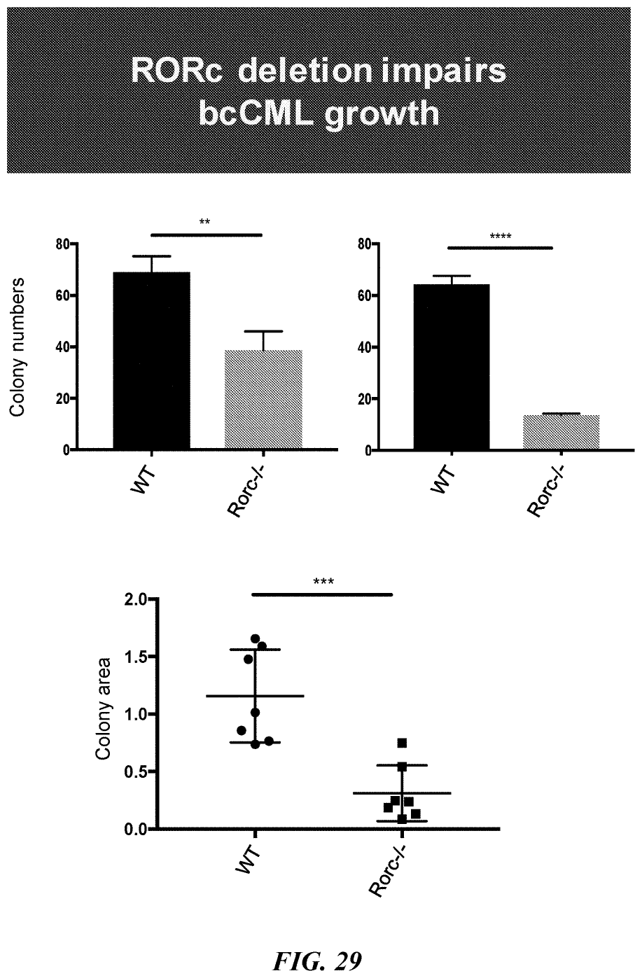

Given the common features and shared molecular dependencies between leukemia and pancreatic cancer stem cells, it was examined whether RORγ was also required for growth of aggressive leukemia, using blast crisis chronic myeloid leukemia (CML) as a model. As shown in FIG. 29, KLS cells were isolated from WT and RORγ knockout (Rorc−/−) mice, retrovirally transduced with BCR-ABL and Nup98-HOXA9, and cultured in primary and secondary colony assays in vitro. Importantly, a significant decrease in both colony number and overall colony area in primary and secondary colony assays was observed, indicating that growth and propagation of blast crisis CML is critically dependent on RORγ. In addition, an impact on acute myelogenous leukemia (AML) growth as well as RORγ expression in lymphoid tumors was observed, suggesting a role for RORγ signaling in these cancers as well.

### Example 4

This working example demonstrates that RORγ also plays an important role in lung cancer, as pharmacological inhibition of RORγ by SR2211 inhibited tumor sphere formation of lung cancer cells, suggesting that therapeutic approaches targeting RORγ can be effective at treating lung cancer.

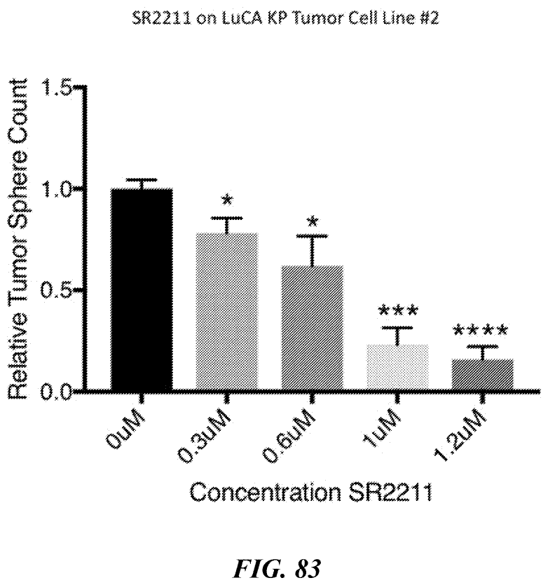

As shown in FIG. 83, LuCA KP lung cancer cells were treated with vehicle or increasing doses of SR2211, including 0.3 μM, 0.6 μM, 1 μM, and 1.2 μM. Then the number of formed tumor spheres were counted and quantified as relative to control. SR211 at all doses tested significantly reduced tumor sphere formation, and the extent of reduction increases with the dosage of SR2211.

### Example 5

This working example demonstrates that AZD-0284, an inhibitor of RORγ, is effective in impairing the growth of mammalian pancreatic cancer and leukemia. The results suggest that AZD-0284 can be an effective therapeutic agent for cancer treatment.

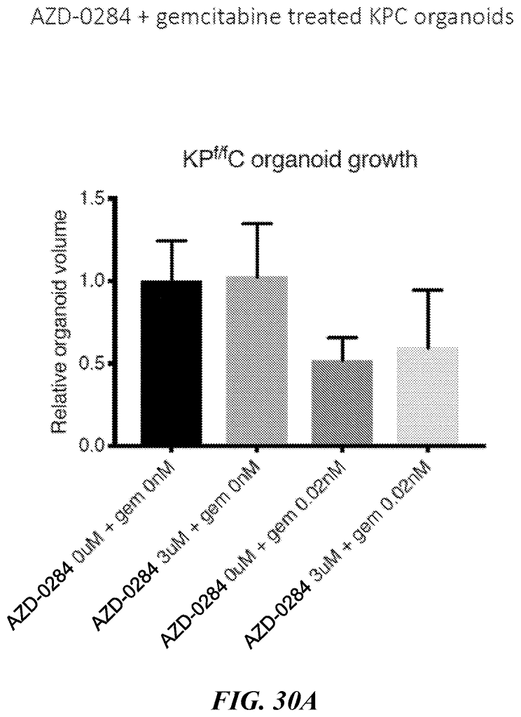

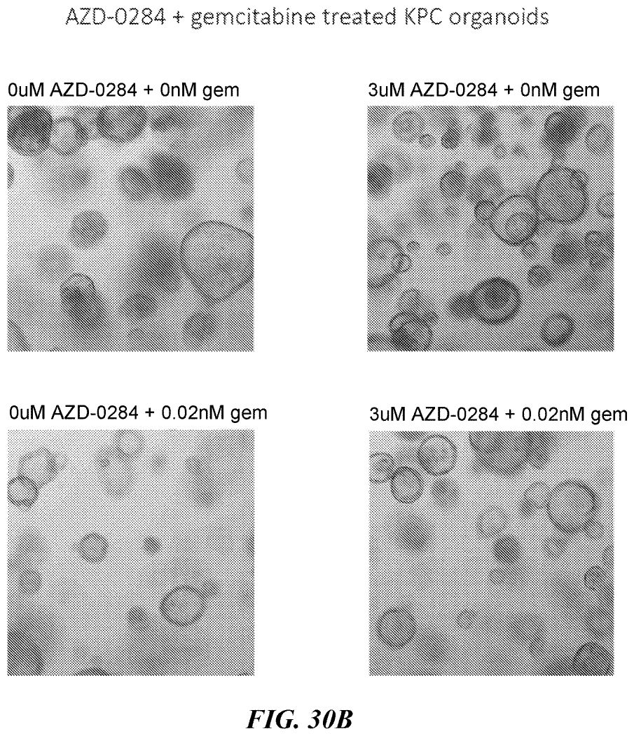

Pharmacologic blockade of RORγ using AZD-0284 in combination with gemcitabine decreased KP^{f/f}C organoid growth (FIG. 30). KP^{f/f}C organoid were derived from the REM2-KP^{f/f}C mice, a germline genetically engineered mouse model for pancreatic ductal adenocarcinoma with the genotype of Msi2^{eGFP}/Kras^{LSL-G12D/+}; Pdx^{CRE/+}; p53^{f/f}. Briefly, tumors from 10-12-week-old end-stage REM2-KP^{f/f}C mice were harvested and dissociated into a single cell suspension. Tumor cells were stained with FC block then 0.2 μg/10⁶ cells anti-EpCAM APC (eBioscience). REM2+/EpCAM+ (stem) and REM2−/EpCAM+ (non-stem) cells were sorted, resuspended in 20 μl Matrigel (BD Biosciences, 354230), and plated as a dome in a pre-warmed 48-well plate. After incubation at 37° C. for 5 min, domes were covered with 300 μl PancreaCult Organoid Growth Media (Stemcell Technologies). Organoids were imaged and quantified 6 days later. All images were acquired on a Zeiss Axiovert 40 CFL. Organoids were counted and measured using ImageJ 1.51s software.

The derived KP^{f/f}C organoid were maintained and passaged at ˜1:2. Briefly, organoids were isolated using Cell Recovery Solution (Corning 354253), then dissociated using Accumax Cell Dissociation Solution (Innovative Cell Technologies AM105), and plated in 20 μl Matrigel (BD Biosciences, 354230) domes on a pre-warmed 48-well plate. After incubation at 37° C. for 5 min, domes were covered with 300 μl PancreaCult Organoid Growth Media (Stemcell Technologies).

The organoid forming capacity of KP^{f/f}C cells grown in the presence of vehicle, 3 μM AZD-0284, 0.02 nM gemcitabine, or both was assessed by imaging and measurements of organoid volume (FIG. 30). The volume of organoids was expressed as relative to control. As shown in FIG. 30, 0.02 nM gemcitabine alone or in combination with 3 μM AZD-0284 visibly decreased organoid growth in volume.

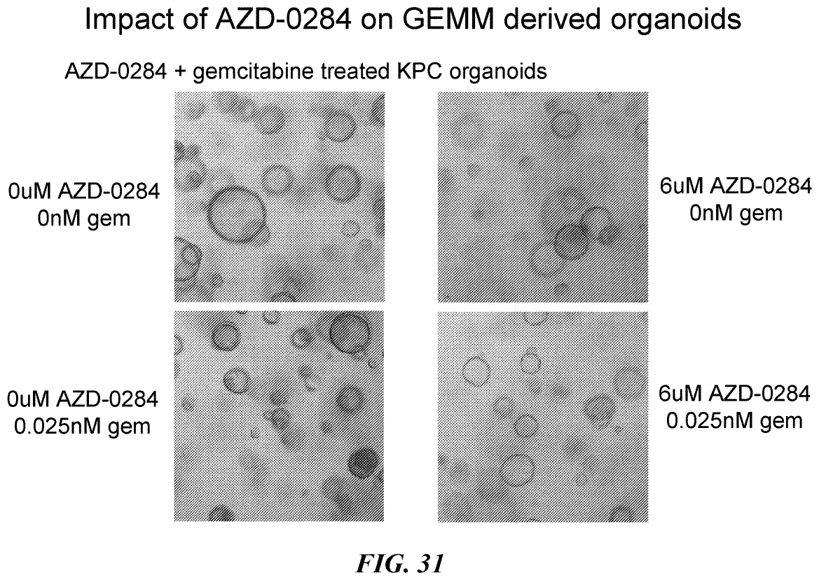

The effect of AZD-0284 at a higher dose on KP^{f/f}C organoid growth was also examined (FIG. 31). KP^{f/f}C organoids were cultured in the presence of vehicle, 6 μM AZD-0284, 0.025 nM gemcitabine, or both, followed by imaging. As shown in FIG. 31, the treatment of AZD-0284 alone, gemcitabine alone, or AZD-0284 and gemcitabine combination each resulted in visibly reduced organoid volume of KP^{f/f}C cells.

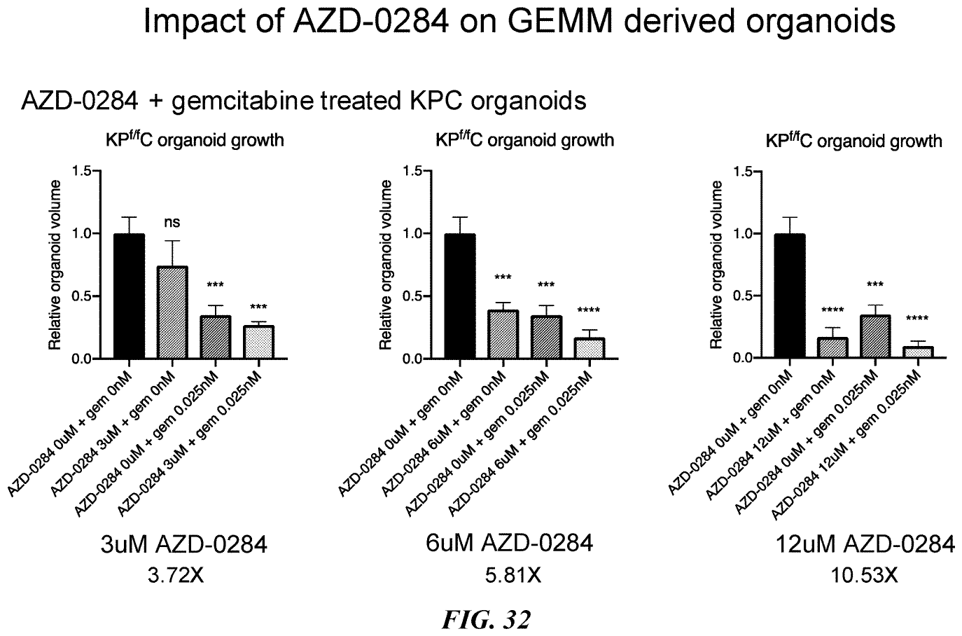

Similarly, the effects of AZD-0284 at different doses were examined on KP^{f/f}C organoids (FIG. 32). Three doses of AZD-0284 were tested: 3 μM, 6 μM, and 12 μM. For each AZD-0284 dose, four conditions were tested: vehicle, AZD-0284 alone, gemcitabine alone (at 0.025 nM), and a combination of AZD-0284 and gemcitabine. Consistent with previously described, 0.025 nM gemcitabine alone resulted in significant inhibition of KP^{f/f}C organoid growth. AZD-0284, when administered alone, had a significant inhibitory effect at higher doses, e.g., 6 μM or 12 μM. On the other hand, AZD-0284, if given in combination with gemcitabine, resulted in the highest inhibitory effect of KP^{f/f}C organoid growth at all doses tested. The combination of 0.025 nM gemcitabine and 3 μM AZD-0284, 6 μM AZD-0284, or 12 μM AZD-0284 led to a 3.72-, 5.81-, or 10.53-fold decrease, respectively, in organoid volume compared to control. Thus, the data suggest a synergistic effect between RORγ inhibition and chemotherapy medication for pancreatic cancer treatment.

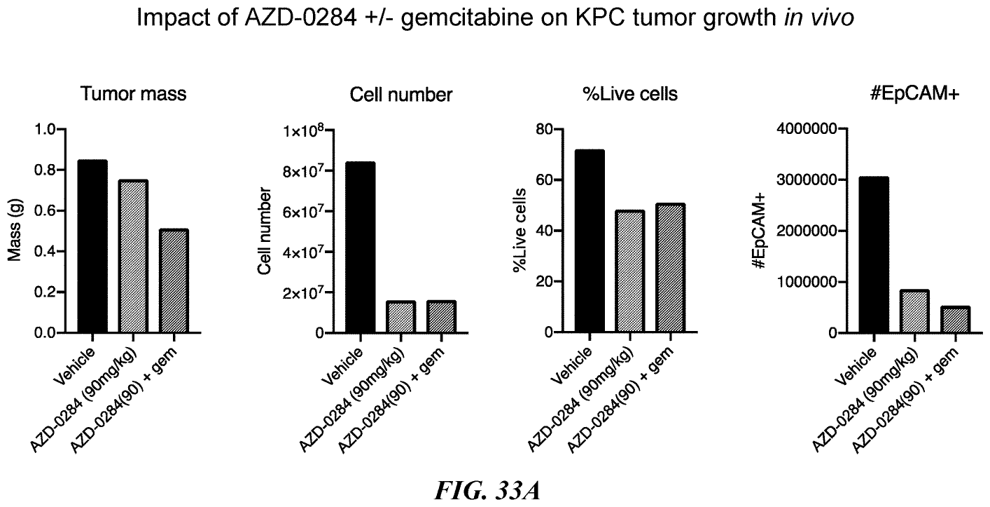

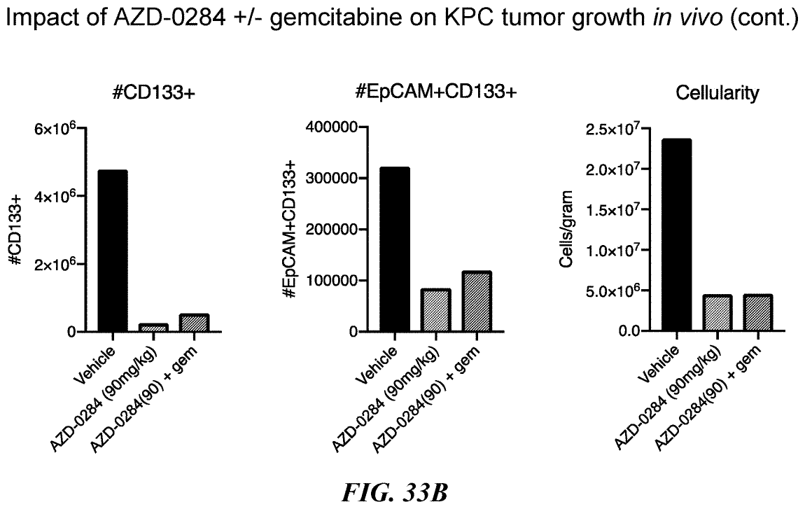

Next, the impact of AZD-0284 was tested on tumor-bearing KP^{f/f}C mice in vivo (FIG. 33). KP^{f/f}C mice was allowed to develop tumor before treatment with vehicle, 90 mg/kg AZD-0284, or 90 mg/kg AZD-0284 in combination with gemcitabine began. As shown in FIG. 33, mice that received 90 mg/kg body weight of AZD-0284 exhibited lower tumor mass, cell number, and a loss of EpCam+ tumor epithelial cells and EpCam+/CD133+ tumor stem cells. A similar effect was observed in mice that received both AZD-0284 and gemcitabine, suggesting that AZD-0284, either given alone or in combination with gemcitabine, was effective at reducing pancreatic tumor in vivo.

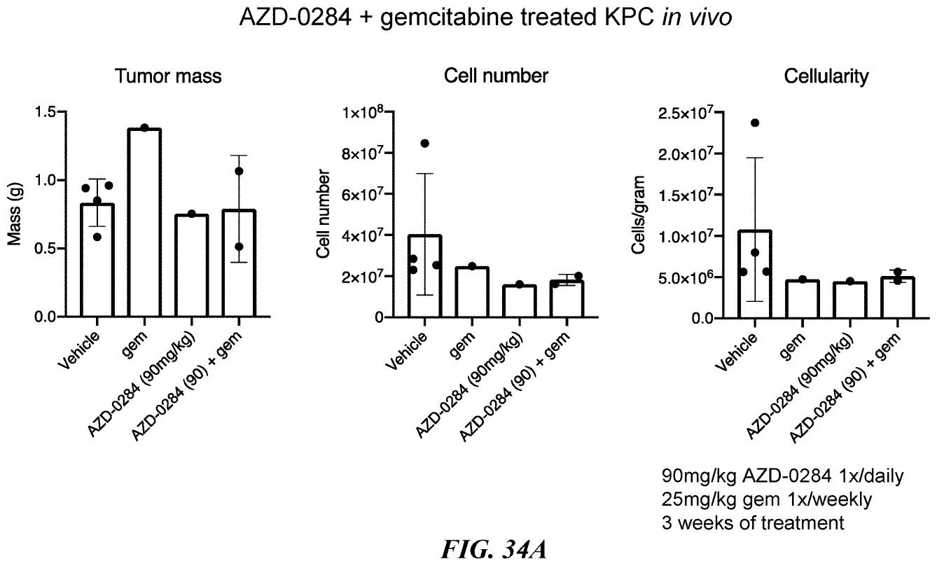

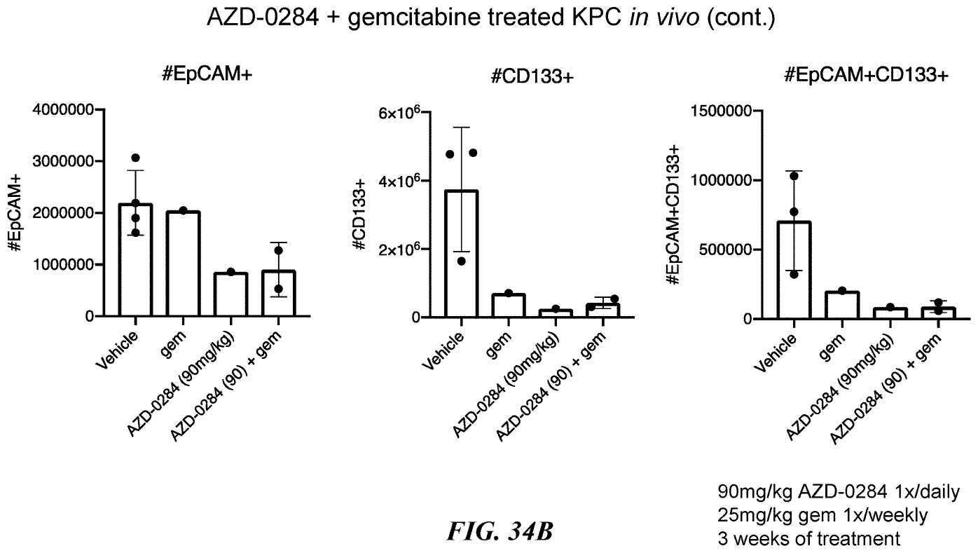

FIG. 34 shows a compilation of tumor-bearing KP^{f/f}C mice treated with gemcitabine alone, AZD-0284 alone, or AZD-0284 plus gemcitabine. AZD-0284 was given at 90 mg/kg once daily, and gemcitabine was given at 25 mg/kg once weekly, for 3 weeks. As previously seen, mice treated with AZD-0284 alone or a combination of AZD-0284 and gemcitabine exhibited lower cell number and a loss of EpCam+ tumor epithelial cells and EpCam+/CD133+ tumor stem cells, suggesting efficacy of RORγ inhibition as cancer treatment therapy, alone or in combination with chemotherapy.

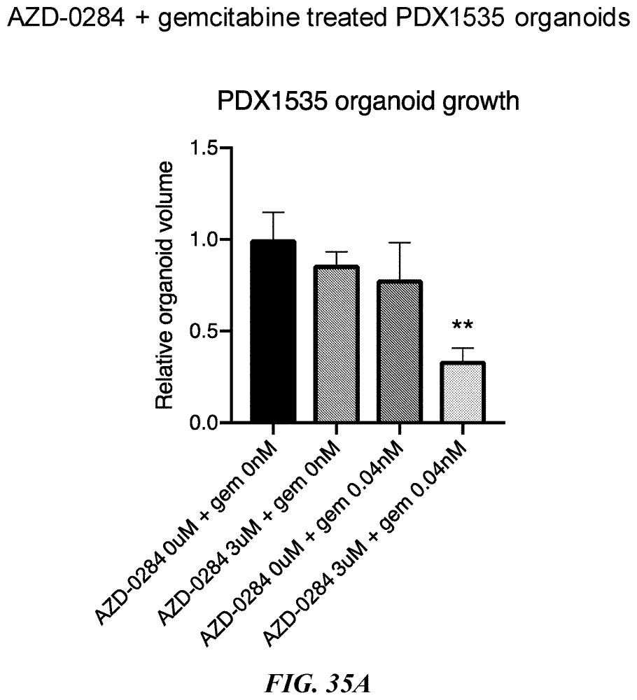

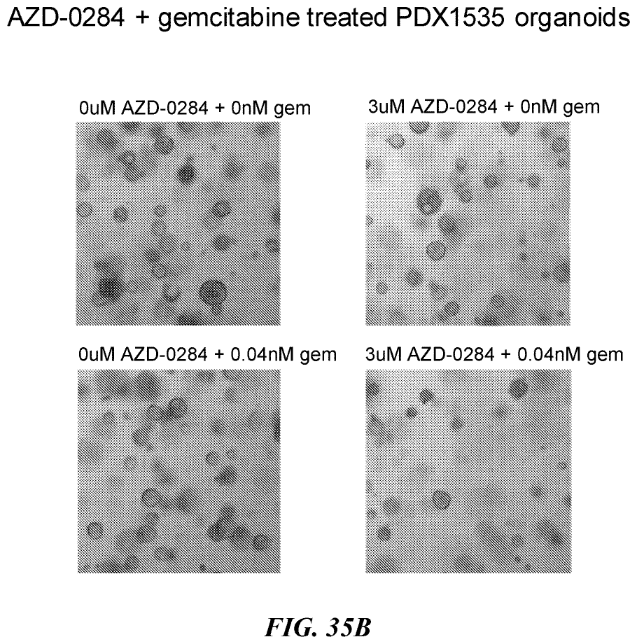

Moreover, the effect of AZD-0284 was assessed on primary patient-derived PDX1535 organoids (FIG. 35). PDX1535 organoids were derived from a patient of pancreatic cancer. Primary patient organoids were established by digesting patient-derived xenografts for 1 hour at 37° C. in RPMI containing 2.5% FBS, 5 mg/ml Collagenase II, and 1.25 mg/ml Dispase II, followed by passage through a 70 μM mesh filter. Cells were plated at a density of 1.5×10⁵ cells per 50 μl Matrigel. After domes were solidified, growth medium was added as follows: RPMI containing 50% Wnt3a conditioned media, 10% RSpondin1-conditioned media, 2.5% FBS, 50 ng/ml EGF, 5 mg/ml Insulin, 12.5 ng/ml hydrocortisone, and 14 μM Rho Kinase Inhibitor. After establishment, organoids were passaged and maintained. Briefly, organoids were isolated using Cell Recovery Solution (Corning 354253), then dissociated into single cell suspension with TrypLE Express (ThermoFisher 12604) supplemented with 25 μg/ml DNase I (Roche) and 14 μM Rho Kinase Inhibitor (Y-27632, Sigma). Cells were split 1:2 into 20 μl domes plated on pre-warmed 48-well plates. Domes were incubated at 37° C. for 5 min, then covered with human complete organoid feeding media without Wnt3a-conditioned media.

The primary patient-derived PDX1535 organoids were grown in the presence of vehicle, 3 μM AZD-0284, 0.04 nM gemcitabine, or both (FIG. 35). The organoids were imaged and measured at the end of treatment. As shown in FIG. 35, the combination of 3 μM AZD-0284 and 0.04 nM gemcitabine resulted in a significant reduction in organoid volume, suggesting that primary patient-derived organoids were also sensitive to RORγ inhibition.

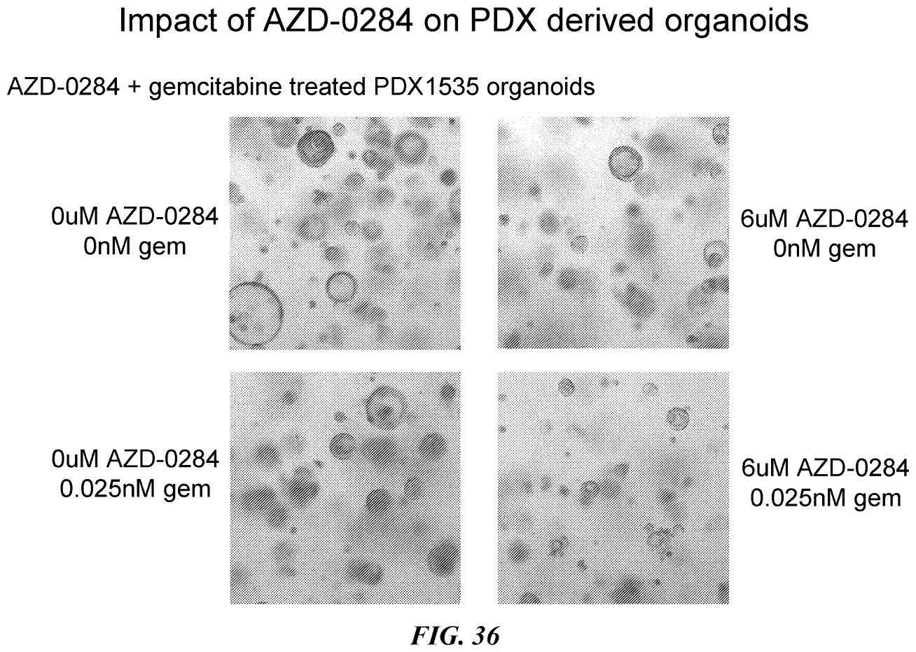

The effect of AZD-0284 at a higher dose was also tested on primary patient-derived PDX1535 organoids (FIG. 36). PDX1535 organoids were cultured in the presence of vehicle, 6 μM AZD-0284, 0.025 nM gemcitabine, or both, followed by imaging. As shown in FIG. 36, 6 μM AZD-0284, alone or in combination with gemcitabine, visibly inhibited growth of PDX1535 organoids.

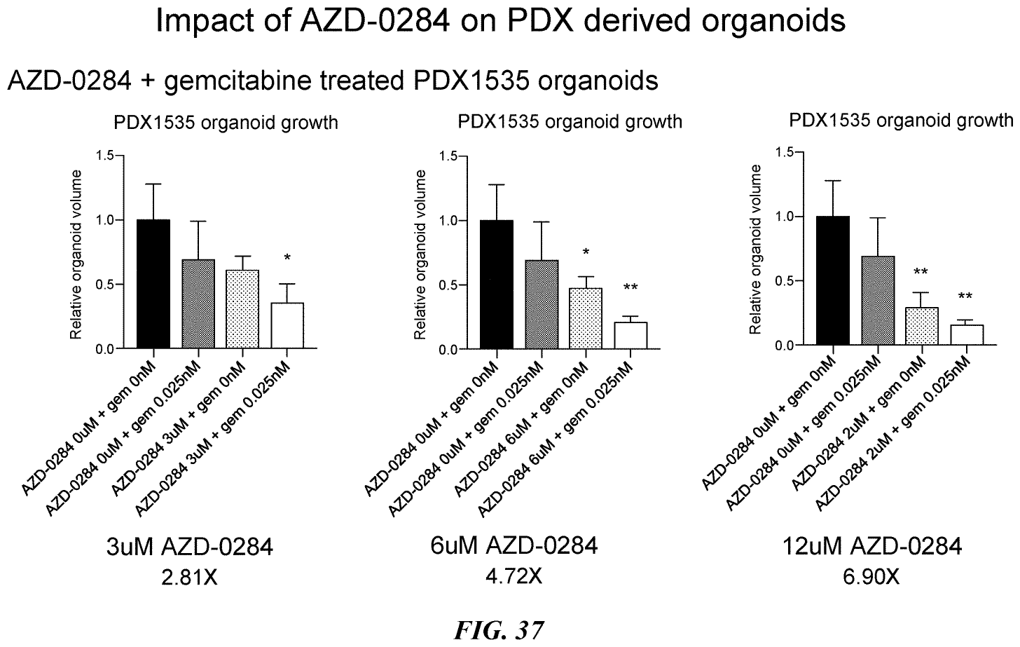

Similarly, the effects of AZD-0284 at different doses were examined on primary patient-derived PDX1535 organoids (FIG. 37). Three doses of AZD-0284 were tested: 3 μM, 6 μM, and 12 μM. For each AZD-0284 dose, four conditions were tested: vehicle, AZD-0284 alone, gemcitabine alone (at 0.025 nM), and a combination of AZD-0284 and gemcitabine. As shown in FIG. 37, 0.025 nM gemcitabine alone decreased PDZ1535 organoid growth, although not statistically significant. Similar to its effect on KP^{f/f}C organoids, AZD-0284, when administered alone, significantly reduced PDX1535 organoid volume at higher doses, e.g., 6 μM or 12 μM. However, if given in combination with gemcitabine, AZD-0284 significantly inhibited PDX1535 organoid growth at all doses tested, to a greater extent than either drug alone. The combination of 0.025 nM gemcitabine and 3 μM AZD-0284, 6 μM AZD-0284, or 12 μM AZD-0284 led to a 2.81-, 4.72-, or 6.90-fold decrease, respectively, in organoid volume compared to control. This result again suggests a synergistic effect between RORγ inhibition and chemotherapy medication for pancreatic cancer treatment.

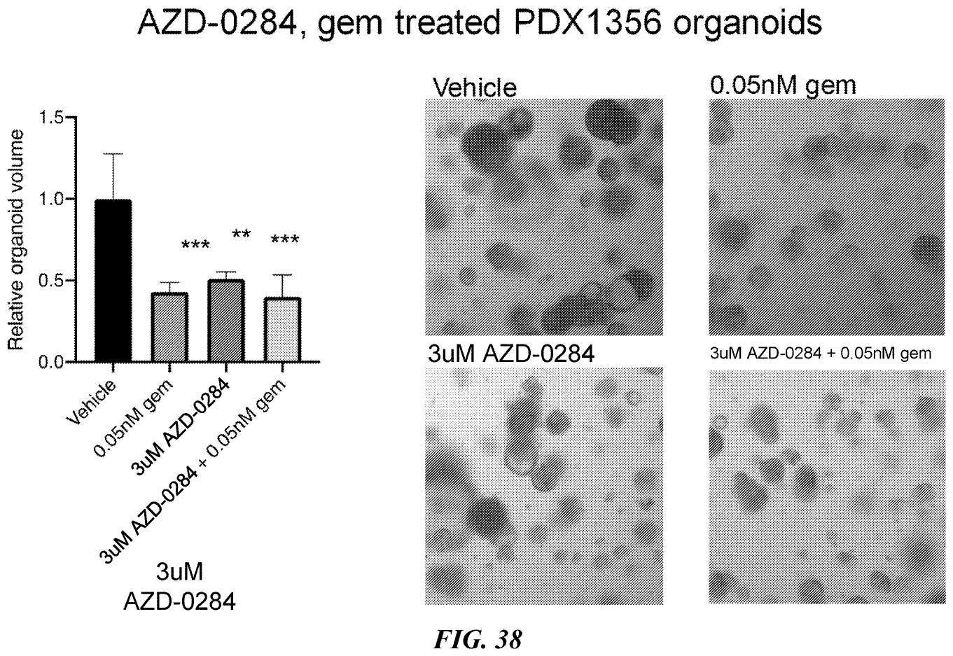

Furthermore, the effect of AZD-0284 was assessed on another primary pancreatic cancer patient-derived cells, PDX1356, using the organoid assay described above (FIG. 38). PDX1356 organoids were grown in the presence of vehicle, 3 μM AZD-0284, 0.05 nM gemcitabine, or both, followed by imaging and measurement of organoid volume at the end of treatment. As shown in FIG. 38, AZD-0284 and gemcitabine, alone or in combination, resulted in a significant reduction in organoid volume, confirming that primary patient-derived organoids were sensitive to RORγ inhibition.

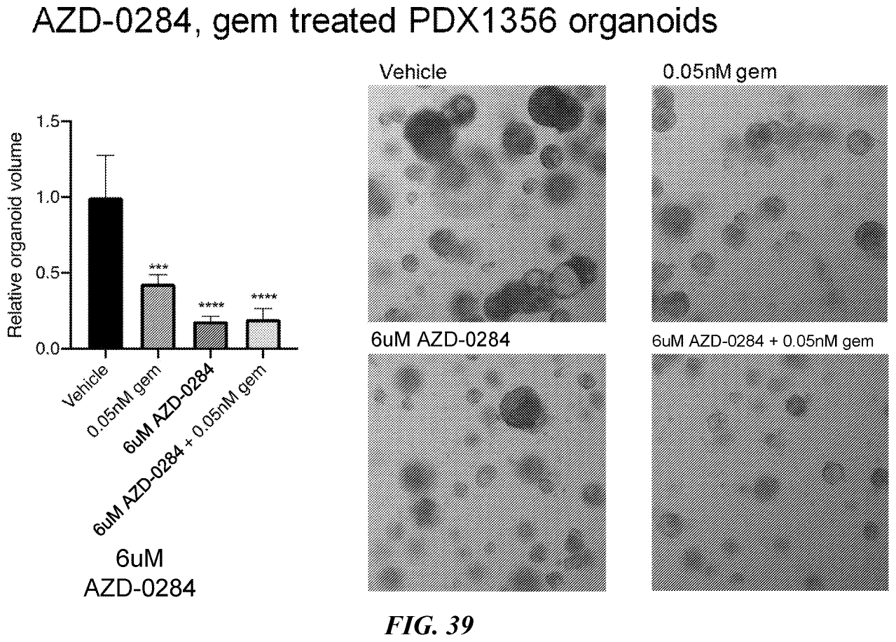

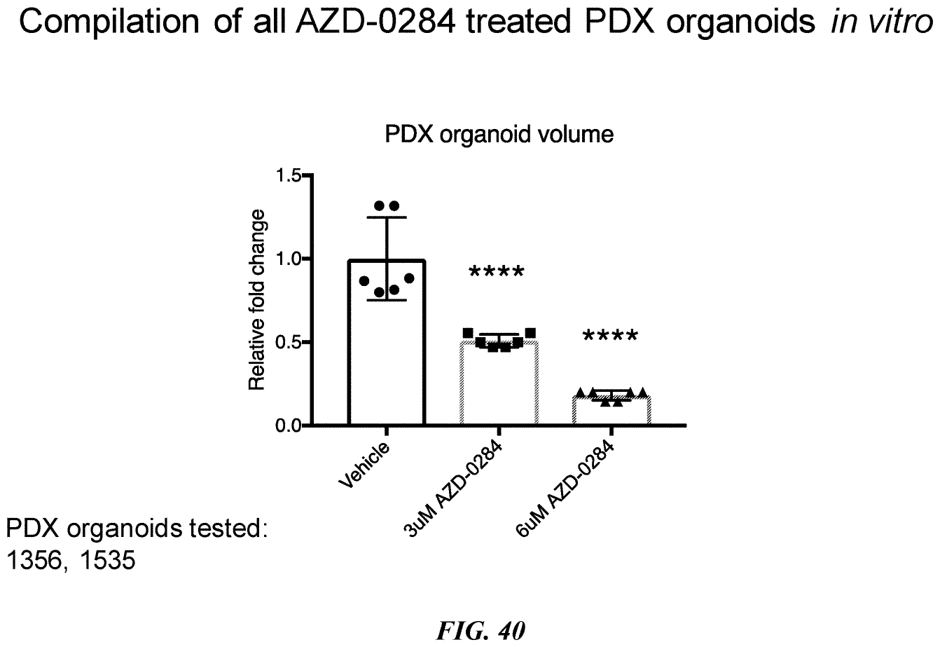

The effect of AZD-0284 at a higher dose was also tested on primary patient-derived PDX1356 organoids (FIG. 39). PDX1356 organoids were cultured in the presence of vehicle, 6 μM AZD-0284, 0.05 nM gemcitabine, or both, followed by imaging. As shown in FIG. 39, AZD-0284 and gemcitabine, alone or in combination, resulted in a significant reduction in organoid volume. FIG. 40 is a compilation of all data from AZD-0284 treated primary patient-derived organoids in vitro, including PDX1356 and PDX1535 organoids, and it shows that AZD-0284, at 3 μM and more so at 6 μM, significantly inhibited organoid growth. Collectively, these data confirmed RORγ as a central regulator of pancreatic cancer progression and identified AZD-0284, an RORγ inhibitor, as an effective anti-tumor therapeutic agent.

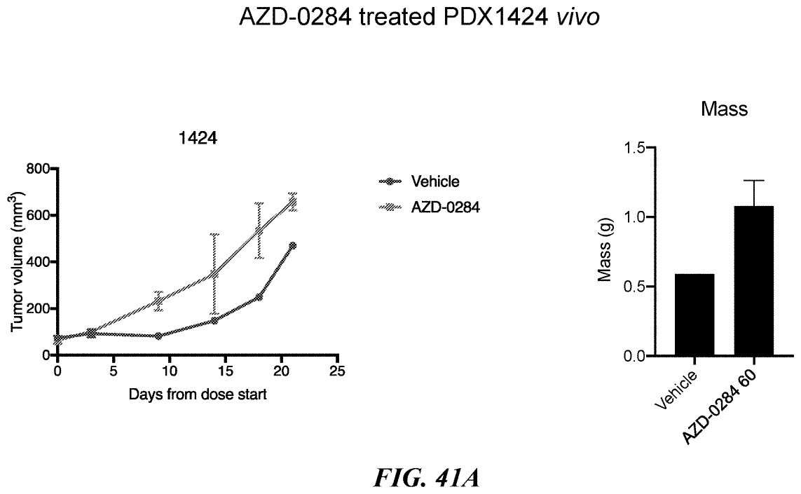

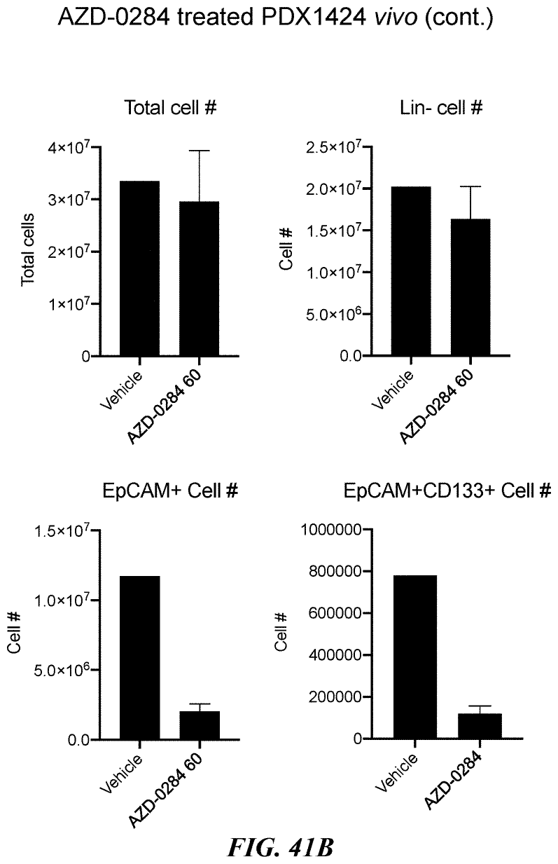

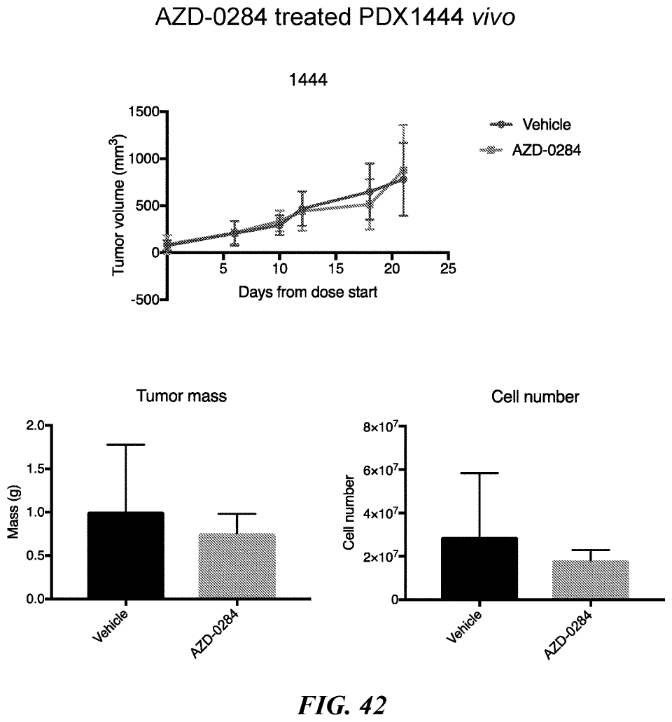

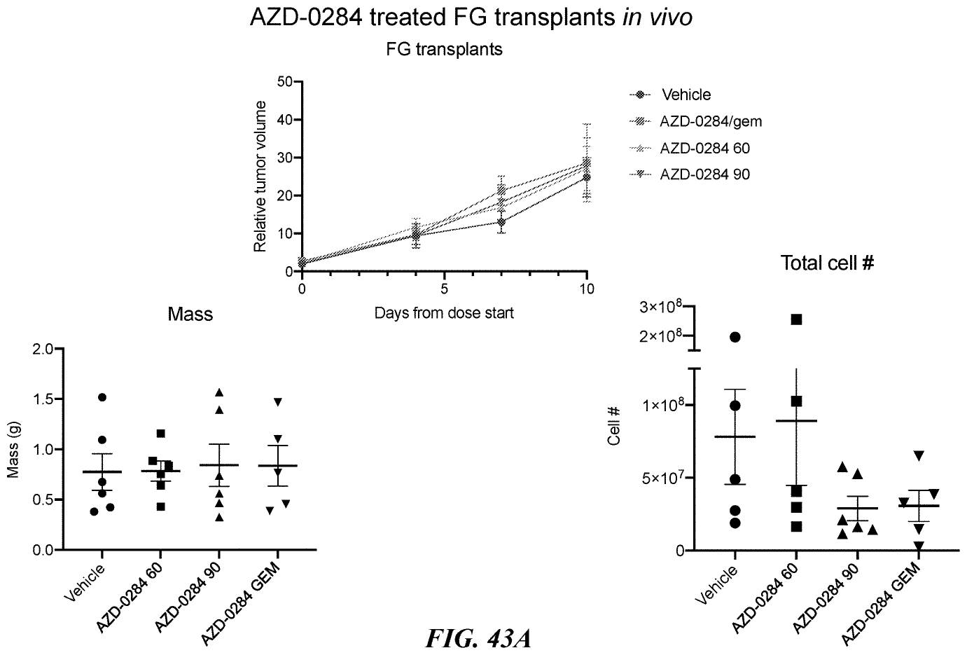

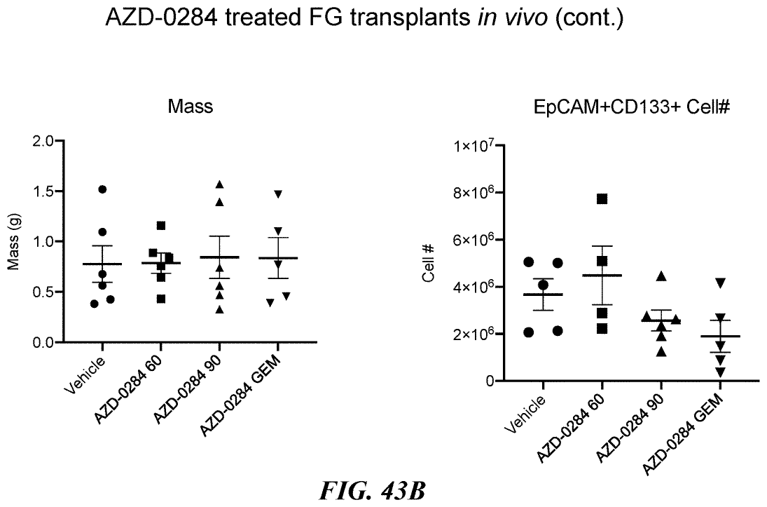

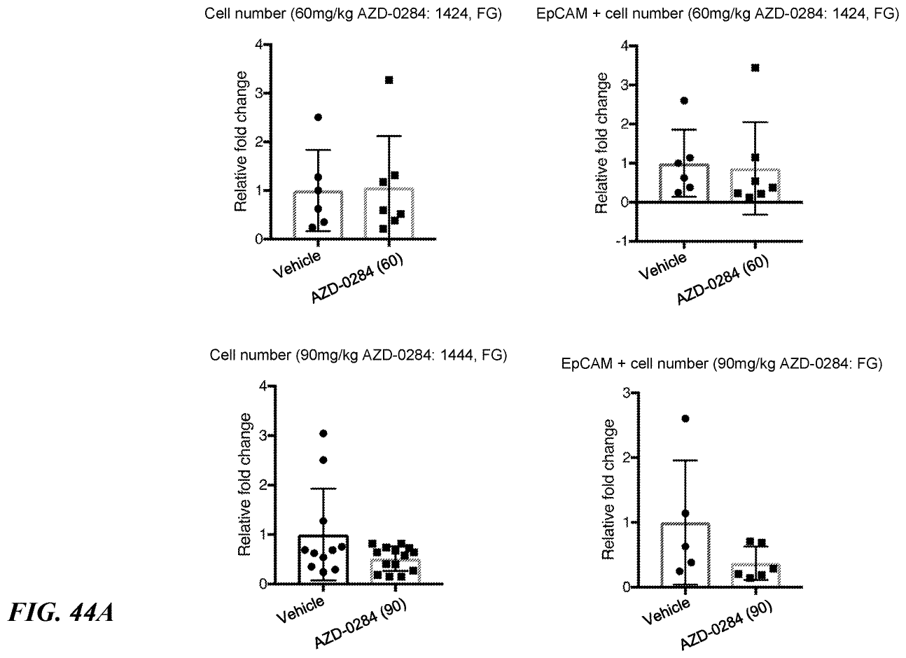

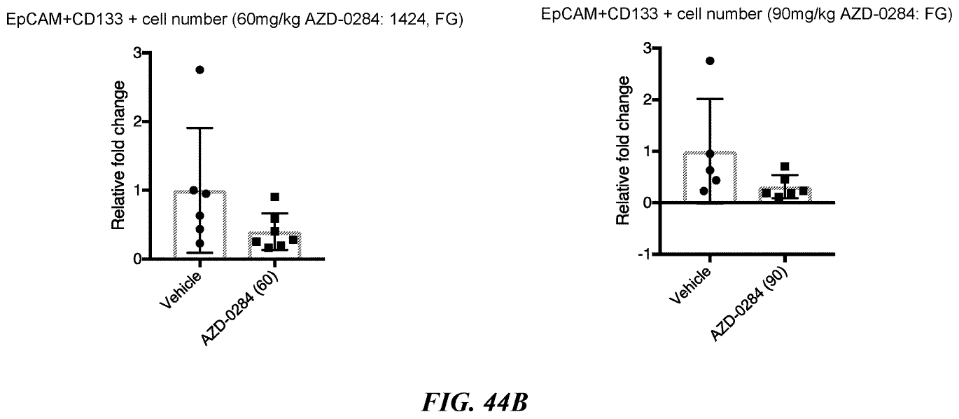

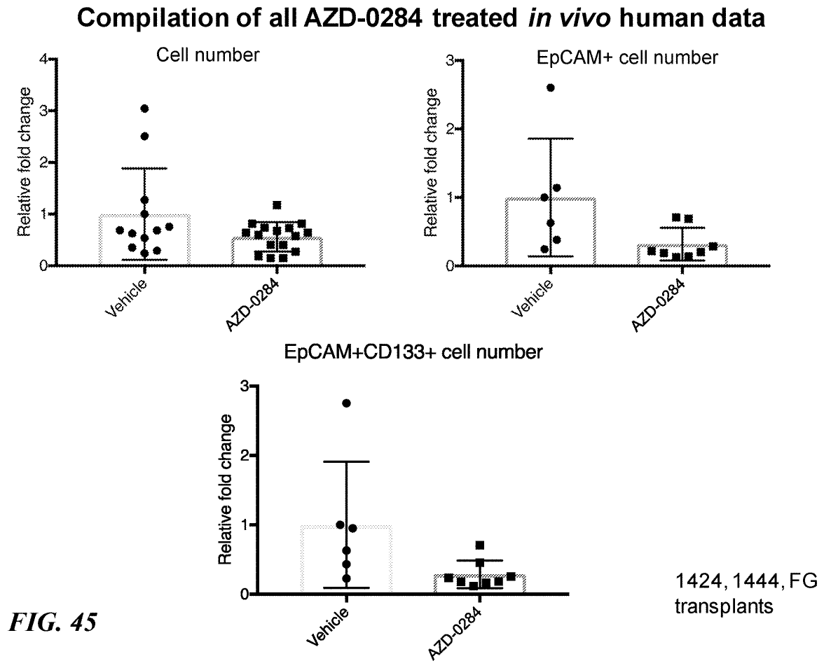

Finally, the impact of AZD-0284 was tested on immunodeficient mice transplanted with primary patient-derived cancer cells in vivo (FIGS. 41-45). As shown in FIG. 41, mice bearing primary patient-derived PDX1424 cancer cells were treated with vehicle or 60 mg/kg AZD-0284 for 3 weeks. AZD-0284 treatment led to a significant reduction of EpCam+ tumor epithelial cells and EpCam+/CD133+ tumor stem cells, although such tumor-inhibitory effect was not observed in another experiment using primary patient-derived PDX1444 cancer cells (FIG. 42). However, a similar inhibitory effect was repeated in an experiment using mice transplanted with Fast Growing (FG) cells that were treated with different doses of AZD-0284, or AZD-0284 in combination with gemcitabine, as reflected by a decrease in total cell number and EpCam+/CD133+ tumor stem cells in mice treated with 90 mg/kg AZD-0284 or the combination therapy (FIG. 43). FIG. 44 shows compilations of data from mice bearing PDX or FG cancer cells, including PDX1424, PDX1444, and FG cells, that received 60 mg/kg AZD-0284 or 90 mg/kg AZD-0284 as indicated in the figures. Especially at higher dosage (i.e., 90 mg/kg), AZD-0284 treatment reduced EpCam+ tumor epithelial cells and EpCam+/CD133+ tumor stem cells. FIG. 45 is a compilation of all data from mice bearing PDX or FG cancer xenographs, including PDX1424, PDX1444, and FG. Consistent with previous observations, AZD-0284 treatment led to a decrease in cell number, EpCam+ tumor epithelial cells, and EpCam+/CD133+ tumor stem cells, suggesting that AZD-0284 was effective at treating pancreatic tumor in vivo.

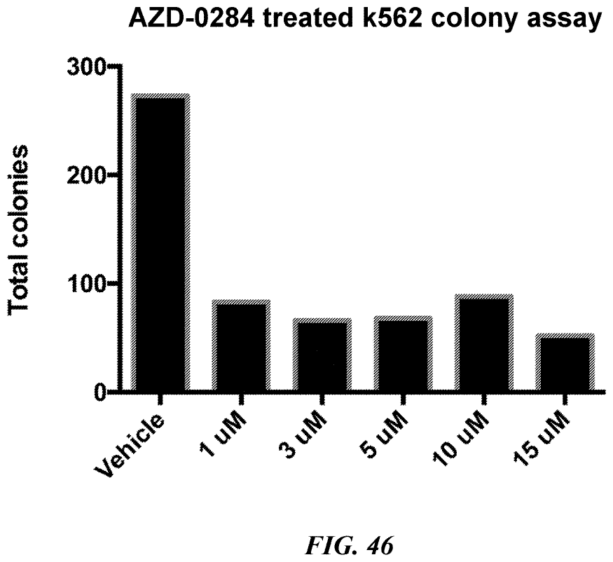

Given the common features and shared molecular dependencies between leukemia and pancreatic cancer stem cells, the effect of AZD-0284 was tested on leukemia cells (FIG. 46). K562 is an aggressive human leukemia cell line generated from blast crisis chronic myeloid leukemia. Colony assays of k562 cells were performed using different doses of AZD-0284. K562 cells were plated at a single cell level in methylcellulose containing AZD-0284. Cells were allowed to grow over the course of 8 days before the numbers of formed colonies were counted. This was used to understand the functionality of k562 cells under different conditions. Cells treated with AZD-0284 formed fewer colonies and their morphology was smaller in comparison to the vehicle-treated cells. As shown in FIG. 46, 1 μM, 3 μM, 5 μM, 10 μM, and 15 μM of AZD-0284 each resulted in significant reduction of the number of colonies formed, suggesting that AZD-0284 is also effective at inhibiting leukemia cell growth.

Taken together, these data show AZD-0284, an RORγ inhibitor, as a promising drug to be used in anti-cancer therapies and/or used in combination with chemotherapy medication for more effective cancer treatment in a variety of types of caners, including pancreatic cancer and leukemia.

### Example 6

This working example demonstrates that JTE-151, another inhibitor of RORγ, is effective in impairing the growth of mammalian pancreatic cancer in vitro and in vivo. The results show that JTE-151 can be used as an effective therapeutic agent for cancer treatment.

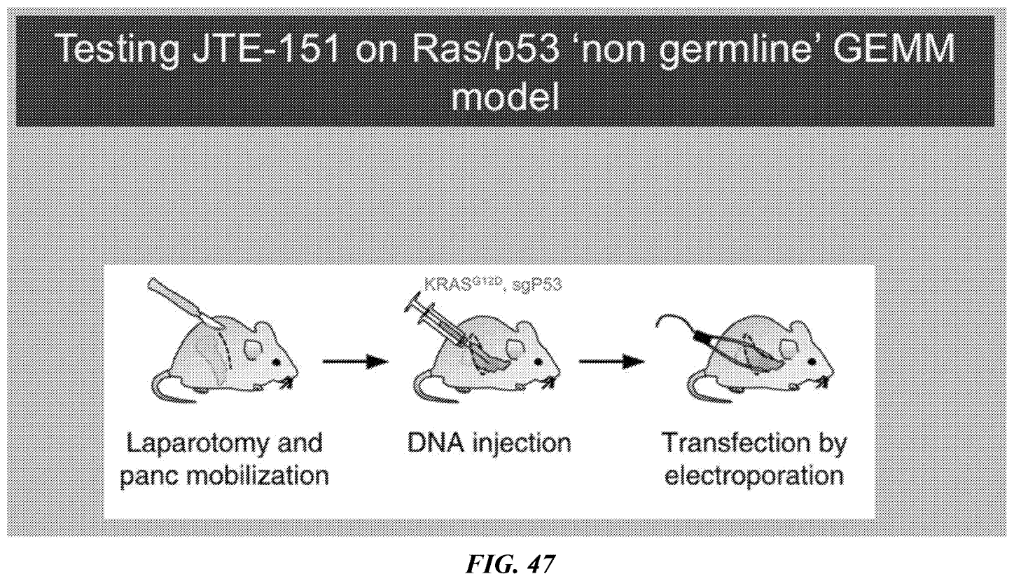

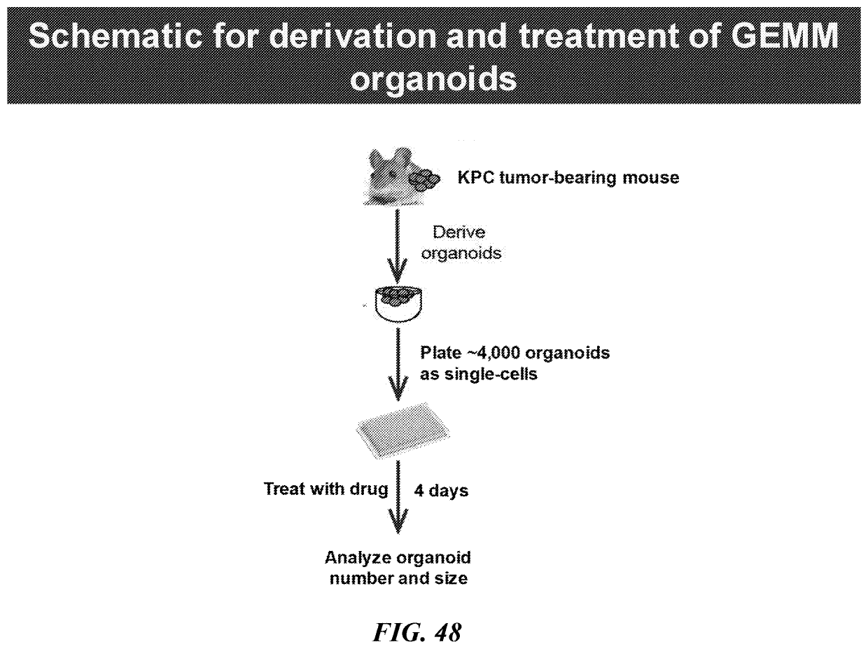

First, pharmacologic blockade of RORγ using JTE-151 was tested on pancreatic cell organoids as described above. Pancreatic cancer cells derived from two genetically engineered mouse models (GEMMS) were used for the organoid studies (FIGS. 47, 48). First, as shown in FIG. 47, a non-germline mouse model of pancreatic cancer was generated by surgical laparotomy and mobilization of the pancreas, followed by DNA injection of KRAS^{G12D} (an activated form of KRAS) and sgP53 (a CRISPR guide targeting p53). Then, electroporation was used to promote incorporation of the DNA into the pancreatic cells. The so generated mouse model had mutations only in the pancreas, thus the label “non-germline.” Second, as shown in FIG. 48, a germline genetically engineered mouse model for pancreatic cancer was used, which had the genotype of Kras^{LSL-G12D/+}; pdx^{CRE/+}; p53^{f/f} (KP^{f/f}C).

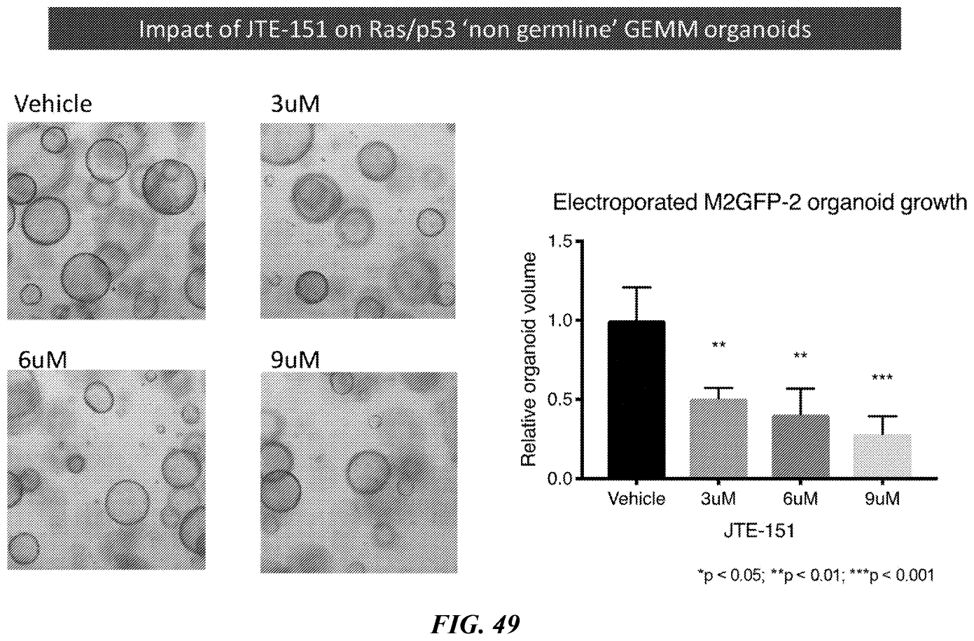

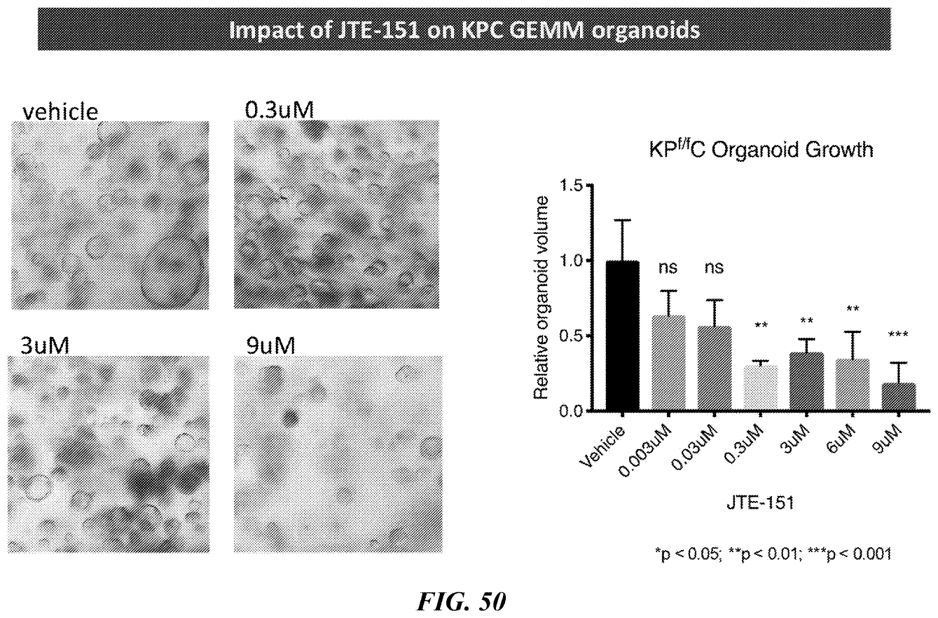

About 4,000 organoids from each of the non-germline and germline mouse models were plated as single cells in multi-well plates, as described above, and treated with JTE-151 for 4 days (FIG. 48). Organoid number and size were analyzed after treatment. A significant impairment in organoid volume was observed in each case (FIGS. 49, 50). As shown in FIG. 49, the organoid forming capacity of non-germline KRAS/p53 cells grown in the presence of vehicle, 3 μM JTE-151, 6 μM JTE-151, or 9 μM JTE-151 was assessed by imaging and measurement of relative organoid volume. In the quantification, different doses of JTE-151 were plotted along the horizontal axis, and the volume of organoids was expressed as relative to control along the vertical axis. JTE-151 at all doses tested visibly and significantly impaired KRAS/p53 organoid growth. Similarly, as shown in FIG. 50, pancreatic cancer cells derived from germline KP^{f/f}C mouse model were grown in the presence of vehicle or different doses of JTE-151. Organoid volume was then analyzed. Different doses of JTE-151 were plotted along the horizontal axis, and the vertical axis represents relative organoid volume to control. At lower doses (0.003 μM and 0.03 μM), JTE-151 reduced organoid volume, although not at a statistically significant level. At higher doses (0.3 μM, 3 μM, 6 μM, and 9 μM), however, JTE-151 significantly inhibited KP^{f/f}C organoid growth, consistent with imaging results.

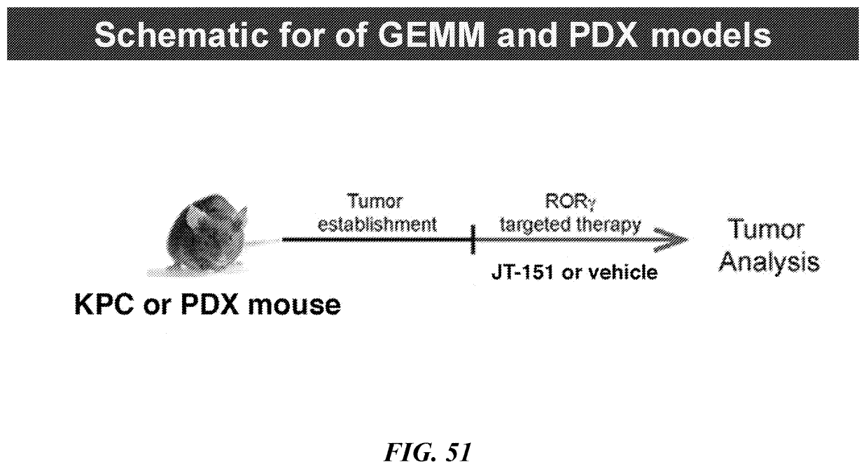

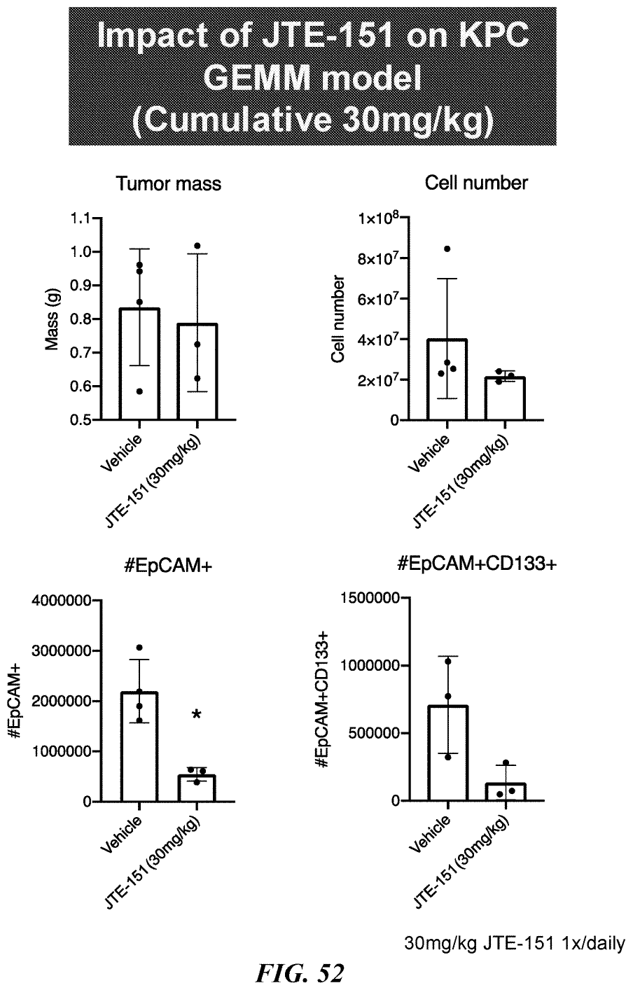

Next, the impact of JTE-151 was tested on tumor-bearing KP^{f/f}C mice in vivo. FIG. 51 is a schematic of the experimental design. KP^{f/f}C mice were allowed to develop tumors, then the tumor-bearing mice received vehicle or JTE-151, followed by analysis of the tumors at the end of the experiments. Different doses of JTE-151, i.e., at 30 mg/kg, 90 mg/kg, and 120 mg/kg body weight, were tested. FIG. 52 is a compilation of data from tumor-bearing KP^{f/f}C mice treated with vehicle or 30 mg/kg JTE-151 once daily for about 3 weeks, and it shows that treatment of JTE-151 resulted in reduced cell number and a loss of EpCam+ tumor epithelial cells and EpCam+/CD133+ tumor stem cells. The decrease in EpCam+ tumor epithelial cells was statistically significant compared to control.

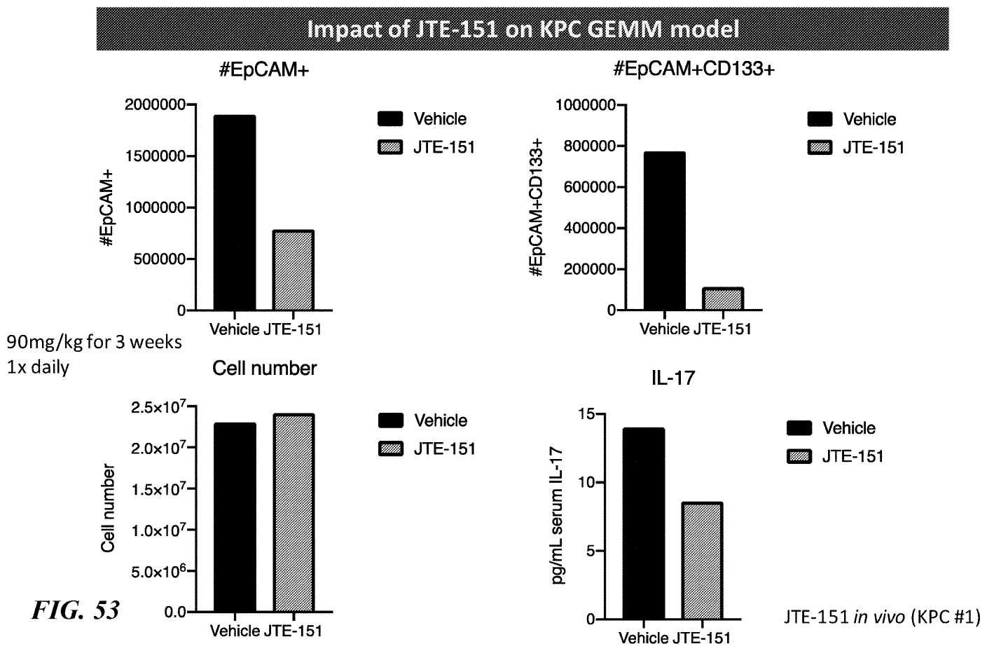

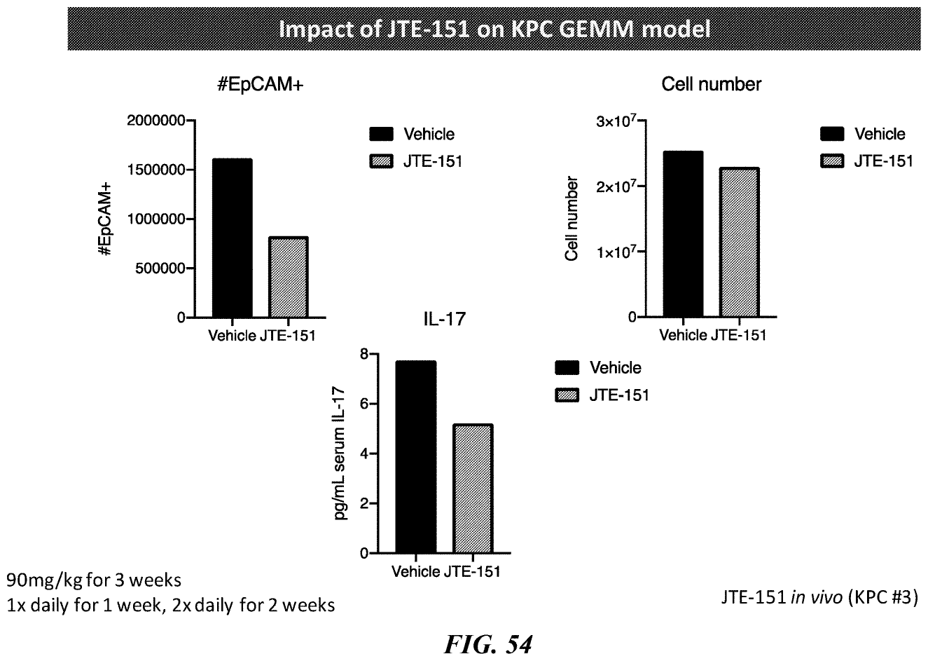

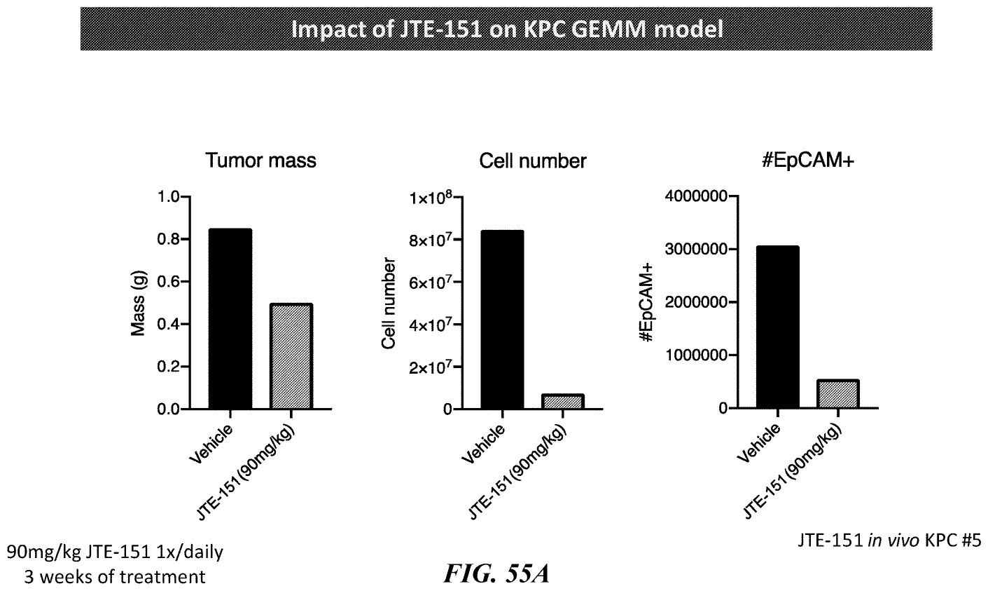

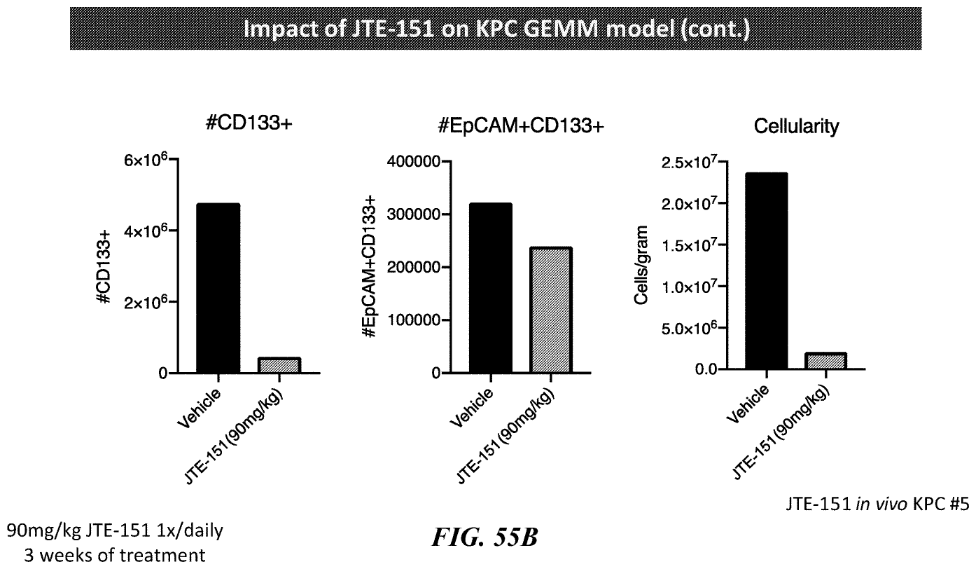

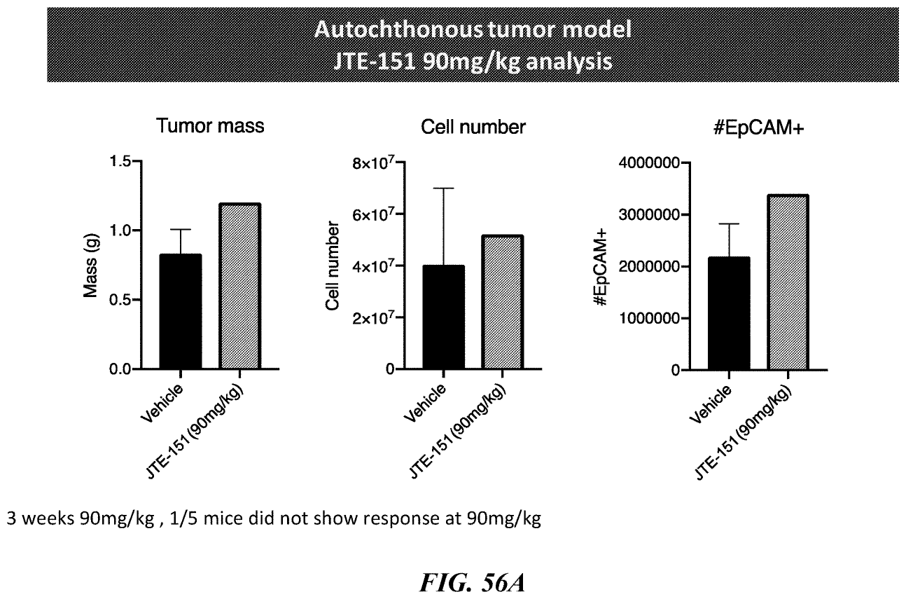

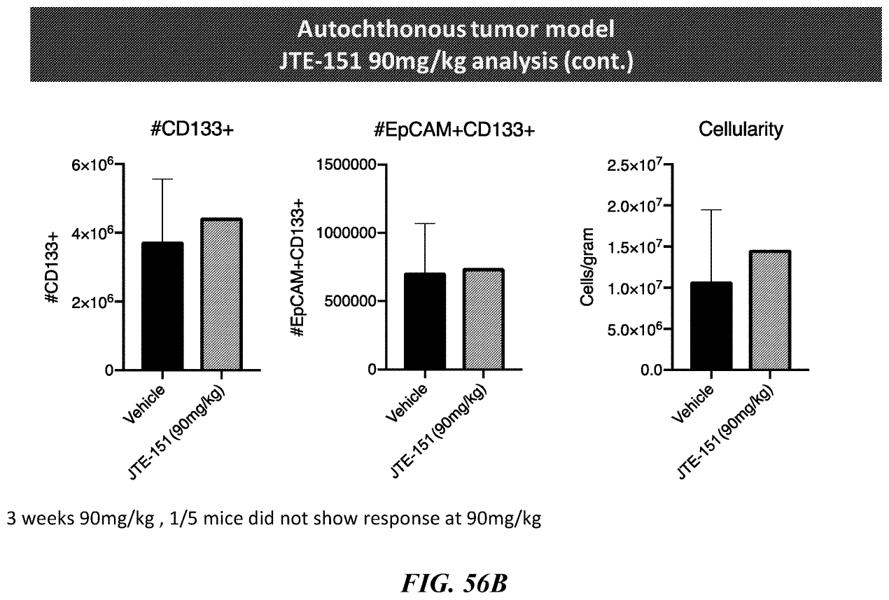

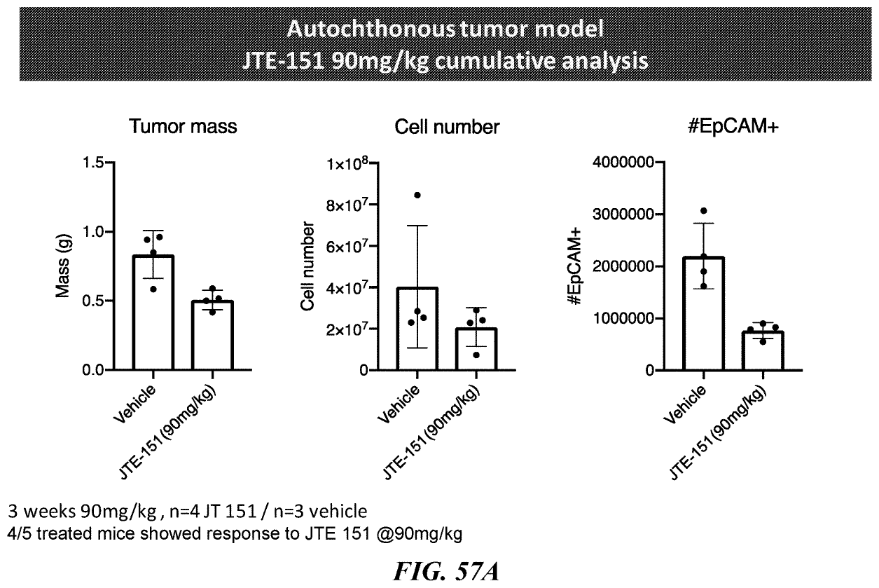

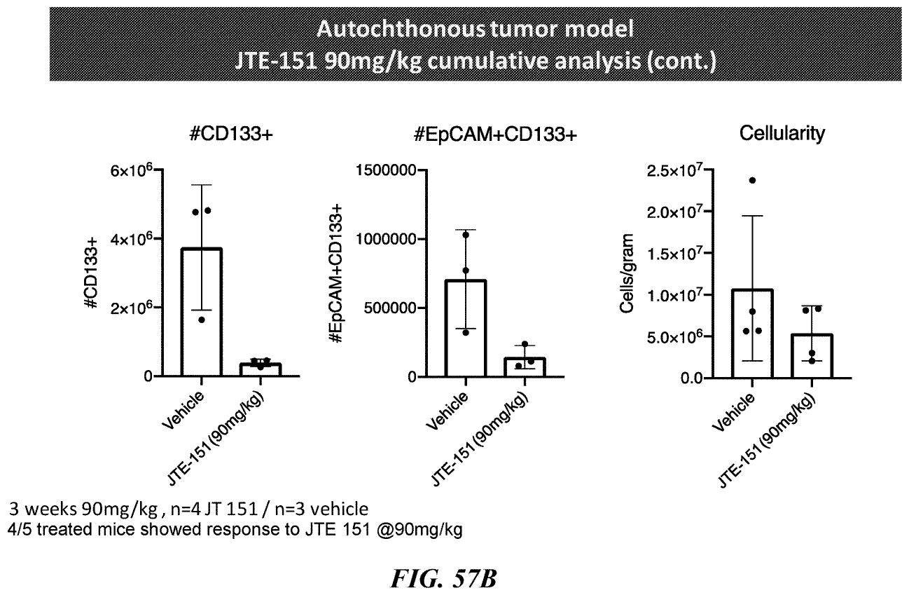

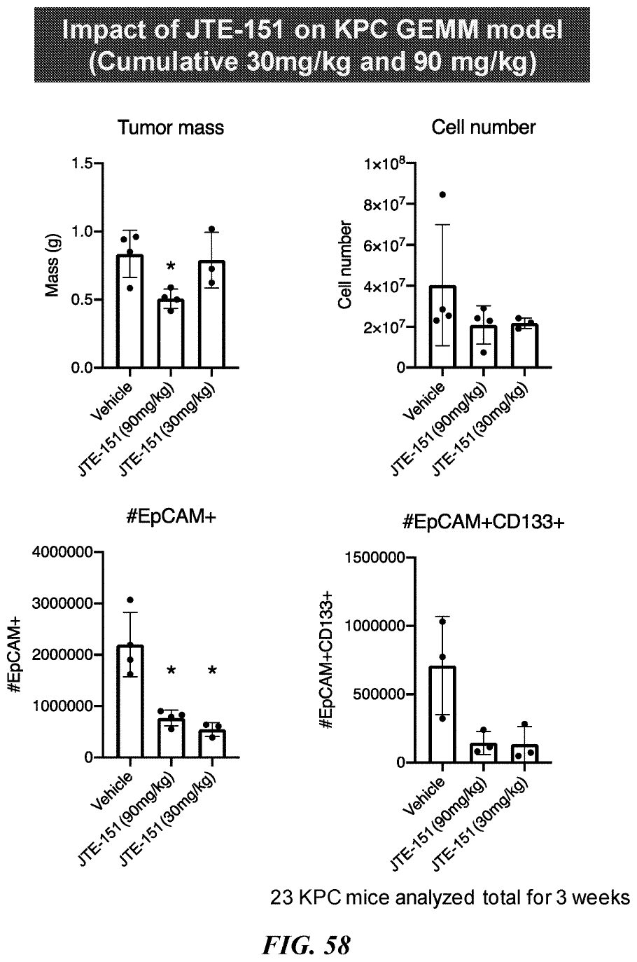

FIGS. 53-56 show examples of individual experiments where tumor-bearing KP^{f/f}C mice was treated with either vehicle or 90 mg/kg JTE-151 for 3 weeks in regimens as specified in the figures. For example, in FIGS. 53 and 55, the mice received 90 mg/kg JTE-151 once daily for 3 weeks. In FIG. 54, the mice received 90 mg/kg JTE-151 once daily for 1 week, followed by twice daily for another 2 weeks. At the end of each experiment, tumors were analyzed for different parameters including tumor mass, cell number, EpCAM positivity, CD133 positivity, EpCAM/CD133 positivity, cellularity, and IL-17 level. As shown in FIGS. 53-55, mice treated with 90 mg/kg JTE-151 exhibited reduced tumor mass, decreased EpCam+ tumor epithelial cells, and/or decreased EpCam+/CD133+ tumor stem cells, suggesting the anti-cancer efficacy of JTE-151. 1 out of 5 mice tested did not show a response to JTE-151 treatment at the dose of 90 mg/kg (FIG. 56). It was not clear whether the initial tumor size of the non-responder mouse was unusually large due to variances between different mice. FIG. 57 is a compilation of data from tumor-bearing KP^{f/f}C mice treated with vehicle (n=3) or 90 mg/kg JTE-151 (n=4) for 3 weeks, and it shows that treatment of JTE-151 resulted in reduced tumor mass, reduced cell number, and a loss of EpCam+ tumor epithelial cells and EpCam+/CD133+ tumor stem cells. Similarly, FIG. 58 is a compilation of data from tumor-bearing KP^{f/f}C mice treated with vehicle, 30 mg/kg JTE-151, or 90 mg/kg JTE-151 (total of 23 mice) for 3 weeks, and it shows that treatment of JTE-151 at either dosage resulted in reduced cell number and a loss of EpCam+ tumor epithelial cells and EpCam+/CD133+ tumor stem cells. JTE-151 at 90 mg/kg also significantly reduced tumor mass.

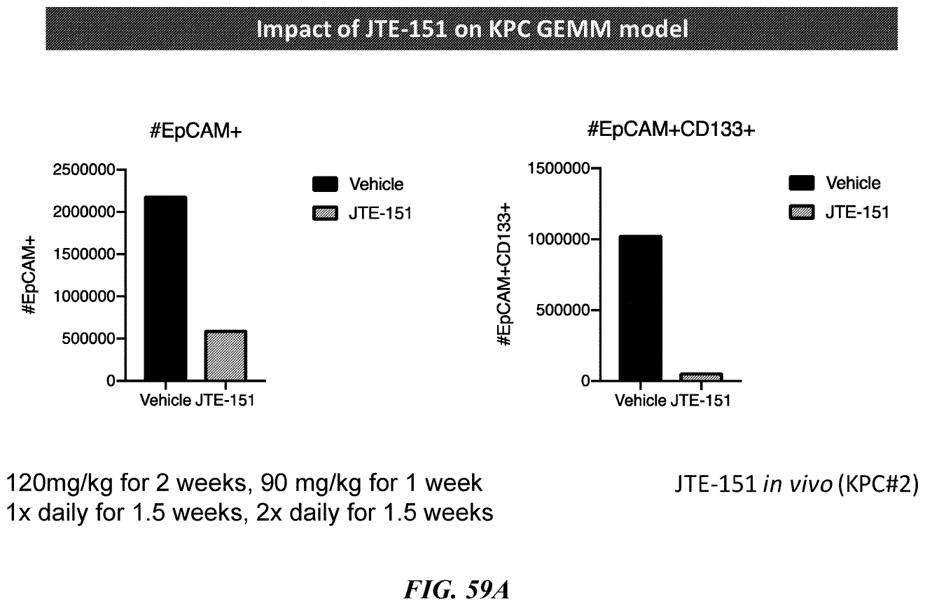

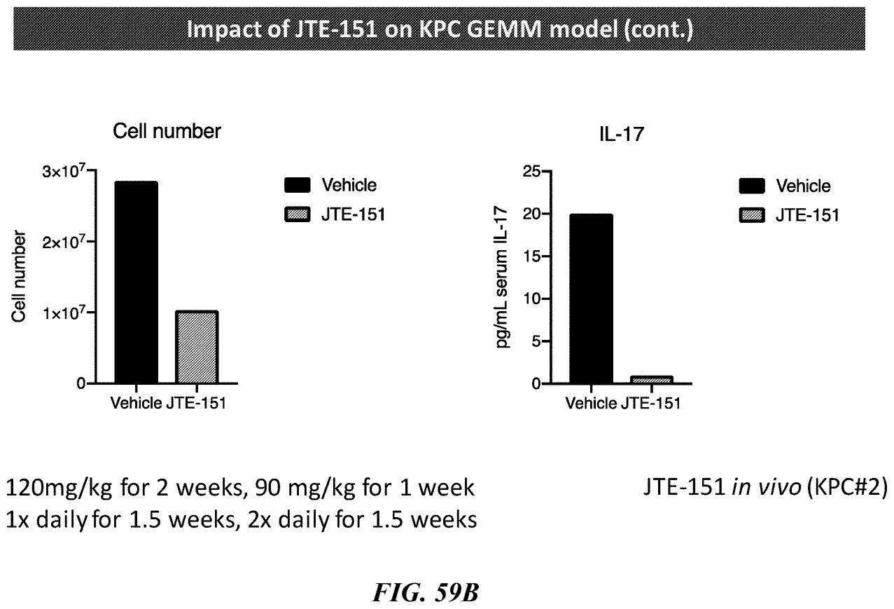

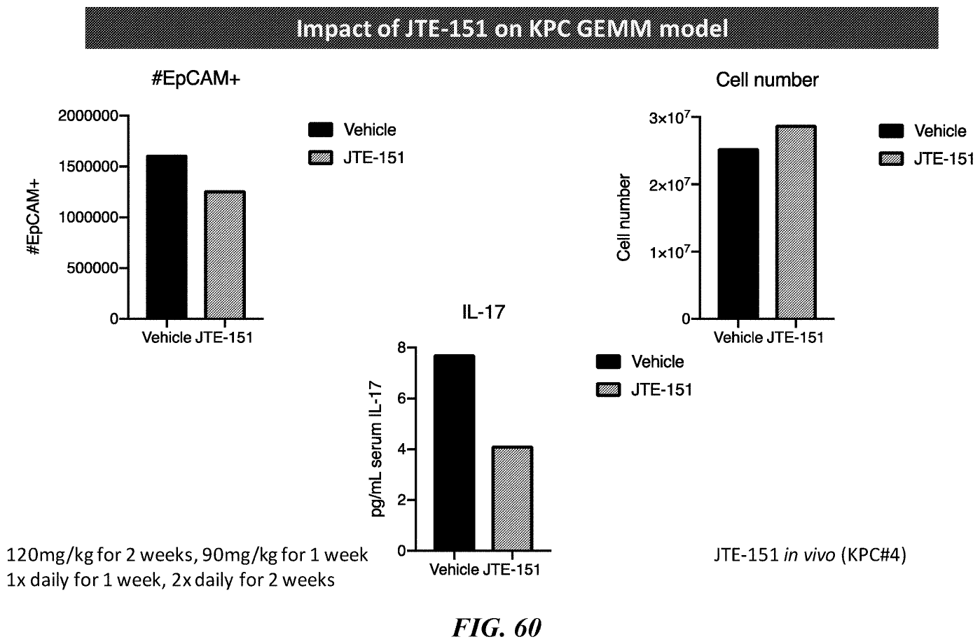

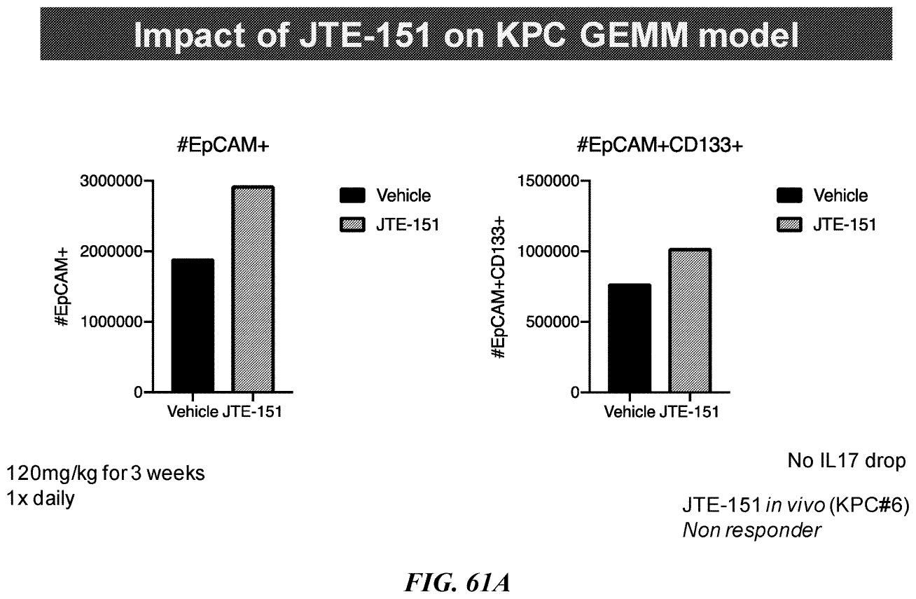

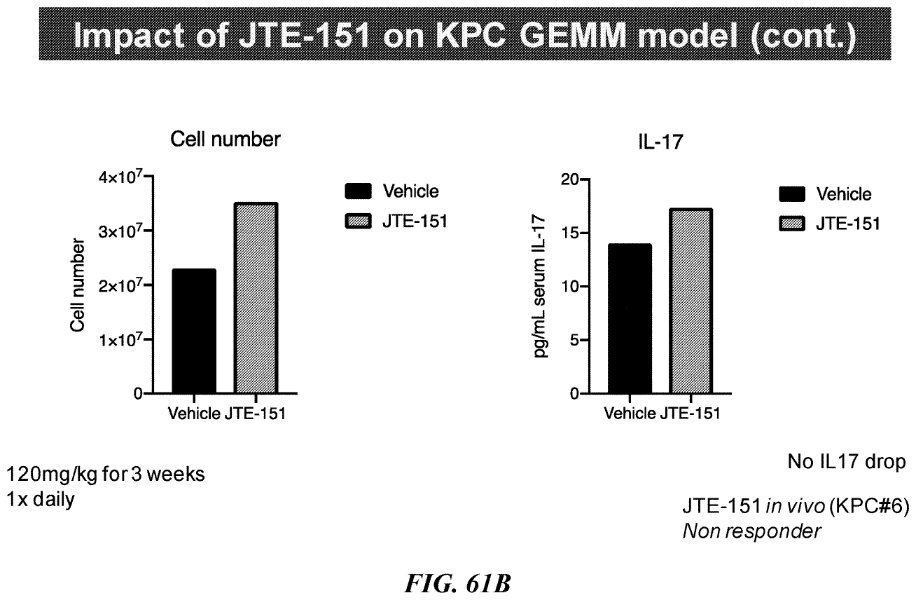

Similarly, the anti-cancer effect of JTE-151 was tested on tumor-bearing KP^{f/f}C mice in vivo at a higher dose of 120 mg/kg (FIGS. 59-61 showing three individual experiments). For each experiment, one mouse was given vehicle treatment, and another mouse was given the JTE-151 regimen as specified in the figures. For example, in FIG. 59, the JTE-151 mouse received 120 mg/kg body weight of JTE-151 for 2 weeks and then 90 mg/kg JTE-151 for 1 week. In the first 1.5 weeks, JTE-151 was given once daily, and in the second 1.5 weeks, JTE-151 was given twice daily. As previously, at the end of each experiment, tumors were analyzed for different parameters including cell number, EpCAM positivity, EpCAM/CD133 positivity, and IL-17 level. In each of FIGS. 59-61, the horizontal axis of each graph represents the target (vehicle vs. JTE-151 mouse), and the vertical axis represents the specified measurement. At least two of the three mice that received JTE-151 responded to the drug, as reflected by a decrease in circulating IL-17 levels (FIGS. 59-60). In the mice that responded to JTE-151, a loss of EpCam+/CD133+ tumor stem cells and/or a loss of EpCam+ tumor epithelial cells were observed consistently, although the change of cell number in the tumor varied (FIGS. 59-60), and 1 of the tested mice did not show a response or a drop in IL-17 level (FIG. 61).

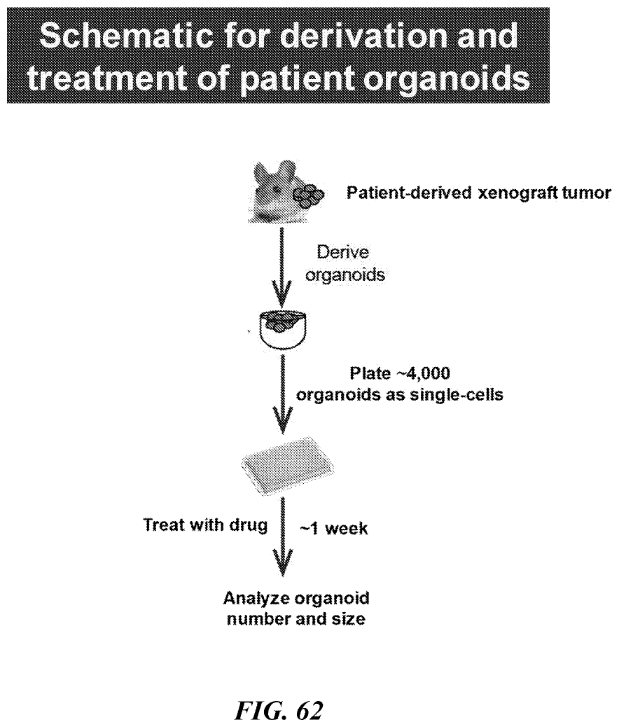

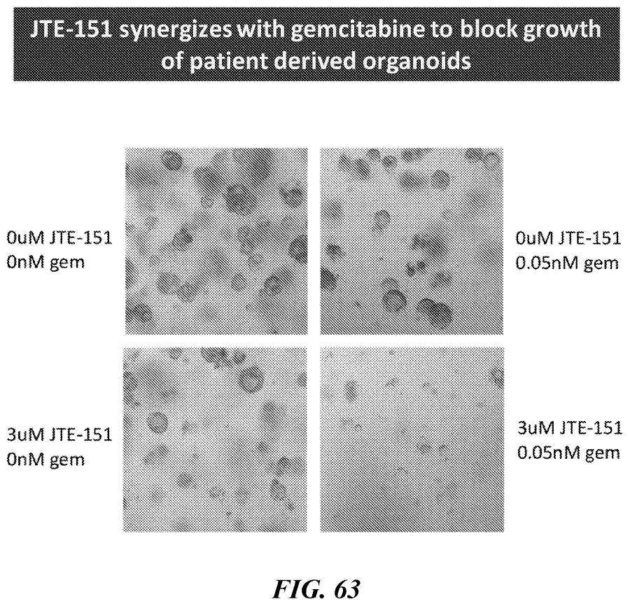

Moreover, the anti-cancer effect of JTE-151 was determined in an organoid assay using pancreatic cancer cells derived from mice bearing primary patient-derived xenografts. A schematic of the experimental design is shown in FIG. 62. Cells derived from the xenograft tumor were plated as single cells and treated with JTE-151 with or without gemcitabine for one week before organoid number and size were analyzed. As shown in FIG. 63, primary patient-derived PDX1535 organoids were treated with vehicle, 3 μM JTE-151, 0.05 nM gemcitabine, or both, followed by imaging. The treatment of JTE-151 alone, gemcitabine alone, or JTE-151 and gemcitabine combination each resulted in visibly reduced organoid volume of PDX1535 organoids.

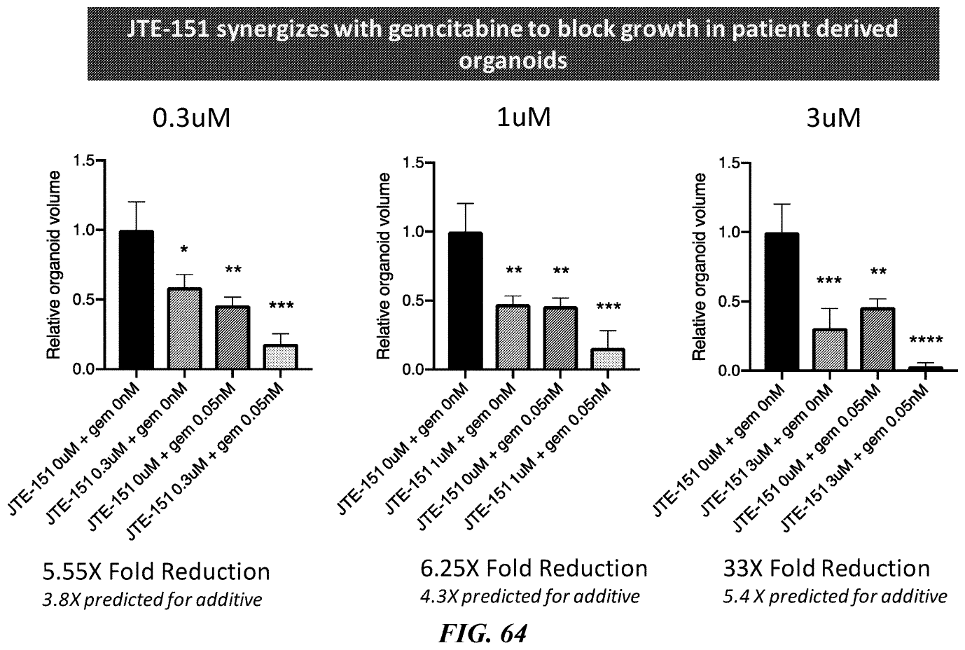

As shown in FIG. 64, the effects of JTE-151 at different doses were examined on PDX1535 organoids. Three doses of JTE-151 were tested: 0.3 μM, 1 μM, and 3 μM. For each JTE-151 dose, four conditions were tested: vehicle, JTE-151 alone, gemcitabine alone (at 0.05 nM), and a combination of JTE-151 and gemcitabine (plotted along the horizontal axis). The vertical axis represents relative organoid volume. At all dose tested, either JTE-151 alone or gemcitabine alone resulted in significant inhibition of PDX1535 organoid growth. However, the combination of JTE-151 and gemcitabine achieved the most significant reduction of PDX1535 organoid growth at all doses tested, ranging from 5.55-fold reduction to 33-fold reduction in a dose-dependent fashion. This suggests that JTE-151 synergizes with gemcitabine to block the growth of patient-derived organoids.

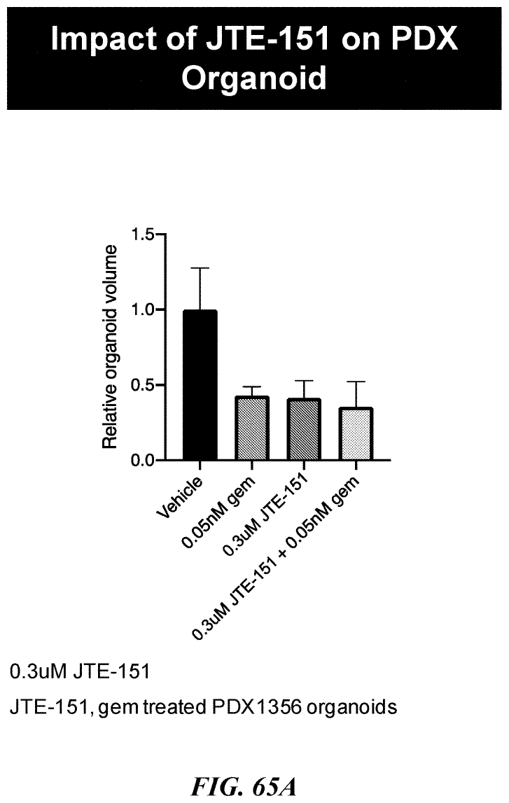

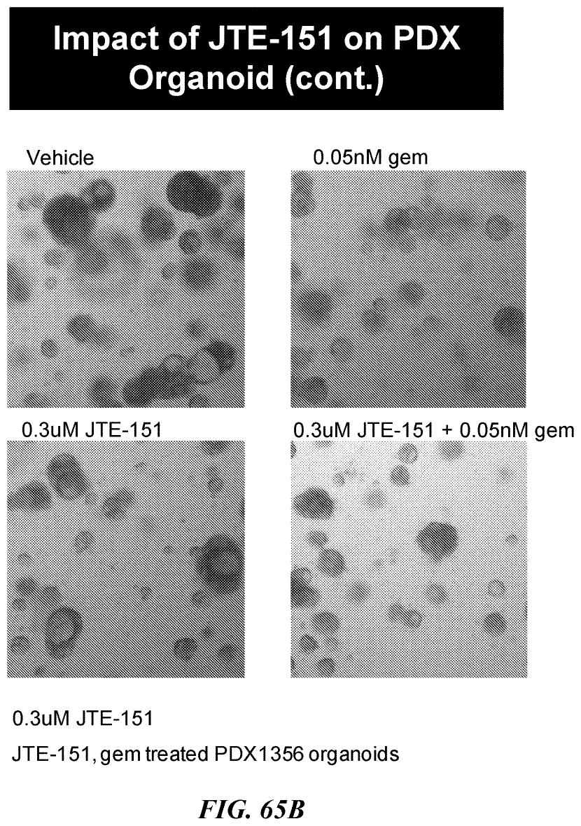

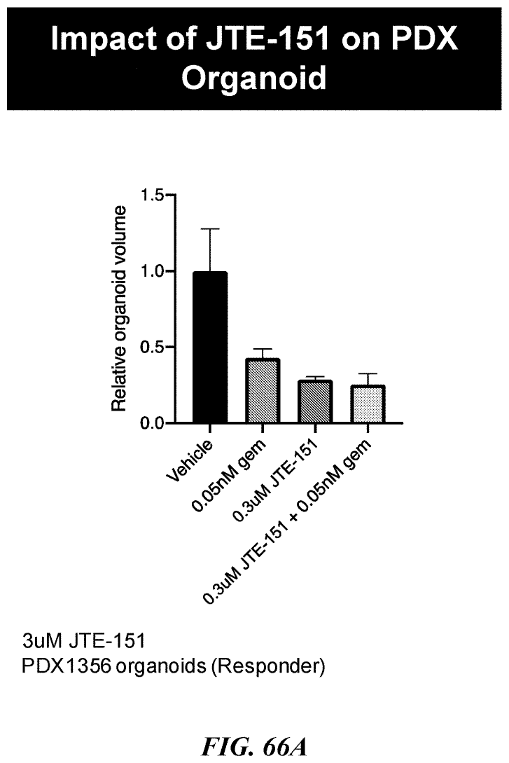

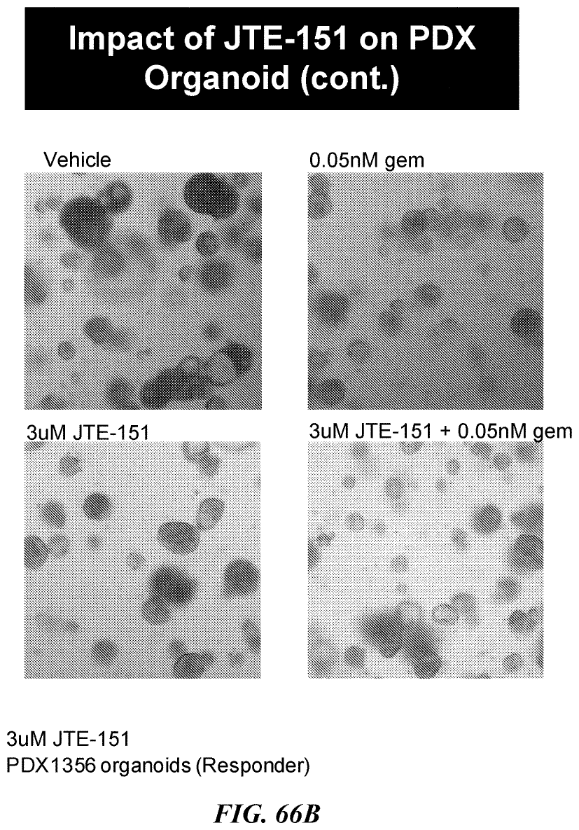

As shown in FIGS. 65-66, the anti-cancer effect of JTE-151 was also tested using the organoid assay on primary patient-derived PDX1356 pancreatic cancer cells. The organoid forming capacity of PDX1356 cells grown in the presence of vehicle, 0.3 μM JTE-151, 0.05 nM gemcitabine, or both was assessed by imaging and measurements of organoid volume (FIG. 65). The volume of organoids was expressed as relative to control. As shown in 65, gemcitabine and JTE-151, either given alone or in combination, visibly decreased organoid growth in volume. As shown in FIG. 66, the effect of JTE-151 at a higher dose on PDX1356 organoid growth was also examined. PDX1356 organoids were cultured in the presence of vehicle, 3 μM JTE-151, 0.05 nM gemcitabine, or both, followed by imaging. Again, as shown in FIG. 66, the treatment of JTE-151 alone, gemcitabine alone, or JTE-151 and gemcitabine combination each resulted in visibly reduced organoid volume of PDX1356 cells.

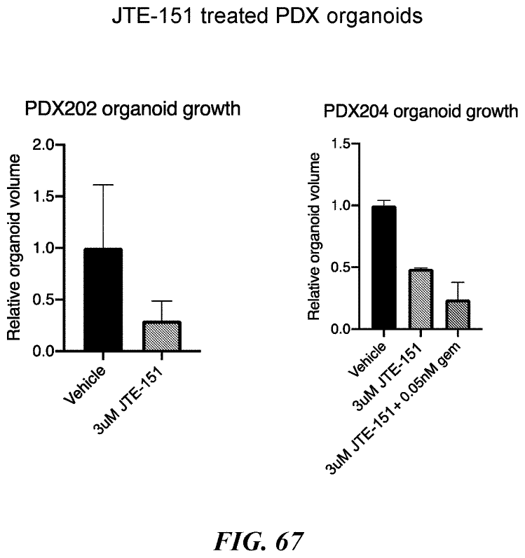

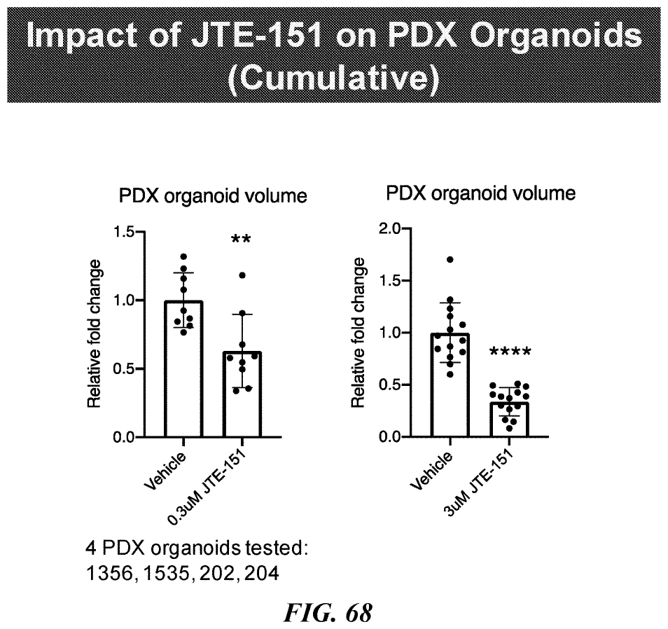

As shown in FIG. 67, the anti-cancer effect of JTE-151 was also tested using the organoid assay on primary patient-derived PDX202 and PDX204 pancreatic cancer cells. 3 μM JTE-151 alone inhibited organoid growth of PDX202 and PDX204 cells, and 3 μM JTE-151 in combination with 0.05 nM gemcitabine inhibited organoid growth of PDX204 cells. FIG. 68 is a compilation of all data from JTE-151 treated primary patient-derived organoids, including PDX1356, PDX1535, PDX202, and PDX204, and it shows that JTE-151, at 0.3 μM and more so at 3 μM, significantly inhibited organoid growth of cells derived from primary pancreatic cancer patients.

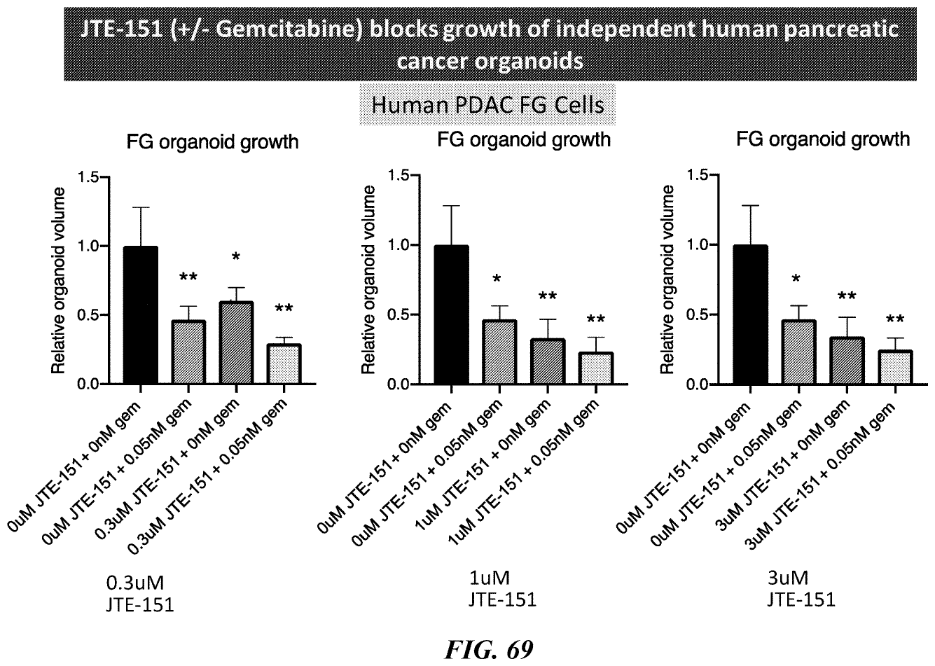

Similarly, the effects of JTE-151 at different doses were examined on human pancreatic cancer Fast Growing (FG) cells using the organoid assay (FIG. 69). Three doses of JTE-151 were tested: 0.3 μM, 1 μM, and 3 μM. For each JTE-151 dose, four conditions were tested: vehicle, gemcitabine alone (at 0.05 nM), JTE-151 alone, and a combination of JTE-151 and gemcitabine. As shown in FIG. 69, JTE-151 at all doses tested, administered either alone or in combination with gemcitabine, resulted in significant inhibition of FG organoid growth. Furthermore, the combination of JTE-151 and gemcitabine resulted in the highest inhibitory effect of FG organoid growth at each dose tested. Collectively, these data confirmed RORγ as a central regulator of pancreatic cancer progression and identified JTE-151, an RORγ inhibitor, as an effective anti-tumor therapeutic agent either alone or in combination with another chemotherapy agent.

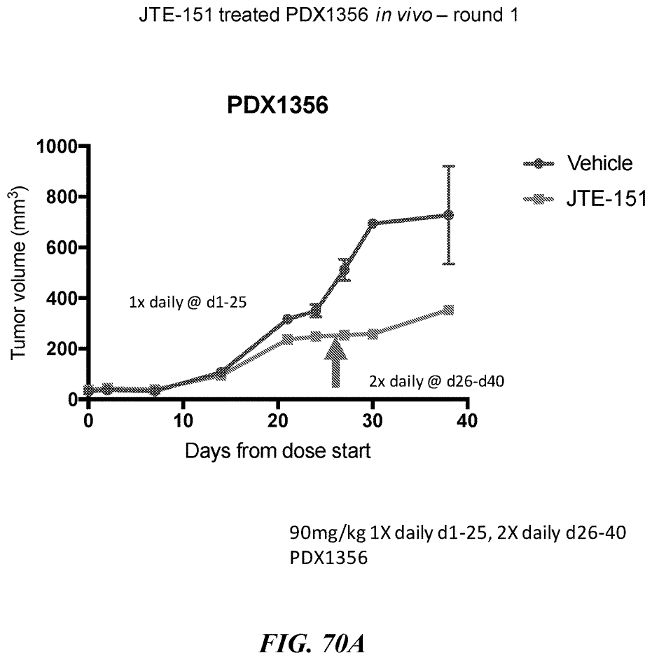

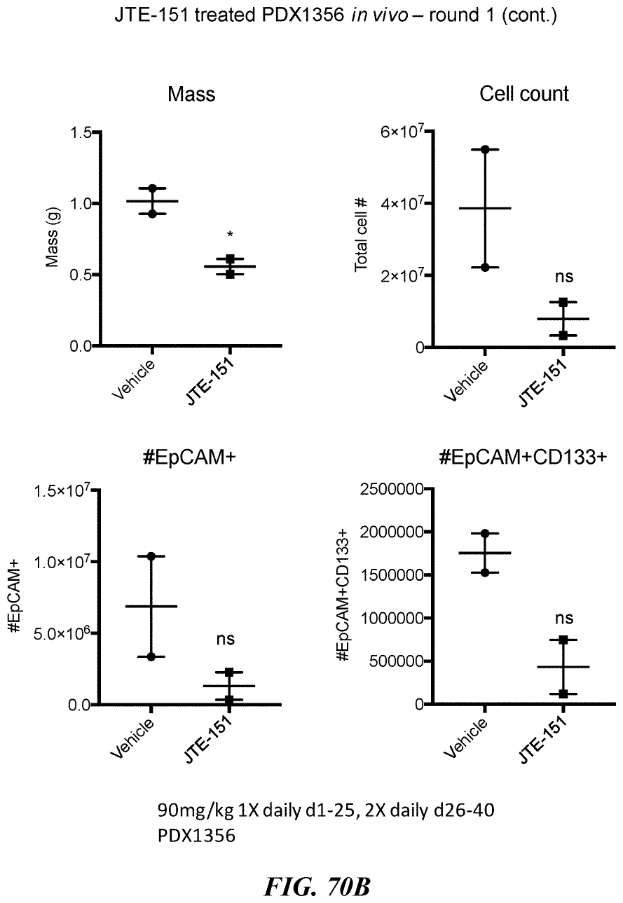

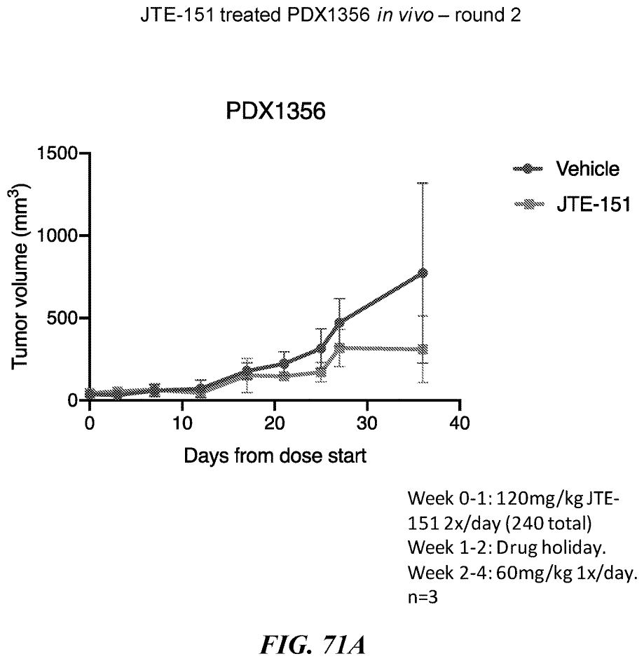

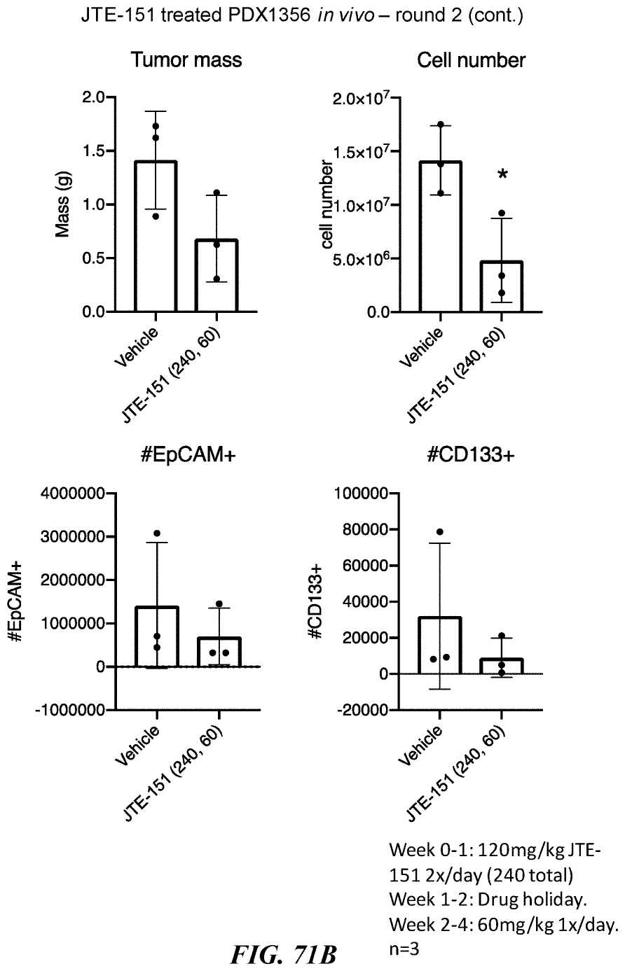

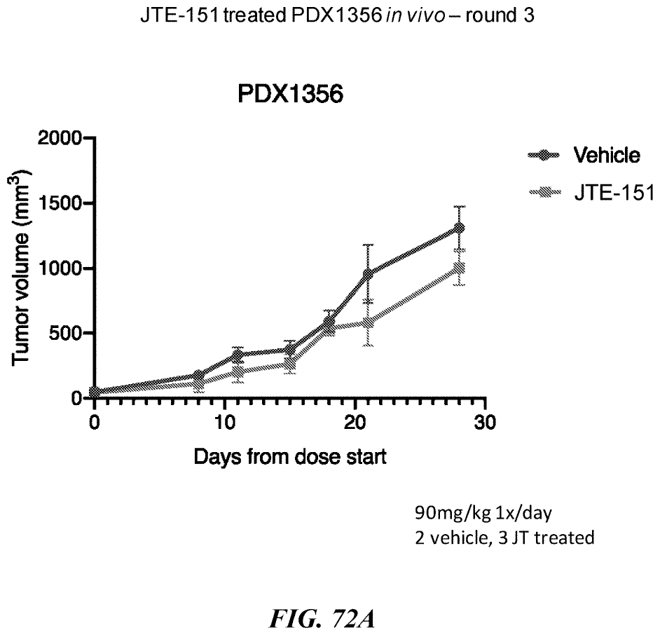

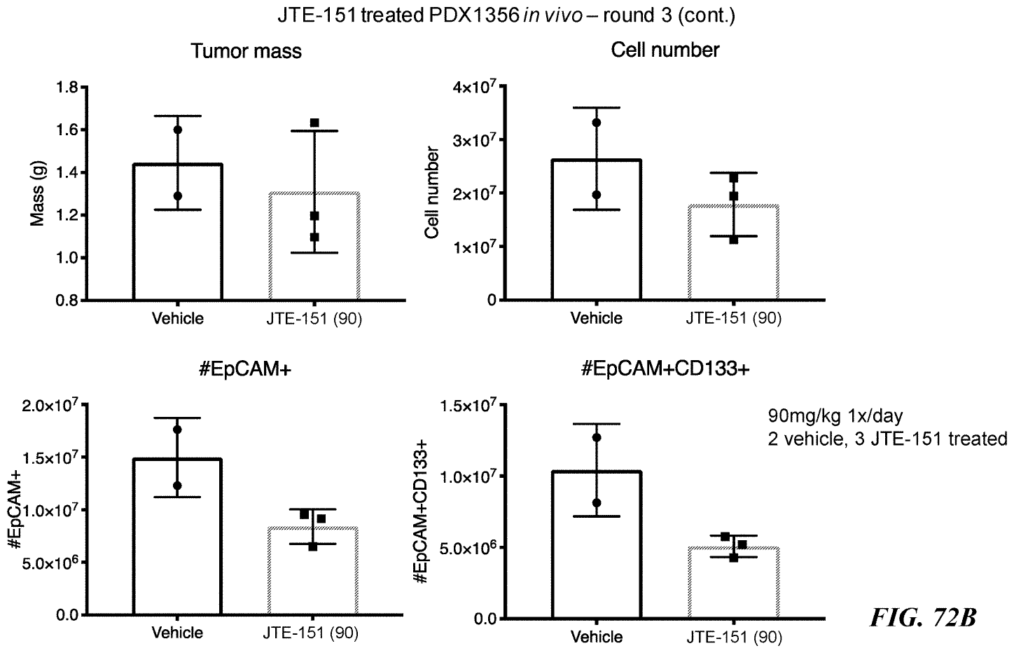

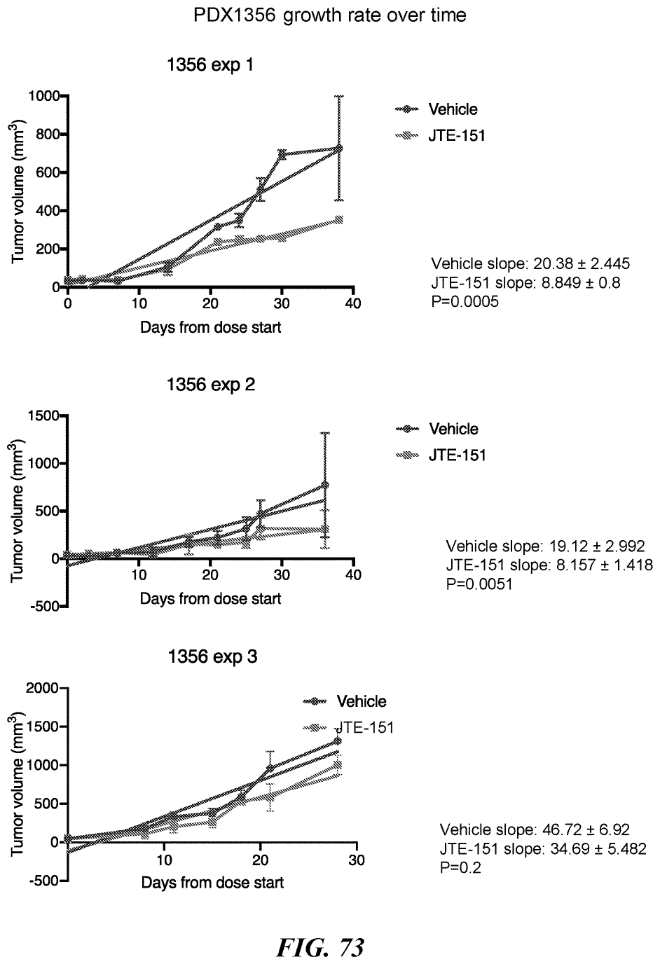

Finally, the impact of JTE-151 was examined in vivo on mice bearing primary patient-derived pancreatic cancer xenografts (FIGS. 70-78). As shown in FIG. 51, which is a schematic of the experimental design, immunodeficient mice transplanted with primary pancreatic cancer patient-derived xenografts were allowed to develop tumors, then the tumor-bearing mice received vehicle or JTE-151, followed by analysis of the tumors at the end of the experiments using different parameters including tumor mass, cell number, EpCAM positivity, CD133 positivity, and EpCAM/CD133 positivity. FIGS. 70-72 show 3 rounds of treatment in an experiment using mice bearing PDX1356 xenographs. The horizontal axis of the first panel in each of FIGS. 70-72 represents days of treatment, and the vertical axis represents tumor volume. The horizontal axis of each of the remaining panels represents the target (vehicle vs. JTE-151 mouse), and the vertical axis represents the specified measurement. JTE-151 was given at the regimen as specified in the figures. For example, in the first round (FIG. 70), JTE-151 was given at 90 mg/kg body weight once per day for the first 25 days, then twice per day from day 26 though day 40. The primary patient xenograft showed reduced tumor growth, decreased cell count, lower EpCam+ tumor epithelial cells, and lower EpCam+/CD133+ tumor stem cells following JTE-151 delivery. In the second round (FIG. 71), JTE-151 was given at 120 mg/kg twice per day (for a total of 240 mg/kg) for the first week, followed by 1 week of drug holiday, then at 60 mg/kg once per day from week 2 to 4, and a similar tumor-reducing effect by JTE-151 was observed. In the third round (FIG. 72), JTE-151 was given at 90 mg/kg once per day, and JTE-151 treatment again resulted in reduced EpCam+ tumor epithelial cells and EpCam+/CD133+ tumor stem cells. FIG. 73 shows a comparison of PDX1356 tumor growth rate over time between vehicle- and JTE-151-treated mice in the 3 experiments. JTE-151 treated tumors showed a generally slower growth rate, as reflected by the decrease in slope compared to control.

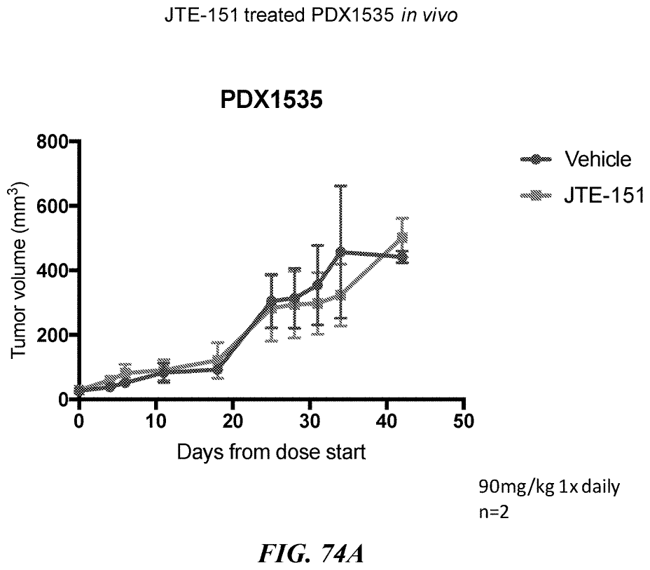

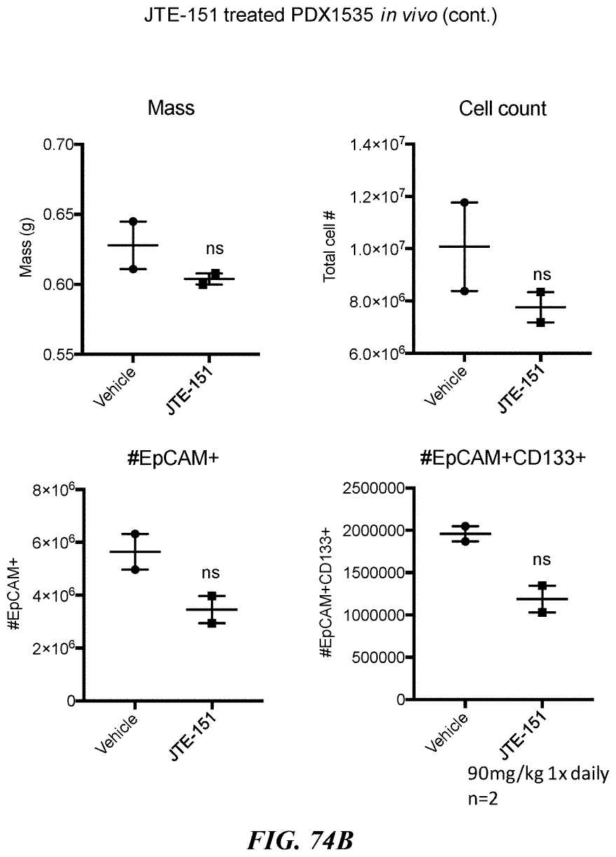

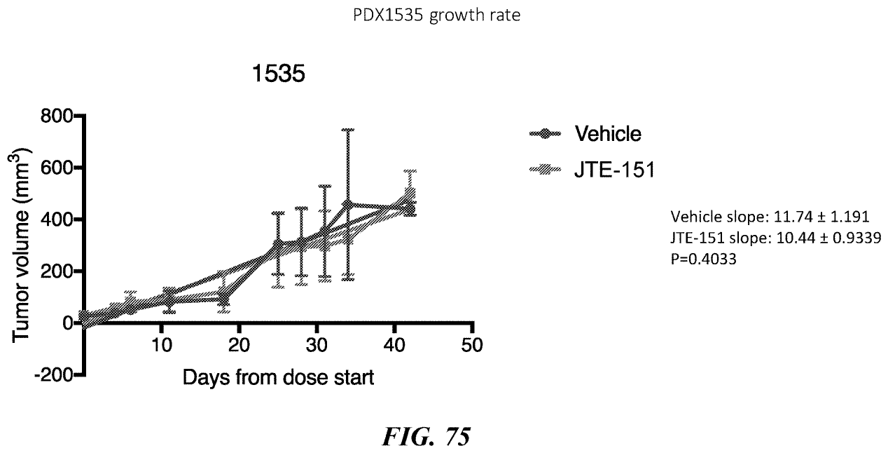

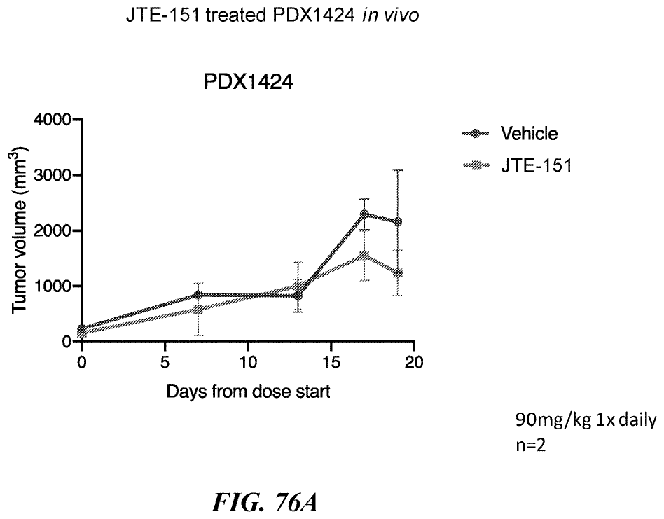

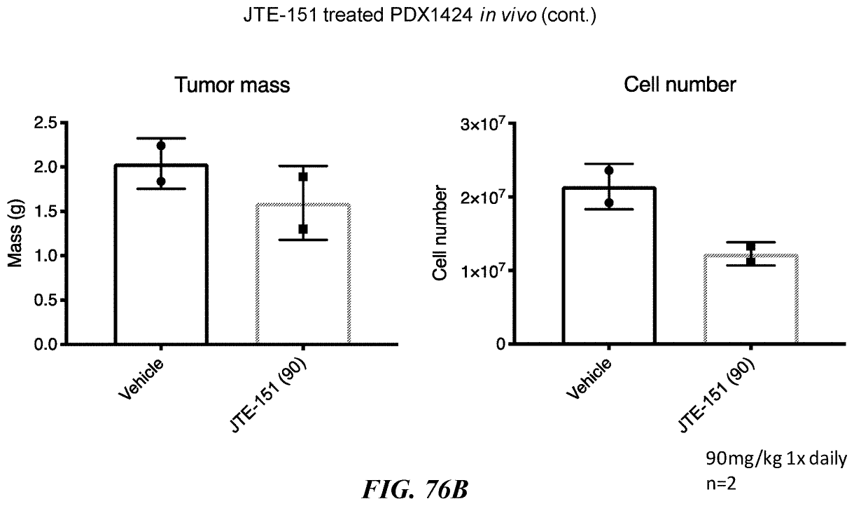

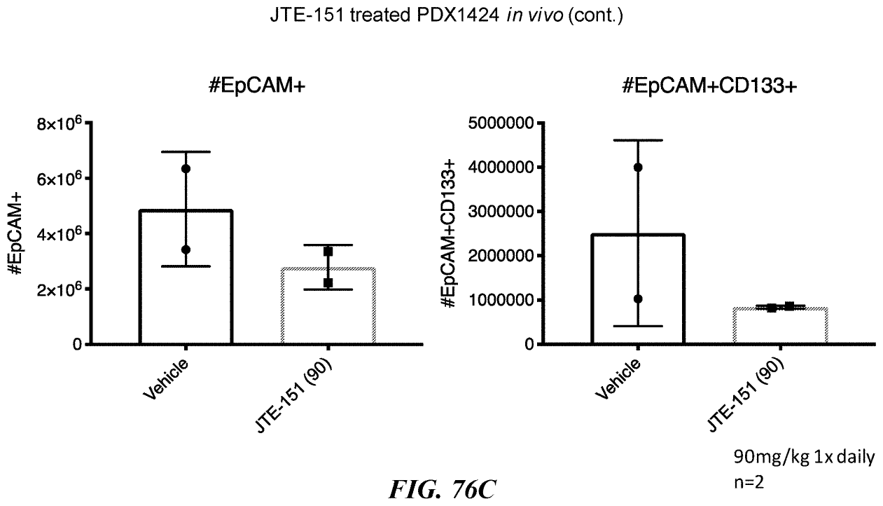

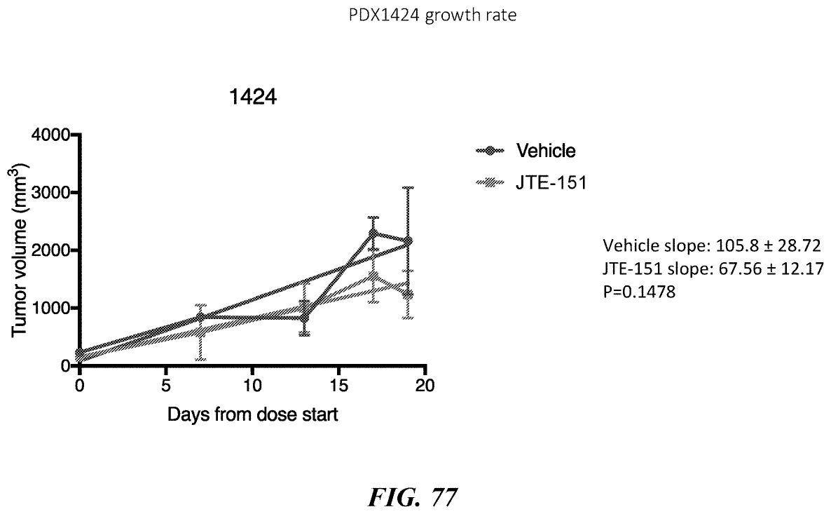

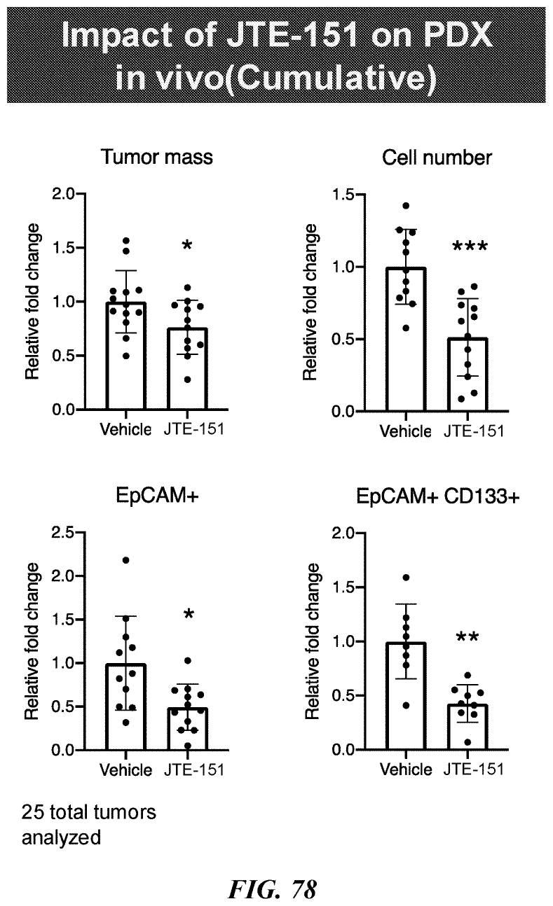

Two other primary patient-derived xenografts, PDX1535 (FIGS. 74 and 75) and PDX1424 (FIGS. 76 and 77), were tested using JTE-151 at 90 mg/kg once per day. As shown in FIGS. 74 and 75, PDX1535 xenograft showed a trend of decreased tumor mass, total cell counts, EpCam+ tumor epithelial cells, and EpCam+/CD133+ tumor stem cells following JTE-151 delivery (FIG. 74), although the tumor volume or the growth rate did not exhibit any significant difference (FIGS. 74, 75). Considering the reduced tumor mass and cell numbers, the absence of a significant change in tumor volume may be due to necrotic cells that remained for a while post drug treatment. As shown in FIG. 76, PDX1424 xenograft also showed a trend of decreased tumor mass, total cell counts, EpCam+ tumor epithelial cells, and EpCam+/CD133+ tumor stem cells following JTE-151 delivery. And JTE-151 treated tumor showed a slower growth rate (FIG. 77). FIG. 78 is a compilation of data from primary patient-derived xerographs treated with vehicle or JTE-151, and it shows that treatment of JTE-151 significantly reduced tumor mass, cell number, EpCam+ tumor epithelial cells, and EpCam+/CD133+ tumor stem cells, suggesting its cancer treatment efficacy.

Taken together, these data show that JTE-151 treatment blocked the growth of primary mammalian pancreatic cancer cells (human and mouse) both in vitro in organoid cultures and in vivo. Collectively, these studies demonstrate that targeting RORγ with JTE-151 is effective at blocking pancreatic cancer growth in vitro and in vivo and can potentially lead to effective new treatments for pancreatic cancer. Considering that inhibition of RORγ has been shown to reduce other types of cancer growth, including leukemia and lung cancer, JTE-151 has great potential to be used generally in anti-cancer therapies either alone or in combination with chemotherapy medication.

TABLE 1
Selected genes from stem cell networks.

| | | | |
|---|---|---|---|
| | RNA-seq fold change | H3K27ac | CRISPR |
| Gene name | (stem/non-stem) | ChIP-seq | screens |

Cell migration/Cell adhesion/Cell matrix interactions

| | | | |
|---|---|---|---|
| Sftpd | 42.427 | Up | — |
| Tff1 | 26.019 | Stem cell SE | — |
| Muc4 | 24.882 | Up | — |
| Crb3 | 10.083 | Up | ✓ 3D |
| Celsr1 | 9.194 | Up | ✓ 3D |
| Cldn6 | 8.211 | Up | ✓ 3D |
| Lama5 | 8.087 | Stem cell SE | |
| Pard6b | 7.549 | Stem cell SE | ✓✓✓ 3D |
| | | | ✓ 2D |
| Cldn3 | 7.254 | Stem cell SE | ✓3D |
| Celsr2 | 5.629 | Up | — |
| Pear1 | 4.417 | Up | ✓ 3D |
| Smo | 4.202 | Up | ✓✓✓ 3D |
| Rhof | 1.789 | Stem cell SE | ✓✓✓ 3D |
| Llgl1 | 1.506 | Up | ✓ 3D |
| Calm1 | −1.239 | Stem cell SE | ✓ 3D |

Development/Pluripotency/Stem cell signals

| | | | |
|---|---|---|---|
| Car2 | 22.3120000 | Up | — |
| Onecut3 | 19.2840000 | Stem SE | — |
| En1 | 12.0350000 | Up | ✓ 2D |
| Sox4 | 7.136 | N.D. | ✓✓✓ 3D |
| Smo | 4.2020000 | Up | ✓✓✓ 3D |
| Mapk11 | 4.032 | Stem SE | ✓ 3D |
| Wnt9a | 1.562 | | ✓ 3D |
| Psmd4 | 1.299 | Up | ✓✓✓ 2D |
| | | | ✓ 3D |
| Psmb1 | 1.275 | Up | ✓✓✓ 2D |
| | | | ✓ 3D |
| Foxo1 | 1.1840000 | Up | ✓✓✓ 3D |
| Psmc3 | 1.045 | Up | ✓✓✓ 2D |
| | | | ✓ 3D |
| Psma7 | −1.110 | N.D. | ✓ 3D |

Cytokine signaling/Immune pathways

| | | | |
|---|---|---|---|
| Gknl | 39.77 | Up | — |
| Gkn3 | 29.339 | Up | — |
| Sult1c2 | 23.634 | Up | — |
| ll34 | 6.586 | Stem cell SE | — |
| Akt1 | −1.400 | Stem cell SE | ✓ 3D |
| ll15 | −1.587 | Down | ✓ 3D |

Lipid metabolism/Nuclear receptor pathways

| | | | |
|---|---|---|---|
| Sptssb | 30.999 | Up | — |
| Rorc | 7.598 | Up | ✓✓✓ 3D |
| Arntl2 | 6.592 | Up | ✓ 3D |
| Med18 | 2.077 | | ✓ 3D |
| Lpin2 | 1.847 | Shared SE | — |
| Bhlhe41 | 1.737 | Stem cell SE | ✓ 3D |

Table 1 shows selected genes from stem cell networks identified by enriched gene expression in stem cells (RNA seq), preferentially open (H3K27ac ChIP-seq), or essential for growth (CRISPR screens). RNA-seq: fold change indicate expression in stem/non-stem. H3K27ac ChIP-seq: up indicates H3K27ac peaks enriched in stem cells; Stem cell SE, super enhancer unique to stem cells; Shared SE, super-enhancer in both stem and non-stem cells; N.D., H3K27ac not detectecd CRISPR screens; 2D, conventional growth conditions; 3D, stem cell conditions; ✓✓✓, p<0.005; ✓, gene ranks in top 10% of depleted guides (p<0.049 for 2D, p<0.092 for 3D); -, gene not in top 10% of depleted.

TABLE 2
Clinical and compound tool antagonists.

| | | | | in vitro sphere | in vivo tumor |
|---|---|---|---|---|---|
| Target | Core program | Known function | Drug/Compound | formation | growth |
| RORγ | Immune/cytokine signaling | nuclear receptor | SR2211 | ✓✓✓✓ | ✓✓ |
| IL-10 | Immune/cytokine signaling | cytokine | AS101 | ✓✓✓ | — |
| Dusp | Developmental pathways | phosphatase | BCl | ✓✓ | — |
| Wnk4 | Developmental pathways | serine/threonine kinase | Wnk463 | ✓✓ | ND |
| Myo5 | Cell motility/migration | myosin | Pentabromopseudilin | ✓✓ | ND |
| IL-7 | Immune/cytokine signaling | cytokine | Anti-1L7 | ✓ | — |
| CD83 | Immune/cytokine signaling | Ig superfamily membrane | GC7 | ✓ | ND |
| | | protein | | | |
| Cxcl2 | Immune/cytokine signaling | chemokine | Danirixin | — | ND |
| Drd2/3 | Immune/cytokine signaling | dopamine receptor | Eticlopride | — | — |

✓✓✓✓: dose response observed; growth suppressed by 8-fold or more relative to control
✓✓✓: dose response observed; growth suppressed between 4-fold and 8-fold relative to control
✓✓: dose response observed; growth suppressed by less than 4-fold relative to control
✓: response observed only at highest drug dose tested
—: no detectable response
ND: not determined

Table 2 includes select novel drug targets in pancreatic cancer, and indicates the impact of target inhibition by the indicated antagonist on in vitro and in vivo pancreatic cancer cell growth. Check marks indicate the extent of growth suppression observed in the indicated assay; -, no detectable response; ND, not determined.

TABLE 3
PDAC patients' characteristics (n = 116)

| Feature | | Frequency N (%) | |
|---|---|---|---|
| Age (years) | Mean (range) | 64.1 | (34-84) |
| Tumor size (cm) | Mean (range) | 3.5 | (1.2-10) |
| Sex | Female | 53 | (45.7) |
| | Male | 63 | (54.3) |
| Chemotherapy | None | 3 | (2.6) |
| | Treated | 99 | (85.3) |
| | Unknown | 14 | (12.1) |
| Radiotherapy | None | 63 | (54.3) |
| | Therapy | 14 | (12.1) |
| | Unknown | 39 | (33.6) |
| Tumor grade | 1 | 16 | (13.8) |
| | 2 | 55 | (47.4) |
| | 3 | 45 | (38.8) |
| pT classification | 1 | 23 | (19.8) |
| | 2 | 63 | (54.3) |
| | 3 | 26 | (22.4) |
| | Unknown | 4 | (3.4) |
| pN classification | 0 | 17 | (14.7) |
| | 1 | 47 | (40.5) |
| | 2 | 24 | (20.7) |
| | Unknown | 28 | (24.1) |
| pM classification | 0 | 104 | (89.7) |
| | 1 | 10 | (8.6) |
| | Unknown | 2 | (1.7) |
| Perineural invasion | Pn0 | 1 | (0.9) |
| | Pn1 | 111 | (95.7) |
| | Unknown | 4 | (3.4) |
| Lymphatic vessel invasion | L0 | 22 | (19.0) |
| | L1 | 92 | (79.3) |
| | Unknown | 2 | (1.7) |
| Venous vessel invasion | V0 | 89 | (76.7) |
| | V1 | 25 | (21.6) |
| | Unknown | 2 | (1.7) |
| Tumor budding 10HPF | Mean (range) | 18.5 | (0-95) |
| R classification | R0 | 79 | (68.1) |
| | R1 | 34 | (29.3) |
| | Unknown | 3 | (2.6) |
| TNM 8^{th} edition | IA | 5 | (4.3) |
| | IB | 7 | (6.0) |
| | IIA | 5 | (4.3) |
| | IIB | 42 | (36.2) |
| | III | 24 | (20.7) |
| | IV | 10 | (8.6) |
| | Unknown | 23 | (19.8) |
| KRAS mutation | WT | 5 | (4.3) |
| | MUT | 48 | (41.4) |
| | Unknown | 63 | (54.3) |
| P53 mutation | WT | 20 | (17.2) |
| | MUT | 33 | (28.4) |
| | Unknown | 63 | (54.3) |
| CDKN2A | WT | 45 | (38.8) |
| | MUT | 8 | (6.9) |
| | Unknown | 63 | (54.3) |

| | | |
|---|---|---|
| Overall survival | Mean (months) | 12.6 |
| Disease-free interval | Mean (months) | 5.9 |

TABLE 4
Primer sequences for the RT-qPCR analysis

| | qPCR primer forward | SEQ ID NO: | qPCR primer reverse | SEQ ID NO: |
|---|---|---|---|---|
| hIL10RB | TGAGAAATCACATTCCGTCAA | 3 | GCCAAAGGGAACCTGACTTT | 4 |
| hPEAR1 | AGCTGTGACGTGTCCTGTTC | 5 | CTGCCAACCTTCCTTGCAGA | 6 |
| mRorc | GGTGATAACCCCGTAGTGGA | 7 | CTGCAAAGAAGACCCACACC | 8 |
| mCsf1r | GCAGTACCACCATCCACTTGTA | 9 | GTGAGACACTGTCCTTCAGTGC | 10 |
| mll10rb | TAAGTTGTCCACGGCTCCAG | 11 | CATGGGCTTACAGAGTGCAA | 12 |
| mCelsr1 | GATGCTGTTGGTCAGCATGT | 13 | CGCTCATGGAGGTGTCTGT | 14 |
| mCelsr2 | GCTGTGTGTGAGCATCTCGT | 15 | CATCATGAGTGTGCTGGTGT | 16 |
| mPear1 | AGGGCACACGGTAACAAAAC | 17 | CACAGAACATCACCTGGCTG | 18 |
| mMyo5b | CCCCTTCTTTGTAGTCCTTGG | 19 | CGTACAGCGAGCTCTACACC | 20 |
| mOnecut3 | TTTGAGCTTGCTCCAGGG | 21 | GAAGCGCTACAGCATCCC | 22 |
| mTdrd3 | CCTTTCCCAGGAGAGCTTGT | 23 | GAGCCTGAGCAGCTAACCAT | 24 |
| mDusp9 | TCAGACTCTCCATGGTCGC | 25 | CACTAGCTGTGGCCAGGAC | 26 |
| mSptssb | AGCGCGTGAAGGAGTATTT | 27 | TGGTCAGTATGATGGTGTTGAG | 28 |
| mLpin2 | GCCCACATAATTCATGGTTTG | 29 | GGTTCAGGAAAGCTCGTTGA | 30 |
| mMyo10 | GAAGACCACGACGCCTTCT | 31 | CAATGGACAGCTTCTTTCCC | 32 |
| mSftpd | GAGAGCCCCATAGGTCCTG | 33 | GTAGCCCAACAGAGAATGGC | 34 |
| mPkp1 | TGGCTATAGGAGCTGAAGCG | 35 | CTTCTCCAAGTTCCAGGCAG | 36 |
| mLama5 | ACCCAAGGACCCACCTGTAG | 37 | TCATGTGTGCGTAGCCTCTC | 38 |
| mMegf10 | CCCAGTGACAGAGCAGTGAG | 39 | ATCACAGCATTTCAGGACCC | 40 |
| mll10 | TGTCAAATTCATTCATGGCCT | 41 | ATCGATTTCTCCCCTGTGAA | 42 |
| mll34 | CGCTTTCTCTGGTTTCTTCG | 43 | AGCTGCTCAAAGCTTCCG | 44 |
| mEn1 | TCCGAATAGCGTGTGCAGTA | 45 | CCTACTCATGGGTTCGGCTA | 46 |
| mCar2 | GTCACTGAGGGGTCCTCCTT | 47 | TGATAAAGCTGCGTCCAAGA | 48 |
| mAno1 | CGGGAGCGTCGAGTACTTCT | 49 | GCAGGAACCCCCAACTCA | 50 |
| mMuc4 | GGACATGGGTGTCTGTGTTG | 51 | CTCACTGGAGAGTTCCCTGG | 52 |
| mElmo3 | TGCTGAGACACAGGATGCTT | 53 | AGCACTATGCCCTGCAGTTT | 54 |
| mTff1 | CCACAATTTATCCTCTCCCG | 55 | GTCCTCATGCTGGCCTTC | 56 |
| mMuc1 | TGCTCCTACAAGTTGGCAGA | 57 | TACCAAGCGTAGCCCCTATG | 58 |
| mCtgf | GCTTGGCGATTTTAGGTGTC | 59 | CAGACTGGAGAAGCAGAGCC | 60 |
| mll1r1 | ATGAGACAAATGAGCCCCAG | 61 | GGAGAAATGTCGCTGGATGT | 62 |
| mll1b | GGTCAAAGGTTTGGAAGCAG | 63 | TGTGAAATGCCACCTTTTGA | 64 |

### REFERENCES

The references, patents and published patent applications listed below, and all references cited in the specification above are hereby incorporated by reference in their entirety, as if fully set forth herein.
Anders, S. and Huber, W. (2010). Differential expression analysis for sequence count data. Genome biology, 11(10), p.R106.
Blondel, V. D., J. L., G., R., L., and E., L. (2008). Fast unfolding of communities in large networks. In Journal of Statistical Mechanics (P10008), pp. arXiv:0803.0476.
Boj, S. F., Hwang, C. I., Baker, L. A., Chio, I I, Engle, D. D., Corbo, V., Jager, M., Ponz-Sarvise, M., Tiriac, H., Spector, M. S., et al. (2015). Organoid models of human and mouse ductal pancreatic cancer. Cell 160, 324-338.
Bray, N. L., Pimentel, H., Melsted, P., and Pachter, L. (2016). Near-optimal probabilistic RNA-seq quantification. Nat Biotechnol 34, 525-527.
Cole, J. J., Robertson, N. A., Rather, M. I., Thomson, J. P., McBryan, T., Sproul, D., Wang, T., Brock, C., Clark, W., Ideker, T., et al. (2017). Diverse interventions that extend mouse lifespan suppress shared age-associated epigenetic changes at critical gene regulatory regions. Genome Biol 18, 58.
Deshpande, A. J., Deshpande, A., Sinha, A. U., Chen, L., Chang, J., Cihan, A., Fazio, M., Chen, C. W., Zhu, N., Koche, R., et al. (2014). AF10 regulates progressive H3K79 methylation and HOX gene expression in diverse AML subtypes. Cancer Cell 26, 896-908.
Dull, T., Zufferey, R., Kelly, M., Mandel, R. J., Nguyen, M., Trono, D. and Naldini, L. (1998). A third-generation lentivirus vector with a conditional packaging system. Journal of virology, 72(11), pp. 8463-8471.
Efron, B., and Tibshirani, R. (2002). Empirical bayes methods and false discovery rates for microarrays. Genet Epidemiol 23, 70-86.
Fox, R. G., Lytle, N. K., Jaquish, D. V., Park, F. D., Ito, T., Bajaj, J., Koechlein, C. S., Zimdahl, B., Yano, M., Kopp, J., et al. (2016). Image-based detection and targeting of therapy resistance in pancreatic adenocarcinoma. Nature 534, 407-411.
Hänzelmann, S., Castelo, R. and Guinney, J. (2013). GSVA: gene set variation analysis for microarray and RNA-seq data. BMC bioinformatics, 14(1), p. 7.
Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
Hu, Y., and Smyth, G. K. (2009). ELDA: extreme limiting dilution analysis for comparing depleted and enriched populations in stem cell and other assays. J Immunol Methods 347, 70-78.
Ivanov, I I, McKenzie, B. S., Zhou, L., Tadokoro, C. E., Lepelley, A., Lafaille, J. J., Cua, D. J., and Littman, D. R. (2006). The orphan nuclear receptor RORgammat directs the differentiation program of proinflammatory IL-17+ T helper cells. Cell 126, 1121-1133.
Kawaguchi, Y., Cooper, B., Gannon, M., Ray, M., MacDonald, R. J., and Wright, C. V. (2002). The role of the transcriptional regulator Ptf1a in converting intestinal to pancreatic progenitors. Nat Genet 32, 128-134.
Kim, D., Pertea, G., Trapnell, C., Pimentel, H., Kelley, R. and Salzberg, S. L. (2013). TopHat2: accurate alignment of transcriptomes in the presence of insertions, deletions and gene fusions. Genome biology, 14(4), p.R36.
Langmead, B., and Salzberg, S. L. (2012). Fast gapped-read alignment with Bowtie 2. Nat Methods 9, 357-359.
Li, H., Handsaker, B., Wysoker, A., Fennell, T., Ruan, J., Homer, N., Marth, G., Abecasis, G. and Durbin, R. (2009). The sequence alignment/map format and SAMtools. Bioinformatics, 25(16), pp. 2078-2079.
Li, W., Xu, H., Xiao, T., Cong, L., Love, M. I., Zhang, F., Irizarry, R. A., Liu, J. S., Brown, M., and Liu, X. S. (2014). MAGeCK enables robust identification of essential genes from genome-scale CRISPR/Cas9 knockout screens. Genome Biol 15, 554.
Lönnstedt, I., and Speed, T. (2002). Replicated microarray data. Statistica Sinica 12, 31-46.
Martin, M. (2011). Cutadapt removes adapter sequences from high-throughput sequencing reads. EMBnet. journal, 17(1), pp. 10-12.
Mollaoglu, G., Guthrie, M. R., Böhm, S., Bragelmann, J., Can I., Balliue, P. M., Marx, A., George, J., Heinen, C, Chalishazar, M. D., et al. (2017). MYC Drives Progression of Small Cell Lung Cancer to a Variant Neuroendocrine Subtype with Vulnerability to Aurora Kinase Inhibition. Cancer Cell 31(2), 270-285.
Morgan, R. T., Woods, L. K., Moore, G. E., Quinn, L. A., McGavran, L. and Gordon, S. G. (1980). Human cell line (COLO 357) of metastatic pancreatic adenocarcinoma. International journal of cancer, 25(5), pp. 591-598.
Olive, K. P., Tuveson, D. A., Ruhe, Z. C., Yin, B., Willis, N. A., Bronson, R. T., Crowley, D. and Jacks, T. (2004). Mutant p53 gain of function in two mouse models of Li-Fraumeni syndrome. Cell, 119(6), pp. 847-860.
Pimentel, H., Bray, N. L., Puente, S., Melsted, P. and Pachter, L. (2017). Differential analysis of RNA-seq incorporating quantification uncertainty. Nature methods, 14(7), p. 687.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47.
Rosenthal, S. B., Len, J., Webster, M., Gary, A., Birmingham, A., and Fisch, K. M. (2018). Interactive network visualization in Jupyter notebooks: visJS2jupyter. Bioinformatics 34, 126-128.
Rovira, M., Scott, S. G., Liss, A. S., Jensen, J., Thayer, S. P., and Leach, S. D. (2010). Isolation and characterization of centroacinar/terminal ductal progenitor cells in adult mouse pancreas. Proc Natl Acad Sci USA 107, 75-80.
Sanjana, N. E., Shalem, O., and Zhang, F. (2014). Improved vectors and genome-wide libraries for CRISPR screening. Nat Methods 11, 783-784.
Sena-Esteves, M., Tebbets, J. C., Steffens, S., Crombleholme, T. and Flake, A. W. (2004). Optimized large-scale production of high titer lentivirus vector pseudotypes. Journal of virological methods, 122(2), pp. 131-139.

Spahn, P. N., Bath, T., Weiss, R. J., Kim, J., Esko, J. D., Lewis, N. E., and Harismendy, O. (2017). PinAPL-Py: A comprehensive web-application for the analysis of CRISPR/Cas9 screens. Sci Rep 7, 15854.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Szklarczyk, D., Franceschini, A., Wyder, S., Forslund, K., Heller, D., Huerta-Cepas, J., Simonovic, M., Roth, A., Santos, A., Tsafou, K. P., et al. (2015). STRING v10: protein-protein interaction networks, integrated over the tree of life. Nucleic Acids Res 43, D447-452.
Trapnell, C., Roberts, A., Goff, L., Pertea, G., Kim, D., Kelley, D. R., Pimentel, H., Salzberg, S. L., Rinn, J. L. and Pachter, L. (2012). Differential gene and transcript expression analysis of RNA-seq experiments with TopHat and Cufflinks. Nature protocols, 7(3), p. 562.
Vanunu, O., Magger, O., Ruppin, E., Shlomi, T., and Sharan, R. (2010). Associating genes and protein complexes with disease via network propagation. PLoS Comput Biol 6, e1000641.
Wang, J., Vasaikar, S., Shi, Z., Greer, M., and Zhang, B. (2017). WebGestalt 2017: a more comprehensive, powerful, flexible and interactive gene set enrichment analysis toolkit. Nucleic Acids Res 45, W130-W137.
Wartenberg, M., Cibin, S., Zlobec, I., Vassella, E., Eppenberger-Castori, S., Terracciano, L., Eichmann, M. D., Worni, M., Gloor, B., Perren, A. and Karamitopoulou, E. (2018). Integrated genomic and immunophenotypic classification of pancreatic cancer reveals three distinct subtypes with prognostic/predictive significance. Clinical cancer research, 24(18), pp. 4444-4454.
Xie, P., Gao, M., Wang, C., Noel, P., Yang, C., Von Hoff, D., Han, H., Zhang, M. and Lin, W. (2018). SuperCT: A supervised-learning-framework to enhance the characterization of single-cell transcriptomic profiles. bioRxiv, p. 416719.
Zang, C., Schones, D. E., Zeng, C., Cui, K., Zhao, K., and Peng, W. (2009). A clustering approach for identification of enriched domains from histone modification ChIP-Seq data. Bioinformatics 25, 1952-1958.
Zhu, L. J., Gazin, C., Lawson, N. D., Pages, H., Lin, S. M., Lapointe, D. S. and Green, M. R. (2010). ChiPpeakAnno: a Bioconductor package to annotate ChIP-seq and ChIP-chip data. BMC bioinformatics, 11(1), p. 237.

1. A method of treating an RORγ-dependent cancer comprising administrating to a subject in need a therapeutically effective amount of a composition comprising one or more RORγ inhibitors.

2. The method of claim 1, further comprising subjecting the subject to one or more additional cancer therapies selected from chemotherapy, radiation therapy, immunotherapy, surgery and a combination thereof, wherein the one or more additional cancer therapies are administered to the subject before, during, or after administration of the composition comprising one or more RORγ inhibitors.

3. (canceled)

4. The method of claim 1, wherein the RORγ-dependent cancer includes pancreatic cancer, leukemia, and lung cancer such as small cell lung cancer (SCLC) and nonsmall cell lung cancer (NSCLC).

5. The method of claim 1, wherein the cancer is a metastatic cancer.

6. The method of claim 1, wherein the RORγ inhibitor includes SR2211, JTE-151, JTE-151A, and AZD-0284, or an analog or derivative thereof represented by any one of formulae I, II, III, IIIA, and IV.

7. A pharmaceutical composition for treating an RORγ-dependent cancer, comprising a therapeutically effective amount of one or more RORγ inhibitors.

8. The pharmaceutical composition of claim 7, further comprising one or more additional therapeutic agents selected from the group consisting of a chemotherapeutic agent, a radiation therapeutic agent, an immunotherapeutic agent, or a combination thereof.

9. The pharmaceutical composition of claim 7, further comprising one or more pharmaceutically acceptable carriers, excipients, preservatives, diluent, buffer, or a combination thereof.

10. The pharmaceutical composition of claim 7, the RORγ-dependent cancer includes pancreatic cancer, leukemia, and lung cancer such as small cell lung cancer (SCLC) and nonsmall cell lung cancer (NSCLC).

11. The pharmaceutical composition of claim 7, wherein the cancer is a metastatic cancer.

12. The pharmaceutical composition of claim 7, wherein the RORγ inhibitor includes SR2211, JTE-151, JTE-151A, and AZD-0284, or an analog or derivative thereof represented by any one of formulae I, II, III, IIIA, and IV.

13. A combinational therapy for treating an RORγ-dependent cancer comprising administering to a subject a composition comprising one or more RORγ inhibitors, and administering an additional cancer therapy including performing surgery, administering one or more chemotherapeutic agents, administering one or more radiotherapies, and/or administering one or more of immunotherapies to the subject before, during, or after administering the composition comprising one or more RORγ inhibitors.

14. The combinational therapy of claim 13, wherein the RORγ-dependent cancer includes pancreatic cancer, leukemia, and lung cancer such as small cell lung cancer (SCLC) and nonsmall cell lung cancer (NSCLC).

15. The combinational therapy of claim 13, wherein the cancer is a metastatic cancer.

16. The combinational therapy of claim 13, wherein the RORγ inhibitor includes SR2211, JTE-151, JTE-151A, and AZD-0284, or an analog or derivative thereof represented by any one of formulae I, II, III, IIIA, and IV.

17. (canceled)

18. (canceled)

19. (canceled)

20. (canceled)


SEQUENCE LISTING (64 sequences)

SEQ ID NO: 1 - DNA, 24 bp, Artificial Sequence
  5′-adapter
tcttgtggaaaggacgaaacaccg

SEQ ID NO: 2 - DNA, 25 bp, Artificial Sequence
  3′-adapter
gttttagagctagaaatagcaagtt

SEQ ID NO: 3 - DNA, 21 bp, Artificial Sequence
  hIL10RB qPCR primer forward
tgagaaatcacattccgtcaa

SEQ ID NO: 4 - DNA, 20 bp, Artificial Sequence
  hIL10RB qPCR primer reverse
gccaaagggaacctgacttt

SEQ ID NO: 5 - DNA, 20 bp, Artificial Sequence
  hPEAR1 qPCR primer forward
agctgtgacgtgtcctgttc

SEQ ID NO: 6 - DNA, 20 bp, Artificial Sequence
  hPEAR1 qPCR primer reverse
ctgccaaccttccttgcaga

SEQ ID NO: 7 - DNA, 20 bp, Artificial Sequence
  mRorc qPCR primer forward
ggtgataaccccgtagtgga

SEQ ID NO: 8 - DNA, 20 bp, Artificial Sequence
  mRorc qPCR primer reverse
ctgcaaagaagacccacacc

SEQ ID NO: 9 - DNA, 22 bp, Artificial Sequence
  mCsf1r qPCR primer forward
gcagtaccaccatccacttgta

SEQ ID NO: 10 - DNA, 22 bp, Artificial Sequence
  mCsf1r qPCR primer reverse
gtgagacactgtccttcagtgc

SEQ ID NO: 11 - DNA, 20 bp, Artificial Sequence
  mIl10rb qPCR primer forward
taagttgtccacggctccag

SEQ ID NO: 12 - DNA, 20 bp, Artificial Sequence
  mIl10rb qPCR primer reverse
catgggcttacagagtgcaa

SEQ ID NO: 13 - DNA, 20 bp, Artificial Sequence
  mCelsr1 primer forward
gatgctgttggtcagcatgt

SEQ ID NO: 14 - DNA, 19 bp, Artificial Sequence
  mCelsr1 qPCR primer reverse
cgctcatggaggtgtctgt

SEQ ID NO: 15 - DNA, 20 bp, Artificial Sequence
  mCelsr2 qPCR primer forward
gctgtgtgtgagcatctcgt

SEQ ID NO: 16 - DNA, 20 bp, Artificial Sequence
  mCelsr2 qPCR primer reverse
catcatgagtgtgctggtgt

SEQ ID NO: 17 - DNA, 20 bp, Artificial Sequence
  mPear1 qPCR primer forward
agggcacacggtaacaaaac

SEQ ID NO: 18 - DNA, 20 bp, Artificial Sequence
  mPear1 qPCR primer reverse
cacagaacatcacctggctg

SEQ ID NO: 19 - DNA, 21 bp, Artificial Sequence
  mMyo5b qPCR primer forward
ccccttctttgtagtccttgg

SEQ ID NO: 20 - DNA, 20 bp, Artificial Sequence
  mMyo5b qPCR primer reverse
cgtacagcgagctctacacc

SEQ ID NO: 21 - DNA, 18 bp, Artificial Sequence
  mOnecut3 qPCR primer forward
tttgagcttgctccaggg

SEQ ID NO: 22 - DNA, 18 bp, Artificial Sequence
  mOnecut3 qPCR primer reverse
gaagcgctacagcatccc

SEQ ID NO: 23 - DNA, 20 bp, Artificial Sequence
  mTdrd3 qPCR primer forward
cctttcccaggagagcttgt

SEQ ID NO: 24 - DNA, 20 bp, Artificial Sequence
  mTdrd3 qPCR primer reverse
gagcctgagcagctaaccat

SEQ ID NO: 25 - DNA, 19 bp, Artificial Sequence
  mDusp9 qPCR primer forward
tcagactctccatggtcgc

SEQ ID NO: 26 - DNA, 19 bp, Artificial Sequence
  mDusp9 qPCR primer reverse
cactagctgtggccaggac

SEQ ID NO: 27 - DNA, 19 bp, Artificial Sequence
  mSptssb qPCR primer forward
agcgcgtgaaggagtattt

SEQ ID NO: 28 - DNA, 22 bp, Artificial Sequence
  mSptssb qPCR primer reverse
tggtcagtatgatggtgttgag

SEQ ID NO: 29 - DNA, 21 bp, Artificial Sequence
  mLpin2 qPCR primer forward
gcccacataattcatggtttg

SEQ ID NO: 30 - DNA, 20 bp, Artificial Sequence
  mLpin2 qPCR primer reverse
ggttcaggaaagctcgttga

SEQ ID NO: 31 - DNA, 19 bp, Artificial Sequence
  mMyo10 qPCR primer forward
gaagaccacgacgccttct

SEQ ID NO: 32 - DNA, 20 bp, Artificial Sequence
  mMyo10 qPCR primer reverse
caatggacagcttctttccc

SEQ ID NO: 33 - DNA, 19 bp, Artificial Sequence
  mSftpd qPCR primer forward
gagagccccataggtcctg

SEQ ID NO: 34 - DNA, 20 bp, Artificial Sequence
  mSftpd qPCR primer reverse
gtagcccaacagagaatggc

SEQ ID NO: 35 - DNA, 20 bp, Artificial Sequence
  mPkp1 qPCR primer forward
tggctataggagctgaagcg

SEQ ID NO: 36 - DNA, 20 bp, Artificial Sequence
  mPkp1 qPCR primer reverse
cttctccaagttccaggcag

SEQ ID NO: 37 - DNA, 20 bp, Artificial Sequence
  mLama5 qPCR primer forward
acccaaggacccacctgtag

SEQ ID NO: 38 - DNA, 20 bp, Artificial Sequence
  mLama5 qPCR primer reverse
tcatgtgtgcgtagcctctc

SEQ ID NO: 39 - DNA, 20 bp, Artificial Sequence
  mMegf10 qPCR primer forward
cccagtgacagagcagtgag

SEQ ID NO: 40 - DNA, 20 bp, Artificial Sequence
  mMegf10 qPCR primer reverse
atcacagcatttcaggaccc

SEQ ID NO: 41 - DNA, 21 bp, Artificial Sequence
  mIl10 qPCR primer forward
tgtcaaattcattcatggcct

SEQ ID NO: 42 - DNA, 20 bp, Artificial Sequence
  mIl10 qPCR primer reverse
atcgatttctcccctgtgaa

SEQ ID NO: 43 - DNA, 20 bp, Artificial Sequence
  mIl34 qPCR primer forward
cgctttctctggtttcttcg

SEQ ID NO: 44 - DNA, 18 bp, Artificial Sequence
  mIl34 qPCR primer reverse
agctgctcaaagcttccg

SEQ ID NO: 45 - DNA, 20 bp, Artificial Sequence
  mEn1 qPCR primer forward
tccgaatagcgtgtgcagta

SEQ ID NO: 46 - DNA, 20 bp, Artificial Sequence
  mEn1 qPCR primer reverse
cctactcatgggttcggcta

SEQ ID NO: 47 - DNA, 20 bp, Artificial Sequence
  mCar2 qPCR primer forward
gtcactgaggggtcctcctt

SEQ ID NO: 48 - DNA, 20 bp, Artificial Sequence
  mCar2 qPR primer reverse
tgataaagctgcgtccaaga

SEQ ID NO: 49 - DNA, 20 bp, Artificial Sequence
  mAno1 qPCR primer forward
cgggagcgtcgagtacttct

SEQ ID NO: 50 - DNA, 18 bp, Artificial Sequence
  mAno1 qPCR primer reverse
gcaggaacccccaactca

SEQ ID NO: 51 - DNA, 20 bp, Artificial Sequence
  mMuc4 qPCR primer forward
ggacatgggtgtctgtgttg

SEQ ID NO: 52 - DNA, 20 bp, Artificial Sequence
  mMuc4 qPCR primer reverse
ctcactggagagttccctgg

SEQ ID NO: 53 - DNA, 20 bp, Artificial Sequence
  mElmo3 qPCR primer forward
tgctgagacacaggatgctt

SEQ ID NO: 54 - DNA, 20 bp, Artificial Sequence
  mElmo3 qPCR primer reverse
agcactatgccctgcagttt

SEQ ID NO: 55 - DNA, 20 bp, Artificial Sequence
  mTff1 qPCR primer forward
ccacaatttatcctctcccg

SEQ ID NO: 56 - DNA, 18 bp, Artificial Sequence
  mTff1 qPCR primer reverse
gtcctcatgctggccttc

SEQ ID NO: 57 - DNA, 20 bp, Artificial Sequence
  mMuc1 qPCR primer forward
tgctcctacaagttggcaga

SEQ ID NO: 58 - DNA, 20 bp, Artificial Sequence
  mMuc1 qPCR primer reverse
taccaagcgtagcccctatg

SEQ ID NO: 59 - DNA, 20 bp, Artificial Sequence
  mCtgf qPCR primer forward
gcttggcgattttaggtgtc

SEQ ID NO: 60 - DNA, 20 bp, Artificial Sequence
  mCtgf qPCR primer reverse
cagactggagaagcagagcc

SEQ ID NO: 61 - DNA, 20 bp, Artificial Sequence
  mIl1r1 qPCR primer forward
atgagacaaatgagccccag

SEQ ID NO: 62 - DNA, 20 bp, Artificial Sequence
  mIl1r1 qPCR primer reverse
ggagaaatgtcgctggatgt

SEQ ID NO: 63 - DNA, 20 bp, Artificial Sequence
  mIl1b qPCR primer forward
ggtcaaaggtttggaagcag

SEQ ID NO: 64 - DNA, 20 bp, Artificial Sequence
  mIl1b qPCR primer reverse
tgtgaaatgccaccttttga